(19) United States
(12) Patent Application Publication
(10) Pub. No.: US 2025/0243482 A1
(43) Pub. Date: Jul. 31, 2025

(54) CLASS 2 TYPE V CRISPR-CAS PRIME EDITING

(71) Applicants: BASF Agricultural Solutions Seed US LLC, Florham Park, NJ (US); Wageningen Universiteit, Wageningen (NL)

(72) Inventors: David DE VLEESSCHAUWER, Gent (BE); Frank MEULEWAETER, Gent (BE); Anne-Marie KUIJPERS, Gent (BE); Raymond Hubert Josèphe STAALS, Wageningen (NL); John VAN DER OOST, Wageningen (NL); Katelijn D'HALLUIN, Mariakerke (Gent) (BE); Thomas SWARTJES, Wageningen (NL); Han VERHAGEN, Wageningen (NL); Ricardo Villegas Warren, Wageningen (NL)

(21) Appl. No.: 18/817,137

(22) Filed: Aug. 27, 2024

(30) Foreign Application Priority Data
Aug. 31, 2023 (EP) 23194514.8
Apr. 30, 2024 (EP) 24173281.7

(51) Int. Cl. C12N 15/11; A61K 48/00; C12N 9/12; C12N 9/22; C12N 15/82; C12N 15/85

(52) U.S. Cl. CPC C12N 15/111; A61K 48/005; C12N 9/1276; C12N 9/226; C12N 15/8201; C12N 15/8242; C12N 15/8261; C12N 15/85; C07K 2319/02; C07K 2319/735; C07K 2319/85; C12N 2310/20

(57) ABSTRACT

The present disclosure relates to the field of gene genome editing. In particular, it relates to the provision of a CRISPR-Cas class 2 type V prime editing system, including a prime editor, a prime editor complex, a prime editing guide RNA system as well as the means and methods for the modification of a nucleic acid sequence of interest with the prime editing system of the present invention.

### CROSS REFERENCE TO RELATED APPLICATIONS

This application claims priority to European Patent Application No. 23194514.8, filed Aug. 31, 2023, and European Patent Application No. 24173281.7, filed Apr. 30, 2024, the disclosures of which are incorporated by reference in their entireties.

### REFERENCE TO SEQUENCE LISTING SUBMITTED ELECTRONICALLY

The sequence listing of the present application is submitted electronically as an XML file named “038771-00006SeqList.xml”, created on Aug. 23, 2024, and having a size of 763,938 bytes. This sequence listing submitted electronically is an integral part of the specification and is incorporated herein by reference in its entirety.

### TECHNICAL FIELD

The present invention relates to the field of gene genome editing. In particular, it relates to the provision of a CRISPR-Cas class 2 type V prime editing system, including a prime editor, a prime editor complex, a prime editing guide RNA system as well as the means and methods for the modification of a nucleic acid sequence of interest with the prime editing system of the present invention.

### BACKGROUND

Prime editing is a “search-and-replace” genome editing tool that mediates targeted insertions, deletions, all 12 possible base-to-base conversions, and combinations thereof without requiring double-stranded breaks (DSBs) or separate donor DNA templates (ss/ds) (Anzalone et al., Nature. 2019 December; 576 (7785): 149-157). For prime editing, a so-called prime editing guide RNA (pegRNA) is used. The pegRNA usually comprises a single guide RNA (sgRNA) with a target-specific sequence (spacer), and an extension that includes a primer binding site (PBS) and a reverse transcriptase template (RTT) sequence that will be introduced to the targeted DNA sequence. The PBS region is complementary to the non-target strand and after base pairing, the non-target strand will create a primer for the reverse transcriptase (RT) that is combined (either through a physical covalent link, or as stand-alone) to a guide RNA binding the Cas protein (usually a dsDNA nuclease or a ssDNA nuclease (nickase), resulting in specific DSB or SSB of the target DNA, respectively). Subsequently, the sequence of the RTT sequence is reverse transcribed from the pegRNA into an edited target DNA sequence. Three generations of prime editors have been used in different target cells: PE1, PE2 and PE3 (Anzalone et al. supra). PE1 is based on the Moloney murine leukemia virus reverse transcriptase (M-MLV RT). PE2 (called pPE2 in plants) is based on the M-MLV RT D200N/L603W/T330P/T306K/W313F variant. PE3 (called pPE3 in plants) uses an additional guide RNA specifically targeting the non-edited strand DNA adjacent to the edited sequence (Marzec et al., Trends Cell Biol. 2020 April; 30 (4): 257-259. doi: 10.1016/j.tcb.2020.01.004; Xu et al. Plant Commun. 2020 Apr. 8; 1 (3): 100043. doi: 10.1016/j.xplc.2020.100043; Lin et al. Nat Biotechnol. 2020 May; 38 (5): 582-585. doi: 10.1038/s41587-020-0455-x.). It has been shown, that the M-MLV RT can be exchanged by different RTs, such as Cauliflower Mosaic Virus (CaMV) RT, or retron-derived RT (Lin et al. supra). PegRNA can be designed and optimized for different target sequences, cells or constructs. For example, prime editing in plants is described in Sretenovic and Qi (Nat Plants. 2022 January; 8 (1): 20-22. doi: 10.1038/s41477-021-01047-0) and optimized prime editing in monocot plants is described in Jin et al. (Science. 2012 Aug. 17; 337 (6096): 816-21).

Classical Prime editors as known in the art rely on a CRISPR-Cas9 (Class 2, type II) Cas system. In this approach, the Cas9 H840A nickase is used to generate a SSB to nick the non-target strand, thereby exposing a 3′-hydroxyl group that primes the reverse transcription of the edit-encoding extension on the pegRNA directly into the target site.

However, while class 2 type V CRISPR-Cas systems are receiving tremendous interest, in particular Cas12a (Cpf1) systems, there is still a great need in the art for functional class 2 type V CRISPR-Cas prime editing systems. One problem for class 2 type V CRISPR-Cas systems is that specific nickases, comparable to the Cas9 H840A nickase, are not available. Unlike Cas9, Cas12a cleaves both DNA strands sequentially using a single catalytic site located in the RuvC domain, while the Nuc domain plays a role in substrate DNA coordination (Swarts et al., 2017 Molecular Cell. 2017 Apr. 20; 66 (2): 221-233.e4.doi: 10.1016/j.molcel.2017.03.016.; Swarts and Jinek, Molecular Cell. 2019 Feb. 7; 73 (3): 589-600.e4. doi: 10.1016/j.molcel.2018.11.021). This difference in structural organization hampers the design of true nickases of Cas12a in comparison to Cas9, the latter CRISPR nuclease having two distinct domains comprising two individual active domains, HNH and RuvC, catalyzing the cleavage of the target and the non-target strand, respectively. Recently, a system called RNA encoded DNA replacement ht of alleles with CRISPR (“REDRAW”) was proposed (Kim et al. https://doi.org/10.1101/2022.12.13.520319). This system tries to circumvent the lack of specific Cas12a nickases by using an altered architecture, wherein the spacer sequence and the primer binding site anneal to the same strand (the target strand). However, the use of a nuclease is associated with an increase in off-target effects and leads to the generation of undesired indels, which signficantly reduces the efficiency of precise editing and necessitates complicated and time-consuming steps to remove these undesired byproducts at a later stage (i.e. by backcrossing in plants or repression of NHEJ in human cells).

It was thus an object of the present invention to provide a functional and efficient class 2 type V CRISPR-Cas prime editing system, preferably a nickase-mediated prime editing system, that allows prime editing as so-far exclusively established for Cas9 systems, thereby extending the classical prime editing tools to modern class 2 type V CRISPR-Cas systems.

### SUMMARY

In a first aspect, there is provided a prime editing complex comprising (a) at least one prime editing guide RNA (pegRNA) or pegRNA system as defined in the second aspect; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally at least one linker covalently or non-covalently linking the at least one Cas12a enzyme, or active fragment thereof, and the at least one reverse transcriptase, or an active fragment thereof; and (iv) optionally at least one organellar localization signal.

In one embodiment, at least one organellar localization signal is located at the N-terminus and/or the C-terminus of the at least one Cas12a enzyme, or active fragment thereof, and/or the at least one reverse transcriptase, or active fragment thereof.

In one embodiment, the at least one Cas12a enzyme, or active fragment thereof, is an RNase dead Cas12a enzyme, or active fragment thereof, optionally comprising a mutation at position H759, including a H759A mutation.

In one embodiment, the Cas12a prime editor comprises at least one ssDNA-binding and/or ssDNA-stabilizing protein, or domain or functional active fragment thereof, preferably being independently selected from Brex27, RPA70-A, RPA70 B, RPA70-C, RPA32-D, BRCA2-OB2, BRCA2-OB3, HNRNPK (Heterogeneous nuclear ribonucleoprotein K) KH (K-homology domain), PUF60RRM, or Rad51DBD.

In a further embodiment, the prime editor complex further comprises in trans (c) a reverse transcriptase protein, preferably comprising at least one organellar localization signal and preferably comprising an MS2 coat protein (MCP).

In yet another embodiment, the at least one Cas12a enzyme, or active fragment thereof of the prime editor complex further comprises an RNA binding factor, preferably an N-terminal domain of La or a MCP.

In one embodiment, the prime editor complex comprises the N-terminal domain of La, optionally comprising the reverse transcriptase protein as defined above comprising an MCP, and the pegRNA system as defined above, and, optionally the nicking crRNA as defined above, wherein a) the crRNA comprises in a 5′ to 3′ direction (i) a CRISPR-Cas class 2 type V scaffold sequence, (ii) a spacer sequence; and optionally (vi) a second scaffold sequence, optionally being the same as the first scaffold sequence; and/or wherein b) the prime editing template RNA (petRNA) comprises in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

In one embodiment, (ii) the at least one reverse transcriptase, or active fragment thereof, is linked, via (iii) the at least one linker, N-terminally or C-terminally, preferably N-terminally, to (i) the at least one Cas12a enzyme, or active fragment thereof.

In one embodiment, the at least one Cas12a enzyme is a nCas12a comprising at least one mutation in the core lid domain conferring nickase activity, optionally wherein the at least one nCas12a comprises or consist of an amino acid sequence selected from SEQ ID NOs: 14 to 29, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, and/or wherein the at least one nCas12a comprises the core lid domain of any one of SEQ ID NOs: 14 to 29 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain.

In a second aspect there is provided, a prime editing guide RNA (pegRNA) or pegRNA system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; (vi) preferably, a second scaffold sequence, optionally being the same as the first scaffold sequence, wherein the primer binding site is complementary to the non-target strand.

In one embodiment, the pegRNA system comprises a) a pegRNA comprising parts (i), (ii), optionally (iii), (iv), (v), and optionally (vi); or b) a crispr RNA (crRNA) and a prime editing template RNA (petRNA), wherein 1) said crRNA comprises parts (i). (ii) and optionally (vi); and 2) said petRNA comprises optionally (iii), (iv) and (v).

In yet another embodiment, the pegRNA system comprises a crRNA and a petRNA, wherein the petRNA is linear.

In one embodiment, the pegRNA system further comprises (vii) optionally at least one 3′ linker sequence; and (viii) at least one structured motif, including at least one hairpin, including at least one MS2 stem loop, preferably at least two MS2 stem loops, and/or at least one pseudoknot.

In one embodiment, (iv) the reverse transcriptase template has a length of 25 to 140 nucleotides, or 30 to 120 nucleotides, or 40 to 100 nucleotides, or 50 to 90 nucleotides, or 60 to 90 nucleotides.

In one embodiment, (v) the primer binding site has a length of 6 to 40 nucleotides, or 6 to 30 nucleotides, or 7 to 20 nucleotides, or 7 to 15, nucleotides, or 9 to 12 nucleotides.

In one embodiment the pegRNA system comprises the parts (i), (ii), optionally (iii), (iv), (v), optionally (vi), and, if present, optionally (vii) and (viii) as defined above, in the following order in 5′ to 3′ direction: a) (i), (ii), optionally (iii), (iv), (v) and optionally (vi); or b) (iv), (v), (iii), (i), (ii), optionally (vi); or c) (i), (ii), optionally (iii), (iv), (v), (vii), (viii) and optionally (vi), or b) the crRNA comprises in a 5′ to 3′ direction parts (i), (ii) and optionally (vi) as defined above; and the petRNA comprises in a 5′ to 3′ direction parts (viii), optionally (iii), (iv), (v), optionally (iii), and (viii) as defined above, wherein the parts are preferably linked consecutively.

In one embodiment, the pegRNA system further comprises a nicking crRNA, said nicking crRNA comprising a CRISPR-Cas class 2 type V scaffold sequence; a spacer sequence, and optionally a second scaffold sequence, wherein the target site of the spacer sequence of said nicking crRNA is in the vicinity of and at the opposite strand of the target site of the spacer sequence of said pegRNA or said petRNA. In a third aspect, there is provided a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably being independently selected from Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP).

In one embodiment, the Cas12a prime editor comprises the reverse transcriptase protein in trans, and further comprises an RNA binding factor, preferably the N-terminal domain of La or of MCP, more preferably wherein the Cas12a prime editor comprises the N-terminal domain of La, optionally comprising the reverse transcriptase protein as defined above comprising an MCP, and the pegRNA system as defined above, and, optionally the nicking crRNA as defined above, wherein a) the crRNA comprises in a 5′ to 3′ direction (i) a CRISPR-Cas class 2 type V scaffold sequence, (ii) a spacer sequence and optionally (vi) a second scaffold sequence, optionally being the same as the first scaffold sequence; b) a prime editing template RNA (petRNA) comprising in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence and an MS2 stem loop.

In one embodiment, (ii) the at least one reverse transcriptase or active fragment thereof, is linked, via (iii) the at least one linker, N-terminally or C-terminally, preferably N-terminally, to (i) the at least one nCas12a, or active fragment thereof.

In one embodiment, the at least one nCas12a comprises at least one mutation in the core lid domain conferring nickase activity, optionally wherein the at least one nCas12a comprises or consist of an amino acid sequence selected from SEQ ID NOs: 14 to 29, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, and/or wherein the at least one nCas12a comprises the core lid domain of any one of SEQ ID NOs: 14 to 29 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain.

In one embodiment, the Cas12A prime editor comprises or consists of an amino acid sequence selected from SEQ ID NO: 65 to 69, 213 or 268 to 277, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In a fourth aspect, there is provided a complex comprising (a) at least one prime editing guide RNA (pegRNA) or pegRNA system as defined in the second aspect; and (b) at least one type 2 class V Cas enzyme, preferably having nickase activity, more preferably having non-target strand (NTS) nickase activity, or an active fragment thereof, optionally comprising an organellar localization signal.

In a fifth aspect, there is provided a nucleic acid molecule or more than one nucleic acid molecules encoding the prime editing complex of the first aspect; and/or the pegRNA system of the second aspect; and/or the Cas12a prime editor of the third aspect; and/or the complex of the fourth aspect.

In a sixth aspect, there is provided an expression vector or construct or more than one expression vector or constructs comprising the one or more nucleic acid molecules of the fifth aspect.

In a seventh aspect, there is provided a cell comprising the prime editing complex of the first aspect; and/or the pegRNA system of the second aspect and/or the Cas12a prime editor of the third aspect; and/or the complex of the fourth aspect; and/or the nucleic acid molecule or more than one nucleic acid molecules of the fifth aspect; and/or the expression vector or construct or more than one expression vector or constructs of the sixth aspect.

In one embodiment, the cell is a eukaryotic cell or a prokaryotic cell, including a bacterial or an archaeal cell.

In one embodiment, the cell is a plant cell, including an algal cell, preferably wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

In another embodiment, the cell is an insect, poultry, fish or crustacea cell, or a mammalian cell, preferably wherein the cell is a mammalian cell being selected from a cell originating from a non-human primate, bovine, porcine, rodent, including mouse, or human cell.

In another embodiment, the cell is a fungal cell, including a yeast cell, preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile.

In another embodiment, the cell is a prokaryotic cell, including a Gram-positive, Gram negative or Gram-variable bacterial cell, preferably Gram-negative bacterial cells, or an archaeal cell, preferably wherein the prokaryotic cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

In an eighth aspect, there is be provided a kit comprising the prime editing complex of the first aspect; and/or the pegRNA system of the second aspect; and/or the Cas12a prime editor of the third aspect; and/or the complex of the fourth aspect; and/or the nucleic acid molecule or more than one nucleic acid molecules of the fifth aspect; and/or the expression vector or construct or more than one expression vector or constructs of the sixth aspect; and/or the cell of the seventh aspect; further comprising a set of reagents; optionally comprising particles, vesicles, or at least one viral vector, or Agrobacterium vector for assisting delivery, wherein said particles comprise a lipid, including lipid nanoparticles, a sugar, a metal or a polypeptide, or a combination thereof, or wherein said vesicles comprise exosomes or liposomes.

In a ninth aspect, there is provided a method for modifying at least one nucleic acid sequence of interest in at least one nucleic acid molecule of at least one cell or construct at or near at least one target site, the method comprising: (a) providing at least one cell or construct comprising the nucleic acid sequence of interest to be modified; (b) providing and/or introducing (b-i) at least one pegRNA system as defined in the second aspect, or at least one nucleic acid molecule or expression vector or construct encoding the same, and (b-ii) at least one Cas12a prime editor, as defined in the first or third aspect; or at least one nucleic acid molecule or expression vector or construct encoding the same; optionally allowing complex formation of (b-i) the at least one pegRNA or crRNA and (b-ii) the at least one Cas12 prime editor before the provision and/or introduction; or providing and/or introducing: (b-iii) at least one prime editor complex as defined in the first aspect, or at least one nucleic acid molecule or expression vector or construct encoding the same; or providing and/or introducing (b-iv) at least one complex as defined in the fourth aspect, or at least one nucleic acid molecule or expression vector or construct encoding the same, and at least one reverse transcriptase as defined in the first or third aspect, or at least one or at least one nucleic acid molecule or expression vector or construct encoding the same; (c) allowing the modification of at least one nucleic acid sequence of interest by (b-i) the at least one pegRNA system and (b-ii) the at least one Cas12 prime editor; or by (b-iii) the at least one prime editor complex; or by (b-iv) the at least one complex as defined in the fourth aspect and the at least one reverse transcriptase as defined in the first or third aspect; (d) optionally, obtaining at least one edited cell or construct comprising a modification at least one nucleic acid sequence of interest at or near a target site; optionally, where the method comprises the following step: (e) regenerating at least one population of edited cells, tissues, organs, materials or whole organisms from the at least one edited cell or construct; optionally wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In a ninth aspect, there is provided a for producing an edited cell, tissue, and/or organism, the method comprising: (a) providing at least one cell, tissue, and/or organism comprising at least one nucleic acid sequence of interest in at least one nucleic acid molecule at or near at least one target site; (b) introducing (b-i) at least one pegRNA system as defined in the second aspect, or at least one nucleic acid molecule or expression vector or construct encoding the same, and (b-ii) at least one Cas12a prime editor, as defined in the first or third aspect; or at least one nucleic acid molecule or expression vector or construct encoding the same; optionally allowing complex formation of (b-i) the at least one pegRNA system and (b-ii) the at least one Cas12 prime editor before the provision and/or introduction; or introducing: (b-iii) at least one prime editor complex as defined in the first aspect, or at least one nucleic acid molecule or expression vector or construct encoding the same; or providing (b-iv) at least one complex as defined in the fourth aspect, or at least one nucleic acid molecule or expression vector or construct encoding the same, and at least one reverse transcriptase as defined in the first or third aspect, or at least one or at least one nucleic acid molecule or expression vector or construct encoding the same; (c) allowing the modification of at least one nucleic acid sequence of interest by (b-i) the at least one pegRNA or crRNA and (b-ii) the at least one Cas12 prime editor; or by (b-iii) the at least one prime editor complex; or by (b-iv) the at least one complex as defined in the fourth aspect and the at least one reverse transcriptase as defined in the first or third aspect; (d) obtaining at least one edited cell or construct comprising a modification at at least one nucleic acid sequence of interest at or near a target site; optionally, where the method comprises the following step: (e) regenerating at least one population of edited cells, tissues, organs, materials or whole organisms from the at least one edited cell or construct; optionally wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In one embodiment of the ninth or tenth aspect, the cell or construct is or originates from a prokaryotic cell, including a bacterial or an archaeal cell, or a eukaryotic cell.

In one embodiment of the ninth or tenth aspect, during step (a) and/or (b), at least one additional effector, or a nucleic acid sequence encoding the same, is provided, the additional effector promoting DNA repair and cell regeneration before, during or upon insertion of at least one nick or double-strand break at the nucleic acid sequence of interest at or near at least one target site.

In an eleventh aspect, there is provided an edited cell, tissue, organ, material or whole organism obtained by or obtainable by a method according to the tenth aspect as disclosed herein.

In a twelfth aspect, there is provided use of a compound selected from a prime editor complex as defined in the first aspect; and/or a pegRNA or pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh aspect; for introducing or modification in a nucleic acid molecule, preferably in a genome, including uses for optimizing or modifying a trait in a plant, including the modification of a yield-related trait, or a disease-resistance related trait, and/or for metabolic engineering in cell, including a prokaryotic or eukaryotic cell, preferably in a plant cell, an algal cell, a fungal cell, including a yeast cell, or an archaeal cell, optionally wherein the use excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In one embodiment, the use is a use for in vitro, in vivo and/or ex vivo modification of a nucleic acid molecule, preferably in a genome of a cell.

In a thirteenth aspect, there is provided a compound selected from a prime editor complex as defined in the first aspect; and/or a pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; for use in a method of treating or preventing a disease in a patient.

In a fourteenth aspect, there is provided a method of treating or preventing a disease, the method comprising using a prime editor complex as defined in the first aspect; and/or a pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; for introducing at least one modification in a genomic locus of interest of at least one cell of a subject in need thereof at or near at least one disease-state related target site.

In one embodiment, the method comprises an ex vivo modification of the at least one disease-state related target site, wherein at least one cell of a subject is provided to perform an ex vivo modification of the at least one disease-state related target site to obtain at least one edited cell.

In a fifteenth aspect, there is provided a method for cell therapy, comprising administering to a patient in need thereof said at least one edited cell of the fourteenth aspect, wherein presence of said at least one edited cell remedies a disease in said patient.

In a sixteenth aspect, there is provided a use of a compound selected from a prime editor complex as defined in the first aspect; and/or a pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; for the manufacture a medicament for treating or preventing a disease in a patient.

### Definitions

An “active fragment” or “functional fragment” as used herein describes a part of a protein that can, without the rest the protein, fulfill at least one desired function, including an enzymatic activity, of the whole-length protein. For instance an active fragment of a CRISPR-Cas enzyme is a part of a CRISPR-Cas enzyme that has (in combination with a cognate guide RNA) site-directed endonuclease activity. An active fragment of a reverse transcriptase is a part of a reverse transcriptase that has reverse transcriptase activity. An “active fragment” or “functional fragment” is not limited to proteins having enzymatic activity. For instance a functional fragment of a DNA-binding protein is a part of a DNA-binding protein that has DNA binding activity.

The term “disease-state-related target site” as used herein refers to any target site for which a certain allele, variant or mutation actually or potentially causes, influences or may be a risk factor for at least one physical and/or mental disease, ailment, disorder or adverse condition or propensity, or the progression or prognosis thereof. A disease-state-related target site may for example be a target site comprising a missense or nonsense mutation within a protein-coding gene or it may be a target site comprising a variant of a polymorphism, such as a single-nucleotide polymorphism, that correlates may be a risk factor for the development of a certain disease.

The term “fused” or “fusion” when referring to polypeptides as used herein refers to a fusion protein that is a polypeptide that is created by joining at least two different polypeptide sequences, which may each be a complete protein existing in nature, a part thereof, or a fully synthetic polypeptide, to form one continuous artificial polypeptide. Commonly, a fusion protein is produced by fusing the nucleic acid sequences of the polypeptides to be joined. Means and methods to clone and express fusion proteins are well-established in the field and available to the skilled person. The polypeptide sequence may be fused directly or via a peptide linker as known in the art and/or as disclosed herein.

The term “guide RNA” may refer to any RNA comprising a Cas-protein-binding region (referred to as “scaffold sequence” herein, also called repeat sequence in the art) and a targeting region (the spacer sequence) and is capable of guiding a Cas protein to a target site being sufficiently complementary to the targeting region of the guide RNA as long as the target site is located next to a PAM sequence suitable for the respective Cas protein. The PAM motif (nucleotides that are specifically recognized by amino acids of the Cas protein) is often located on the non-target strand (for Cas12 and many Cas9 proteins), sometimes on both strands (for some Cas9 proteins). For Cas12a systems, the terms “guide RNA”, “crRNA”, gRNA” or “sgRNA” are used interchangeably. For systems and/or approaches using a two-molecule guide RNA in the natural environment as known in the art, such as a crRNA and a tracrRNA, the term guide RNA refers to both RNA molecules. Once a CRISPR effector system including a Cas enzyme and the cognate guide RNA (crRNA, or crRNA: tracrRNA) is described, the skilled person is thus aware which type of guide RNA is used for which type of Cas enzyme, for instance a Cas12a system uses a single crRNA, whereas a Cas12e system uses a crRNA::tracrRNA duplex similar to a Cas9 system, wherein a crRNA::tracrRNA duplex may however be mimicked by a synthetic single guide RNA molecule. A few Cas12 varants, such as Cas12c or Cas12d systems, have a crRNA and a so-called scout RNA. Accordingly, a prime editing guide RNA of the present invention may consist of one or two RNA molecules. Further, the skilled person is well aware of designing, expressing/synthesizing and adapting guide RNAs for the purposes needed. Particularly, the mutations to (n) Cas12a enzymes and (n) Cas12 orthologs thereof as disclosed herein will not have an influence on the overall design and the mode of interaction of the cognate guide RNA for a given nCas12a enzyme, or for a nCas12 ortholog. In some embodiments relating to prime editing, a prime editor and/or prime editor complex, the guide RNA is a pegRNA (prime editing guide RNA). A pegRNA will typically further include a primer binding site (PBS) and a reverse transcriptase template sequence. Naturally a pegRNA of the present disclosure is to be designed to be a cognate guide RNA for the desired CRISPR-Cas effector, such as a prime editor of the present invention. For a prime editor complex of the present invention as well as any method and/or use according to the present invention that relates to the prime editing using at least one pegRNA and at least one prime editor, the pegRNA is a cognate guide RNA for the CRISPR-Cas effector comprised by said prime editor. The design of guide RNAs, suitable for various Cas systems is well known to the skilled person. A pegRNA that is a cognate guide RNA for a desired CRISPR-Cas effector will comprise a scaffold sequence suitable for the desired CRISPR-Cas effector linked to the spacer sequence as known in the art. The particulars of designing a functional CRISPR-Cas type II class V pegRNA are disclosed herein, wherein the spacer sequence for targeting the sequence of interest, and the scaffold sequence for binding to the cognate CRISPR-Cas effector may be designed as it is known in the art (for design of spacer and scaffold sequences cf. e.g. Grissa et al., BMC Bioinformatics. 2007 May 23; 8:172. doi: 10.1186/1471-2105-8-172).

In some embodiments relating to prime editing, a crRNA is used in combination with a prime editing template RNA (petRNA). A petRNA will typically include a primer binding site (PBS) and a reverse transcriptase template sequence. A petRNA may be linear or circular. For a prime editor complex of the present invention as well as any method and/or use according to the present invention that relates to the prime editing using at least one crRNA, at least one petRNA and at least one prime editor, the crRNA is a cognate guide RNA for the CRISPR-Cas effector comprised by said prime editor. A crRNA that is a cognate guide RNA for a desired CRISPR-Cas effector will comprise a scaffold sequence suitable for the desired CRISPR-Cas effector linked to the spacer sequence as known in the art. The particulars of designing a functional CRISPR-Cas type II class V crRNA are disclosed herein, wherein the spacer sequence for targeting the sequence of interest, and the scaffold sequence for binding to the cognate CRISPR-Cas effector may be designed as it is known in the art (for design of spacer and scaffold sequences cf. e.g. Grissa et al., BMC Bioinformatics. 2007 May 23; 8:172. doi: 10.1186/1471-2105-8-172).

The term prime editing guide RNA (pegRNA) system is specifically used to denote a complex according to the second aspect of the present invention, wherein the pegRNA is a complex molecule or system, so that the term “pegRNA system” is used herein in addition to the term pegRNA to denote a pegRNA comprising or consisting of several functional moieties or effector parts. The pegRNA system may thus consist of a classical pegRNA, or it may encompass a “split” pegRNA consisting of a crRNA and a petRNA portion as further disclosed and illustrated herein below. Thus, functional moieties present in a pegRNA system as described herein may be present on the pegRNA, or on the crRNA and/or the petRNA as well.

“Identity” when used in respect to the comparison of two or more nucleic acid or amino acid molecules designates the degree of exact nucleic acid, base pair or amino acid matches between the sequences of said molecules.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases (or nt(s))
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

“Indel” is a term for the random insertion or deletion of bases in the genome of an organism associated with the repair of a DSB by non-homologous end joining (NHEJ). It is classified among small or sometimes larger genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term in vitro as used herein refers to the state or quality of a method or application or procedure of not being performed inside of a living cell, preferably in a cell-free system. In vitro methods, applications or procedures are typically performed with biological material, such as nucleic acids, polypeptides and the like that have been purified from cells and/or were artificially processed or synthesized, usually in a reaction tube or reaction compartment comprising a suitable buffer system and suitable reaction components.

The term in vivo as used herein refers to the state or quality of a method, application or procedure of comprising the manipulation of at least one living cell (including cells grown in cell culture), such as the introduction of CRISPR components into living cells and potential genomic nicking, double-strand cleavage and/or modification within said cells. In vivo methods, applications or procedures may be followed by in vitro analysis of e.g. purified DNA after cell lysis.

The term ex vivo as used herein refers to the state or quality of a method, application or procedure to be directed at living cells and/or living tissue extracted from an organism, wherein said living cells and/or living tissue may be re-inserted into the organism, from which it was extracted, or inserted into a different organism typically of the same species, after the ex vivo method, application or procedure.

The term “linked” when referring to nucleic acids, designates the covalent or non-covalent, preferably covalent, attachment of one polynucleotide or part thereof to another polynucleotide or part thereof, typically via the sugar-phosphate backbone. A consecutive linkage of one part to another part, describes that no different polynucleotide part is present between the parts. When referring to polypeptides, the term “linked” designates the covalent or non-covalent attachment of one polypeptide or part thereof to another polypeptide or part thereof. Covalent polypeptide linkage may be directly or indirectly via a linker, wherein the linker may be a peptide linker (referred tp as “fused” herein) or different linker.

“Nickase activity” as used herein refers to the capability to generate single-strand DNA breaks (nicks) in a dsDNA molecule, preferably wherein these single-strand DNA breaks are generated at a higher rate and/or percentage than double-stranded breaks (through residual cleavage of the second strand), optionally wherein the residual nuclease activity is less than approximately 20%, more preferably less than approximately 15%, even more preferably less than approximately 10%, and most preferably less than approximately 5% of total enzyme activity, wherein the total enzyme activity is the sum of nickase activity and nuclease activity of a given Cas12a enzyme having nickase activity or catalytically active fragment thereof, wherein the nickase activity and nuclease activity of a given Cas12a enzyme having nickase activity or catalytically active fragment thereof are determined and compared with the same detection system and/or method in a suitable cellular and/or in vitro system using suitable and reasonable reaction conditions and further using the same target site(s) under the same conditions within reasonable limits of said cellular and/or in vitro system. The skilled person is well aware of various different suitable methods to determine nickase and nuclease activity of a Cas12a enzyme, including methods disclosed herein. Cas12a nickases of the present invention show reduced cleavage of the second strand. Extending the incubation time (for in vitro experiments) and/or increasing the amount and/or concentration and/or expression of nCas12a (and the cognate guide RNA) relative to the amount and/or concentration of target nucleic acid may increase the percentage of second strand cleavage relative to the nicking events. Therefore, optimization of amounts, concentrations, expression rates, and/or incubation times and the likes may be used for achieving highest possible rates of nicks relative to DBSs. The term “nuclease activity” as used herein refers to endonucleolytic activity wherein one nuclease effector is able to generate a double-strand break, whereas for a nickase—to achieve a double-strand break-two individual nicks (requiring at least two different guide RNAs for CRISPR-Cas nickases) are needed. Target strand (TS) nickase activity as used herein refers to nickase activity as described above, wherein at least 90% of the nicking occurs in the target strand. Non-target strand (NTS) nickase activity as used herein refers to nickase activity as described above, wherein at least 90% of the nicking occurs in the non-target strand.

The term “plant” as used herein encompasses whole plants, ancestors and progeny of the plants and plant parts, including seeds, shoots, stems, leaves, roots (including tubers), flowers, and tissues and organs. The term “plant” also encompasses plant cells, suspension cultures, callus tissue, embryos, meristematic regions, gametophytes, sporophytes, pollen and microspores.

A “target site” as used herein refers to both strands of a double-stranded DNA, i.e. a target strand (TS)—to which a guide RNA anneals—and a complementary non-target strand (NTS), wherein the target site is the stretch of DNA for with a guide RNA has suitable complementarity to the target strand. “At or near a target site” as used herein refers to the part of DNA that is within the target site or up to 10 bp, up to 20 bp, up to 30 bp, or up to 40 bp next to the target site, including both directions.

### BRIEF DESCRIPTION OF FIGURES

FIG. 1 (FIG. 1) shows successful editing of an extrachromosomal dGFP reporter with an RT-nLbCas12a prime editor (SEQ ID NO: 65) in oilseed rape (Brassica napus) protoplasts. 35S>eGFP denotes a positive control in which cells are transformed with eGFP under control of a 35S promoter. pUbiAt denotes the ubiquitin10 promoter from Arabidopsis thaliana (SEQ ID NO: 149), RT (5M) denotes an engineered M-MLV reverse transcriptase (SEQ ID NO: 146), RTT denotes a reverse transcription template, PBS denotes a primer binding site. (R denotes the red channel (autofluorescence) and G denotes the green channel (GFP)).

FIG. 2A and FIG. 2B (FIG. 2A and FIG. 2B) shows RT-nLbCas12a-mediated dGFP reporter prime editing rates (dGFP>GFP) in oilseed rape protoplasts at different incubation temperatures 2 days post transfection (dpt). Y-axes show % of GFP-positive cells. FIG. 2A shows results using the pegRNA of SEQ ID NO: 33 and FIG. 2B shows results using the pegRNA of SEQ ID NO: 34.

FIG. 3 (FIG. 3) shows successful editing of an extrachromosomal dGFP reporter in oilseed rape (Brassica napus) protoplasts expressing an N-terminal fusion of M-MLV RT (5M) to nLbCas12a (SEQ ID NO: 65) and the pegRNA of SEQ ID NO: 33. Transfected protoplasts were successively incubated for 24 h at 37° C. and 28° C. (R denotes the red channel (autofluorescence) and G denotes the green channel (GFP)).

FIG. 4 (FIG. 4) shows dGFP editing rates in oilseed rape (Brassica napus) protoplasts expressing the pegRNAs of SEQ ID NO: 33 and SEQ ID NO: 34 along with C-terminal fusions of the ssDNA binding protein Brex27 and RT (5M)-nLbCas12a. Y-axis shows % of GFP-positiv cells.

FIG. 5 (FIG. 5) shows a graphic depicting the target region for prime editing of the bacterial neo gene. Shown is a partial sequence of the neo gene with, at the top, the amino acid sequence and accompanying positions. Intact denotes the DNA sequence of the wildtype neo gene, disrupted denotes the DNA sequence of the disrupted neo gene in pTarget_neo_disrupted, restored denotes the DNA sequence of the neo gene after successful prime editing. The bases that are altered are shown in dark gray. The target strand of the LbCas12a protospacer is shown in light gray in underlined letters and bases opposite of the LbCas12a PAM are shown in light gray without underlining. DNA sequences are shown from 5′ to 3′. Depicted sequences are included as SEQ ID NOs: 174 to 178.

FIG. 6 (FIG. 6) shows the number and position of different silent mutations introduced in the RTT of a Cas12a pegRNA targeting a dGFP reporter plasmid. Sequences are included in the shown order as SEQ ID NOs: 207 to 212.

FIG. 7 (FIG. 7) shows the impact of different culture conditions on the editing efficiency of an RT-nLbCas12a fusion protein in oilseed rape (Brassica napus) protoplasts co-transformed with a dGFP reporter plasmid. Editing efficiencies are expressed as the percentage of GFP-positive cells three days post transfection as determined by fluorescence imaging.

FIG. 8 (FIG. 8) shows successful editing of an extrachromosomal dGFP reporter in oilseed rape (Brassica napus) protoplasts expressing an N-terminal fusion of M-MLV RT (5M) to nLbCas12a (D156R/R1138A) (SEQ ID NO: 199) and the pegRNA of SEQ ID NO: 203 together with the nicking crRNA of SEQ ID NO: 201 and the MCP-RT (5M) fusion of SEQ ID NO: 200. Transfected protoplasts were successively incubated for 24 h at 37° C. and 28° C. (R denotes the red channel (autofluorescence) and G denotes the green channel (GFP). FIG. 9 (FIG. 9A to C) shows the secondary structures of three Cas12a pegRNAs targeting a dGFP reporter plasmid. Shown as FIG. 9A is a canonical pegRNA with predicted auto-inhibitory base pairing between the spacer and PBS sequences. The FIG. 9B and FIG. 9C are pegRNAs engineered to disrupt spacer-PBS interactions through addition of different stem structures in between the spacer and RTT-PBS. Depicted sequences are included as SEQ ID NO: 33 (FIG. 9A), SEQ ID NO: 214 (FIG. 9B) and SEQ ID NO: 217 (FIG. 9C).

FIG. 10 (FIG. 10) shows successful editing of an extrachromosomal dGFP reporter in oilseed rape (Brassica napus) protoplasts co-expressing a Cas12a prime editor composed of the nLbCas12a (D156R/R1138A) nickase with N-terminal RT and C-terminal MCP fusions (SEQ ID NO: 269) together with the MCP-RT (5M) fusion of SEQ ID NO: 200, a dGFP-targeting Cas12a crRNA (SEQ ID NO: 223) and MS2-tagged linear template RNAs (SEQ ID NO: 220). R denotes the red channel (autofluorescence) and G denotes the green channel (GFP).

### DETAILED DESCRIPTION

The inventors of the present disclosure have developed class 2 type V CRISPR-Cas nickases, with specific non-target strand nickase activity, which allow a prime editing system that functions similar to the established Cas9 prime editing architecture and shows superior and reliable performance in a variety of different eukaryotic and prokaryotic target cells, particularly including plant cells and human cells.

In a first aspect, there is provided a prime editing complex comprising (a) at least one prime editing guide RNA (pegRNA) or pegRNA system as defined in the second aspect; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof; and (iv) optionally: at least one organellar localization signal.

A Cas12a, according to the present invention may be selected from, but is not limited to LbCas12a, Lb2Cas12a, Lb3Cas12a, AsCas12a, BpCas12a, CMtCas12a, EeCas12a, FnCas12a, LiCas12a, MbCas12a, PbCas12a, PcCas12a, PdCas12a, PeCas12a, PmCas12a, SsCas12a, enAsCas12a.

In certain embodiment according to the first, third or fourth aspect, the at least one Cas12a enzyme may be based on a wild-type Cas12a sequence according to any one of SEQ ID NOs: 1 to 12, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding wild-type sequence as reference sequence, or an ortholog or homolog of a sequence according to any one of SEQ ID NOs: 1 to 12 having at least 95%, 96%, 97%, 98% or at least 99% sequence identity to the corresponding ortholog or homolog sequence as reference sequence.

A Cas12a enzyme, optionally a Cas12a enzyme having nickase activity, according to the first, third or fourth aspect herein, may comprise at least one mutation that modifies the PAM-specificity and/or the thermotolerance of the engineered Cas12a enzyme.

Most wild type Cas12a proteins have a relatively strict requirement for a PAM sequence of TTTV—with some variation between different Cas12a orthologs.

Suitable PAM variants expanding the PAM constraint have been described for various Cas12a orthologs (see for example WO2018195545, WO2020033774 and WO2018022634).

According to the various aspects and embodiments disclosed herein, at least one mutation leading to a PAM variant with amended PAM specificity, preferably to expand or alter the PAM constraint of the respective wild-type Cas12a enzyme, can be combined with the nCas12a enzymes as disclosed herein.

Mutants that modify the PAM specificity and/or thermotolerance include, for example, LbCas12a-RR (G532R/K595R), LbCas12a-RVR (G532R/K538V/Y542R), LbCas12a-RVRR (G532R/K538V/Y542R/K595R), enLbCas12a (D156R/G532R/K538R), ttLbCas12a (D156R), FnCas12a-RR (N607R/N617R), FnCas12a-RVR (N607R/K613V/N617R), FnCas12a-RVRR (N607R/K613V/N617R/K671R), AsCas12a-RR (S542R/N552R), AsCas12a-RVR (S542R/K548V/N552R), AsCas12a-RVRR (S542R/K548V/N552R/K607R), enAsCas12a-HF (E174R/N282A/S542R/K548R), MbCas12a-RR (N576R/N582R), MbCas12a-RVR (N576R/K578V/N582R), MbCas12a-RVRR (N576R/K578V/N582R/K634R), Mb2Cas12a-RVR (Mb2Cas12a N563R/K569V/N573R), Mb2Cas12a-RVRR (Mb2Cas12a N563R/K569V/N573R/K625R), BsCas12a-3Rv (K155R/N512R/K518R), PrCas12a-3Rv (E162R/N519R/K525R), Mb3Cas12a-3Rv (D180R/N581R/K587R) (WO2018195545, WO2020033774, WO201822634).

In some embodiments according to the first, third or fourth aspect, the at least one mutation in the core lid domain according to the present invention may be present in a Cas12a variant with one of the following amino acid reference sequences: SEQ ID NO: 167, SEQ ID NO: 168, SEQ ID NO: 169, SEQ ID NO: 170, SEQ ID NO: 171, SEQ ID NO: 172 or SEQ ID NO: 173.

All wild-type Cas12a enzymes provided so far disclosed in the prior art as suitable for genome editing can qualify as sources for a Cas12a enzyme, preferably having nickase activity (nCas12a), more preferably having non-target strand (NTS) nickase activity, as disclosed herein. As orthologs, for example, closely related FnCas12a, ErCas12a sequences can qualify-without having these included in the independent claims.

In one embodiment of the first, third or fourth aspect, a Cas12a ortholog enzyme, preferably having nickase activity (nCas12a), more preferably having non-target strand (NTS) nickase activity, may include Cas12e (also referred to as CasX), including DpbCas12e and PlmCas12e (Selkova et al. RNA Biol. (2020); 17 (10): 1472-1479; doi: 10.1080/15476286.2020.1777378).

In another embodiment of the first, third or fourth aspect, a Cas12a ortholog enzyme, preferably having nickase activity (nCas12a), more preferably having non-target strand (NTS) nickase activity, may include Cas12f variants, including Cas12f1 (Cas14a and type V-U3), including AsCas12f1 and Un1Cas12f1, Cas12f2 (Cas14b) and Cas12f3 (Cas14c, type V-U2 and U4) (Kim et al. Nat Biotechnol. (2022); 40 (1): 94-102; doi: 10.1038/s41587-021-01009-z; Karvalis et al. Nucleic Acids Res. (2020); 48 (9): 5016-5023. doi: 10.1093/nar/gkaa208).

Nicking of the non-target strand was shown to improve prime editing for Cas9 prime editing systems (Anzalone et al., supra). For Class 2 type V CRISPR-Cas prime editing systems of the present invention, nicking of the non-target strand may also be used to improve prime editing as shown in the examples herein.

Other species sources are: Cas12a variants or any Cas12 ortholog selected from the group consisting of Francisella tularensis, Prevotella albensis, Lachnospiraceae bacterium, Butyrivibrio proteoclasticus, Peregrinibacteria bacterium, Parcubacteria bacterium, Smithella sp., Acidaminococcus sp., Candidatus Methanoplasma termitum, Eubacterium eligens, Eubacterium rectale, Moraxella bovoculi, Leptospira inadai, Porphyromonas crevioricanis, Prevotella disiens and Porphyromonas macacae, Succinivibrio dextrinosolvens, Prevotella disiens, Flavobacterium sp., Flavobacterium branchiophilum, Helcococcus kunzii, Eubacterium sp., Microgenomates (Roizmanbacteria) bacterium, Prevotella brevis, Moraxella caprae, Bacteroidetes oral, Porphyromonas cansulci, Synergistes jonesii, Prevotella bryantii, Anaerovibrio sp., Butyrivibrio fibrisolvens, Candidatus Methanomethylophilus, Butyrivibrio sp., Oribacterium sp., Pseudobutyrivibrio ruminis and Proteocatella sphenisci., Acidibacillus spp., including Acidibacillus sulfuroxidans, Deltaproteobacteria spp, Planctomycetes spp.

In one embodiment of the first, third or fourth aspect, the at least one nCas12a enzyme is an Cas12a comprising a R1138A mutation or an AsCas12a comprising a R1226A mutation.

In one embodiment of the first or fourth aspect, the at least one nCas12a enzyme is an LbCas12A comprising a R1138A mutation, or it is an AsCas12a comprising a R1226A mutation, wherein the at least one pegRNA is a linear pegRNA as defined in the second aspect.

In certain embodiment of the first, third or fourth aspect, the at least one Cas12a enzyme or active fragment thereof and/or at least one reverse transcriptase or active fragment thereof may comprise, by covalent and/or non-covalent attachment to the at least one Cas12a enzyme or active fragment thereof and/or the at least one reverse transcriptase or active fragment thereof, at least one cell-penetrating polypeptide and/or at least one organellar localization signal, selected from a nuclear localization signal (NLS), a mitochondrion localization signal, or a chloroplast localization signal, preferably, in case the at least one further polypeptide is covalently attached to the at least one Cas12a or active fragment thereof and/or the at least one reverse transcriptase or active fragment thereof, the at least one further polypeptide is covalently attached N-terminally and/or C-terminally and/or internally to the at least Cas12a or active fragment thereof and/or at least on reverse transcriptase or active fragment thereof. In embodiments relating to a non-covalent attachment of the at least one reverse transcriptase to the at least one Cas12a enzyme, all protein components may each be (covalently and/or non-covalently) attached to the same type of, or identical, organellar localization signals.

Preferably the at least one organellar localization signal and/or the at least one cell penetrating peptide is covalently attached to the at least one Cas12a enzyme or active fragment thereof and/or the at least one reverse transcriptase or active fragment thereof, more preferably the covalent attachment is an attachment via a peptide linker so that the at least one organellar localization signal and/or the at least one cell penetrating peptide forms a continuous polypeptide with the at least one Cas12a enzyme or active fragment thereof and/or the at least one reverse transcriptase or active fragment thereof.

An organelle localization signal according to the first, third or fourth aspect, may be a nuclear localization signal (NLS), a mitochondrion localization signal and/or a chloroplast localization signal. In preferred embodiments of the first, third or fourth aspect, the at least one organelle localization signal is at least one nuclear localization signal. Nuclear localization signals, mitochondrion localization signals and chloroplast localization signals are well known in the art, any such sequence as disclosed herein and/or known in the art may be used as part of a prime editor of the third aspect or a prime editor complex of the second aspect.

In one embodiment, at least one organellar localization signal is located at the N-terminus and/or the C-terminus of the at least one Cas12a enzyme, or active fragment thereof, and/or the at least one reverse transcriptase, or active fragment thereof.

An NLS may be a monopartite NLS or a bipartite NLS, for example, without being limited thereto, an SV40 NLS, a nucleoplasmin NLS, an EGL-13 NLS or a c-Myc NLS (cf. e.g. Bioconjug Chem. 2015 Jun. 17; 26 (6): 1004-7. doi: 10.1021/acs.bioconjchem.5b00141). “At least one organellar localization” when referring to an NLS many comprise or consist of two or three or more repetitions of the same NLS.

In one embodiment, the at least one organellar localization signal is at least one bipartite NLS comprising or consisting of the sequence according to SEQ ID NO: 130, or a sequence having 99% sequence identity thereto.

In one embodiment, the at least one organellar localization signal is at least one SV40 NLS.

In one embodiment, at least two or at least three, for example, three repeats of an SV40 NLS, may be located at the N-terminus and/or the C-terminus of the at least one Cas12a enzyme, or active fragment thereof, and/or the at least one reverse transcriptase, or active fragment thereof.

Organellar localization may be improved by having at least one organellar localization signal located at the N-terminus and/or the C-terminus of the at least one Cas12a enzyme, or active fragment thereof, and at the N-terminus and/or the C-terminus of the at least one reverse transcriptase, or active fragment thereof.

In embodiments, wherein the at least one reverse transcriptase, or active fragment thereof is fused to the at least one Cas12a enzyme, or active fragment thereof, it is preferred, that at least one organellar localization signal is located at the N-terminus and/or at the C-terminus, more preferably at the N-terminus and the C-terminus of the fusion protein.

In one embodiment, the at least one Cas12a enzyme, or active fragment thereof, is an RNase dead Cas12a enzyme, or active fragment thereof, optionally comprising a mutation at position H759, including a H759A mutation. An RNase dead Cas12a, which lacks crRNA processing activity (cf. Fonfara et al., Nature. 2016 Apr. 28; 532 (7600): 517-21. doi: 10.1038/nature17945; Swarts et al., Mol Cell. 2017 Apr. 20; 66 (2): 221-233.e4. doi: 10.1016/j.molcel.2017.03.016.), may be used to improve the prime editing according to the present invention. The H759 refers to the numbering according to LbCas12a and will thus be at a different position for Cas12a orthologs (e.g. H843 fo FnCas12a). Other position for mutations, instead or in addition, may for example, without being limited thereto, be K785 (numbering according to LbCas12a), or K852 and/or K869 (numbering according to FnCas12a; FnCas12a K869 A LbCas12a K785). Typical loss-of-function (here loss of RNase activity) mutations are amino acid exchanges to alanine, but may also be different amino acid exchanges.

In one embodiment, the Cas12a prime editor comprises at least one ssDNA-binding and/or ssDNA-stabilizing protein, or domain or functional active fragment thereof, preferably being independently selected from any one of Brex27, RPA70-A, RPA70 B, RPA70-C, RPA32-D, BRCA2-OB2, BRCA2-OB3, HNRNPK (Heterogeneous nuclear ribonucleoprotein K) KH (K-homology domain), PUF60RRM, Rad51DBD, optionally being selected from a sequence according to SEQ ID NO: 71 (Brex27), 72 (RPA70-A), 73 (RPA70 B), 74 (RPA70-C), 75 (RPA32-D), 76 (BRCA2-OB2), 77 (BRCA2-OB3), 78 (HNRNPK KH), 79 (PUF60RRM) and 80 (Rad51DBD), or a sequence having at least 80%, 81%, 82%, 83%, 84%, 85%, 86%, 87%, 88%, 89%, 90%, 91%, 92%, 93%, 94%, 95%, 96%, 97%, 98% or at least 99% sequence identity thereto. A nucleic acid sequence encoding the at least one ssDNA-binding and/or ssDNA-stabilizing protein, or domain or functional active fragment thereof may for example, without being limited thereto, be selected from a sequence according to SEQ ID NO: 81 to 90.

In a further embodiment, the prime editor complex further comprises in trans (c) a reverse transcriptase protein, preferably comprising at least one organellar localization signal and preferably comprising an MS2 coat protein (MCP).

In yet another embodiment, the at least one Cas12a enzyme, or active fragment thereof the prime editor complex further comprises an RNA binding factor, preferably an N-terminal domain of La or a MCP. As illustrated in Examples 5 and 9, these configurations may be particularly suitable for prime editing in certain target cells.

In one embodiment, the prime editor complex comprises the N-terminal domain of La, optionally comprising the reverse transcriptase protein as defined above comprising an MCP, and the pegRNA system as defined above, and, optionally the nicking crRNA as defined above, wherein a) the crRNA comprises in a 5′ to 3′ direction (i) a CRISPR-Cas class 2 type V scaffold sequence, (ii) a spacer sequence; and optionally (vi) a second scaffold sequence, optionally being the same as the first scaffold sequence; and/or wherein b) the prime editing template RNA (petRNA) comprises in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

In a preferred embodiment, there is provided a fusion with La, a split linear pegRNA system and optionally the MCT-RT in trans and a nicking crRNA.

Suitable ssDNA binding proteins have been described in the literature (cf. e.g. Dickey et al., Structure. 2013 Jul. 2; 21 (7): 1074-84. doi: 10.1016/j.str.2013.05.013).

In certain embodiment of the first or third aspect, the at least one Cas12a enzyme or active fragment thereof and/or at least one reverse transcriptase or active fragment thereof may comprise, by covalent and/or non-covalent attachment to the at least one Cas12a enzyme or active fragment thereof and/or the at least one reverse transcriptase or active fragment thereof, at least one further protein, active fragment or domain thereof having enzymatic activity that modifies at least one target nucleic acid e.g., nuclease activity, e.g. exonuclease activity or endonuclease activity (e.g. such as that provided by a restriction enzyme, Fokl nuclease, Clo051 nuclease, or homing endonucleases), methyltransferase activity, demethylase activity, DNA repair activity, DNA damage activity, deamination activity including cytosine deaminase and/or a adenine deaminase activity, dismutase activity, alkylation activity, depurination activity, oxidation activity, pyrimidine dimer forming activity, helicase activity (e.g. SF1/2, SF3, SF4), integrase activity such as that provided by an integrase and/or resolvase (e.g., Gin integrase such as the hyperactive mutant of the Gin integrase, GinH106Y; human immunodeficiency virus type 1 integrase (IN); Tn3 resolvase; and the like), telomerase activity, topoisomerase activity, e.g. gyrase activity, transposase activity, recombinase activity such as that provided by a recombinase (e.g., catalytic domain of Gin recombinase, Cre recombinase, Hin recombinase, Tre recombinase, FLP recombinase, RecA, RadA, Rad51), polymerase activity, e.g. RNA polymerase activity or DNA polymerase activity e.g. Pol theta activity, ligase activity, photolyase activity or glycosylase activity, ligase activity, helicase activity, hotolyase activity or glycosylase activity. Preferably the at least one further protein, active fragment or domain thereof is covalently attached, more preferably via a peptide linker.

In some embodiments according to the first or third aspect, the at least one Cas12a enzyme or active fragment thereof and/or at least one reverse transcriptase or active fragment thereof may comprise, by covalent and/or non-covalent attachment to the at least one Cas12a enzyme or active fragment thereof and/or the at least one reverse transcriptase or active fragment thereof, at least one further protein, active fragment or domain thereof having enzymatic activity that modifies at least one protein and/or polypeptide (e.g., a histone) associated with at least one target nucleic acid. Examples of enzymatic activity that modifies at least one protein and/or polypeptide associated with at least one target nucleic acid include but are not limited to: methyltransferase activity, such as that provided by a histone methyltransferase (HMT) (e.g., suppressor of variegation 3-9 homolog 1 (SUV39H1 or KMT1A), euchromatic histone lysine methyltransferase 2 (G9A, KMT1C, EHMT2), SUV39H2, ESET/SETDB 1, and the like, SET1A, SET1B, MLL1 to 5, ASH1, SYMD2, NSD1, DOT1L, Pr-SET7/8, SUV4-20H1, EZH2), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity, such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HB01/MYST2, HMOF/MYST1, SRC1, ACTR, P160, CLOCK and the like), deacetylase activity, such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity. Preferably the at least one further protein, active fragment or domain thereof is covalently attached, more preferably via a peptide linker.

In one embodiment, the at least one further protein, active fragment or domain thereof, is at least one mismatch repair inhibitor and/or at least one 5′ flap endonuclease, such as Fen1.

In some embodiments of the first or third aspect, the at least one Cas12a enzyme or active fragment thereof and/or at least one reverse transcriptase or active fragment thereof may comprise at least one detectable label. Suitable detectable labels and/or moieties that can provide a detectable signal can include, but are not limited to, an enzyme, a radioisotope, a member of a specific binding pair, a fluorophore, a fluorescent protein, a quantum dot, and the like. Preferably, in case of a detectable label that is a protein or active fragment or domain thereof, the at least one detectable label is covalently attached, more preferably via a peptide linker.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFPI, pocilloporin, Renilla GFP, Monster GFP, paGFP, Kaede protein and kindling protein, Phycobiliproteins and Phycobiliprotein conjugates including BPhycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. Nat Methods. 2005 December; 2 (12): 905-9. doi: 10.1038/nmeth819), and the like.

Suitable enzymes that may function as a detectable label include, but are not limited to, horse radish peroxidase (HRP), alkaline phosphatase (AP), beta-galactosidase (GAL), glucose-6-phosphate dehydrogenase, beta-Nacetylglucosarninidase, β-glucuronidase, invertase, Xanthine Oxidase, firefly luciferase, glucose oxidase (GO), and the like.

Further suitable proteins or fragments or domains thereof that may be non-covalently or covalently, optionally via a peptide linker, attached to the at least one Cas12a enzyme or active fragment thereof and/or at least one reverse transcriptase or active fragment thereof are boundary elements (e.g., CTCF), proteins and fragments thereof that provide periphery recruitment (e.g., Lamin A, Lamin B, etc.), protein docking elements (e.g., FKBP/FRB, Pill/Abyl, etc.).

A reverse transcriptase (RT) according to the first or third aspect may be a wild type reverse transcriptase or a synthetic reverse transcriptase (cf. Heller et al. Nucleic Acids Research, 47 (7) 3619-3630 (2019)). A variety of RTs are known to the skilled person in the field of genome editing (cf. Martín-Alonso et al. in biotechnology 39, 2 Trends vol. (2021): 194-210. doi: 10.1016/j.tibtech.2020.06.008; and Huber et al., Chembiochem: a European journal of chemical biology vol. 24, 5 (2023): e202200521. doi: 10.1002/cbic.202200521) and the RT according to the present invention may be selected therefrom. A reverse transcriptase may for instance be selected from, without being limited thereto, a Moloney Murine Leukemia Virus reverse transcriptase (M-MLV RT), a Cauliflower Mosaic Virus (CaMV) RT, or retron-derived RT (cf. e.g. Lin et al. supra).

Example reverse transcriptases may comprise or consist of a polypeptide having a sequence selected from Seq ID NO: 146, 147 or 148, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In one embodiment according to the first or third embodiment, the reverse transcriptase may be a Moloney Murine Leukemia Virus reverse transcriptase (M-MLV RT), optionally being a M-MLV RT D200N/L603W/T330P/T306K/W313F variant.

In one embodiment, (ii) the at least one reverse transcriptase, or active fragment thereof, is linked, via (iii) the at least one linker, N-terminally or C-terminally, preferably N-terminally, to (i) the at least one Cas12a enzyme, or active fragment thereof.

A covalent linker according to the first or third aspect, linking the (i) at least one Cas12a enzyme, or active fragment thereof, and the (ii) at least one reverse transcriptase, or an active fragment thereof, may be a non-peptide linker, for instance, but not limited to, a linker generated by “click chemistry” as known in the art, or a peptide linker.

A peptide linker may be a GS linker, such as a peptide linker comprising or consisting of an amino acid sequence of (GGS) n, S (GGS) n, or SGGS (SEQ ID NO: 131), wherein n is a number of 1-20 (e.g., 1, 2, 3, 4, 5, 6, 7, 8, 9, 10, 11, 12, 13, 14, 15, 16, 17, 18, 19, or 20). A peptide linker may for example, without being limited thereto, comprise or consist of an amino acid sequence according to SEQ ID NO: 132, or SEQ ID NO: 133, also referred to as XTEN linker, or SEQ ID NO: 134, also called GS-XTEN-GS linker (32aa), or SEQ ID NO: 135, also called GS-XTEN-GS linker (48aa). Peptide linkers are well known in the art (cf. e.g. Chen et al. Adv Drug Deliv Rev. 2013 October; 65 (10): 1357-69. doi: 10.1016/j.addr.2012.09.039).

A peptide linker may be about 2 to about 100 or more amino acids long, preferably about 2 to about 50 amino acids long, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids.

A linker for non-covalent binding may comprise or consist of two different parts that can non-covalently interact with each other in a specific manner, i.e. a binding pair. Non covalent binding may be achieved by any binding pair, such as affinity tags, biotin-streptavidin interaction or e.g. FRB-FKBP (Inobe and Nukina, 2016), allowing a specific interaction. Non-covalent binding may also be achieved by non-covalent protein-RNA interaction with the pegRNA. In case of non-covalent protein-RNA interaction with the pegRNA, the at least one Reverse transcriptase comprises at least one RNA-binding motif and/or domain (referred to as “RBD” herein) and the peg RNA comprises at least one binding sequence suitable for said at least one RBD (RBD binding sequence). Non-covalent binding may also be achieved by non-covalent protein-RNA interaction of the reverse transcriptase with the petRNA and the Cas12a enzyme with the crRNA, and non-covalent DNA-RNA interaction of the petRNA and the crRNA with the target site through complementary base pairing. An RBD of the present invention is capable of binding one or more RNA sequences (RBD binding sequences) in a sequence- and/or structure-specific manner, or at least in a partially in a sequence- and/or structure-specific manner. Systems of RBD and RBD binding sequence may for example be, without being limited thereto, phage base systems such as the MS2 MCP system or the BoxB Lamnda N system as known in the art. An RBD and the corresponding RBD binding sequence may for example, without being limited thereto, be selected from SEQ ID NOS: 156 and 157 or 158, 159 and 160, 161 and 162, 163 and 164, and/or 165 and 166.

In one embodiment according to the first aspect, the (iii) at least one linker linking the at least one nCas12a, or a catalytically active fragment thereof, and the at least one reverse transcriptase enzyme, or a catalytically active fragment thereof, comprises or consists of at least one RBD linked to the at least one reverse transcriptase, or active fragment thereof, and the at least one pegRNA comprises at least one corresponding RBD binding domain, optionally wherein said RBD binding domain is part (viii) of the pegRNA according to the second aspect.

In one embodiment of the first aspect, the (iii) at least one linker is an MS2 bacteriophage coat protein (MCP), and the pegRNA or the pegRNA system according to the second aspect comprises at least one MS2 loop sequence, optionally at the 5′ end and/or the 3′end of the pegRNA.

In one embodiment, the at least one Cas12a enzyme is a nCas12a comprising at least one mutation in the core lid domain conferring nickase activity, optionally wherein the at least one nCas12a comprises or consist of an amino acid sequence selected from SEQ ID NOs: 14 to 29, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, and/or wherein the at least one nCas12a comprises the core lid domain of any one of SEQ ID NOs: 14 to 29 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain.

The conformationally flexible portion of the lid domain following the catalytically active residue E925 (numbering according to LbCas12a of SEQ ID NO: 1) is highly conserved within all Cas12a orthologs. Mutants of this motif, called the “core lid domain” herein (cf. SEQ ID NO: 13 for the overall consensus sequence) were shown to be highly functional Cas12a-nickases having an intact catalytically active site (PCT/EP2023/055130). The core lid domain of LbCas12a as reference sequence (SEQ ID NO: 1) comprises the core lid domain as defined herein starting with position L927 and ending at position V942. The homologous positions in conserved Cas12a homologs/orthologs known to the skilled person and disclosed herein (e.g., SEQ ID NOS: 1 to 12) can be determined by the skilled person based on the information provided herein.

SEQ ID NO: 13 was identified as a core lid domain (PCT/EP2023/055130). This core lid domain corresponds to 927 to 942 according to SEQ ID NO: 1 (LbCas12a) as reference sequence and it was shown to represent a suitable consensus sequence or motif to characterize and identify Cas12 variants. Therefore, the skilled person can easily identify a Cas12a protein having a core lid domain based on the disclosure presented herein. The X positions in SEQ ID NO: 13 may correspond to the following sequences in a Cas12a wild-type enzyme in the various aspects and embodiments disclosed herein. X at position 2 of SEQ ID NO: 13 can be a N or S or an amino acid having a similar polarity, the Xaa at position 3 of SEQ ID NO: 13 can be F, H, or Y or an amino acid having a similar polarity, the X at position 7 of SEQ ID NO: 13 can be S, A, K, R, N, or an amino acid having a similar polarity, the X at position 8 of SEQ ID NO: 13 can be K or G, or an amino acid having a similar polarity, the X at position 10 of SEQ ID NO: 13 can be T, S, F, V, Q, or an amino acid having a similar polarity, the X at position 11 of SEQ ID NO: 13 can be G or K, or an amino acid having a similar polarity, the X at position 12 of SEQ ID NO: 13 can be I or V, or an amino acid having a similar polarity, the X at position 13 of SEQ ID NO: 13 can be present or absent, if present, it can be A, or an amino acid having a similar polarity, the X at position 15 of SEQ ID NO: 13 can be K, R, S, or an amino acid having a similar polarity, the X at position 16 of SEQ ID NO: 13 can be A, G, S, or an amino acid having a similar polarity, and the X at position 17 of SEQ ID NO: 13 can be V or I, or an amino acid having a similar polarity. Suitable combinations are, for example, K932G/N933G/S934A/R935G, and C931E (numbering according to LbCas12a of SEQ ID NO: 1).

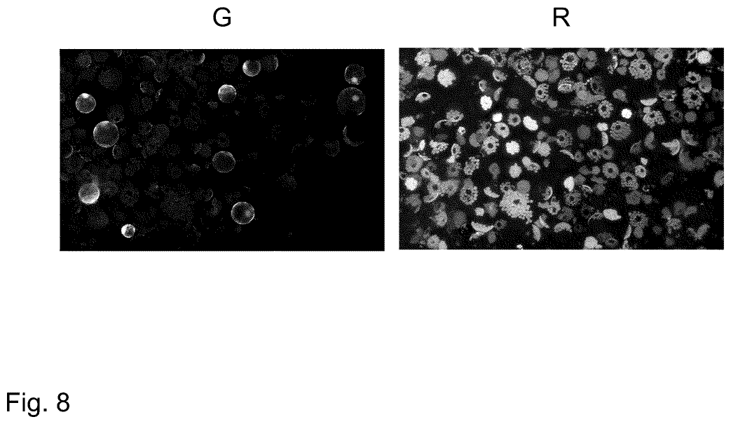

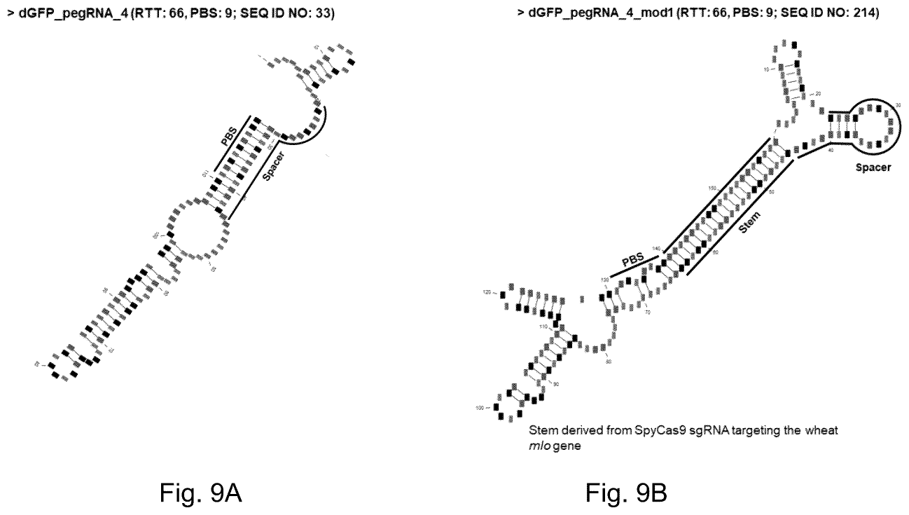

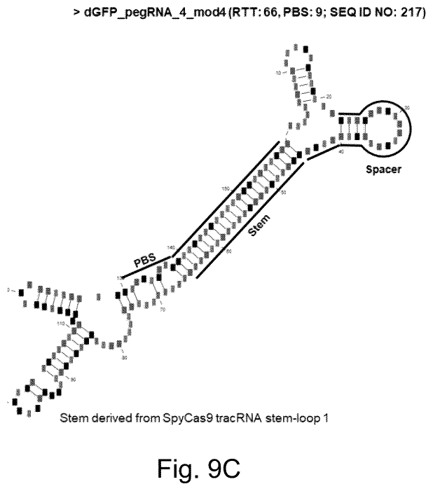

In certain embodiments according to the first, third or fourth aspect, a Cas12a enzyme as disclosed herein having nickase activity and comprising a flexible lid domain may also be selected from an ortholog of Cas12a having—in its natural environment—the same overall functionality as a Class 2 type V CRISPR nuclease and having the same overall fold and mechanistic action as Cas12a. Particularly, such an ortholog will have a lid domain dynamically opening and closing upon substrate binding exactly in a way as Cas12a (Stella al., et Cell, 2018, vol. 175 (7), https://doi.org/10.1016/j.cell.2018.10.045) so that also the lid domains of these Cas12a ortholog nickase effectors can be modified and used as disclosed herein. As shown in Zhang et al. for the Cas12a ortholog Cas12i (Nat Struct Mol Biol, (2020), 27 (11): 1069-1076, doi: 10.1038/s41594-020-0499-0; cf. Extended Suppl. Data FIG. 8), a lid domain seems to be conserved in Cas12a orthologs of class 2 type V CRISPR effectors so that the findings herein can be extended to a sub-motif within the core lid domain as defined herein.

In one embodiment according to the first, third or fourth aspect, the at least one mutation in the core lid domain is within positions 5 to 15 with reference to SEQ ID NO: 13.

X or Xaa positions as defined in SEQ ID NO: 13 may be present in similar polarity in another wild-type Cas12a ortholog or homolog. A “similar polarity” as used herein in this context means a polarity according to a standard polarity (that is, the distribution of electric charge) of the side chain of an amino acid, wherein a similar polarity implies that an amino acid residue at a given position may be exchanged against an amino acid within the same polarity group, wherein the polarity groups are selected from: Group I comprising nonpolar amino acids selected from glycine, alanine, valine, leucine, isoleucine, proline, phenylalanine, methionine, and tryptophan; Group II comprising polar, uncharged amino acids, being selected from amino acids serine, cysteine, threonine, tyrosine, asparagine, and glutamine; Group III comprising acidic amino acids selected from aspartic acid and glutamic acid; Group IV comprising basic amino acids selected from arginine, histidine, and lysine.

In one embodiment according to according to the first, third or fourth aspect, 1, 2, 3, 4, 5, 6, 7 or all 8 positions 6 to 13 with reference to SEQ ID NO: 13 may be deleted or have a point mutation or a combination thereof.

In one embodiment according to the first, third or fourth aspect, 1, 2, 3, 4, 5, 6, 7, 8, 9, 10 or all 11 positions 5 to 15 with reference to SEQ ID NO: 13 may be deleted, or they may have a point mutation or a combination thereof.

In one embodiment according to the first, third or fourth aspect, 1, 2, 3, 4, 5, 6, 7, 8, 9, 10, 11, 12, 13, 14, 15, 16 or all 17 positions of the core lid domain with reference to SEQ ID NO: 13 are deleted or have a point mutation or a combination thereof.

In certain embodiments according to the first, third or fourth aspect, the at least one point mutation in the core lid domain according to the present invention may comprise or consist of, at least three point mutations of three positions within the core lid domain, preferably wherein the mutation comprises or consists of (a) a first point mutation at a first position or a first stretch of at least two point mutations at consecutive positions, (b) a second point mutation at a second position or a second stretch of at least two point mutations at consecutive positions, (c) a third point mutation at a third position or a third stretch of at least two point mutations at consecutive positions, and optionally (d) at least one further point mutation at at least one further position or at least one further stretch of at least two point mutations at consecutive positions, wherein the first position or first stretch of positions, the second position or second stretch of positions, the third position or third stretch of positions, and optionally the at least one further position or at least one further stretch of positions are not in consecutive order to each other.

In one embodiment according to the first, third or fourth aspect, the at least one point mutation in the core lid domain according to the present invention may comprise or consist of one deletion at a first position or at least two deletions of a first stretch of consecutive positions, and a second deletion of a second position, or a second stretch of consecutive deletions, and optionally at least one further deletion of least one further position, or at least one further stretch of consecutive deletions, wherein the position of the second deletion or the second stretch of deletions is not in consecutive order with the first deletion or first stretch of consecutive deletions, and optionally wherein the positions of the at least one further deletion or the at least one further stretch of deletions is not in consecutive order with the first position or the first stretch of consecutive positions and the second position or second stretch of consecutive deletions.

In certain embodiments according to the first, third or fourth aspect, the at least one point mutation in the core lid domain may comprise or consist of (a) one deletion of one position, two deletions, three deletions, four deletions, five deletions, six deletions, seven deletions, eight deletions, or nine deletions, or in certain embodiments more than nine deletions, of a stretch of consecutive positions, preferably wherein the position or stretch of positions is within positions 5 to 15 with reference to SEQ ID NO: 13, (optionally) in combination with 1, 2, 3, 4, 5, 6, 7, 8, 9, 10, 11, 12, 13, 14, 15, or 16 point mutations, wherein some or all positions of the point mutations may be in consecutive order and may optionally be in consecutive order with the position or stretch of positions of the deletion(s); or (b) a first deletion of a first position or, a first stretch of two, three, four, or five, consecutive deletions of a first stretch of positions, preferably wherein the first position or first stretch of positions is within positions 5 to 15 with reference to SEQ ID NO: 13, and a second deletion of a second position, preferably at least one second stretch of (in total) two, three, four, or five, consecutive deletions of at least one second stretch of positions, preferably wherein the second position or the at least one second stretch of positions is within positions 5 to 15 with reference to SEQ ID NO: 13, optionally wherein the second deletion or at least one second stretch of consecutive deletions is not in consecutive order with the first deletion or first stretch of consecutive deletions, optionally in combination with 1, 2, 3, 4, 5, 6, 7, 8, 9, 10, 11, 12, 13, 14 or 15 point mutations, wherein some or all positions of the point mutations may be in consecutive order and may optionally be in consecutive order with the position or stretch of positions of the deletion of any of the deletions.

In another embodiment according to the first, third or fourth aspect, the at least three point mutations in three consecutive amino acids may be positioned within positions 2 to 16 with reference to SEQ ID NO: 13, and/or wherein the deletion is a deletion of at least two, at least three, at least four, at least five, at least six, at least seven, at least eight, at least nine, at least ten, at least eleven, at least twelve, at least thirteen, at least fourteen, at least fifteen, at least sixteen, or at least seventeen consecutive positions within the core lid domain.

In another embodiment according to the first, third or fourth aspect, the mutation may be a deletion of at least four, at least five, at least six, at least seven, or at least all eight positions 6 to 13 with reference to SEQ ID NO: 13, and/or wherein the mutation is at least a mutation of three point mutations of three consecutive positions within positions 6 to 13 with reference to SEQ ID NO: 13.

In one embodiment according to the first, third or fourth aspect, at least one mutation, preferably exactly one, mutation introduced into the core lid domain motif may insert a Cys residue instead of the wild-type amino acid, wherein the at least one inserted Cys residue, preferably the exactly one inserted Cys residue, may be introduced in combination with one or more other point mutation(s) and/or deletion(s) according to the present invention. Without wishing to be bound by theory, it is assumed that the introduction of an additional cysteine residue can favorably change the dynamic lid domain reassortment upon binding of the DNA target site so that the nickase activity is promoted.

In one embodiment, the Cas12a prime editor comprises or consist of an amino acid sequence selected from SEQ ID NO: 65 to 69, 213 or 268 to 277, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In one embodiment, according to the first, third or fourth aspect, the at least one Cas12a enzyme is an nCas12a comprising at least one mutation in the core lid domain conferring nickase activity as defined above; and the at least one pegRNA is a linear pegRNA as defined in the second aspect.

In another embodiment, according to the first, third or fourth aspect, the at least one Cas12a enzyme is an nCas12a comprising at least one mutation in the core lid domain conferring nickase activity as defined above; and the at least one pegRNA or the pegRNA system is or comprises a circular pegRNA or a circular petRNA as defined in the second aspect. This embodiment may be particularly suitable for certain therapeutic and plant editing purposes, as the highly active and reliable core lid domain nCas12a enzyme having a low off-target rate and excellent site-directed nickases activity can be specifically combined with a compact and stable circular pegRNA to allow efficient in situ genome editing in a higher eukaryotic cell of interest.

In a second aspect there is provided, a prime editing guide RNA (pegRNA) or a pegRNA system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides.

In one embodiment, the pegRNA system comprises a) a pegRNA comprising parts (i), (ii), optionally (iii), (iv), (v), and optionally (vi); or b) a crispr RNA (crRNA) and a prime editing template RNA (petRNA), wherein 1) said crRNA comprises parts (i). (ii) and optionally (vi); and 2) said petRNA comprises optionally (iii), (iv) and (v).

In yet another embodiment, the pegRNA system comprises a crRNA and a petRNA, wherein the petRNA is linear.

In one embodiment, the pegRNA system further comprises (vii) optionally at least one 3′ linker sequence; and (viii) at least one structured motif, including at least one hairpin, including at least one MS2 stem loop, preferably at least two MS2 stem loops, and/or at least one pseudoknot.

In certain embodiments, the pegRNA and/or the petRNA may thus comprise at least ine structured motif, preferably at least one, more preferably at least two MS2 loops.

In one embodiment the pegRNA system comprises the parts (i), (ii), optionally (iii), (iv), (V), optionally (vi), and, if present, optionally (vii) and (viii) as defined above, in the following order in 5′ to 3′ direction: a) (i), (ii), optionally (iii), (iv), (v) and optionally (vi); or b) (iv), (v), (iii), (i), (ii), optionally (vi); or c) (i), (ii), optionally (iii), (iv), (v), (vii), (viii) and optionally (vi), or b) the crRNA comprises in a 5′ to 3′ direction parts (i), (ii) and optionally (vi) as defined above; and the petRNA comprises in a 5′ to 3′ direction parts (viii), optionally (iii), (iv), (v), optionally (iii), and (viii) as defined above, wherein the parts are preferably linked consecutively.

In one embodiment, the (viii) at least one structured motif, including at least one hairpin, including at least one MS2 stem loop, preferably at least two MS2 stem loops, and/or at least one pseudoknot may be located between the spacer and the RTT-PBS part of a pegRNA (cf. Example 7).

In one embodiment, the pegRNA system further comprises a nicking crRNA, said nicking crRNA comprising a CRISPR-Cas class 2 type V scaffold sequence; a spacer sequence, and optionally a second scaffold sequence, wherein the target site of the spacer sequence of said nicking crRNA is in the vicinity of and at the opposite strand of the target site of the spacer sequence of said pegRNA or said petRNA.

As understood herein, a pegRNA system may thus represent one linear or circular pegRNA as described herein, or it may represent a split non-covalently associated pegRNA optionally including a linear or circular petRNA and crRNA.

In one specific embodiment, the reverse transcriptatse may thus be is non-covalently linked to an MS2-tagged pegRNA or petRNA. The pegRNA will form an RNP (ribonucleoparticle) with the Cas12a nickases via a non-covalent bond. The petRNAs will usually not be directly bound by Cas12a, but they may bind to the target site through complementary base pairing.

Recruiting petRNAs to a Cas12a-MCP fusion protein does, however, further increase editing as demonstrated by the present inventors (cf. Example 9).

In yet another embodiment of the second aspect there is provided a prime editing guide RNA (pegRNA) system comprising the parts (i) to (v) as defined above and additionally comprising a part (vi) being a second scaffold sequence, optionally being the same as the first scaffold sequence, wherein the primer binding site is complementary to the non-target strand. This second scaffold sequence can help to boost Cas12a gRNA activitiy in certain systems, for example, when using transfected plasmid DNA.

The scaffold sequence, may be a scaffold sequence of a wild-type CRISPR-Cas class 2 type V CRISPR-Cas effector or may be a synthetic scaffold sequence designed to function in a class 2 type V CRISPR-Cas system (cf. e.g. Jedrzejczyk et al., Sci Rep. 2022 Jul. 16; 12 (1): 12193. doi: 10.1038/s41598-022-15388-z.). The scaffold sequence will typically form a pseudoknot structure, sometimes referred to as the “handle” for the CRISPR-Cas enzyme binding. The scaffold sequence may be a single RNA molecule or may be two separate RNA molecules that can bind to each other through base pairing. The scaffold sequence may for instance, without being limited thereto, comprise or consist of a Cas12a scaffold sequence, such as, but not limited to, a sequence according to SEQ ID NO: 91 or a sequence having at least 90%, 91%, 92%, 93%, 94%, 95%, 96%, 97%, 98% or at least 99% sequence identity thereto. Various scaffold sequences suitable for different CRISPR-Cas class 2 type V effectors are known in the art and may be used as part of the present invention. Naturally, the scaffold sequence has to be selected to be suitable for the desired CRISPR-Cas class 2 type V effector or vice versa. The scaffold sequence may be about 10 to about 100 nucleotides, preferably about 10 to about 50 nucleotides, more preferably about 15 to about 30 nucleotides, most preferably about 15 to about 25 nucleotides, long.

The spacer sequence is complementary to the desired target site, wherein the complementarity may be a full complementarity or a partial complementarity, for example 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementarity. The spacer may, for example comprise 1, 2, 3, 4 or 5 mismatches in relation to the desired target site. Without wishing to be bound by theory, it is hypothesized that mutants with reduced flexibility, as for instance achieved by substitutions with proline, together with target DNA mismatches are sufficient to limit conformational changes and block target strand cleavage. Preferably, the spacer comprises at least 15 or at least 16 or at least 17 or at least 18 nucleotides with 100% complementarity to the desired target site. In certain embodiments, the spacer sequence may be 12 to about 30 nucleotides, or 15 to about 30 nucleotides, or 16 to about 28 nucleotides or 17 to about 28, or 18 to about 25 nucleotides long.

A pegRNA linker sequence, according to part (iii) of the second aspect, may about 5 to about 100 nucleotides long, for instance or about 10 to about 30, or about 30 to about 50, or about 50 to about 70 or about 70 to about 90 nucleotides long. An example linker sequence is disclosed as SEQ ID NO: 94.

The second scaffold sequence (vi), may be about 10 to about 100 nucleotides, preferably about 10 to about 50 nucleotides, more preferably about 15 to about 30 nucleotides, most preferably about 15 to about 25 nucleotides, long. Like (i) the (first) scaffold sequence, (vi) the second scaffold sequence, if present, may likewise be a scaffold sequence of a wild-type CRISPR-Cas class 2 type V CRISPR-Cas effector or synthetic scaffold sequence. The second scaffold sequence, may be identical to (i) the (first) scaffold sequence, thus forming direct repeats.

In some embodiments, the second scaffold sequence may, instead of a CRISPR-Cas scaffold, be any structured motif, including at least one hairpin and/or pseudoknot sequence and similar structural motifs, as known in the art, for example a sequence selected from SEQ ID NOs: 93 to 129 or a sequence having at least 90%, 91%, 92%, 93%, 94%, 95%, 96%, 97%, 98% or at least 99% sequence identity thereto.

In one embodiment, the pegRNA or pegRNA system further comprises (vii) optionally at least one 3′ linker sequence; and (viii) at least one structured motif, including at least one hairpin and/or at least one pseudoknot. The structured motif of part (viii) may be identical to (vi) the second scaffold sequence, if present, or may differ from (vi) the second scaffold sequence. For Cas9 prime editing systems, it could be shown that such 3′ structures can enhance prime editing (Nat Biotechnol. 2022 March; 40 (3): 402-410. doi: 10.1038/s41587-021-01039-7). Such hairpins may also be used to enhance prime editing using the class 2 type V CRISPR-Cas prime editing system of the present invention. The structured motif, if present, is located at the 3′ end of the pegRNA and may be connected to the remaining pegRNA sequence via (vii) the 3′ linker sequence, wherein in embodiments in which both (vi) a second scaffold sequence and (viii) a structured motif are present, (vi) the a second scaffold sequence may be located even 3′ of (viii) the structured motif (the a second scaffold sequence thus forming the 3′ end instead), or located 5′ of (viii) the structured motif and if (vii) a 3′ linker sequence is present, between (viii) the structured motif and (vii) the 3′ linker sequence, or 5′ of (vii) the 3′ linker sequence.

The pegRNA 3′ linker sequence, may be 1 to about 100 nucleotide long, for instance or about 1 to about 30, or about 30 to about 50, or about 50 to about 70 or about 70 to about 90 nucleotides long. In some embodiments, the 3′ linker sequence is 1 to about 20 or 5 to about 15 nucleotides long. The 3′ linker sequence may be identical to the linker sequence of part (iii), if present, or different. A 3′ linker sequence may for example, without being limited thereto be TCTCTCTC.

The hairpin sequence may for example, without being limited thereto, be selected from a sequence comprising or consisting of a sequence according to SEQ ID NO: 93 to 129 or a sequence having at least 90%, 91%, 92%, 93%, 94%, 95%, 96%, 97%, 98% or at least 99% sequence identity thereto.

In certain embodiments, the (viii) at least one hairpin comprises one or two or more RBD binding sequences, such as one or two or more boxB or MS2 stem loops. In another embodiment, one or two or more RBD binding sequences, such as one or two or more boxB or MS2 stem loops are located at the 5′ end of the pegRNA, optionally linked via a linker sequence as disclosed herein. RBD-binding sequences may be used for non-covalently binding of the reverse transcriptase, via the pegRNA to the CRISPR-Cas effector or may be used for non-covalent binding of further proteins, domains or functional fragments thereof as disclosed herein or known in the art.

In certain embodiments, the reverse transcriptase (RT) will thus be covalently linked to the CRISPR-Cas effector, preferably a Cas12a effector, in other embodiments, the RT and the CRISPR-Cas effector, preferably the Cas12a effector can be non-covalently associated.

In one embodiment, the (viii) at least one hairpin comprises one or two or more MS2 stem-loops, optionally two or more copies of the same MS2 stem loop. In another embodiment, the pegRNA comprises one or two or more MS2 stem loops, optionally two or more copies of the same MS2 stem loop, located at the 5′ end of the pegRNA, optionally linked via a linker sequence as disclosed herein.

An MS2 stem-loop may for example, without being limited thereto, be a sequence selected from SEQ ID NO: 126, or 136-141, or a sequence having at least 90%, 91%, 92%, 93%, 94%, 95%, 96%, 97%, 98% or at least 99% sequence identity thereto.

In one embodiment, the pegRNA or pegRNA system comprises two MS2 stem loops, for example without being limited thereto, a sequence selected from SEQ ID NO: 142 to 145, or a sequence having at least 90%, 91%, 92%, 93%, 94%, 95%, 96%, 97%, 98% or at least 99% sequence identity thereto, wherein said two MS2 stem loops may be present at the 5′ end or the 3′ end of the pegRNA.

In one embodiment, the pegRNA is a 3′ extended pegRNA as disclosed herein, wherein one or two or more MS2 stem-loops, optionally comprising at least one sequence selected from SEQ ID NOS: 126, or 136-145, or a sequence having at least 90%, 91%, 92%, 93%, 94%, 95%, 96%, 97%, 98% or at least 99% sequence identity thereto, linked to the 5′ end of the scaffold sequence, optionally via a linker sequence as disclosed herein.

In one embodiment, the pegRNA is a 5′ extended pegRNA as disclosed herein, wherein one or two or more MS2 stem-loops, optionally comprising at least one sequence selected from SEQ ID NOs: 126, or 136-145, or a sequence having at least 90%, 91%, 92%, 93%, 94%, 95%, 96%, 97%, 98% or at least 99% sequence identity thereto, linked to the 3′ end of the scaffold sequence, optionally via a linker sequence as disclosed herein.

In one embodiment, the pegRNA or petRNA is a 5′ extended pegRNA or petRNA as disclosed herein, wherein one or two or more MS2 stem-loops, optionally comprising at least one sequence selected from SEQ ID NOs: 126, or 136-145, or a sequence having at least 90%, 91%, 92%, 93%, 94%, 95%, 96%, 97%, 98% or at least 99% sequence identity thereto, linked to the 5′ end of the pegRNA or petRNA, optionally via a linker sequence as disclosed herein.

In one embodiment, (iv) the reverse transcriptase template has a length of 25 to 140 nucleotides, or 30 to 120 nucleotides, or 40 to 100 nucleotides, or 50 to 90 nucleotides, or 60 to 90 nucleotides.

The RTT comprises the desired edited sequence, such as the insertion, deletion and/or exchange of at least one nucleotide relative to the nucleic acid sequence of interest, to which the nucleic acid sequence of interest is to be modified. In certain embodiment, the RTT will, in addition to the desired modification, comprise 1, 2, 3, 4, 5, or more silent mutations. The introduction of silent mutation into the RTT may be used to even further improve class 2 type V CRISPR-Cas prime editing according to the present invention. The strategic integration of silent mutations near the intended edit can enhance the Prime Editing efficiency. Strategies for such inclusion of silent mutations are known to the skilled person for Cas9-based systems (Li et al., 2022, Nat Commun 13, 1669, https://doi.org/10.1038/s41467-022-29339-9; Chen et al., 2021, Cell 184 (22): 5635-5652.e29. doi: 10.1016/j.cell.2021.09.018) and can be applied for the Prime Editing system of the present invention as shown in Example 5 and Table 8.

In one embodiment, (v) the primer binding site has a length of 6 to 40 nucleotides, or 6 to 30 nucleotides, or 7 to 20 nucleotides, or 7 to 15, nucleotides, or 9 to 12 nucleotides.

The primer binding site (PBS) is designed to be complementary to the nucleic acid sequence at the free 3′ end resulting from the double-stranded or, preferably, single-stranded cut from the CRISPR-Cas enzyme. Thus, the free 3′ end serves as a primer for the reverse transcript which will extend over the RTT. Typically, the PBS has 100% complementarity to the sequence at said free 3′ end, but in some embodiments, the complementarity may be partial, for example 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementarity.

For a pegRNA or pegRNA system of the present invention, the parts (i) and (ii) of the pegRNA, as defined above, are always present in that order ((i)-(ii) in 5′ to 3′ direction, preferably linked consecutively (i.e. without a different sequence in between). Likewise, the parts (iv) and (v) are always present in that order ((iv)-(v)) in 5′ to 3′ direction, preferably linked consecutively. The parts (iv)-(v) may be present 3′ to the parts (i)-(ii), herein referred to as “3′ extended guide RNA”, or the parts (iv)-(v) may be present 5′ to the parts (i)-(ii) (“5′ extended guide RNA”). The parts (iv)-(v) may be linked to the parts (i)-(ii), consecutively or may be linked via (iii) the linker sequence.

In preferred embodiments, the pegRNA is a 3′ extended guide RNA, with (iv)-(v) be present 3′ to the parts (i)-(ii), optionally linked via (iii) a linker sequence.

In one embodiment the pegRNA or pegRNA system comprises the parts (i), (ii), optionally (iii), (iv), (v), optionally (vi), and, if present, optionally (vii) and (viii) as defined above, in the following order in 5′ to 3′ direction: a) (i), (ii), optionally (iii), (iv), (v) and optionally (vi); or b) (iv), (v), (iii), (i), (ii), optionally (vi); or c) (i), (ii), optionally (iii), (iv), (v), (vii), (viii) and optionally (vi), wherein the parts are preferably linked consecutively.

In one embodiment the peg RNA is a linear pegRNA. In another embodiment, the 5′ end and the 3′ end of the pegRNA are linked, optionally via a circularization linker sequence, thus forming a circular pegRNA. All specifications above relating to the 5′ end or the 3′ end of the pegRNA or the 5′-3′ direction within the pegRNA equally apply to embodiments of circular pegRNAs with the proviso that the parts referred to above as 5′ end and 3′ end are additionally linked to form the circularization, even if due to the circularization these parts in effect do not form ends of the pegRNA. Means of circularizing RNA, such as ligase-mediated circularization, are well known to the skilled person.

Recently, Cas12a mediated Prime Editing was demonstrated in human cells using a circular pegRNA design. (Liang et al., Nat Biotechnol (2024). https://doi.org/10.1038/s41587-023-02095-x). However, classical linear pegRNA design was reported as failing to show effective Cas12a Prime Editing. In contrast, the present invention provides a pegRNA design as described in the second aspect that is superior to the design as used in Liang et al. supra as it also allows effective Cas12a Prime editing using linear pegRNA constructs in higher eukaryotic cell, as the linear pegRNA design of the present invention allows for a stable construct with a sophisticated architecture allowing interaction with all necessary enzymes and the target DNA in a Prime Editing bubble. In particular, the present invention comprises pegRNA architectures that include the strategic combination of long RTTs with a short PBSs, whereas the design of Liang et al. supra only envisions relatively short RTTs with short PBSs. Without wishing to be bound by theory, a long RTT may ensure sufficient flexibility in the secondary structure, while the specific combination with a short PBS may prevent inhibitory interactions between the spacer and the PBS of the pegRNA. Therefore, the linear pegRNAs of the present invention are particularly suitable for any kind of robust prime editing using a nuclease or nickases, specifically a Cas12a nickases, specifically in a higher eukaryotic cell, as the pegRNAs can be conveniently designed and produced ex vivo for later introduction and in vivo on suitable and safe constructs.

Throughout the various aspects and embodiments herein, for prime editing of plant cells the pegRNA may be a linear pegRNA as defined in the second aspect.

In a third aspect, there is provided a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof preferably being independently selected from Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP).

In one embodiment, the Cas12a prime editor comprises the reverse transcriptase protein in trans, and further comprises an RNA binding factor, preferably the N-terminal domain of La or of MCP, more preferably wherein the Cas12a prime editor comprises the N-terminal domain of La, optionally comprising the reverse transcriptase protein as defined above comprising an MCP, and the pegRNA system as defined above, and, optionally the nicking crRNA as defined above, wherein a) the crRNA comprises in a 5′ to 3′ direction (i) a CRISPR-Cas class 2 type V scaffold sequence, (ii) a spacer sequence and optionally (vi) a second scaffold sequence, optionally being the same as the first scaffold sequence; b) a prime editing template RNA the (petRNA) comprising in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

In one embodiment, the at least one nCas12a comprises at least one mutation in the core lid domain conferring nickase activity, optionally wherein the at least one nCas12a comprises or consist of an amino acid sequence selected from SEQ ID NOs: 14 to 29, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, and/or wherein the at least one nCas12a comprises the core lid domain of any one of SEQ ID NOs: 14 to 29 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain.

In one embodiment, the Cas12a prime editor comprises or consists of an amino acid sequence selected from SEQ ID NO: 65 to 69, 213 or 268 to 277, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In one embodiment according to the first or third aspect, the (iii) at least one linker linking the at least one nCas12a, or a catalytically active fragment thereof, and the at least one reverse transcriptase enzyme, or a catalytically active fragment thereof, comprises or consists of at least one RBD linked to the at least one reverse transcriptase, or active fragment thereof. A suitable pegRNA or pegRNA system will comprise at least one corresponding RBD binding sequence, for example said RBD binding sequence may be part (viii) of the pegRNA or pegRNA system according to the second aspect.

In one embodiment of the first or third aspect, the (iii) at least one linker is an MS2 bacteriophage coat protein (MCP). A suitable pegRNA or pegRNA system comprises at least one MS2 loop sequence, for example as part (viii) of the pegRNA or pegRNA system according to the second aspect.

In a fourth aspect, there is provided a complex comprising (a) at least one prime editing guide RNA (pegRNA) or pegRNA system as defined in the second aspect; and (b) at least one type 2 class V Cas enzyme, preferably having nickase activity, more preferably having non-target strand (NTS) nickase activity, or an active fragment thereof; optionally comprising an organellar localization signal.

In one embodiment, the at least one type 2 class V Cas enzyme is at least one Cas12 enzyme.

In another embodiment, the at least one type 2 class V Cas enzyme is at least one Cas12a enzyme.

In a fifth aspect, there is provided a nucleic acid molecule or more than one nucleic acid molecules encoding the prime editing complex of the first aspect; and/or the pegRNA or the pegRNA system of the second aspect; and/or the Cas12a prime editor of the third aspect; and/or the complex of the fourth aspect.

In some embodiments, the nucleic acid sequence is codon-optimized for a fungal cell, including a yeast cell, or a prokaryotic cell, including a bacterial cell or an archaeal cell, or a plant cell, including an algal cell, or an animal cell, including a rodent or human cell, in particular for a fungal cell, a prokaryotic, a plant cell, or an animal cell disclosed herein.

The nucleic acid sequence may be operably linked to a promoter sequence and/or a terminator sequence that is suitable for a desired target cell in which the provided nucleic acid sequence might be expressed.

In a sixth aspect, there is provided an expression vector or construct or more than one expression vector or constructs comprising the one or more nucleic acid molecules of the fifth aspect.

Expression constructs or vectors suitable for a multitude of different target cells as well as means and methods to design such expression constructs or vectors, including a large variety of suitable markers, are well known to the skilled person. Naturally, the promoter and terminator have to be selected depending on the target cell or the desired in vitro expression systems and is different for RNA expression compared to protein expression. The skilled person is well aware of different promoters and terminators suitable for a variety of different target cells and/or suitable for in vitro expression for both protein expression as well as RNA expression.

Non-limiting examples of classes of expression constructs and vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector.

In a seventh aspect, there is provided a cell comprising the prime editing complex of the first aspect; and/or the pegRNA or the pegRNA system of the second aspect and/or the Cas12a prime editor of the third aspect; and/or the complex of the fourth aspect; and/or the nucleic acid molecule or more than one nucleic acid molecules of the fifth aspect; and/or the expression vector or construct or more than one expression vector or constructs of the sixth aspect.

In one embodiment, the cell is a eukaryotic cell or a prokaryotic cell, including a bacterial or an archaeal cell.

In one embodiment, the cell is a plant cell, including an algal cell, preferably wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants may be independently selected from Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Other preferred plants may be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In another embodiment, the cell is an insect, poultry, fish or crustacea cell, or a mammalian cell, preferably wherein the cell is a mammalian cell being selected from a cell originating from a non-human primate, bovine, porcine, rodent, including mouse, or human cell.

In another embodiment, the cell is a fungal cell, including a yeast cell, preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile.

In another embodiment, the cell is a prokaryotic cell, including a Gram-positive, Gram negative or Gram-variable bacterial cell, preferably Gram-negative bacterial cells, or an archaeal cell, preferably wherein the prokaryotic cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

In one embodiment according to the various aspects as disclosed herein, the cell may be a eukaryotic cell or a prokaryotic cell, wherein the cell is selected from a cell originating from Rhodococcus rhodochrous, Aerococcus sp., Ashbya gossypii, Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica, wherein the cell is prefererably selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae or Yarrowia lipolytica.

In another embodiment, the cell may be a eukaryotic cell or a prokaryotic cell, wherein the cell is selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica, Phakopsora spec, e.g. Phakopsora pachyrhizi, Zymoseptoria spec, e.g. Zymoseptoria tritici, Septoria, Mycosphaerella, Phythopthora spec., e.g. Phytopthora infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, or Ashbya.

In an eighth aspect, there is be provided a kit comprising the prime editing complex of the first aspect; and/or the pegRNA or the pegRNA system of the second aspect; and/or the Cas12a prime editor of the third aspect; and/or the complex of the fourth aspect; and/or the nucleic acid molecule or more than one nucleic acid molecules of the fifth aspect; and/or the expression vector or construct or more than one expression vector or constructs of the sixth aspect; and/or the cell of the seventh aspect; further comprising a set of reagents; optionally comprising particles, vesicles, or at least one viral vector, or Agrobacterium vector for assisting delivery, wherein said particles comprise a lipid, including lipid nanoparticles, a sugar, a metal or a polypeptide, or a combination thereof, or wherein said vesicles comprise exosomes or liposomes.

In a ninth aspect, there is provided a method for modifying at least one nucleic acid sequence of interest in at least one nucleic acid molecule of at least one cell or construct at or near at least one target site, the method comprising: (a) providing at least one cell or construct comprising the nucleic acid sequence of interest to be modified; (b) providing and/or introducing (b-i) at least one pegRNA or the pegRNA system as defined in the second aspect, or at least one nucleic acid molecule or expression vector or construct encoding the same, and (b-ii) at least one Cas12a prime editor, as defined in the first or third aspect; or at least one nucleic acid molecule or expression vector or construct encoding the same; optionally allowing complex formation of (b-i) the at least one pegRNA or crRNA and (b-ii) the at least one Cas12 prime editor before the provision and/or introduction; or providing and/or introducing: (b-iii) at least one prime editor complex as defined in the first aspect, or at least one nucleic acid molecule or expression vector or construct encoding the same; or providing and/or introducing (b-iv) at least one complex as defined in the fourth aspect, or at least one nucleic acid molecule or expression vector or construct encoding the same, and at least one reverse transcriptase as defined in the first or third aspect, or at least one or at least one nucleic acid molecule or expression vector or construct encoding the same; (c) allowing the modification of at least one nucleic acid sequence of interest by (b-i) the at least one pegRNA or the pegRNA system and (b-ii) the at least one Cas12 prime editor; or by (b-iii) the at least one prime editor complex; or by (b-iv) the at least one complex as defined in the fourth aspect and the at least one reverse transcriptase as defined in the first or third aspect; (d) optionally, obtaining at least one edited cell or construct comprising a modification at least one nucleic acid sequence of interest at or near a target site; optionally, where the method comprises the following step: (e) regenerating at least one population of edited cells, tissues, organs, materials or whole organisms from the at least one edited cell or construct; optionally wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In a ninth aspect, there is provided a for producing an edited cell, tissue, and/or organism, the method comprising: (a) providing at least one cell, tissue, and/or organism comprising at least one nucleic acid sequence of interest in at least one nucleic acid molecule at or near at least one target site; (b) introducing (b-i) at least one pegRNA or the pegRNA system as defined in the second aspect, or at least one nucleic acid molecule or expression vector or construct encoding the same, and (b-ii) at least one Cas12a prime editor, as defined in the first or third aspect; or at least one nucleic acid molecule or expression vector or construct encoding the same; optionally allowing complex formation of (b-i) the at least one pegRNA or crRNA and (b-ii) the at least one Cas12 prime editor before the provision and/or introduction; or introducing: (b-iii) at least one prime editor complex as defined in the first aspect, or at least one nucleic acid molecule or expression vector or construct encoding the same; or providing (b-iv) at least one complex as defined in the fourth aspect, or at least one nucleic acid molecule or expression vector or construct encoding the same, and at least one reverse transcriptase as defined in the first or third aspect, or at least one or at least one nucleic acid molecule or expression vector or construct encoding the same; (c) allowing the modification of at least one nucleic acid sequence of interest by (b-i) the at least one pegRNA or the pegRNA system and (b-ii) the at least one Cas12 prime editor; or by (b-iii) the at least one prime editor complex; or by (b-iv) the at least one complex as defined in the fourth aspect and the at least one reverse transcriptase as defined in the first or third aspect; (d) obtaining at least one edited cell or construct comprising a modification at at least one nucleic acid sequence of interest at or near a target site; optionally, where the method comprises the following step: (e) regenerating at least one population of edited cells, tissues, organs, materials or whole organisms from the at least one edited cell or construct; optionally wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In certain embodiments, the method of the ninth or tenth aspect of the present invention does not lead to the introduction of a DSB in the genomic locus of interest, which is achieved by the outstanding specific nickase activity (and the lack of the wild-type DSB activity) of the nCas12a variants as disclosed herein.

In one embodiment of the ninth or tenth aspect, the method is performed in vitro or in vivo and/or ex vivo.

Throughout the various embodiments, the introduction into a cell according to step (b) of the ninth or tenth aspect may be achieved by any suitable method known in the art. The skilled person is well aware that a variety of different transformation or transfection (used interchangeably herein) techniques depending on the desired target cell. Introduction may comprise methods, such as but not limited to calcium-phosphate-mediated transfection, cationic-polymer-mediated transfection, liposome-mediated transfection, PEG-mediated transfection, dendrimer transfection, heat shock transfection, magnetofection, electroporation, particle, including nanoparticle, uptake or bombardment, or microinjection.

In embodiments of the ninth or tenth aspect in which the cell is a plant cell, introduction into the plant cell may be a method such as, but not limited to, particle bombardment, particle uptake, whiskers mediated transformation, Agrobacterium transformation, including Agrobacterium-mediated introduction of virus-based vectors, PEG-mediated transformation, liposome-mediated transformation, electroporation, cell-penetrating peptides, microinjection or viral-vector-mediated introduction. As the skilled person is well aware, for some introduction techniques, for example PEG-mediated transformation, liposome-mediated transformation, electroporation or cell-penetrating peptides, the plant cell wall may be removed to produce protoplasts prior to the introduction. In embodiments comprising introduction into at least one protoplast, step (e) of the method of the tenth aspect may comprise regeneration from the at least one protoplast.

In embodiments of the ninth or tenth aspect, in which the cell is a fungal cell, including a yeast cell, introduction into the fungal cell, including a yeast cell, may comprise partial or complete digestion of the cell wall and/or may comprise protoplast transformation.

In some embodiments, the introduction comprises nuclear transformation. In some embodiments, the introduction comprises plastid transformation, such as chloroplast or mitochondrial transformation.

The at least one Cas12a prime editor, or the at least one Cas enzyme, and the at least one pegRNA or the pegRNA system may be introduced/provided (in) to the at least one cell or construct separately or together, optionally as a prime editor complex. A prime editor complex may be pre-formed before introduction/provision by incubation in any suitable buffer.

The at least one Cas12a prime editor, or the at least one Cas enzyme, and the at least one pegRNA or the pegRNA system may be introduced/provided directly or as one or more nucleic acid molecules, vectors and/or expression constructs encoding the same, wherein the at least one Cas12a prime editor, or at least one Cas enzyme, and the at least one pegRNA may be encoded on the same nucleic acid molecule, vector and/or expression construct or on different nucleic acid molecules, vectors and/or expression constructs.

In certain embodiments, the method of the ninth or tenth aspect may further comprise during step b) and/or step c) the provision and or introduction of at least one Mismatch repair inhibitor.

In certain embodiments, the method of the ninth or tenth aspect may further comprise during step b) and/or step c) the provision and or introduction of at least one 5′ flap endonuclease, such as Fen1.

In one embodiment of the ninth or tenth aspect, the cell or construct is or originates from a prokaryotic cell, including a bacterial or an archaeal cell, or a eukaryotic cell.

In one embodiment of the ninth or tenth aspect, the cell is a plant cell, including an algal cell, preferably wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants may be independently selected from Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Other preferred plants may be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In embodiments of the ninth or tenth aspect in which the at least one cell is at least one plant cell, the incubation of said at least one plant cell after and/or during the introduction step (b) according to the method of the tenth aspect may be performed at a temperature of about 22 to 37° C., preferably of about 24 to 32° C., more preferably of about 25 to about 30° C., more preferably of about 26 to about 30° C., most preferably of about 27 to about 29 C.

In another embodiment of the ninth or tenth aspect, the cell is an insect, poultry, fish or crustacea cell, or a mammalian cell, preferably wherein the cell is a mammalian cell being selected from a cell originating from a non-human primate, bovine, porcine, rodent, including mouse, or human cell.

In another embodiment of the ninth or tenth aspect, the cell is a fungal cell, including a yeast cell, preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile.

In another embodiment of the ninth or tenth aspect, the cell is a prokaryotic cell, including Gram-positive, Gram negative and Gram-variable bacterial cells, preferably Gram-negative bacterial cells, or an archaeal cell, preferably wherein the prokaryotic cell or an archaeal cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

In one embodiment according to the various aspects as disclosed herein, the cell may be a eukaryotic cell or a prokaryotic cell, wherein the cell is selected from a cell originating from Rhodococcus rhodochrous, Aerococcus sp., Ashbya gossypii, Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica, wherein the cell is preferrably selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae or Yarrowia lipolytica.

In another embodiment of the ninth or tenth aspect, the cell may be a eukaryotic cell or a prokaryotic cell, wherein the cell is selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica, Phakopsora spec, e.g. Phakopsora pachyrhizi, Zymoseptoria spec, e.g. Zymoseptoria tritici, Septoria, Mycosphaerella, Phythopthora spec., e.g. Phytopthora infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, or Ashbya.

In one embodiment of the ninth or tenth aspect, during step (a) and/or (b), at least one additional effector, or a nucleic acid sequence encoding the same, is provided, the additional effector promoting DNA repair and cell regeneration before, during or upon insertion of at least one nick or double-strand break at the nucleic acid sequence of interest at or near at least one target site. The additional effector, may be selected from, but is not restricted to, at least one additional effector having an enzymatic activity that modifies at least one target nucleic acid (e.g., nuclease activity, e.g. exonuclease activity, methyltransferase activity, demethylase activity, DNA repair activity, DNA damage activity, deamination activity, dismutase activity, alkylation activity, depurination activity, oxidation activity, pyrimidine dimer forming activity, helicase activity (e.g. SF1/2, SF3, SF4), integrase activity, telomerase activity, topoisomerase activity, e.g. gyrase activity, transposase activity, transcriptase or reverse transcriptase activity, recombinase activity, polymerase activity, e.g. RNA polymerase activity or DNA polymerase activity e.g. Pol theta activity, ligase activity, photolyase activity or glycosylase activity).

In certain embodiments, during step (b) an additional guide RNA that hybridizes to the edited sequence is provided for nicking the non-edited strand. Such PE3 approaches may be used to improve the repair of the mismatch in favor of the edited strand.

In an eleventh aspect, there is provided an edited cell, tissue, organ, material or whole organism obtained by or obtainable by a method according to the tenth aspect as disclosed herein.

In certain embodiments, the edited cell, tissue, organ, material or whole organism is not a plant or animal edited cell, tissue, organ, material or whole organism exclusively obtained by means of an essentially biological process.

In a twelfth aspect, there is provided, use of a compound selected from a prime editor complex as defined in the first aspect; and/or a pegRNA or the pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh aspect; for introducing or modification in a nucleic acid molecule, preferably in a genome, including uses for optimizing or modifying a trait in a plant, including the modification of a yield-related trait, or a disease-resistance related trait, and/or for metabolic engineering in cell, including a prokaryotic or eukaryotic cell, preferably in a plant cell, an algal cell, a fungal cell, including a yeast cell, or an archaeal cell, optionally wherein the use excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

In one embodiment, the use is a use for in vitro, in vivo and/or ex vivo modification of a nucleic acid molecule, preferably in a genome of a cell.

Optimizing or modifying a trait in a plant may for instance comprise genetic modification leading to the comprisal of an endogenous gene or a transgene that confers herbicide resistance, such as the bar or pat gene, which confer resistance to glufosinate ammonium (Liberty®, Basta® or Ignite®; EP0242236 and EP0242246); or any modified EPSPS gene, such as the 2mEPSPS gene from maize (EP0508909 and EP0507698), or glyphosate acetyltransferase, or glyphosate oxidoreductase, which confer resistance to glyphosate (RoundupReady®), or glyphosate resistant EPSPS, such as a CP4 EPSPS, or such as an N-acetyltransferase (gat) gene, or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola; and/or an endogenous gene or a transgene that confers increased oil content or improved oil composition, such as a 12:0 ACP thioesteraseincrease to obtain high laureate, which confers pollination control, such as barnase under control of an anther-specific promoter to obtain male sterility, or barstar under control of an anther-specific promoter to confer restoration of male sterility, or such as the Ogura cytoplasmic male sterility and nuclear restorer of fertility; and/or an endogenous gene or a transgene that confers resistance to glufosinate ammonium (Liberty®, Basta® or Ignite®); and/or a gene coding for a phosphinothricin-N-acetyltransferase (PAT) enzyme, such as a coding sequence of the bialaphos resistance gene (bar) of Streptomyces hygroscopicus. Such plants may, for example, comprise the elite events MS-BN1 and/or RF-BN1 as described in WO01/41558, or elite event MS-B2 as described in WO01/31042, or any combination of these events.

Examples of technically induced mutants in Brassica napus, as a result of optimizing of modifying a trait, are mutants in the FATB gene as described in WO2009007091 or in the FAD3 genes as described in WO2011/060946, or may be podshatter resistant mutants such as mutants described in WO2009068313 or in WO2010006732, or mutations conferring herbicide tolerance such as the PM1 and PM2 mutations conferring imidazolinone tolerance (Tan et al. Pest Manag Sci. 2005 March; 61 (3): 246-57. doi: 10.1002/ps.993.; U.S. Pat. No. 5,545,821).

In a thirteenth aspect, there is provided a compound selected from a prime editor complex as defined in the first aspect; and/or a pegRNA or the pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh or fifteenth aspect; for use in a method of treating or preventing a disease in a patient.

In a fourteenth aspect, there is provided a method of treating or preventing a disease, the method comprising using a prime editor complex as defined in the first aspect; and/or a pegRNA or the pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh aspect for introducing at least one modification in a genomic locus of interest of at least one cell of a subject in need thereof at or near at least one disease-state related target site.

In one embodiment, the method comprises an ex vivo modification of the at least one disease-state related target site, wherein at least one cell of a subject is provided to perform an ex vivo modification of the at least one disease-state related target site to obtain at least one edited cell.

In a fifteenth aspect, there is provided a method for cell therapy, comprising administering to a patient in need thereof said at least one edited cell of the fourteenth aspect, wherein presence of said at least one edited cell remedies a disease in said patient.

In a sixteenth aspect, there is provided a use of a compound selected from a prime editor complex as defined in the first aspect; and/or a pegRNA or the pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh or fifteenth aspect for the manufacture a medicament for treating or preventing a disease in a patient.

All methods disclosed herein exclude processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes, optionally, where the method comprises the following step: (g) regenerating at least one population of edited cells, tissues, organs, materials or whole organisms from the at least one edited cell or construct.

According to the various aspects and embodiments disclosed herein relating to a compound selected from a prime editor complex as defined in the first aspect; and/or a pegRNA or the pegRNA system as defined in the second aspect; and/or a Cas12a prime editor as defined in the third aspect; and/or a complex as defined in the fourth aspect; and/or a nucleic acid molecule or more than one nucleic acid molecules as defined in the fifth aspect; and/or an expression vector or construct or more than one expression vector or constructs as defined in the sixth aspect; and/or a cell as defined in the seventh or fifteenth aspect the compound is provided in a functional form, e.g., including stabilizers, cofactors, means for introducing the same into a target cell or tissue and the like.

The present invention is further illustrated in the following, non-limiting examples.

### EXAMPLES

### Example 1: Analysis of Cas12a Prime Editing in Oilseed Rape Protoplasts Using a GFP Reporter System

To test the feasibility of Cas12a-mediated prime editing in plants, experiments using oilseed rape (Brassica napus) protoplasts were performed. Oilseed rape protoplasts were isolated from the leaves of 4- to 7-week-old aseptically grown plants. Healthy leaves were cut into fine strips with a sharp razor blade. The strips were infiltrated with cell wall-dissolving enzyme solution and incubated overnight in the dark with gentle shaking (40 rpm) at 24° C. After enzymatic digestion, the released protoplasts were collected by filtering the mixture through 40-μm nylon meshes and resuspended in W5 solution. The resuspended protoplasts were kept on ice and allowed to settle by gravity, after which the cell pellet was resuspended in MMG. Protoplasts were transfected in microtiter plates using a semi-automated robotic platform. In brief, 30 μl of cells (5×10⁴ cells) were mixed with 6 μl total plasmid DNA (10 μg) before adding 32 μl of PEG 1500 solution. The plates were then centrifuged for 1 min at 1000 rpm and incubated at room temperature for 15 min. After removing the PEG solution, transfected protoplasts were resuspended in 140 μl of W5 solution and incubated in the dark.

Cas12a prime editing was first evaluated using a GFP reporter system. This assay detects restoration of an inactivated GFP reporter (hereafter referred to as dGFP) harboring an early stop codon resulting from changing Isoleucine codon 15 to TAG (SEQ ID NO: 70). Successful prime editing will change the stop codon into the original ATC codon and restore GFP expression. Oilseed rape protoplasts were co-transfected with plasmids encoding an engineered M-MLV RT (D200N+L603W+T330P+T306K+W313; SEQ ID NO: 146; hereafter referred to as RT (5M)) fused to either the N- or C-terminus of a previously developed LbCas12a nickase variant harboring an in-frame deletion in the RuvC core lid domain (SEQ ID NO: 15; PCT/EP2023/055130) along with a plasmid encoding the dGFP reporter under control of the strong Cauliflower mosaic virus 35S promoter (SEQ ID NO: 151) and plasmids encoding a series of dGFP-targeting Cas12a pegRNAs with primer binding site (PBS) lengths ranging from 9-20 bases/nt and reverse transcription template (RTT) lengths ranging from 15-86 bases/nt. All prime editor constructs included the Arabidopsis ubiquitin10 promoter for constitutive expression (SEQ ID NO: 149), while expression of the pegRNAs was driven by the polymerase III-type promoter of the Arabidopsis U6 snRNA gene (SEQ ID NO: 150). All pegRNAs contained two mature direct repeats (SEQ ID NO: 91) 5′ and 3′ of the spacer and PBS, respectively.

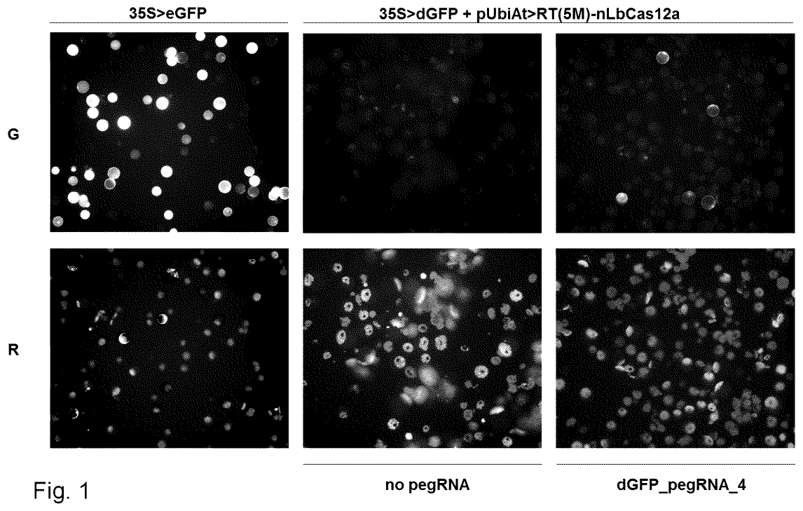

An overview of exemplary pegRNAs tested, including both 5′ and 3′ extended gRNA designs, is shown in Table 1. Transfected protoplasts were incubated at 24° C. and prime editing efficiencies were measured by quantifying the number of GFP-positive cells using fluorescence imaging. As a positive control, protoplasts were transfected with a construct expressing wild type eGFP under control of the 35S promoter. As a negative control, the Cas12a prime editor construct was tested without pegRNA. As shown in Table 1 and FIG. 1, fluorescence imaging at 2 days post transfection revealed approximately 70% GFP-fluorescent cells in the positive control and up to 3% in the samples transfected with the dGFP reporter. Importantly, no GFP-positive cells could be observed in the absence of the pegRNA (FIG. 1).

| | | | | | GFP- |
|---|---|---|---|---|---|
| | | SEQ | | | positive |
| | | ID | RTT | PBS | cells |
| Type | Name | NO | (nt) | (nt) | (%) |
| 3′extended | dGFP_pegRNA_1 | 30 | 44 | 9 | 0 |
| pegRNA | dGFP_pegRNA_2 | 31 | 44 | 12 | 0 |
| | dGFP_pegRNA_3 | 32 | 44 | 20 | 0 |
| | dGFP_pegRNA_4 | 33 | 66 | 9 | 3.38 ± 0.35 |
| | dGFP_pegRNA_5 | 34 | 66 | 12 | 1.46 ± 0.32 |
| | dGFP_pegRNA_6 | 35 | 66 | 20 | 0 |
| | dGFP_pegRNA_7 | 36 | 86 | 9 | 1.48 ± 0.24 |
| | dGFP_pegRNA_8 | 37 | 86 | 12 | 1.45 ± 0.27 |
| | dGFP_pegRNA_9 | 38 | 86 | 20 | 0 |
| 5′extended | dGFP_pegRNA_10 | 39 | 9 | 9 | 0 |
| pegRNA | dGFP_pegRNA_11 | 40 | 9 | 20 | 0 |
| | dGFP_pegRNA_12 | 41 | 15 | 9 | 0 |
| | dGFP_pegRNA_13 | 42 | 15 | 20 | 0 |
| | dGFP_pegRNA_14 | 43 | 22 | 9 | 0 |
| | dGFP_pegRNA_15 | 44 | 22 | 20 | 0 |

Table 1 shows the editing efficiency of different pegRNAs and RT-nLbCas12a fusion proteins in oilseed rape (Brassica napus) protoplasts co-transformed with a dGFP reporter plasmid. Editing efficiencies are expressed as the number of GFP-positive cells two days post transfection as determined by fluorescence imaging.

The highest frequency of fluorescent cells (average 3.38% at 2 dpt) was obtained when combining RT (5M) fused to the N-terminus of nLbCas12a (SEQ ID NO: 65) and a dGFP-targeting pegRNA with an RTT of 66 bases and a PBS of 9 bases on the 3′end of the RNA (SEQ ID NO: 33). Interestingly, enhancing RTT and PBS lengths up to 86 and 20 bases, respectively, gradually reduced editing efficiencies whereas an RTT of 44 bases completely abolished editing. Also, no GFP-positive cells could be observed with neither C-terminal fusions of RT (5M) nor 5′ extended pegRNAs. Deep amplicon sequencing of extracted plasmid DNAs confirmed the precise nature of the introduced edits (data not shown), further demonstrating that specific nCas12a-RT architectures and pegRNA structures can support efficient editing. Corroborating previous findings with Cas9 editors (Anzalone et al., 2019), these results also highlight the importance of testing a series of pegRNAs when editing a novel target site.

### Example 2: Influence of Incubation Temperature on Cas12a Prime Editing Activity

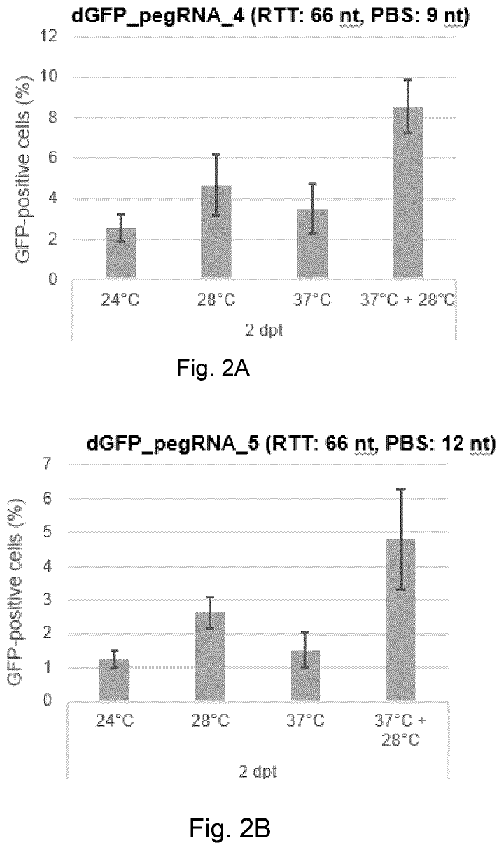

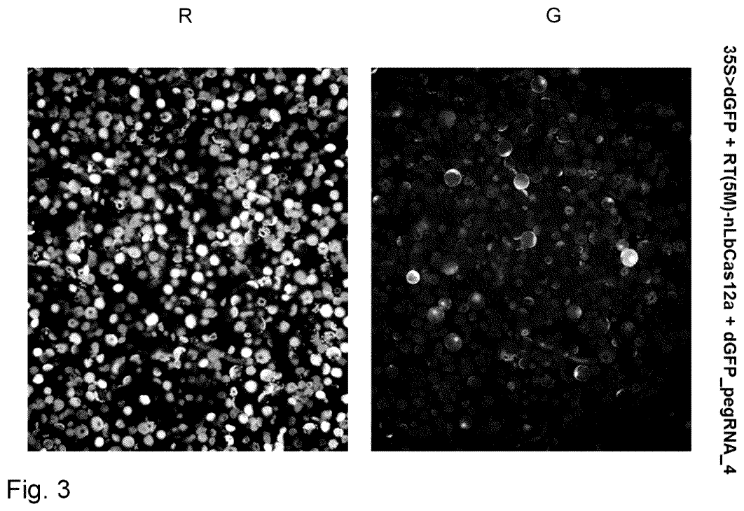

In a first attempt to optimize the efficiency of nCas12a-mediated prime editing, oilseed rape cells transfected with the dGFP reporter construct along with a plasmid encoding the N-terminal fusion of RT (5M) to nLbCas12a (SEQ ID NO: 65) and two different pegRNAs with RT templates of 66 bases and PBS lengths of 9 bases (SEQ ID NO: 33) or 12 nt (SEQ ID NO: 34) were incubated at three different temperatures, i.e. 24° C., 28° C. or 37° C. FIG. 2 shows GFP editing efficiencies as measured by fluorescence imaging at 1, 2 and 3 days post transfection (dpt). For both pegRNAs tested, editing activity was significantly higher at 28° C. (average 4.55% at 2 dpt) than at 24° C. (average 2.53% at 2 dpt) or 37° C. (average 3.48% at 2 dpt). Moreover, exposing transfected cells to 37° C. for 24h prior to incubation at 28° C. further boosted editing efficiencies (up to 8.2% GFP-positive cells at 2 dpt), resulting in a 3.4-fold average improvement compared to the 24° C. treatment (see FIG. 2 and FIG. 3). These findings demonstrate that the efficiency of nCas12a prime editing can be optimized by testing different incubation temperatures.

### Example 3: Prime Editing in Oilseed Rape and Soybean Protoplasts with the LbCas12a-R1138A Nickase

The R1226A mutant of AsCas12a and the corresponding R1138A variant of LbCas12a were previously described as nickases that, at least in vitro, predominantly cleave the non-target DNA strand (Yamano et al., 2016; Kim et al., 2022). While the R1138A-LbCas12a nickase is known to facilitate Redraw editing in human cells (Kim et al., 2022), there are no reports yet of prime editing with this nickase in plants. To evaluate the efficiency of prime editors harboring the R1138A mutation, an expression construct encoding a prime editor comprising an RT (5M) fused N-terminally to LbCas12a-D156R/R1138A (SEQ ID NO: 199) under control of the Arabidopsis ubiquitin10 promoter (SEQ ID NO: 149) was generated and transfected into oilseed rape protoplasts along with the dGFP reporter described in Example 1 and one of three pegRNAs containing an RTT of 66 or 86 nt and a PBS of 9 or 12 nt, respectively (i.e. dGFP_pegRNA_4, dGFP_pegRNA_5 and dGFP_pegRNA_7 in Table 1; SEQ ID NO: 33; SEQ ID NO: 34; SEQ ID NO: 36). As a positive control, protoplasts were transfected with a plasmid encoding the prime editor comprising an RT (5M) fused N-terminally to the ‘RuvC lid deletion’ (RuvC^{lidΔ}) nickase described in Example 1 (SEQ ID NO: 65). The transfected cells were incubated at 37° C. for 24h prior to incubation at 28° C. Editing efficiencies were quantified at three days post transfection using fluorescence imaging.

As shown in Table 2, all treatments yielded multiple GFP-fluorescent cells per well, indicating successful prime editing of the reporter plasmid with both prime editors and all the exemplary pegRNAs. Consistent with previous findings on the ‘RuvC lid deletion’ nickase variant (see Example 1), dGFP_pegRNA_4 demonstrated the highest levels of GFP editing, reaching up to 7.52%. These trends were also observed in soybean leaf protoplasts (see Table 3), further illustrating that the LbCas12a-R1138A variant can induce effective prime editing in plant cells. However, the LbCas12a-RuvC^{lidΔ} variant showed good editing results for all three tested pegRNA, whereas LbCas12a-R1138A showed considerably lower editing rates with dGFP_pegRNA_5.

| | | | GFP-positive cells (%) | |
|---|---|---|---|---|
| pegRNA | | | RT(5M)- | RT(5M)- |
| | RTT | PBS | nLbCas12a | nLbCas12a |
| Name | (nt) | (nt) | (D156R/RuvC^{lidΔ}) | (D156R/R1138A) |
| dGFP_pegRNA_4 | 66 | 9 | 2.42 ± 0.41 | 7.52 ± 1.60 |
| dGFP_pegRNA_5 | 66 | 12 | 1.27 ± 0.26 | 0.37 ± 0.14 |
| dGFP_pegRNA_7 | 86 | 9 | 0.11 ± 0.15 | 0.3 ± 0.14 |

Table 2 shows the editing efficiency of different pegRNAs and RT-nLbCas12a fusion proteins in oilseed rape (Brassica napus) protoplasts co-transformed with a dGFP reporter plasmid. Editing efficiencies are expressed as the percentage of GFP-positive cells three days post transfection as determined by fluorescence imaging.

| | | GFP-positive |
|---|---|---|
| Prime editor plasmid | pegRNA | cells/wells |
| RT(5M)-nLbCas12a | dGFP_pegRNA_4 | 5.33 ± 2.87 |
| (D156R/RuvC^{lidΔ}) | dGFP_pegRNA_5 | 7 ± 1.41 |
| (non codon-optimized) | dGFP_pegRNA_7 | 4.67 ± 2.87 |
| RT(5M)-nLbCas12a | dGFP_pegRNA_4 | 11.33 ± 2.05 |
| (D156R/RuvC^{lidΔ}) | dGFP_pegRNA_5 | 15.33 ± 3.68 |
| (soybean codon-optimized) | dGFP_pegRNA_7 | 8.33 ± 0.94 |
| RT(5M)-nLbCas12a | dGFP_pegRNA_4 | 17.67 ± 4.50 |
| (D156R/R1138A) | dGFP_pegRNA_5 | 5 ± 3.74 |
| (soybean codon-optimized) | dGFP_pegRNA_7 | 2 ± 1.63 |

Table 3 shows the editing efficiency of different pegRNAs and RT-nLbCas12a fusion proteins in soybean (Glycine max) leaf protoplasts co-transformed with a dGFP reporter plasmid. Editing efficiencies are expressed as the total number of GFP-positive cells four days post transfection as determined by fluorescence imaging.

### Example 4: Analysis of Cas12a Prime Editing at Chromosomal Target Sites

To examine nCas12a-mediated prime editing at endogenous genes, a plasmid encoding the RT (5M)-nLbCas12a (D156R/R1138A) prime editor described in Example 3 (SEQ ID NO: 199) was co-transfected into oilseed rape (Brassica napus) protoplasts along with Cas12a pegRNAs targeting the BnFAD2 or BnALS3 genes, respectively (SEQ ID NO: 153 and SEQ ID NO: 154). For each target, a series of 3′ extended pegRNAs with PBS lengths ranging from 9-20 bases and RTT lengths ranging from 44-86 bases was tested (SEQ ID NOS: 224 to 229 and SEQ ID NOS: 232 to 246). All BnFAD2-targeting pegRNAs encoded a 3-nt substitution at the +2 position, while the ALS3 pegRNAs were designed to introduce a 6-nt substitution at the +9 position (counting the first base 3′ of the pegRNA-induced nick as position +1). Transfected oilseed rape protoplasts were incubated at 37° C. for 24 h prior to incubation at at 28° C. Editing efficiencies were determined at three days post transfection by deep amplicon sequencing. As shown in Table 4 and Table 5, precise editing could be detected at both sites with editing efficiency dependent on the length of the PBS and RTT. In common with the results obtained in the dGFP reporter assay, the highest efficiencies (average 0.16% at the FAD2 site and 0.13% at the ALS3 site) were achieved with pegRNAs containing a relatively long RTT of 66 bases and a relatively short PBS of 9 to 11 bases. Interestingly, these findings were further confirmed with a set of pegRNAs that drive a 6-nucleotide deletion at the +3 position in the thioredoxin H2-encoding BnTRX gene (SEQ ID NOS: 411 to 415). Editing efficiencies at this site reached as high as 2.01% when using a pegRNA with an RTT of 66 nt and a PBS of 10 nt (see Table 6). Collectively these findings show the ability of nLbCas12a prime editors to install different types of edits at various genomic sites in plant cells.

| | | | | | NGS results | |
|---|---|---|---|---|---|---|
| | pegRNA | | | | Total read | Editing |
| Prime editor | Name | RTT (nt) | PBS (nt) | Edit | count | efficiency (%) |
| RT(5M) | none | n/a | n/a | n/a | 52,221 | 0.00% |
| nLbCas12a | Non-targeting pegRNA | 66 | 9 | n/a | 63,505 | 0.00% |
| (D156R/R1138A) | BnFAD2_pegRNA_16 | 66 | 9 | +2-4 CAT to | 62,701 | 0.16% |
| | | | | GCA | | |
| | BnFAD2_pegRNA_17 | 66 | 9 | +2-4 CAT to | 48,653 | 0.08% |
| | | | | GCA | | |
| | BnFAD2_pegRNA_18 | 66 | 9 | +2-4 CAT to | 49,963 | 0.08% |
| | | | | GCA | | |
| | BnFAD2_pegRNA_19 | 66 | 12 | +2-4 CAT to | 73,086 | 0.05% |
| | | | | GCA | | |
| | BnFAD2_pegRNA_20 | 66 | 20 | +2-4 CAT to | 39,528 | 0.00% |
| | | | | GCA | | |
| | BnFAD2_pegRNA_21 | 86 | 9 | +2-4 CAT to | nd | nd |
| | | | | GCA | | |
| | BnFAD2_pegRNA_22 | 86 | 12 | +2-4 CAT to | 107,849 | 0.00% |
| | | | | GCA | | |
| | BnFAD2_pegRNA_23 | 86 | 20 | +2-4 CAT to | 54,217 | 0.00% |
| | | | | GCA | | |
| | BnFAD2_pegRNA_24 | 44 | 9 | +2-4 CAT to | 46,486 | 0.16% |
| | | | | GCA | | |
| | BnFAD2_pegRNA_25 | 44 | 12 | +2-4 CAT to | 58,668 | 0.00% |
| | | | | GCA | | |
| | BnFAD2_pegRNA_26 | 44 | 20 | +2-4 CAT to | 66,923 | 0.00% |
| | | | | GCA | | |
| | BnFAD2_pegRNA_27 | 44 | 10 | +2-4 CAT to | 59,917 | 0.04% |
| | | | | GCA | | |
| | BnFAD2_pegRNA_28 | 44 | 11 | +2-4 CAT to | 52,573 | 0.11% |
| | | | | GCA | | |
| | BnFAD2_pegRNA_29 | 44 | 8 | +2-4 CAT to | 41,291 | 0.12% |
| | | | | GCA | | |
| | BnFAD2_pegRNA_30 | 44 | 7 | +2-4 CAT to | 53,582 | 0.01% |
| | | | | GCA | | |

Table 4 above shows the efficiency of prime editing induced by different pegRNAs and the RT-nLbCas12a (D156R/R1138A) fusion protein at an endogenous BnFAD2 target site in oilseed rape (Brassica napus) protoplasts at three days post transfection. Editing efficiencies are expressed as the percentage of total NGS reads having the desired +2-4 CAT to GCA edit.

| | | | | | NGS results | |
|---|---|---|---|---|---|---|
| | pegRNA | | | | Total | Editing |
| | | RTT | PBS | | read | efficiency |
| Prime editor | Name | (nt) | (nt) | Edit | count | (%) |
| RT(5M)- | none | n/a | n/a | n/a | 117,981 | 0.00% |
| nLbCas12a | Non-targeting | 66 | 9 | n/a | 76,806 | 0.00% |
| (D156R/R1138A) | pegRNA | | | | | |
| | BnALS3_pegRNA_1 | 66 | 10 | +9−14 GACCGT to AACCAC | 83,984 | 0.04% |
| | BnALS3_pegRNA_2 | 66 | 11 | +9−14 GACCGT to AACCAC | 94,211 | 0.13% |
| | BnALS3_pegRNA_3 | 66 | 12 | +9−14 GACCGT to AACCAC | 96,882 | 0.09% |
| | BnALS3_pegRNA_4 | 44 | 10 | +9−14 GACCGT to AACCAC | 79,078 | 0.00% |
| | BnALS3_pegRNA_5 | 44 | 11 | +9−14 GACCGT to AACCAC | 89,787 | 0.00% |
| | BnALS3_pegRNA_6 | 44 | 12 | +9−14 GACCGT to AACCAC | 100,483 | 0.03% |

Table 5 above shows the efficiency of prime editing induced by different pegRNAs and the RT-nLbCas12a (D156R/R1138A) fusion protein at an endogenous BnALS3 target site in oilseed rape (Brassica napus) protoplasts at three days post transfection. Editing efficiencies are expressed as the percentage of total NGS reads having the desired +9-14 GACCGT to AACCAC edit.

Table 6 above shows the efficiency of prime editing induced by different pegRNAs and the RT-nLbCas12a (D156R/R1138A) fusion protein at an endogenous BnTRX target site in oilseed rape (Brassica napus) protoplasts at three days post transfection. Editing efficiencies are expressed as the percentage of amplicons having the desired +3 GTTGCA deletion as determined by droplet digital PCR.

| | pegRNA | | | | Editing |
|---|---|---|---|---|---|
| Prime editor | Name | RTT (nt) | PBS (nt) | Edit | efficiency (%) |
| RT(5M)- | Non-targeting pegRNA | 66 | 9 | n/a | 0.00 |
| nLbCas12a | BnTRX_pegRNA_1 | 66 | 10 | +3 GTTGCAdel | 2.01 ± 0.57 |
| (D156R/R1138A) | BnTRX_pegRNA_2 | 66 | 11 | +3 GTTGCAdel | 0.25 ± 0.12 |
| | BnTRX_pegRNA_3 | 66 | 12 | +3 GTTGCAdel | 1.42 ± 0.21 |
| | BnTRX_pegRNA_4 | 44 | 10 | +3 GTTGCAdel | 0.82 ± 0.05 |
| | BnTRX_pegRNA_5 | 44 | 11 | +3 GTTGCAdel | 0.29 ± 0.07 |

To further evaluate nCas12a-mediated prime editing at endogenous target sites, pAtUbi10>nCas12a-PE constructs are co-transfected in soybean (Glycine max) protoplasts along with a series of Cas12a pegRNAs targeting the GmFAD2 gene (SEQ ID NO: 155). The transfected cells are then incubated in WI solution for at least 72 hours and analyzed via droplet digital PCR and/or amplicon deep sequencing. Aiming to install a variety of single- and multi-base precise edits, RT templates encoding different transversions, insertions and deletions are tested.

In an alternative approach, a transgenic oilseed rape line harboring a stably integrated single-copy dGFP reporter is used. The chromosomal dGFP reporter contains an early stop codon resulting from changing codon 110 from CGA (arginine) to TAG (SEQ ID NO: 152) as well as an engineered TTTC Cas12a PAM site created by a silent AAG to AAA mutation at the K114 position. Precise editing of the chromosomal reporter restores the GFP coding sequence which can be detected by fluorescence imaging and/or flow cytometry. Protoplasts are isolated from the dGFP transgenic line and co-transfected with various nLbCas12a-PE expression constructs and dGFP-targeting pegRNA-encoding plasmids as described above. Different PBS lengths varying from 9-48 bases and RTT lengths ranging from 12-94 bases are tested and editing outcomes are verified by high-throughput sequencing of cells harvested 14 or 21 days after alginate embedding.

For evaluation of prime editing efficiency in monocot plants, rice mesophyll protoplasts are isolated exactly as described before (see EP22159465) and co-transfected with plasmids encoding RT (5M) fused to nLbCas12a in N- or C-terminal orientations together with a series of pegRNAs targeting different sites in the OsCDC48 gene (LOC_Os03g05730). Different PBS lengths varying from 9-48 bases and RTT lengths ranging from 12-94 bases are evaluated (for examples see SEQ ID NO: 53 to SEQ ID NO: 64). Transfected cells are harvested by centrifugation at 3 dpt and editing efficiencies are determined via combined droplet digital PCR analysis and deep amplicon sequencing.

Finally, to compare the efficiency of nCas12a-mediated prime editing to that of Cas12a-mediated homology-directed repair (HDR) and the Cas12a-based Redraw technology (Kim et al., 2022), RT-nLbCas12a and pegRNAs encoding different transition and transversion mutations are transfected into rice and/or oilseed rape protoplasts and their editing activity is compared to that of RT-Cas12a nuclease fusions and so-called tagRNAs (Kim et al., 2022), on the one hand, and Cas12a nuclease and gRNAs mixed with DNA repair templates, on the other. Both tagRNAs and DNA repair templates are designed to encode the same edits as the pegRNAs. Editing is studied at endogenous target sites and in the dGFP reporter system and efficiencies and product purity are compared using deep amplicon sequencing as described before.

Finally, to compare the efficiency of nCas12a-mediated prime editing to that of Cas12a-mediated homology-directed repair (HDR) and the Cas12a-based Redraw technology (Kim et al., 2022), RT-nLbCas12a and pegRNAs encoding different transition and transversion mutations are transfected into rice and/or oilseed rape protoplasts and their editing activity is compared to that of RT-Cas12a nuclease fusions and so-called tagRNAs (Kim et al., 2022), on the one hand, and Cas12a nuclease and gRNAs mixed with DNA repair templates, on the other. Both tagRNAs and DNA repair templates are designed to encode the same edits as the pegRNAs. Editing is studied at endogenous target sites and in the dGFP reporter system and efficiencies and product purity are compared using deep amplicon sequencing as described before.

### Example 5: Optimization of Cas12a Prime Editing Activity

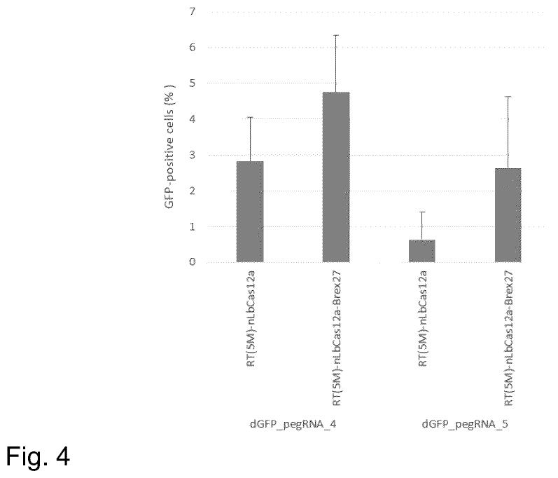

Stabilization of the ssDNA intermediate generated upon binding of the Cas protein and the pegRNA to the target sequence by addition of a ssDNA-binding protein domain was previously shown to enhance the efficiency of Cas9 prime editing and Cas12a-mediated Redraw (Nelson et al., 2022; Kim et al., 2022). To evaluate whether ssDNA binding proteins similarly influence the outcome of nCas12a-mediated prime editing, C-terminal fusions of the Brex27 peptide motif to RT (5M)-nLbCas12a were generated and tested in oilseed rape protoplasts using the dGFP reporter system described in Example 1. Encoded by BRCA2 Exon 27, Brex27 (SEQ ID NO: 71) is a minimal motif consisting of 36 amino acids that was previously shown to stabilize ssDNA nucleoprotein filaments during homologous recombination via recruitment of RAD51 (Davies et al., 2007). Plasmids encoding the fusion construct (SEQ ID NO: 68) and the dGFP reporter (SEQ ID NO: 70) were co-transfected in OSR protoplasts together with two dGFP-targeting pegRNAs (dGFP_pegRNA_4 and dGFP_pegRNA_5 in Table 1), and the GFP editing efficiency was compared against that of RT (5M)-n nLbCas12a (D156R/RuvClidA) without Brex27 (SEQ ID NO: 65). As shown in FIG. 4, fusion of the 36aa Brex27 peptide resulted in an average 1.7- and 4.2-fold increase in editing efficiency with dGFP_pegRNA_4 and dGFP_pegRNA_5, respectively. Encouraged by these results, additional ssDNA binding domains are tested, including DNA-binding domains from different human proteins such as RPA70, RPA32, BRCA2, the HNRNPK KH domain, the PUF60 RNA recognition motif and/or the DNA-binding domain from Rad51 (SEQ ID NO: 72 to SEQ ID NO: 80).

In a next round of optimization, the effect of stabilizing pegRNAs by incorporating structured RNA motifs is evaluated. To this end, different RNA hairpins and pseudoknots are added to the 3′ end of the pegRNA PBS, including the PP7 hairpin from bacteriophages and the pseudoknot evoPreQ1, both of which were previously shown to enhance prime editing efficiencies at a variety of target sites in multiple cell lines (Nelson et al., 2022; Kim et al., 2022). To reduce potential interference with pegRNA function, a DNA linker is inserted between the PBS and the RNA motif. Linker sequences are designed using ViennaRNA or pegLIT to avoid potential base pairing interactions between the linker and the PBS or pegRNA spacer (Nelson et al., 2022; Lorenz et al., 2011). Plasmids encoding nCas12a prime editors along with the engineered pegRNAs targeting different loci are co-transfected in oilseed rape and/or rice protoplasts and the editing efficiency is measured by deep amplicon sequencing as described previously.

Recently, multiple strategies to increase the efficiency of Cas9-mediated prime editing have been published, including the introduction of silent mutations near the edit site to evade DNA mismatch repair (Chen et a., 2021) and nicking of the non-edited strand to improve repair of the mismatch in favor of the edited strand (Anzalone et al., 2019). Also, the use of split Cas9 prime editors with an untethered M-MLV RT has attracted increasing interest (Liu et al., 2022; Zheng et al., 2022; Feng et al., 2023). To evaluate whether these strategies can also be utilized to improve the efficiency of nCas12a-mediated prime editing, the LbCas12a-R1138A prime editor (as described in Example 4; SEQ ID NO: 199) was co-expressed with dGFP-targeting pegRNA_4 (RTT: 66 bases, PBS: 9 bases; see Example 1; SEQ ID NO: 33) together with a C-terminal fusion of the bacteriophage MS2 N55K coat protein (hereafter referred to as MCP) and NLS-tagged RT (5M) (SEQ ID NO: 200) as well as a second Cas12a crRNA that introduces a nick in the non-edited strand of the dGFP reporter plasmid (SEQ ID NO: 201). Quantification of GFP-positive cells at 3 dpt showed that co-transfection of MCP-RT (5M) enhanced editing efficiencies of nLbCas12a-R1138A by more than 1.5-fold (average of 8.15% GFP-positive cells, see Table 7). Furthermore, by co-delivering MCP-RT (5M) and the nicking gRNA, the editing efficiencies were further improved (reaching up to 16.2% GFP-positive cells at 3 dpt) provided that the nicking gRNA was delivered at a concentration three times lower than that of the pegRNA. When using equal ratios of nicking gRNA to pegRNA, the overall editing rates did not show a significant increase. This could be attributed to the fact that Cas12a preferentially binds the sgRNA over the pegRNA, leading to a reduced number of active ribonucleoprotein complexes for prime editing. Additionally, there may be steric hindrance or competition between the pegRNA and nicking gRNA RNPs, limiting their access to the target site.

| | | | | GFP- |
|---|---|---|---|---|
| | | | MCP- | positive |
| | | Nicking | RT | cells |
| Prime editor | dGFP_pegRNA_4 | gRNA | (5M) | (%) |
| RT(5M)- | 3 μg | No | No | 5.5 ± 1.22 |
| nLbCas12a | | No | Yes | 8.15 ± 1.15 |
| (D156R/R1138A) | | 3 μg | Yes | 9.47 ± 2.58 |
| | | 1 μg | Yes | 16.24 ± 2.59 |

Table 7 above shows the influence of second-strand nicking and in trans expression of an MCP-RT (5M) fusion protein on the efficiency of nLbCas12a-mediated prime editing in oilseed rape (Brassica napus) protoplasts co-transformed with a dGFP reporter plasmid. Editing efficiencies are expressed as the percentage of GFP-positive cells three days post transfection as determined by fluorescence imaging.

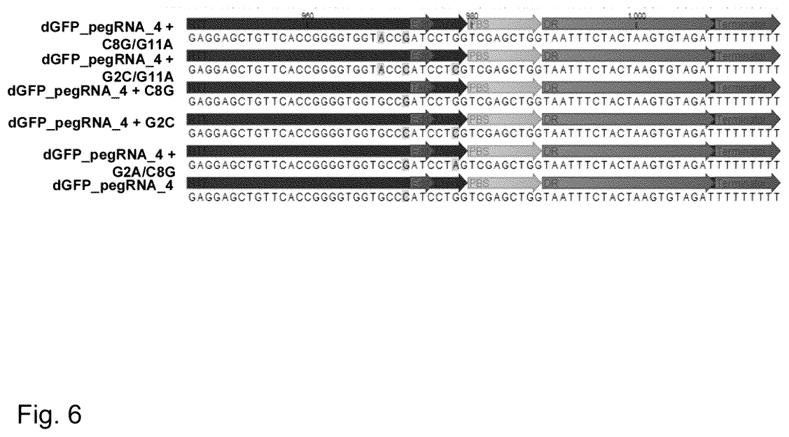

In a separate experiment, the impact of installing silent mutations near the desired edit was evaluated. To this end, either one or two synonymous mutations were introduced in the RTT of dGFP_pegRNA_4 (SEQ ID NO: 33) less than 5 bp away from the TGA>ATC edit that restores GFP fluorescence (see FIG. 6). The pegRNA constructs were co-transfected into oilseed rape protoplasts along with the dGFP reporter and a plasmid encoding the R1138A or the ‘RuvC lid deletion’ nLbCas12a prime editors described above (SEQ ID NO: 199 and SEQ ID NO: 65) and editing efficiencies were quantified by recording the number of GFP-positive cells at 3 dpt. While most of the pegRNAs that contain additional mutations (SEQ ID NO: 202; SEQ ID NO: 203; SEQ ID NO: 204; SEQ ID NO: 205; SEQ ID NO: 206) induced editing efficiencies similar to those induced by the regular pegRNA (data not shown), adding silent mutations at positions 2 and 11 in the RTT (3′-end to 5′-end, counting the 3′ base of RTT as position 1, ie +2 G-to-C and +11 G-to-A; SEQ ID NO: 203) significantly improved the level of editing by up to 3.7- and 2.17-fold for the RuvC^{lidΔ′} and R1138A prime editors, respectively (see Table 8). Conspicuously, no editing could be detected when transfecting a prime editor containing the LbCas12a nuclease harboring the D156R mutation alone. Consistent with earlier findings in human cells (Chen et al., 2021; Xi et al., 2022), these results show that introducing same-sense mutations in the RTT to potentially evade DNA mismatch repair can enhance Cas12a PE (Prime Editing) efficiency.

| | GFP-positive cells (%) | |
|---|---|---|
| | | dGFP_pegRNA_4 + |
| Prime Editor | dGFP_pegRNA_4 | G2C/G11A |
| RT(5M)-LbCas12a(D156R) | 0 | 0 |
| RT(5M)-nLbCas12a(D156R/RuvC^{lidΔ}) | 1.91 ± 0.13 | 7.02 ± 0.67 |
| RT(5M)-nLbCas12a(D156R/RuvC^{lidΔ}/C931E) | 1.73 ± 0.19 | 6.4 ± 0.23 |
| RT(5M)-nLbCas12a(D156R/R1138A) | 3.94 ± 0.66 | 8.56 ± 0.73 |

Table 8 shows the impact of introducing silent mutations in the RTT of a pegRNA on the efficiency of different RT-nLbCas12a fusion proteins in oilseed rape (Brassica napus) protoplasts co-transformed with a dGFP reporter plasmid. Editing efficiencies are expressed as the percentage of GFP-positive cells three days post transfection as determined by fluorescence imaging.

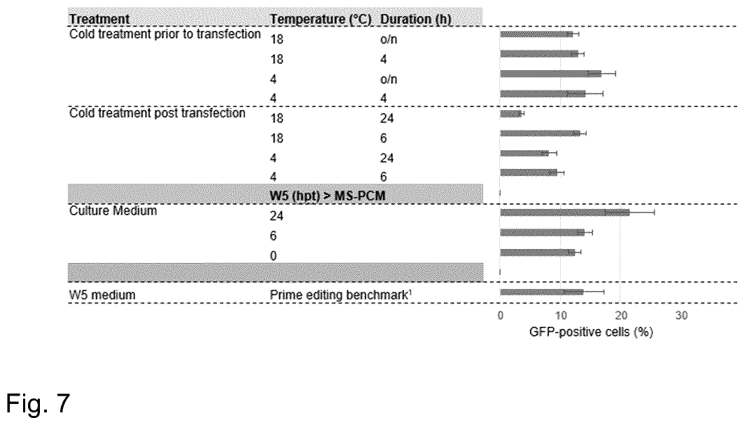

Finally, it was tested whether Cas12a PE rates can be further enhanced by boosting the division rate of the transfected protoplasts. This hypothesis was borne out by the observation that protoplasts exhibiting bright GFP fluorescence after successful editing often display elongated, bulging, or non-spherical shapes. These responses suggest regeneration of the cell wall and subsequent cell division. As a first strategy, transfected oilseed rape cells were cultured for several hours in standard W5 buffer and then transferred to MS-PCM, a culture medium frequently used for plant regeneration from protoplast cultures (Reed and Bargmann, 2021). In a second approach, the cell cycle was regulated and cell cycle synchronization was induced by exposing the protoplasts to a transient cold shock at 4° C. or 18° C., either before or after transfection. Regardless of the medium exchange or cold shock treatments, all cells were transfected with the dGFP reporter, R1138A-LbCas12a prime editor, MCP-RT (5M) and dGFP_pegRNA_4 (see Table 1). After transfection, the cells were cultured at 37° C. for 24 hours and subsequently transferred to 28° C. until 72 hours post-transfection. At this time point, the efficiency of GFP editing was quantified using fluorescence imaging. As shown in FIG. 7, incubating transfected cells in MS-PCM culture medium starting 24 hpt (hours post transfection) nearly doubled PE efficiency compared to the standard editing conditions (average 1.74-fold increase).

### Example 6: Removal of RNAse H Domain in M-MLV RT and N-Terminal Fusion of Viral Nucleocapsid Protein Fails to Enhance nCas12a-PE Efficiency

Previous findings showed that engineering the Moloney-murine leukemia virus reverse transcriptase (RT(5M)) by removing its ribonuclease H domain and incorporating a viral nucleocapsid protein with nucleic acid chaperone activity synergistically enhanced the efficiency of base substitutions, deletions and insertions at multiple endogenous sites in rice protoplasts transformed with a Cas9 prime editor (Zong et al., 2022). To test if these modifications also boost prime editing with Cas12a nickases, a new expression construct was generated comprising a nucleocapsid protein and engineered RT (5M) lacking the RNaseH domain fused at the N-terminus of the nLbCas12a (D156R/RuvC^{lidΔ}) nickase (SEQ ID NO: 213). A plasmid encoding this novel prime editor was introduced in oilseed rape protoplasts together with the above-described dGFP reporter (SEQ ID NO: 70), a dGFP-targeting pegRNA with or without silent mutations in the RTT (see dGFP_pegRNA_4 and dGFP_pegRNA_4+G2C/G11A in Table 8, SEQ ID NO: 33 and SEQ ID NO: 203), and optionally the MCP-RT (5M) fusion and nicking crRNA described in Example 5 (SEQ ID NO: 200 and SEQ ID NO: 201). The transfected cells were incubated in W5 medium at 37° C. for 24 h and thereafter transferred to MS-PCM culture medium at 28° C. GFP editing efficiencies were determined at three days post transfection and compared against that of the RT (5M)-nLbCas12a (D156R/R1138A) and RT (5M)-nLbCas12a (D156R/RuvC^{lidΔ}) prime editors (SEQ ID NO: 199 and SEQ ID NO: 65). While both editors induced substantial levels of GFP-positive cells in combination with unmodified pegRNAs (4.93% and 6.94% at 3 dpt, respectively), introducing same-sense mutations in the RTT, second-strand nicking and in trans expression of MCP-RT (5M) all further enhanced editing activity, with combinatorial treatments yielding editing efficiencies up to 17.27% and 12.55% (for R1138A and RuvC^{lidΔ} editors, respectively; see Table 9 and FIG. 8). In contrast, fusion of the NC protein and removal of the RT RNAseH domain strongly reduced prime editing activity across all treatments, which may point to improper folding of the fusion protein.

| | | dGFP_ | MCP- | | GFP-positive |
|---|---|---|---|---|---|
| | | pegRNA_4 + | RT | nicking | cells |
| Prime editor | dGFP_pegRNA_4 | G2C/G11A | (5M) | crRNA | (%) |
| RT(5M)-nLbCas12a(D156R/R1138A) | + | − | − | − | 6.94 ± 0.9 |
| RT(5M)-nLbCas12a(D156R/RuvC^{lidΔ}) | + | − | − | − | 4.93 ± 1.8 |
| NC-RT(5M/RNaseΔ)-nLbCas12a(D156R/RuvC^{lidΔ}) | + | − | − | − | 0.27 ± 0.3 |
| RT(5M)-nLbCas12a(D156R/R1138A) | − | + | − | − | 12.33 ± 3.36 |
| RT(5M)-nLbCas12a(D156R/RuvC^{lidΔ}) | − | + | − | − | 12.33 ± 1.28 |
| NC-RT(5M/RNaseΔ)-nLbCas12a(D156R/RuvC^{lidΔ}) | − | + | − | − | 3.53 ± 1.64 |
| RT(5M)-nLbCas12a(D156R/R1138A) | + | − | + | + | 13.64 ± 2.26 |
| RT-nLbCas12a(RuvC lid delta) | + | − | + | + | 9.62 ± 1.64 |
| NC-RT(5M/RNaseΔ)-nLbCas12a(D156R/RuvC^{lidΔ}) | + | − | + | + | 0.37 ± 0.3 |
| RT(5M)-nLbCas12a(D156R/R1138A) | − | + | + | + | 17.27 ± 2.07 |
| RT(5M)-nLbCas12a(D156R/RuvCMA) | − | + | + | + | 12.55 ± 0.96 |
| NC-RT(5M/RNaseΔ)-nLbCas12a(D156R/RuvC^{lidΔ}) | − | + | + | + | 0.73 ± 0.6 |

Table 9 shows the influence of RT engineering, pegRNA modifications, second-strand nicking and in trans expression of an MCP-RT (5M) fusion protein on the editing efficiencies of different RT-nCas12a prime editors in oilseed rape (Brassica napus) protoplasts co-transformed with a dGFP reporter plasmid. Editing efficiencies are expressed as the percentage of GFP-positive cells three days post transfection as determined by fluorescence imaging.

### Example 7: Modifying Secondary Structure of pegRNAs to Reduce Auto-Inhibitory Spacer-PBS Interactions Improves Efficiency of nCas12a-PE

Recent studies have shown that inherent complementarity and association between the PBS and spacer sequences within a pegRNA negatively influences Cas9 prime editing activity in human cells (Liu et al., 2021; Ponnienselvan et al., 2023). Because RNA-RNA duplexes are typically more stable than RNA-DNA duplexes (Kankia and Marky, 1999), one may hypothesize that the formation of a PBS-spacer RNA duplex not only prevents or disrupts R-loop formation but also interferes with reverse transcription by the RT (Liu et al., 2022; Feng et al., 2023; Ponnienselvan et al., 2023). Reducing PBS-spacer complementary base pairing is thus expected to improve target recognition, nicking and reverse transcription.

In this context, we sought to destabilize auto-inhibitory PBS-spacer interactions by incorporating one of two stem structures in between the spacer and RTT-PBS regions of the pegRNA: either a 20-nt stem derived from the stem-loop 1 structure of SpyCas9 tracRNA or a 20-nt stem derived from the spacer of a SpyCas9 gRNA targeting the wheat TaMLO gene. Whereas canonical pegRNAs (cf. SEQ ID NO: 33, s. FIG. 9A) have predicted auto-inhibitory base pairing between the spacer and PBS sequences, the engineered pegRNAs of SEQ ID NOs 214 (FIG. 9B) and 217 (s. FIG. 9C) are specifically engineered to disrupt spacer-PBS interactions through addition of different stem structures in between the spacer and RTT-PBS contributing to the functionality of these engineered pegRNAs as evident from the secondary structures of these engineered pegRNAs.

The resulting engineered pegRNAs (SEQ ID NOS: 214 to 219) were then tested with the RT (5M)-nLbCas12a (D156R/R1138A) prime editor (SEQ ID NO: 199) in the dGFP reporter assay in oilseed rape protoplasts and their efficiency was compared against that of the unmodified dGFP_pegRNA_4 (SEQ ID NO: 33). As shown in Table 10, the two backbone modifications displayed an average 2.56-fold (average 12% GFP-positive cells) and 2.74-fold (average 12.8% GFP-positive cells) improvement in editing compared with the unmodified pegRNA harboring an RTT of 66 nt and a PBS of 9 nt, respectively. Furthermore, the backbone-modified designs also supported efficient editing in combination with RTTs of 46 nt and 26 nt, despite these shorter RTT lengths completely abolishing prime editing activity of unmodified pegRNAs (see Table 1 in comparison). Together, these data not only illustrate the level of auto-inhibitory intramolecular base pairing in pegRNAs but also suggest that this auto-inhibition can be relieved through secondary structure modifications.

| | pegRNA | | | | | GFP-positive |
|---|---|---|---|---|---|---|
| Prime Editor | SEQ ID NO | Name | RTT (nt) | PBS (nt) | Stem | cells (%) |
| RT(5M)- | n/a | Negative Ctrl | n/a | n/a | n/a | 0 |
| nLbCas12a(D156R/ | 33 | dGFP pegRNA_4 | 66 | 9 | none | 4.67 ± 1.7 |
| R1138A) | 214 | dGFP pegRNA_4_mod1 | 66 | 9 | TaMLO-sgRNA | 12 ± 2.72 |
| | 215 | dGFP pegRNA_4_mod2 | 46 | 9 | TaMLO-sgRNA | 8 ± 1.91 |
| | 216 | dGFP pegRNA_4_mod3 | 26 | 9 | TaMLO-sgRNA | 5.5 ± 1.5 |
| | 217 | dGFP pegRNA_4_mod4 | 66 | 9 | Cas9 tracRNA stem-loop 1 | 12.8 ± 4.38 |
| | 218 | dGFP pegRNA_4_mod5 | 46 | 9 | Cas9 tracRNA stem-loop 2 | 7.17 ± 1.91 |
| | 219 | dGFP pegRNA_4_mod6 | 26 | 9 | Cas9 tracRNA stem-loop 3 | 5.08 ± 1.61 |

Table 10 shows the editing efficiency of different backbone-modified Cas12a pegRNAs in oilseed rape (Brassica napus) protoplasts co-transfected with a dGFP reporter plasmid and an RT-nCas12a prime editor. Editing efficiencies are expressed as the percentage of GFP-positive cells three days post transfection as determined by fluorescence imaging.

### Example 8: Impact of Circular and Linear Split pegRNAs on the Level of nCas12a-Mediated Prime Editing

The results shown in Examples 1 to 7 were obtained using pegRNAs that consist of a 5′ crRNA and a 3′extension containing the RTT and PBS. Despite their simplicity, these so-called ‘all-in-one’ pegRNAs are prone to RNA misfolding due to inevitable base pairing between the spacer in the crRNA and the PBS. Moreover, since the 3′ extension is exposed, ‘all-in-one’ pegRNAs are susceptible to degradation by exonucleases (Liu et al., 2022; Feng et al., 2023). Recent studies have shown that pegRNAs can be split into a single guide RNA and a second circular RNA containing the RTT and PBS (Liu et al., 2022; Feng et al., 2023; Liang et al., 2024). Because circular RNAs lack dissociative ends, they are generally more stable and less prone to inhibitory associations between the spacer and the PBS (Liu et al., 2022; Feng et al., 2023). Similarly, prime editors can be split such that the nickase remains untethered from the reverse transcriptase. Smaller in size and showing reduced molecular complexity, these untethered prime editors were found to exhibit similar editing activities as the parental tethered editors in human cells (Liu et al., 2022; Liang et al., 2024).

To assess the potential of split prime editing systems in plants, experiments were initiated in oilseed rape (Brassica napus) protoplasts using the dGFP reporter assay described above. To this end, the best-performing dGFP-targeting pegRNA identified in previous examples (dGFP_pegRNA_4 in Table 1; SEQ ID NO: 33) was split into a crRNA containing the spacer and a separate RTT-PBS sequence, referred to as the prime editing template RNA (petRNA). In addition, an MS2 aptamer was added to both 5′ and 3′ ends of the RTT-PBS sequence to tether it to an MCP-RT fusion protein (SEQ ID NO: 220). The MS2-tagged RTT-PBS was also engineered into a circular form (SEQ ID NO: 221) by flanking it with 5′ and 3′ ligation sequences and self-cleaving Twister ribozymes, according to the previously described Tornado circular RNA expression system (Litke et al., 2019). The dGFP-targeting crRNA (SEQ ID NO: 223) and circular petRNAs were expressed either separately or as a bicistronic transcript (SEQ ID NO: 222) under control of a single Arabidopsis U6 promoter (SEQ ID NO: 150). The crRNA in the bicistronic transcript was flanked by two mature direct repeats (SEQ ID NO: 91) such that Cas12a would process the tandem RNAs, cleaving the 5′ end of the crRNA and releasing it together with the petRNA. As control for the petRNAs, an ‘all-in-one’ pegRNA with flanking MS2 tags was devised. Plasmids expressing the pegRNA, crRNA and the respective petRNAs were co-transfected into oilseed rape protoplasts together with expression constructs coding for the dGFP reporter (SEQ ID NO: 70), the NLS-tagged ‘RuvC^{lidΔ}’ nickase (SEQ ID NO: 266) and MCP-RT (5M) (SEQ ID NO: 200). Transfected cells were incubated in W5 for 24 h at 37° C. and then transferred to 28° C. Editing frequencies were quantified at 3 days post transfection through fluorescence imaging. As shown in Table 11, editing by the different petRNAs was several-fold higher than that by the MS2-pegRNA control, suggesting that the anticipated reduction in auto-inhibition is positively correlated with the editing efficiency. Interestingly and opposite to findings in human cells, linear petRNAs outperformed the mono- and bicistronic circular petRNAs, with linear petRNA-mediated editing efficiencies reaching as high as 20.96% As expected, no GFP fluorescence could be detected when using pegRNAs without flanking MS2 hairpins (data not shown), which is consistent with the need for active recruitment of RT to the target site via MS2-MCP tethering.

Table 11 shows the editing efficiency of different split prime editing systems in oilseed rape (Brassica napus) protoplasts co-transfected with a dGFP reporter plasmid. Editing efficiencies are expressed as the percentage of GFP-positive cells three days post transfection as determined by fluorescence imaging.

| | | gRNA | | | | | |
|---|---|---|---|---|---|---|---|
| | | | | | | bicistronic | |
| | | | | | | MS2- | |
| | | | | MS2- | | tagged | |
| | | | MS2- | tagged | MS2-tagged | circular | |
| | | | tagged | linear | circular | petRNA + | GFP-positive |
| Prime Editor | RT | crRNA | pegRNA | petRNA | petRNA | crRNA | cells (%) |
| nLbCas12a | MCP-RT(5M) | − | + | − | − | − | 1.01 ± 0.36 |
| D156R/RuvC^{lidΔ}) | | + | − | + | − | − | 20.96 ± 4.77 |
| | | + | − | − | + | − | 9.43 ± 3.0 |
| | | − | − | − | − | + | 12.46 ± 1.79 |

Next, the results obtained in the GFP reporter assay were validated at the BnFAD2 target site. To this end, plasmids encoding MCP-RT (5M) (SEQ ID NO: 200) and the RT-nLbCas12a (D156R/R1138A) prime editor (SEQ ID NO: 199) were co-transfected into oilseed rape (Brassica napus) protoplasts along with different FAD2-targeting peg- and petRNAs with a PBS length of 9 bases and an RTT of either 44 or 66 bases. For each RTT, the editing efficiency of ‘all-in-one’ pegRNAs containing both the crRNA and RTT-PBS (SEQ ID NO: 232 and SEQ ID NO: 240) was compared against that of engineered pegRNAs with inserted stem structures (hereafter referred to as ‘backbone-modified’ pegRNAs, SEQ ID NOS: 253 to 256), on the one hand, and split RNA designs comprising separately expressed crRNA and MS2-tagged linear or circular petRNAs, on the other hand (SEQ ID NOS: 257 to 260).

Transfected oilseed rape protoplasts were soon after transfection embedded in thin alginate layers to promote cell division and incubated at 28° C. Seven days post transfection, alginate half layers containing dividing cells were harvested and editing frequencies were determined by deep amplicon sequencing. While only low levels of precise edits (<0.05%) could be detected with the canonical pegRNAs, using modified pegRNAs harboring one of two backbone modifications improved editing activity, with efficiencies reaching up to 0.18% (see Table 12).

Moreover, consistent with the results from the dGFP reporter assay, splitting pegRNAs into a crRNA and a separate petRNA triggered an even larger increase in editing efficiency compared to the canonical pegRNAs, averaging 23.19- and 6.8-fold higher editing with the MS2-tagged linear and circular templates, respectively. Testing the split RNAs at a different target site (BnALS3) and with a different prime editor (nLbCas12a (D156R/RuvC^{lidΔ}; SEQ ID NO: 266) yielded very similar results, with the MS2-tagged linear petRNAs strongly improving editing over that of both the unsplit pegRNAs and circular petRNAs, reaching editing frequencies of up to 2.68% (see Table 13 below).

Collectively, these results show that Cas12a prime editing can be severely boosted by splitting the pegRNA into a crRNA and a separate MS2-tagged linear petRNA harboring the RTT and PBS.

| | | | | | NGS results | |
|---|---|---|---|---|---|---|
| | RNA | | | Alginate | Total read | Editing |
| Prime Editor | Description | RTT (nt) | PBS (nt) | layer | count | efficiency (%) |
| RT(5M)- | Non-targeting pegRNA | 66 | 9 | 1 | 254,564 | 0.00 |
| nLbCas12a(D156R/ | “all-in-one” pegRNA | 66 | 9 | 1 | 141,052 | 0.04 |
| R1138A | Backbone-modified “all-in-one” pegRNA_1 | 66 | 9 | | 122,818 | 0.03 |
| | Backbone-modified “all-in-one” pegRNA_2 | 66 | 9 | | 141,866 | 0.00 |
| | crRNA + MS2-tagged linear petRNA | 66 | 9 | | 176,426 | 0.77 |
| | crRNA + MS2-tagged circular petRNA | 66 | 9 | | 155,412 | 0.31 |
| | crRNA Ctrl | n/a | n/a | | 166,893 | 0.00 |
| | “all-in-one” pegRNA | 66 | 9 | 2 | 83,277 | 0.00 |
| | Backbone-modified “all-in-one” pegRNA_1 | 66 | 9 | | 87,061 | 0.01 |
| | Backbone-modified “all-in-one” pegRNA_2 | 66 | 9 | | 39,823 | 0.01 |
| | crRNA + MS2-tagged linear petRNA | 66 | 9 | | 140,461 | 0.79 |
| | crRNA + MS2-tagged circular petRNA | 66 | 9 | | 115,760 | 0.34 |
| | crRNA Ctrl | n/a | n/a | | 65,251 | 0.00 |
| | “all-in-one” pegRNA | 44 | 9 | 1 | 166,766 | 0.03 |
| | Backbone-modified “all-in-one” pegRNA_1 | 44 | 9 | | 152,508 | 0.17 |
| | Backbone-modified “all-in-one” pegRNA_2 | 44 | 9 | | 145,148 | 0.18 |
| | crRNA + MS2-tagged linear petRNA | 44 | 9 | | 138,902 | 0.55 |
| | crRNA + MS2-tagged circular petRNA | 44 | 9 | | 129,916 | 0.14 |
| | crRNA Ctrl | n/a | n/a | | 189,653 | 0.00 |
| | “all-in-one” pegRNA | 44 | 9 | 2 | 8,074 | 0.01 |
| | Backbone-modified “all-in-one” pegRNA_1 | 44 | 9 | | 81,720 | 0.08 |
| | Backbone-modified “all-in-one” pegRNA_2 | 44 | 9 | | 109,598 | 0.26 |
| | crRNA + MS2-tagged linear petRNA | 44 | 9 | | 122,293 | 0.64 |
| | crRNA + MS2-tagged circular petRNA | 44 | 9 | | 75,613 | 0.16 |
| | crRNA Ctrl | n/a | n/a | | 111,308 | 0.00 |

Table 12 shows the editing efficiency of different split and unsplit Cas12a prime editing systems at an endogenous BnFAD2 target site in alginate-embedded oilseed rape (Brassica napus) protoplasts at three days post transfection. Editing efficiencies are expressed as the percentage of total NGS reads having the desired +2 CAT to GCA edit.

| | | | | | | | Editing efficiency (%) | | | |
|---|---|---|---|---|---|---|---|---|---|---|
| | | | | | | | crRNA + | crRNA + | | |
| | | | | | | | MS2- | MS2-tagged | | non- |
| | Detection | | | LbCas12a | RTT | PBS | tagged linear | circular | all-in-one | targeting |
| Sample | method | Target | Edit | nickase | (nt) | (nt) | petRNA | petRNA | pegRNA | pegRNA |
| | NGS | BnALS3 | +9 | R1138A | 66 | 11 | 0.29 | 0.19 | 0 | 0 |
| Alginate layer | NGS | BnALS3 | GACCGT to | R1138A | 66 | 11 | 0.36 | 0.21 | 0 | 0 |
| Liquid culture | ddPCR | BnALS3 | AACCAC | RuvClidΔ | 66 | 11 | 0.54 | nd | 0 | 0 |
| | ddPCR | BnFAD2 | | RuvClidΔ | 44 | 9 | 1.06 | 0.22 | 0 | 0 |
| Alginate layer | ddPCR | BnFAD2 | +2 CAT to | RuvClidΔ | 44 | 9 | 1 | 0.23 | 0 | 0 |
| | ddPCR | BnFAD2 | GCA | R1138A | 44 | 9 | 2.68 | nd | 0.09 | 0 |
| | ddPCR | BnFAD2 | | R1138A | 66 | 9 | 1.6 | nd | 0.24 | 0 |
| | ddPCR | BnFAD2 | | RuvClidΔ | 44 | 9 | 0.97 | nd | nd | 0.02 |
| Liquid culture | ddPCR | BnFAD2 | | RuvClidΔ | 66 | 9 | 1.13 | nd | nd | 0.02 |

Table 13 shows the efficiency of precise editing induced by different nLbCas12a prime editors and split or unsplit pegRNAs at the endogenous BnALS3 and BnFAD2 target sites in oilseed rape (Brassica napus) protoplasts at three days post transfection. Editing efficiencies are expressed as the percentage of total NGS reads having the desired edits.

### Example 9: Impact of Small RNA-Binding Exonuclease Protection Factor La and MCP-nCas12a Fusions on the Level of nCas12a-Mediated Prime Editing

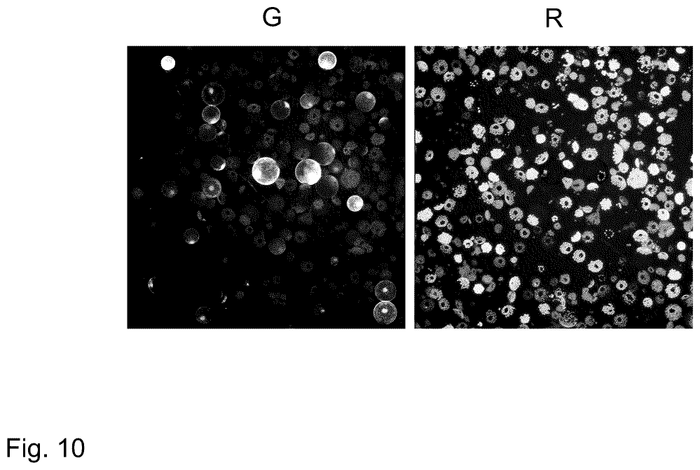

Having shown that engineered petRNAs can strongly enhance Cas12a editing efficiency, we next sought to optimize the prime editor design. La, a ubiquitously expressed eukaryotic protein, is involved in diverse aspects of RNA metabolism, including binding polyuridine (polyU) tracts at the 3′ ends of nascent RNA polymerase III (Pol III) transcripts and protecting them from exonucleases. Recently, it was reported that fusing the N-terminal domain of La (hereafter referred to as La (1-194)) to nCas9-RT proteins strongly improved prime editing across a variety of target sites in human cells (Yan et al., 2024). To assess the impact of La on the level of nCas12a-PE, a new expression construct was generated encoding the La (1-194) domain fused to the C-terminus of the RT-nLbCas12a (D156R/R1138A) protein (SEQ ID NO: 270). In a different design, MS2 coat protein was fused to the C-terminus of the prime editor (SEQ ID NO: 269). Plasmids encoding the respective fusion proteins were co-delivered in oilseed rape protoplasts together with constructs encoding the dGFP reporter (SEQ ID NO: 70), MCP-RT (5M) (SEQ ID NO: 200), dGFP-targeting crRNA (SEQ ID NO: 223) and either circular or linear MS2-tagged petRNAs (SEQ ID NO: 221 and SEQ ID NO: 220, respectively). Editing efficiencies of the La- and MCP-fused RT-nLbCas12a-R1138A editors was determined at 2 days post transfection by quantifying the percentage of GFP-positive cells and their efficacy was compared against that of nLbCas12a (D156R/R1138)-expressing constructs with or without N-terminal RT (SEQ ID NO: 199; SEQ ID NO: 267; SEQ ID NO: 268) as well as that of an untethered editor containing the nLbCas12a-‘RuvC^{lidΔ}’ nickase (SEQ ID NO: 266). In common with the results shown in Example 8, editing by the linear petRNAs was generally more efficient than that by the circular petRNAs, irrespective of the prime editor used. Comparing the R1138A and ‘RuvC^{lidΔ}’ nickases, we found the editor comprising the ‘RuvC^{lidΔ}’ nickase to exhibit a strong increase in reporter editing as compared to the R1138A editor, with the former inducing GFP frequencies as high as 16.8% with the linear petRNAs (see Table 14). The activity of the R1138 editor could, however, be strongly enhanced by covalently linking the RT to the N-terminus of the nickase protein, while prime editor variants with either XTEN or (GGGGS)6x linkers in between the RT and the nickase exhibited comparable editing efficiencies. Moreover, consistent with a benefit from tethering the MS2-tagged petRNAs to the prime editor protein, fusing MCP to the C-terminus of the RT-LbCas12a (R1138A) strongly enhanced editing activity, with the percentage of GFP-positive cells reaching up to 42.3% (average 31.38%) and 24.2% (average 20.1%) with the linear and circular petRNAs, respectively (see FIG. 10). Interestingly, C-terminal fusions of La(1-194) resulted in a comparable increase in editing activity, albeit only for the linear petRNAs, which strongly suggests that La predominantly acts by protecting the RTT and PBS from degradation by exonucleases.

| | | | MS2- | MS2- | GFP- |
|---|---|---|---|---|---|
| | | | tagged | tagged | positive |
| | | | linear | circular | cells |
| Prime Editor | MCP-RT | crRNA | petRNA | petRNA | (%) |
| None | − | + | − | − | 0 |
| nLbCas12a | + | + | + | − | 16.83 ± 2.49 |
| (D156R/RuvC^{lidΔ}) | + | + | − | + | 9.32 ± 2.78 |
| nLbCas12a | + | + | + | − | 1.05 ± 0.37 |
| (D156R/R1138A) | + | + | − | + | 0 |
| RT(5M)-XTEN-nLbCas12a | + | + | + | − | 8.66 ± 0.79 |
| (D156R/R1138A) | + | + | − | + | 3.54 ± 0.39 |
| RT(5M)-(GGGGS)6x-nLbCas12a | + | + | + | − | 8.4 ± 2.26 |
| (D156R/R1138A) | + | + | − | + | 3.54 ± 0.39 |
| RT(5M)-XTEN-nLbCas12a | + | + | + | − | 31.38 ± 10.9 |
| (D156R/R1138A)-MCP | + | + | − | + | 20.08 ± 4.17 |
| RT(5M)-XTEN-nLbCas12a | + | + | + | − | 24.18 ± 6.46 |
| (D156R/R1138A)-La | + | + | − | + | 3.15 ± 0.79 |

Table 14 shows the editing efficiency of linear and circular split pegRNAs in oilseed rape (Brassica napus) protoplasts co-transfected with a dGFP reporter plasmid and different nCas12a prime editors. Editing efficiencies are expressed as the percentage of GFP-positive cells three days post transfection as determined by fluorescence imaging.

FIG. 10 demonstrates successful editing of an extrachromosomal dGFP reporter in oilseed rape (Brassica napus) protoplasts co-expressing a Cas12a prime editor composed of the nLbCas12a (D156R/R1138A) nickase with N-terminal RT and C-terminal MCP fusions (SEQ ID NO: 269) together with the MCP-RT (5M) fusion of SEQ ID NO: 200, a dGFP-targeting Cas12a crRNA (SEQ ID NO: 223) and MS2-tagged linear template RNAs (SEQ ID NO: 220). R denotes the red channel (autofluorescence) and G denotes the green channel (GFP).

Next, the La and MCP prime editors were examined for editing with MS2-tagged circular or linear RNAs at the endogenous BnFAD2 target site in oilseed rape protoplasts. Similar prime editing systems were tested as in the dGFP reporter experiment described above. Co-transfected crRNA without petRNA served as negative control and the best-performing all-in-one pegRNA from Example 4 (SEQ ID NO: 240) was included as a positive control. The transfected protoplasts were embedded in alginate as described before and editing efficiencies were determined in samples taken from 7-day-old protoplast cultures. In common with the data from the dGFP reporter experiment, MS2-tagged linear petRNAs consistently outperformed not only the all-in-one pegRNAs but also the circular petRNAs, while use of the ‘RuvC^{lidΔ}’ nickase led to an almost 3-fold increase in editing compared to the R1138A variant (average 0.57% precise edits versus 0.2% with linear petRNAs, respectively; see Table 15). Moreover, whereas nLbCas12a (D156R/R1138)-containing prime editors with XTEN or (GGGGS)6x linkers in between the RT and the nickase displayed similar editing frequencies, appending MCP or La (1-194) to the C-terminus of the R1138 prime editor strongly improved editing by an average 5.16- and 8.04-fold, respectively, with the La-fused R1138 editors inducing efficiencies as high as 1.24% with linear petRNAs.

| | | | NGS results | | | |
|---|---|---|---|---|---|---|
| | | | Alginate layer 1 | | Alginate layer 2 | |
| | | | Total | Editing | Total | Editing |
| | MCP- | | read | efficiency | read | efficiency |
| Prime editor | RT | RNA | count | (%) | count | (%) |
| None | − | non-targeting pegRNA | 57,793 | 0.00 | 62,547 | 0.00 |
| nLbCas12a(D156R/RuvC^{lidΔ}) | + | crRNA + MS2-tagged linear petRNA | 63,468 | 0.64 | 19,777 | 0.64 |
| | + | crRNA + MS2-tagged circular petRNA | 100,860 | 0.15 | 51,998 | 0.14 |
| | + | crRNA Ctrl | 75,213 | 0.00 | 51,636 | 0.00 |
| | + | “all-in-one” pegRNA | 51,740 | 0.0 | 21,686 | 0.00 |
| nLbCas12a(D156R/R1138A) | + | crRNA + MS2-tagged linear petRNA | 66,947 | 0.17 | 53,258 | 0.22 |
| | + | crRNA + MS2-tagged circular petRNA | 62,018 | 0.06 | 29,747 | 0.09 |
| | + | crRNA Ctrl | 57,215 | 0.01 | 42,734 | 0.00 |
| | + | “all-in-one” pegRNA | 67,394 | 0.06 | 15,048 | 0.00 |
| RT(5M)-XTEN- | + | crRNA + MS2-tagged linear petRNA | 89,037 | 0.02 | 56,236 | 0.28 |
| nLbCas12a(D156R/R1138A) | + | crRNA + MS2-tagged circular | 91,471 | 0.04 | 79,694 | 0.05 |
| | | petRNA | | | | |
| | + | crRNA Ctrl | 75,453 | 0.01 | 23,821 | 0.00 |
| | + | “all-in-one” pegRNA | 103,050 | 0.07 | 57,866 | 0.02 |
| RT(5M)-(GGGGS)6x- | + | crRNA + MS2-tagged linear petRNA | 121,415 | 0.18 | 66,818 | 0.15 |
| nLbCas12a(D156R/R1138A) | + | crRNA + MS2-tagged circular petRNA | 78,712 | 0.05 | 40,474 | 0.06 |
| | + | crRNA Ctrl | 65,239 | 0.00 | 51,067 | 0.00 |
| | + | “all-in-one” pegRNA | 70,110 | 0.27 | 68,323 | 0.02 |
| RT(5M)-XTEN- | + | crRNA + MS2-tagged linear petRNA | 89,372 | 0.84 | 64,441 | 0.70 |
| nLbCas12a(D156R/R1138A)- | + | crRNA + MS2-tagged circular | 69,301 | 0.41 | 66,006 | 0.34 |
| MCP | | petRNA | | | | |
| | + | crRNA Ctrl | 52,803 | 0.00 | 30,661 | 0.00 |
| | + | “all-in-one” pegRNA | 32,937 | 0.0 | 31,161 | 0.06 |
| RT(5M)-XTEN- | + | crRNA + MS2-tagged linear petRNA | 91,268 | 1.16 | 44,778 | 1.24 |
| nLbCas12a(D156R/ | + | crRNA + MS2-tagged circular petRNA | 66,230 | 0.09 | 46,906 | 0.10 |
| R1138A)-La | + | crRNA Ctrl | 72,558 | 0.00 | 44,208 | 0.00 |
| | + | “all-in-one” pegRNA | 83,494 | 0.01 | 41,359 | 0.02 |

Table 15 shows the editing efficiency of different split and unsplit Cas12a prime editing systems at an endogenous BnFAD2 target site in alginate-embedded oilseed rape (Brassica napus) protoplasts at three days post transfection. Editing efficiencies are expressed as the percentage of total NGS reads having the desired +2 CAT to GCA edit.

In view of the enhanced editing observed with the untethered ‘RuvC^{lidΔ}’ editor, next steps will focus on assessing the efficiency of ‘RuvC^{lidΔ}’-containing editors harboring single or combinatorial fusions of MCP and/or La (SEQ ID NOS: 271 to 275 and SEQ ID NO: 277). In addition, the impact of introducing silent mutations in the RTT of the BnFAD2-, BnALS3- and dGFP-targeting peg- and petRNAs will be assessed (for examples see SEQ ID NOs: 261 to 265).

### Example 10: Analysis of Cas12a Prime Editing in E. coli

Since editing experiments in bacterial systems could provide options for high-throughput optimization of both pegRNA and prime editor designs, prime editing is also studied in E. coli using combined selection and screening strategies. While selections isolate correctly edited bacterial cells through selective replication and thus offer unparalleled throughput, screens leverage prime editing to directly measure changes in cellular phenotypes, thus enabling accurate quantification of prime editing.

### General Approach

For both types of E. coli experiments, three classes of plasmid are used: i/ pCas plasmids drive expression of the nickase fusions with reverse transcriptase (RT) and associated controls (such as a nickase Cas9 fused to an RT, or Cas12a without RT); ii/ pGuide plasmids encode the different pegRNAs, all of which comprise a spacer, a PBS for reverse transcription and an RTT encoding the desired edits, and iii/ pTarget plasmids provide targets for prime editing.

For the pCas plasmids, a p15A origin of replication (ORI) and an ampicillin resistance marker is used. Different pCas plasmids are generated including pCas_nCas9-RT pCas_nCas12a, pCas_nCas12a-RT, pCas_RT-nCas12a. As a positive control for prime editing, the pCas_nCas9-RT plasmid is used; this construct is based on the PE2 construct for Cas9-based prime editing, including a SpCas9 nickase (disrupted HNH-site, H840A) fused to a modified version of the M-MLV Reverse Transcriptase (Anzalone et al., 2019). The pCas_nCas12a plasmid contains a nickase version of LbCas12a (nCas12a; SEQ ID NO: 15) without an RT and is intended as a negative control to verify that editing outcomes are resulting from reverse transcription. pCas_nCas12a-RT encodes a C-terminal fusion of the RT to nCas12a, while pCas_RT-nCas12a encodes an N-terminal fusion of the same RT domain. Both fusions use a protein linker made up of an XTEN linker sequence flanked by glycine serine residues on either side (SGGSSGGSSGSETPGTSESATPESSGGSSGGS; SEQ ID NO: 134). At a later stage, different linkers are tested to evaluate the impact on editing efficiency.

In case of pGuide plasmids, a ColE1 ORI is used in combination with a spectinomycin resistance marker. pGuide plasmids encode either different variants of pegRNAs to test different prime editing efficiencies, or as negative controls only crRNAs. For pegRNAs, different configurations are tested with different lengths of the PBS and RTT. Aiming to improve RNA stability, pegRNAs with and without addition of the PP7 hairpin at the 3′ end of the PBS will be evaluated. Moreover, to minimize heterogeneity of editing outcomes within single cells, the low copy number pBeloBAC11 backbone is selected for the pTarget plasmids, this backbone including genes providing active partitioning of the plasmid DNA and chloramphenicol resistance.

To allow prime editing, E. coli DH10B cells are first transformed with different combinations of pCas and pGuide plasmids. Next, the resulting transformants are transformed with a pTarget plasmid. When all three plasmids are present, the prime editor (or nickase control) is expressed from pCas and guided to the target sequence by the pegRNA (or crRNA control), where a nick is created in the non-target strand. In the case of pegRNAs, the primer binding site (PBS) is expected to bind the target site DNA upstream from the nick site. Subsequently, the RT will extend the DNA as dictated by the RTT on the pegRNA, resulting in two DNA intermediates containing a 3′ or a 5′ DNA flap, respectively, the latter constituting the RTT-dictated DNA containing the desired edit. The 5′ DNA flap is then resolved by endogenous factors, completing the process. Although most applications described for prime editing focus on eukaryotes, successful Cas9-based prime editing has previously been shown in E. coli (Tong et al., 2021), indicating that the 5′ DNA flap can also be efficiently resolved in bacteria.

### Selective Approach: pTargets

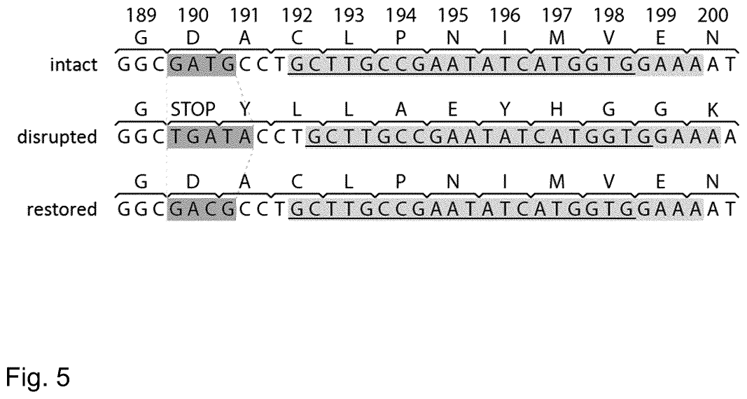

For the selective approach, the pTarget plasmid contains the Aminoglycoside 3′-phosphotransferase (APH(3′))-encoding neo gene, which provides resistance to the antibiotic kanamycin. Beside the pTarget with the intact neo gene (pTarget_neo_intact), a plasmid is constructed labelled ‘pTarget_neo_disrupted’ that encodes a mutated APH(3′) where 4 bp-including those encoding the active site residue D190—is replaced by a 5 bp edit encoding an in-frame stop codon and causing a frameshift downstream of the edit (FIG. 5). Prime editing is used to repair the neo gene by replacing the 5 bp disruption with the native 4 bp sequence that corrects the D190 codon and restores the reading frame. Compared to pTarget_neo_intact, the repaired neo gene contains a silent mutation in the wobble base of the D190 codon to differentiate prime editing events from potential contamination with the pTarget_neo_intact plasmid (FIG. 5).

### Selective Approach: Experimental Steps

To allow prime editing of the neo gene, competent E. coli cells are co-transformed with pCas, pGuide, and pTarget plasmids. The transformed bacteria are then incubated for 1 h at 37° C. with vigorous shaking in LB medium (10 g/L NaCl, 10 g/L Peptone, 5 g/L yeast extract) without antibiotics, allowing the cells to build resistance to chloramphenicol (Cam), spectinomycin (Spec), and ampicillin (Amp). Next, 5 μL from this recovery culture is used to inoculate 50 ml conical tubes containing 5 ml of LB with Spec (50 μg/mL), Amp (100 μg/mL), and Cam (25 μg/mL). The 5 mL cultures are incubated for 16 h at 37° C. with vigorous shaking. During this incubation, prime editing can take place in the absence of kanamycin (Kan). Next, 2 μL of each culture is transferred to 198 μL LB with Cam, Spec, and Amp in a transparent flat-bottom 96-well plate. In addition, 2 μL from the 5 mL culture is transferred to LB with Cam, Spec, Amp, and Kan (50 μg/mL) on the same 96-wells plate.

The 96-well plate is then incubated at 37° C. for 24h in a plate reader, measuring the OD600 of each well every 4 min. Samples without Kan are expected to grow normally. In the presence of Kan, bacterial growth (as assessed through OD600) is only expected in conditions with an intact or restored neo gene. This includes control samples with the pTarget_neo_intact plasmid as well as samples with pTarget_neo_disrupted where prime editing has been successful. Prime-edited samples are expected to show a delay in OD600 as compared to the samples without Kan or with the pTarget_neo_intact plasmid since editing of the pTarget plasmid will likely only happen in a fraction of the cells before addition of Kan.

### Selective Approach: Plasmid Enrichment

In a second stage, the selective approach is combined with the use of pegRNA and/or RT-nCas12a diversity libraries enabling testing of large suites of pegRNA and/or Cas12a prime editor variants. Transformation of plasmid libraries is performed exactly as described above. Following addition of Kan, only those cells with prime edited pTarget plasmids are expected to grow, resulting in selective enrichment of pCas and pGuide variants based on their editing efficiencies.

### Quantitative Approach: pTargets

For the quantitative approach, a GFP reporter assay is used. In this assay, a pTarget plasmid harboring an intact GFP gene (pTarget_GFP_intact) produces functional GFP, resulting in fluorescence. Like the pTarget plasmids used for the selective approach, an additional pTarget_GFP_disrupted plasmid expressing defective GFP is constructed that does not yield fluorescence but provides a basis for monitoring prime editing that repairs the inactivated GFP gene such that the restored coding sequence can be differentiated from the original DNA sequence.

### Quantitative Approach: Experimental Steps

The experimental setup of the quantitative approach is similar to that of the selective approach. pGuide plasmids are generated with pegRNAs that allow prime editing-based restoration of GFP on the pTarget_disrupted plasmid. E. coli cells are transformed with pCas, pGuide, and pTarget. To enable prime editing, transformed cells are incubated for 1 h at 37° C. with vigorous shaking in LB without antibiotics, 5 μL is used to inoculate 5 mL LB with Cam, Spec, and Amp which is then incubated for 16 h at 37° C. with shaking. Next, various concentrations of the culture are plated on LB agar (LB with 15 g/L agar) with Cam, Spec, and Amp. Prime editing efficiency is calculated by scoring the number of fluorescent and non-fluorescent colonies and expressed as the percentage of colonies showing fluorescence (on a LB agar plate with 50-500 colonies in total). In addition, the percentage of GFP-positive cells is quantified using flow cytometry.

By comparing various pCas and pGuide plasmids, prime editing efficiencies can be assessed for a large set of different pegRNA designs and different nCas12a-RT architectures. Both N- and C-terminal RT fusions as well as a suite of linker sequences of different lengths and flexibility are tested. Results in E. coli are compared to those in human and plant cells to assess to what extent the results obtained from E. coli translate to other contexts.

### Example 11: Analysis of Cas12a Prime Editing in Human Cells

To evaluate the performance of nCas12a prime editors in human cells, HEK293T or K562 cells were transfected with a subset of the different prime editing systems described in Examples 1 to 9 via commonly established viral or non-viral methods, including lentivirus transduction, lipofection and DNA or RNA nucleofection. Synthetic RNA molecules were purchased from IDT DNA, while mRNAs were produced through in vitro transcription (IVT).

### 11.1 Methods

Cloning of nLbCas12a (D156R/RuvC Lid Delta) Expression Constructs

Different constructs enabling expression of an engineered M-MLV RT (D200N+L603W+T330P+T306K+W313; SEQ ID NO: 146; referred to as RT (5M)) fused to the N-terminus of the nLbCas12a (D156R/RuvC^{lidΔ}) nickase (SEQ ID NO: 15) were generated. First, a construct designated pRT026 (SEQ ID NO: 324) was established encoding the RT (5M)-RuvC^{lidΔ}′ prime editor flanked by N- and C-terminal SV40 nuclear localization signals under control of both the CMV and a mutated T7 promoter such that the same plasmid can be used for expression in mammalian cells and synthesis of mRNA through in vitro transcription (IVT). Next, a lentiviral expression construct was engineered by cloning the coding sequence of the SV40-tagged ‘RuvC^{lidΔ}’ prime editor into the lenti-CRISPR V2 plasmid (Addgene 52961) through Gibson assembly using the primers of SEQ ID NOS: 318 to 323, resulting in plasmid Lenti-LbCas12a_PE (SEQ ID NO: 325).

In Vitro Transcription (IVT) Synthesis of mRNA

mRNAs encoding the RT (5M)-nLbCas12a (D156R/R1138) prime editor (SEQ ID NO: 199) or Cas nuclease controls were prepared as previously described (Gaudelli et al., 2020). For each editor or control, a plasmid was constructed based on Addgene plasmid #178113. These plasmids all contained an inactivated T7 promoter, a 5′ untranslated region (UTR), Kozak sequence, open reading frame of the editor or nuclease, and a 3′ untranslated region. The nuclease genes were codon optimized for use in human cells. From these plasmids, the region described above was PCR amplified, using the forward and reverse primers of SEQ ID NO: 197 and SEQ ID NO: 198 and Q5 (NEB) or Phusion (Thermo Fisher Scientific) polymerase 2× mastermix according to the manufacturer's protocol. The forward primer was designed to correct the mutation in the T7 promoter while the reverse primer was used to add a polyA tail to the 3′ UTR. For expression of RT (5M)-nLbCas12a (D156R/RuvC^{lidΔ}) mRNA, 1 μg pRT026 was linearized using Ndel restriction enzyme. Then, 50 ng linearized pRT026 was amplified via PCR using Q5 2× polymerase mix (NEB) and the forward and reverse primers of SEQ ID NO: 327 and SEQ ID NO: 328. The PCR products were then purified using Zymo Research kit D4004 or Qiagen Gel Cleanup Kit (28704) and used as template for IVT.

mRNA was synthesized in vitro by T7 RNA polymerase-mediated transcription at 37° C. for 2-4 hours using 100% substituted N1-Methylpseudouridine-5′-triphosphate (TriLink Biotechnologies) and 1 μg of purified PCR product as template according to Table 16. CleanCap® Reagent AG (NEB) was included for co-transcriptional capping of mRNA resulting in a Cap1 structure. After removal of the PCR template through DNaseI digestion, the transcribed mRNAs were precipitated by adding 2.5 M Lithium Chloride, incubating at 20° C. for 30 min, and centrifuging at top speed in a tabletop centrifuge for 15 min. After discarding the supernatant, the obtained pellet was washed with ice-cold 70% ethanol twice, and finally resuspended in nuclease-free water. Alternatively, the mRNAs were purified using the Monarch RNA cleanup kit (NEB, T2040L). The purified mRNA was quantified using Nanodrop or Qubit RNA BR (Thermo Fisher Scientific) and stored at −70° C. Average yields were around 100 μg per reaction.

| | Volume | Final | |
|---|---|---|---|
| Component | (μl) | concentration | |
| 10× Reaction Buffer | 2 | 0.5× | |
| DTT (100 mM) | 2 | 5 | mM |
| Urea (8M) | 4 | 0.8M | |
| ATP (100 mM) | 2 | 5 | mM |
| GTP (100 mM) | 2 | 5 | mM |
| p-UTP (100 mM) (Tri-link) | 2 | 5 | mM |
| CTP (100 mM) | 2 | 5 | mM |
| CleanCap AG (40 mM) | 4 | 4 | mM |
| Template DNA (100 ng/ul) | 10 | 25 | ng/μL |

| | | |
|---|---|---|
| DNAse/RNAse free water | 6 | n/a |
| T7 RNA Polymerase mix | 4 | n/a |

Table 16 shows the composition of the T7 transcription mixture used for in vitro synthesis of Cas12a prime editor mRNAs.

Generation of Lentiviral Cell Lines Expressing the nLbCas12a (D156R/RuvC^{lidΔ}) Prime Editor

To produce lentivirus harboring the nLbCas12a (D156R/RuvC^{lidΔ}) prime editor, 4 μg of plasmid Lenti-LbCas12a_PE was co-transfected together with 3 μg of packaging plasmid (Addgene 12260) and 1 μg of envelope plasmid (Addgene 12259) into HEK293T cells as detailed below:
Day 0. Start new cell culture by seeding HEK293T cells in a 10 cm tissue culture plate.
Day 1. Mix prime editor, packaging and envelope plasmids with 1 ml Optimem (Gibco, 3198506) and 25 μg PEI (Polysciences, 23966) and incubate for 30 min. Then, add the transfection mix to the pre-seeded HEK293 cells at 70% confluence.
Day 2. Replenish the culture medium with DMEM supplemented with 10% FBS
Day 4. Harvest virus by collecting the medium supernatant into a 50 ml conical tube, centrifuge and filter the supernatant through a 0.45 μm filter unit to remove large cellular debris. Finally, concentrate the lentiviral mixture, flash freeze, and store at −80° C.

HEK293T and K562 cells were transduced with the stored lentiviral particles using Retronectin-coated plates following standard procedures. Four days post transduction, 1 μg puromycin selection was applied to kill the untransfected cells.

### Culture Conditions for K562 Cells

K562 cells were cultured in Roswell Park Memorial Institute 1640 medium (Thermo Fisher Scientific) supplemented with 10% fetal bovine serum, 100 U/mL penicillin, 100 μg/mL streptomycin, and 292 mg/mL L-Glutamine. The cultures were split at cell densities of 7-8×10⁶, and the displaced volume was replaced with an equal volume of fresh pre-warmed medium. Cultures were incubated at 37° C. with 5% CO₂.

### Nucleofection and Genomic DNA Extraction

0.25×10⁶ to 5.00×10⁶ K562 cells, 4 μg mRNA, and 1 μg pegRNA were resuspended in 100 μL of homemade electroporation solution (Bak et al., 2018) and transfected using program FF-120 (Amaxa 4D). After electroporation, the cells were transferred to 6-well plates with Roswell Park Memorial Institute 1640 medium supplemented with 10% FBS and 292 μg/mL L-Glutamine. The transfected cells were then cultured for 72 h, washed with phosphate buffered saline, and incubated with lysis buffer (10 mM Trish-HCl, pH 8.0, 0.05% SDS, 800 U/L proteinase K) at 37° C. for 2h, followed by enzyme inactivation at 80° C. for 30 min. Genomic DNA was extracted using a Nucleospin 8 DNA isolation kit (Macherey-Nagel).

### Sanger Sequencing-Based Quantification of Editing Outcomes

Sanger sequencing was used for rapid assessment of editing outcomes. To this end, a 1-2 kb region with the target positioned roughly in the middle was amplified in a 25 μL PCR reaction with Q5 High-Fidelity 2× Master Mix (NEB), using 1 μL of genomic DNA as template. Next, PCR products were purified using Zymo kit D4004, and analysed by Sanger sequencing with primers that are 100-500 bp removed from the target sequence. The sequencing results were analysed by aligning the obtained Sanger reads to the unedited sequence and by TIDE analysis (Brinkman et al., 2014).

### Illumina Sequencing of Target Amplicons

The regions targeted for prime editing were PCR amplified in 25 L reactions with Q5 High-Fidelity 2× Master Mix (NEB), using 1 μL of genomic DNA as template. The forward primers in these reactions add 6 bp long DNA barcodes that allow differentiation of conditions in the sequencing data. Both the forward and reverse primers also include adapters for Illumina sequencing. Successful amplification was verified using (capillary) gel electrophoresis. The resulting amplicons were then pooled in equal concentrations and purified using Zymo kit D4033. The pooled amplicons were analysed by Illumina paired-end 150 bp NovaSeq sequencing.

### Illumina Sequencing-Based Quantification of Editing Outcomes

The paired-end sequencing data was first merged using the program seqprep (version 1.6.14 or later). Based on their barcodes, the reads were attributed to the corresponding condition. Reads were then filtered for containing valid primer combinations and sufficient confidence for base-calls between the primers (error probability lower than 0.01). Insertions, deletions, and substitutions were scored for each read using CRISPResso2 software (Clement et al., 2019) or in-house python and bash scripts.

### 11.2 Results

### Editing in the GFP Reporter Assay

Cas12a prime editing in human cells was first examined using a similar extrachromosomal dGFP reporter assay as used in plant cells. In this system, cells produce green fluorescence signals when Cas12a prime editors precisely edit a transiently expressed dGFP reporter harboring a premature TAG stop codon. Based on the results obtained in oilseed rape protoplasts, different prime editing systems were evaluated in HEK293T cells, including the RT (5M)-nLbCas12a (D156R/R1138A) or RT (5M)-nLbCas12a (D156R/RuvC^{lidΔ}) prime editors described before (SEQ ID NO: 199 and SEQ ID NO: 65) and a variety of pegRNAs ranging from all-in-one pegRNAs with or without backbone modifications and same-sense mutations in the RTT to split linear or circular petRNAs co-delivered with separate crRNAs. In a separate set of reactions, the impact of second-strand nicking (with the nicking crRNA of SEQ ID NO: 201) and in trans expression of an MCP-RT (5M) fusion protein (SEQ ID NO: 200) was also assessed. An overview of all treatments tested is shown in Table 17.

All prime editor constructs included the CAGGS promoter for strong constitutive expression (SEQ ID NO: 407), while expression of the peg-, pet-, cr- and nicking RNAs was driven by the human U6 promoter (SEQ ID NO: 326). The pegRNA, crRNA and nicking gRNAs contained two mature direct repeats (SEQ ID NO: 91) positioned at the 5′ and 3′ ends of the sequences, respectively. The respective prime editor, RT-, pegRNA-, petRNA-, crRNA- and nicking gRNA-expressing plasmids were co-transfected into HEK293T cells using jetOptimus DNA transfection reagent and editing efficiencies were assessed by flow cytometry at three days post transfection. As a transfection control, HEK293T cells were co-transfected with a construct constitutively expressing a fluorescent TagBFP2 reporter under control of the CAGGS promoter. As a negative control, the Cas12a prime editor constructs were transfected without pegRNAs.

As shown in Table 17, flow cytometry analysis at three days post transfection revealed high levels of editing both with the ‘RuvC^{lidΔ}’ and the ‘R1138A’ editors inducing comparable levels of GFP-positive cells irrespective of the type of pegRNA used. Consistent with previous findings in oilseed rape (see Table 1), all-in-one pegRNAs with an RTT of 66 nt and an PBS of 9 nt yielded up to 8.6% and 12% editing with the ‘RuvC^{lidΔ}’ and ‘R1138A’ prime editors, respectively, whereas decreasing the RTT length to less than 20 nt or extending the RTT to 86 nt strongly reduced editing efficiencies. Overall, the highest levels of editing (reaching up to 25.8%) were observed when using split PE systems with MS2-tagged circular petRNAs performing slightly better than their linear counterparts. Conversely,

| | | | | | | | GFP- |
|---|---|---|---|---|---|---|---|
| | | gRNA | | | MCP- | nicking | positive |
| Prime editor | Description | SEQ ID NO | RTT (nt) | PBS (nt) | RT | gRNA | cells (%) |
| RT(5M)- | Negative Ctrl | n/a | n/a | n/a | − | − | 0.4 ± 0.2 |
| nLbCas 12a(D156R/ | pegRNA | 33 | 66 | 9 | − | − | 12 ± 1.3 |
| R1138A) | pegRNA | 33 | 66 | 9 | + | − | 15 ± 1.2 |
| | pegRNA | 33 | 66 | 9 | − | + | 10.6 ± 1.3 |
| | pegRNA | 33 | 66 | 9 | + | + | 13.5 ± 1.9 |
| | pegRNA | 34 | 66 | 12 | + | + | 9.4 ± 1.1 |
| | pegRNA | 36 | 86 | 9 | + | + | 0.7 ± 0.2 |
| | pegRNA | 37 | 86 | 12 | + | + | 1.3 ± 0.3 |
| | pegRNA | 408 | 9 | 9 | + | + | 1.4 ± 0.3 |
| | pegRNA | 409 | 15 | 9 | + | + | 3.1 ± 0.6 |
| | pegRNA | 410 | 22 | 9 | + | + | 3.0 ± 0.7 |
| | pegRNA with silent mutations in RTT | 203 | 66 | 9 | − | − | 12.1 ± 1.1 |
| | Backbone-modified pegRNA | 214 | 66 | 9 | − | − | 1.7 ± 0.1 |
| | Backbone-modified pegRNA | 217 | 66 | 9 | − | − | 1.9 ± 0.3 |
| | pegRNA with silent mutations in RTT | 203 | 66 | 9 | + | + | 15.4 ± 2.7 |
| | Backbone-modified pegRNA | 214 | 66 | 9 | + | + | 8.2 ± 0.6 |
| | Backbone-modified pegRNA | 217 | 66 | 9 | + | + | 10.5 ± 1.1 |
| | crRNA + MS2-tagged linear petRNA | 223 + 220 | 66 | 9 | + | − | 17.7 ± 1.3 |
| | crRNA + MS2-tagged circular petRNA | 223 + 221 | 66 | 9 | + | − | 25.2 ± 2.5 |
| RT(5M)- | Negative Ctrl | n/a | n/a | n/a | − | − | 0.5 ± 0.2 |
| nLbCas 12a(D156R/ | pegRNA | 33 | 66 | 9 | − | − | 8.6 ± 0.8 |
| RuvClid4) | pegRNA | 33 | 66 | 9 | + | − | 9.4 ± 0.9 |
| | pegRNA | 33 | 66 | 9 | − | + | 6.1 ± 0.8 |
| | pegRNA | 33 | 66 | 9 | + | + | 7.2 ± 1.3 |
| | pegRNA | 34 | 66 | 12 | + | + | 7.9 ± 0.7 |
| | pegRNA | 36 | 86 | 9 | + | + | 1.0 ± 0.4 |
| | pegRNA | 37 | 86 | 12 | + | + | 1.3 ± 0.3 |
| | pegRNA | 408 | 9 | 9 | + | + | 0.4 ± 0.1 |
| | pegRNA | 409 | 15 | 9 | + | + | 1.7 ± 0.4 |
| | pegRNA | 410 | 22 | 9 | + | + | 0.9 ± 0.3 |
| | pegRNA with silent mutations in RTT | 203 | 66 | 9 | − | − | 12.1 ± 1.1 |
| | Backbone-modified pegRNA | 214 | 66 | 9 | − | − | 1.7 ± 0.1 |
| | Backbone-modified pegRNA | 217 | 66 | 9 | − | − | 1.9 ± 0.3 |
| | pegRNA with silent mutations in RTT | 203 | 66 | 9 | + | + | 9.2 ± 1.5 |
| | Backbone-modified pegRNA | 214 | 66 | 9 | + | + | 1.5 ± 0.4 |
| | Backbone-modified pegRNA | 217 | 66 | 9 | + | + | 1.5 ± 0.2 |
| | crRNA + MS2-tagged linear petRNA | 223 + 220 | 66 | 9 | + | − | 21.7 ± 0.6 |
| | crRNA + MS2-tagged circular petRNA | 223 + 221 | 66 | 9 | + | − | 25.8 ± 1.4 |

second-strand nicking or introducing silent mutations in the RTT had only minor effects on the level of editing. These findings not only demonstrate the functionality of the engineered prime editing systems in human cells but also highlight the effectiveness of specific pegRNA architectures that combine long RTTs and short PBSs across diverse experimental conditions.

Table 17 shows the editing efficiency of different split and unsplit nCas12a prime editing systems in HEK293T cells co-transfected with a dGFP prime editing and a TagBFP2 control reporter. Editing efficiencies are expressed as the number of GFP-positive cells over the number of TagBFP2-positive cells at three days post transfection as determined by flow cytometry.

### Editing at Endogenous Sites

Next, nCas12a prime editing was also tested at three endogenous genes: FANCF, DNMT1, and VEGFA. For each of these genes, one target sequence was selected for Cas9 editing and two target sequences for Cas12a editing (see Table 18 and Table 19). For Cas9-editing, previously described prime editing target sites were selected (Anzalone et al., 2019; see Table 19). For Cas12a-based prime editing, target sites were selected for which successful non-homologous end-joining (NHEJ)-based editing (Toth et al., 2018) or base editing (Kim et al., 2016) has been reported (see Table 18). All target sites were first tested for indel formation upon expression of wild-type SpyCas9 and LbCas12a using previously published sgRNAs or crRNAs (see Table 18 and Table 19). To examine Cas12a prime editing, a series of Cas12a pegRNAs with varying PBS and RTT lengths and with or without 3′ structural RNA elements were designed, while previously published Cas9 pegRNAs served as positive controls (see Table 20). Negative controls for Cas12a prime editing included pegRNAs with sequence-scrambled PBS sequences that should impair priming for reverse transcription.

| | | SEQ ID | Spacer sequence | |
|---|---|---|---|---|
| Target | Gene | NO | (5′ to 3′) | Edit |
| 1 | DNMT1 | 182 | CTGATGGTCCATGTCTGTTA | +5 substitution |
| 2 | VEGFA | 184 | TGACCTCCCAAACAGCTACA | +5 substitution |
| 3 | FANCF | 180 | GCGGATGTTCCAATCAGTAC | +5 substitution |
| 4 | DNMT1 | 324 | CCTCACTCCTGCTCGGTGAA | +1−7 substitution |
| 5 | VEGFA | 325 | CTAGGAATATTGAAGGGGGCAGG | +1−7 substitution |
| 6 | FANCF | 326 | GCGGATGTTCCAATCAGTACGCA | +1−7 substitution |

Table 18 shows an overview of the target sites selected for analysis of nCas12a prime editing in human cells.

| Target | Gene | SEQ ID NO | Spacer sequence (5′ to 3′) | Reference |
|---|---|---|---|---|
| 7 | FANCF | 179 | GGAATCCCTTCTGCAGCACC | Anzalone et al., 2019 |
| 8 | DNMT1 | 181 | GATTCCTGGTGCCAGAAACA | Anzalone et al., 2019 |
| 9 | VEGFA | 183 | GATGTCTGCAGGCCAGATGA | Anzalone et al., 2019 |

Table 19 above shows an overview of the target sites selected for analysis of nCas9 prime editing in human cells

| | | | sequence (5′ to 3′) |
|---|---|---|---|
| Target site | RNA type | Reference | SEQ ID NOs: 185 to 196 |
| FANCF_Cas9 | sgRNA | | GGAATCCCTTCTGCAGCACCGTTTTAGAG |
| | | | CTAGAAATAGCAAGTTAAAATAAGGCTAG |
| | | | TCCGTTATCAACTTGAAAAAGTGGCACCG |
| | | | AGTCGGTGC |
| FANCF_Cas9 | FANCF_3c_ | Anzalone et al., | GGAATCCCTTCTGCAGCACCGTTTTAGAG |
| | 5GtoT | 2019 | CTAGAAATAGCAAGTTAAAATAAGGCTAG |
| | | | TCCGTTATCAACTTGAAAAAGTGGCACCG |
| | | | AGTCGGTGCGGAAAAGCGATCaAGGTGC |
| | | | TGCAGAAGGGA |
| DNMT1_Cas9 | sgRNA | | GATTCCTGGTGCCAGAAACAGTTTTAGAG |
| | | | CTAGAAATAGCAAGTTAAAATAAGGCTAG |
| | | | TCCGTTATCAACTTGAAAAAGTGGCACCG |
| | | | AGTCGGTGC |
| DNMT1_Cas9 | DNMT1_ED5b_ | Anzalone et al., | GATTCCTGGTGCCAGAAACAGTTTTAGAG |
| | 15 | 2019 | CTAGAAATAGCAAGTTAAAATAAGGCTAG |
| | | | TCCGTTATCAACTTGAAAAAGTGGCACCG |
| | | | AGTCGGTGCTCCCGTCACCaCTGTTTCTG |
| | | | GCACCAGG |
| VEGFA_Cas9 | sgRNA | | GATGTCTGCAGGCCAGATGAGTTTTAGAG |
| | | | CTAGAAATAGCAAGTTAAAATAAGGCTAG |
| | | | TCCGTTATCAACTTGAAAAAGTGGCACCG |
| | | | AGTCGGTGC |
| VEGFA_Cas9 | VEGFA_ED5a_ | Anzalone et al., | GATGTCTGCAGGCCAGATGAGTTTTAGAG |
| | 17 | 2019 | CTAGAAATAGCAAGTTAAAATAAGGCTAG |
| | | | TCCGTTATCAACTTGAAAAAGTGGCACCG |
| | | | AGTCGGTGCGCCATCTGGAGCaCTCATC |
| | | | TGGCCTGCAGA |
| FANCF_Cas12a | crRNA | Toth et al., | TAATTTCTACTAAGTGTAGATGCGGATGT |
| | | 2018 | TCCAATCAGTAC |
| FANCF_Cas12a | pegRNA_RTT44_ | | TAATTTCTACTAAGTGTAGATGCGGATGT |
| | PBS09 | | TCCAATCAGTACGGCCCTACTTCCGCTTT |
| | | | CACCTTGGAGACGGCGACTCTCaGCGTA |
| | | | CTGATTGG |
| DNMT1_Cas12a | crRNA | Kim et al., | TAATTTCTACTAAGTGTAGATCTGATGGTC |
| | | 2016 | CATGTCTGTTA |
| DNMT1_Cas12a | pegRNA_RTT43_ | | TAATTTCTACTAAGTGTAGATCTGATGGTC |
| | PBS09 | | CATGTCTGTTATCTGGGGAACACGCCCG |
| | | | GTGTCACGCCACTTGACAGGCCAGTAAC |
| | | | AGACATG |
| VEGFA_Cas12a | crRNA | | TAATTTCTACTAAGTGTAGATTGACCTCCC |
| | | | AAACAGCTACA |
| VEGFA_Cas12a | pegRNA_RTT44_ | Toth et al., | TAATTTCTACTAAGTGTAGATTGACCTCCC |
| | PBS09 | 2018 | AAACAGCTACAGCCCTGGGCTCTCTGTA |
| | | | CATGAAGCAACTCCAGTCCCAAtTATGTA |
| | | | GCTGTTT |

Table 20 provides an overview of the sgRNAs, crRNAs, and pegRNAs used for prime editing in human cells. One representative Cas12a pegRNA design is shown for each target. Spacer sequence is shown in italic, RTT is highlighted in bold characters with changes compared to wt shown in lower case, PBS is underscored. Sequences are included in the shown order as SEQ ID NOs: 185 to 196 from top to bottom in the last column of Table 20 above, respectively.

In a first experiment, Cas12a prime editing is evaluated at the six different target sites using chemically modified synthetic pegRNAs with different lengths of the RTT and PBS. An overview of the target sites, type of edits, and pegRNAs used is provided in Table 21. The synthetic pegRNAs are ordered from IDT DNA Technologies and contain 2′-O-methyl modifications and phosphorothioate linkages between the first and last three nucleotides. Lentivirus-transduced K562 cells stably expressing RT (5M)-nLbCas12a (D156R/RuvC^{lidΔ}) (SEQ ID NO: 65) are transfected with the synthetic pegRNAs using the Lonza Amaxa 4D-Nucleofector system as described above and cultured in RMPI medium containing 10% FBS. Genomic DNA is isolated four days after transfection using a Nucleospin 8 Plant II kit (Machery Nagel, 740664). PCR products containing the genomic sites of interest are generated using the primers of SEQ ID NOs: 338 to 349. Next, the amplified DNA products are purified and subjected to amplicon deep sequencing. For a quick readout of editing efficiency, target sites are also amplified using edit-specific capture PCR with the phosphorothioated primers of SEQ ID NOs: 350 to 361.

| | | | pegRNA | | | |
|---|---|---|---|---|---|---|
| | | | RTT | PBS | SEQ | |
| Target | Gene | Edit | (nt) | (nt) | ID NO | Sequence |
| 1 | DNMT1 | +5 | 66 | 9 | 332 | mU*mA*mA*rUrUrUrCrUrArCrUrArArGrUrGrUrArGrArU |
| | | substitution | | | | rCrUrGrArUrGrGrUrCrCrArUrGrUrCrUrGrUrUrArArGrGr |
| | | | | | | GrArArArUrArArArArGrGrArArArArGrUrCrArCrUrCrUr |
| | | | | | | GrGrGrGrArArCrArCrGrCrCrCrGrGrUrGrUrCrArCrGrCr |
| | | | | | | CrArCrUrUrGrArCrArGrGrCrCrArGrUrArArCrArGrArC* |
| | | | | | | mA*mU*mG |
| 2 | VEGFA | +5 | 65 | 9 | 333 | mU*mA*mA*rUrUrUrCrUrArCrUrArArGrUrGrUrArGrArU |
| | | substitution | | | | rUrGrArCrCrUrCrCrCrArArArCrArGrCrUrArCrArArCrAr |
| | | | | | | CrUrGrUrGrGrCrCrCrCrUrGrUrGrCrCrCrArGrCrCrCrUr |
| | | | | | | GrGrGrCrUrCrUrCrUrGrUrArCrArUrGrArArGrCrArArCr |
| | | | | | | UrCrCrArGrUrCrCrCrArArUrUrArUrGrUrArGrCrUrG*mU |
| | | | | | | *mU*mU |
| 3 | FANCF | +5 | 66 | 9 | 334 | mU*mA*mA*rUrUrUrCrUrArCrUrArArGrUrGrUrArGrArU |
| | | substitution | | | | rGrCrGrGrArUrGrUrUrCrCrArArUrCrArGrUrArCrGrGrG |
| | | | | | | rArUrUrCrCrArUrGrArGrGrUrGrCrGrCrGrArArGrGrCrC |
| | | | | | | rCrUrArCrUrUrCrCrGrCrUrUrUrCrArCrCrUrUrGrGrArGr |
| | | | | | | ArCrGrGrCrGrArCrUrCrUrCrArGrCrGrUrArCrUrGrArU* |
| | | | | | | mU*mG*mG |
| 4 | DNMT1 | +1−7 | 73 | 10 | 335 | mU*mA*mA*rUrUrUrCrUrArCrUrArArGrUrGrUrArGrArU |
| | | substitution | | | | rCrCrUrCrArCrUrCrCrUrGrCrUrCrGrGrUrGrArArArArAr |
| | | | | | | CrGrGrUrCrCrCrCrArGrArGrGrGrUrUrCrUrArGrArCrCr |
| | | | | | | CrArGrArGrGrCrUrCrArArGrUrGrArGrCrArGrCrUrGrAr |
| | | | | | | GrGrCrArGrGrUrGrCrCrUrGrCrUrGrArGrGrArUrUrUrCr |
| | | | | | | GrCrArCrCrGrArG*mC*mA*mG |
| 5 | VEGFA | +1−7 | 66 | 15 | 336 | mU*mA*mA*rUrUrUrCrUrArCrUrArArGrUrGrUrArGrArU |
| | | substitution | | | | rCrUrArGrGrArArUrArUrUrGrArArGrGrGrGrGrCrArGrGr |
| | | | | | | CrArCrUrCrCrArGrGrArUrUrCrCrArArUrArGrArUrCrUr |
| | | | | | | GrUrGrUrGrUrCrCrCrUrCrUrCrCrCrCrArCrCrCrGrUrCr |
| | | | | | | CrCrUrGrUrCrCrGrGrCrUrCrUrCrCrGrCrCrUrUrGrArUr |
| | | | | | | GrGrArUrCrCrCrCrUrUrCrArArUrArU*mU*mC*mC |
| 6 | FANCF | +1−7 | 66 | 15 | 337 | mU*mA*mA*rUrUrUrCrUrArCrUrArArGrUrGrUrArGrArU |
| | | substitution | | | | rGrCrGrGrArUrGrUrUrCrCrArArUrCrArGrUrArCrGrCrAr |
| | | | | | | GrGrGrArUrUrCrCrArUrGrArGrGrUrGrCrGrCrGrArArGr |
| | | | | | | GrCrCrCrUrArCrUrUrCrCrGrCrUrUrUrCrArCrCrUrUrGr |
| | | | | | | GrArGrArCrGrGrCrGrArCrUrCrArUrArCrUrCrArArCrUr |
| | | | | | | GrArUrUrGrGrArArC*mA*mU*mC |

Table 21 provides an overview of the synthetic pegRNAs used for prime editing in human cells.

To assess the importance of pegRNA architectures and test the potential of split RNA systems, different plasmids expressing either all-in-one pegRNAs with PBS lengths ranging from 9-20 bases and RTT lengths ranging from 15-86 bases (SEQ ID NOs: 362 to 394) or MS2-tagged linear and circular petRNAs (SEQ ID NOS: 395 to 400) are constructed via Golden Gate cloning and tested in lentivirus-transduced HEK293T or K562 cells stably expressing RT (5M)-nLbCas12a (D156R/RuvC^{lidΔ}) (SEQ ID NO: 65). In a next round of optimization, the effect of stabilizing pegRNAs by incorporating structured RNA motifs is evaluated. To this end, different RNA hairpins and pseudoknots are added to the 3′ end of the pegRNA PBS, including the PP7 hairpin from bacteriophages and the pseudoknot evoPreQ1, both of which were previously shown to enhance prime editing efficiencies at a variety of target sites in multiple cell lines (Nelson et al., 2022; Kim et al., 2022). Plasmids encoding the engineered pegRNAs (SEQ ID NOs: 401 to 406) targeting the different loci listed in Table 18 are transfected in HEK293T or K562 cells expressing RT (5M)-nLbCas12a (D156R/RuvC^{lidΔ}) from either a lentiviral vector, co-transfected mRNA or plasmid DNA. Three days post transfection, genomic DNA is isolated, and the editing efficiency is measured by deep amplicon sequencing as described previously. An overview of exemplary circular and linear petRNAs and pegRNAs with and without 3′ appended evoPreQ1 is shown in Table 22 and Table 23.

| | | | 5′ | 5′ | 5′ | | | 3′ | 3′ | 3′ |
|---|---|---|---|---|---|---|---|---|---|---|
| Target | Gene | Edit | ribozyme | ligation | MS2 | RTT | PBS | MS2 | ligation | ribozyme |
| 4 | DNMT1 | +1−7 AATTTGG | GCCATCAG | AACCATGC | GCACAT | AGGCAG | CACCGA | CAAGGC | AAATTAACA | AACACTGCC |
| | | to CGAAATC | TCGCCGGT | CGACTGAT | GAGGAT | GTGCCT | GCAG | ACATGA | CTGCCATCA | AATGCCGGT |
| | | | CCCAAGCC | GGCAGAAC | CACCCA | GCTGAG | | GGATCA | GTCGGCGT | CCCAAGCCC |
| | | | CGGATAAA | TAA | TGTGC | gatttcg | | CCCATG | GGACTGTAG | GGATAAAAG |
| | | | ATGGGAGG | | | | | TGC | | TGGAGGGTA |
| | | | GGGCGGG | | | | | | | CAGTCCACG |
| | | | AAACCGCC | | | | | | | C |
| | | | T | | | | | | | |
| 5 | VEGFA | +1−7 GCAGGGG | GCCATCAG | AACCATGC | GCACAT | TGTCCG | CCCCTT | CAAGGC | AAATTAACA | AACACTGCC |
| | | to ATCCATC | TCGCCGGT | CGACTGAT | GAGGAT | GCTCTC | CAATATT | ACATGA | CTGCCATCA | AATGCCGGT |
| | | | CCCAAGCC | GGCAGAAC | CACCCA | CGCCTT | CC | GGATCA | GTCGGCGT | CCCAAGCCC |
| | | | CGGATAAA | TAA | TGTGC | gatggat | | CCCATG | GGACTGTAG | GGATAAAAG |
| | | | ATGGGAGG | | | | | TGC | | TGGAGGGTA |
| | | | GGGCGGG | | | | | | | CAGTCCACG |
| | | | AAACCGCC | | | | | | | C |
| | | | T | | | | | | | |
| 6 | FANCF | +1−7 ACGCAGA | GCCATCAG | AACCATGC | GCACAT | ACCTTG | ACTGATT | ACATGA | AAATTAACA | AACACTGCC |
| | | to TGAGTAT | TCGCCGGT | CGACTGAT | GAGGAT | GAGACG | GGAACA | CAAGGC | CTGCCATCA | AATGCCGGT |
| | | | CCCAAGCC | GGCAGAAC | CACCCA | GCGACT | TC | GGATCA | GTCGGCGT | CCCAAGCCC |
| | | | CGGATAAA | TAA | TGTGC | Catactca | | CCCATG | GGACTGTAG | GGATAAAAG |
| | | | ATGGGAGG | | | | | TGC | | TGGAGGGTA |
| | | | GGGCGGG | | | | | | | CAGTCCACG |
| | | | AAACCGCC | | | | | | | C |
| | | | T | | | | | | | |
| 4 | DNMT1 | +1−7 AATTTGG | n/a | n/a | GCACAT | AGGCAG | CACCGA | CAAGGC | n/a | n/a |
| | | to CGAAATC | | | GAGGAT | GTGCCT | GCAG | ACATGA | | |
| | | | | | CACCCA | GCTGAG | | GGATCA | | |
| | | | | | TGTGC | gatttcg | | CCCATG | | |
| | | | | | | | | TGC | | |
| 5 | VEGFA | +1−7 GCAGGGG | n/a | n/a | GCACAT | TGTCCG | CCCCTT | CAAGGC | n/a | n/a |
| | | to ATCCATC | | | GAGGAT | GCTCTC | CAATATT | ACATGA | | |
| | | | | | CACCCA | CGCCTT | CC | GGATCA | | |
| | | | | | TGTGC | gatggat | | CCCATG | | |
| | | | | | | | | TGC | | |
| 6 | FANCF | +1−7 ACGCAGA | n/a | n/a | GCACAT | ACCTTG | ACTGATT | ACATGA | n/a | n/a |
| | | to TGAGTAT | | | GAGGAT | GAGACG | GGAACA | CAAGGC | | |
| | | | | | CACCCA | GCGACT | TC | GGATCA | | |
| | | | | | TGTGC | Catactca | | CCCATG | | |
| | | | | | | | | TGC | | |

Table 22 provides an overview of the MS2-tagged circular and linear petRNAs used for prime editing in human cells. Changes compared to wt are shown in lower case. Sequences are included in the shown order as SEQ ID NOs: 395 to 400.

| Target | Gene | Edit | Spacer | RTT | PBS | 3′motif |
|---|---|---|---|---|---|---|
| 4 | DNMT1 | +1−7 AATTTGG | CCTCACTCCTGCT | AAACGGTCCCCAGAGGGT | CACCGAGCAG | none |
| | | to CGAAATC | CGGTGAA | TCTAGACCCAGAGGCTCA | | |
| | | | | AGTGAGCAGCTGAGGCAG | | |
| | | | | GTGCCTGCTGAGgatttcg | | |
| 4 | DNMT1 | +1−7 AATTTGG | CCTCACTCCTGCT | AAACGGTCCCCAGAGGGT | CACCGAGCAG | ACTAGAA |
| | | to CGAAATC | CGGTGAA | TCTAGACCCAGAGGCTCA | | CGCGGTTCTA |
| | | | | AGTGAGCAGCTGAGGCAG | | TCTAGTTACG |
| | | | | GTGCCTGCTGAGgatttcg | | CGTTAAACCA |
| 5 | VEGFA | +1−7 | CTAGGAATATTGAA | CACTCCAGGATTCCAATA | CCCCTTCAAT | none |
| | | GCAGGGG to | GGGGGCAGG | GATCTGTGTGTCCCTCTCC | ATTCC | |
| | | ATCCATC | | CCACCCGTCCCTGTCCGG | | |
| | | | | CTCTCCGCCTTgatggat | | |
| 5 | VEGFA | +1−7 | CTAGGAATATTGAA | CACTCCAGGATTCCAATA | CCCCTTCAAT | CGCGGTTCTA |
| | | GCAGGGG to | GGGGGCAGG | GATCTGTGTGTCCCTCTCC | ATTCC | TCTAGTTACG |
| | | ATCCATO | | CCACCCGTCCCTGTCCGG | | CGTTAAACCA |
| | | | | CTCTCCGCCTTgatggat | | ACTAGAA |
| 6 | FANCF | +1−7 | GCGGATGTTCCAAT | TTTCACCTTGGAGACGGC | ACTGATTGGA | none |
| | | ACGCAGA to | CAGTACGCA | GGGATTCCATGAGGTGCG | ACATC | |
| | | TGAGTAT | | CGAAGGCCCTACTTCCGC | | |
| | | | | GACTCatactca | | |
| 6 | FANCF | +1−7 | GCGGATGTTCCAAT | TTTCACCTTGGAGACGGC | ACTGATTGGA | CGCGGTTCTA |
| | | ACGCAGA to | CAGTACGCA | GGGATTCCATGAGGTGCG | ACATC | TCTAGTTACG |
| | | TGAGTAT | | CGAAGGCCCTACTTCCGC | | CGTTAAACCA |
| | | | | GACTCatactca | | ACTAGAA |

Table 23 provides an overview of pegRNAs with and without 3′ structured motifs used for prime editing in human cells. Changes compared to wt are shown in lower case. Sequences are included in the shown order as SEQ ID NOs: 401 to 406.

### REFERENCES FOR EXAMPLES

Anzalone A V, Randolph P B, Davis J R, Sousa A A, Koblan L W, Levy J M, et al. Search-and-replace genome editing without double-strand breaks or donor DNA. Nature. 2019; 1-1. doi: 10.1038/s41586-019-1711-4
Bak R O, Dever D P, Porteus M H. CRISPR/Cas9 genome editing in human hematopoietic stem cells. Nat Protoc. 2018; 13:358-376. doi: 10.1038/nprot.2017.143
Brinkman E K, Chen T, Amendola M, van Steensel B. Easy quantitative assessment of genome editing by sequence trace decomposition. Nucleic Acids Research. 2014; 42: e168. doi: 10.1093/nar/gku936
Chen, Peter J et al. “Enhanced prime editing systems by manipulating cellular determinants of editing outcomes.” Cell vol. 184, 22 (2021): 5635-5652.e29. doi: 10.1016/j.cell.2021.09.018
Chen P J, Hussmann J A, Yan J, Knipping F, Ravisankar P, Chen P F, Chen C, Nelson J W, Newby G A, Sahin M, Osborn M J, Weissman J S, Adamson B, Liu D R. Enhanced prime editing systems by manipulating cellular determinants of editing outcomes. Cell. 2021 Oct. 28; 184 (22): 5635-5652.e29. doi: 10.1016/j.cell.2021.09.018. Epub 2021 Oct. 14. PMID: 34653350; PMCID: PMC8584034
Clement K, Rees H, Canver M C, Gehrke J M, Farouni R, Hsu J Y, Cole M A, Liu D R, Joung J K, Bauer D E, Pinello L. CRISPResso2 provides accurate and rapid genome editing sequence analysis. Nat Biotechnol. 2019 March; 37 (3): 224-226. doi: 10.1038/s41587-019-0032-3. PMID: 30809026; PMCID: PMC6533916
Davies, Owen Richard, and Luca Pellegrini. “Interaction with the BRCA2 C terminus protects RAD51-DNA filaments from disassembly by BRC repeats.” Nature structural & molecular biology vol. 14, 6 (2007): 475-83. doi: 10.1038/nsmb1251
Feng Y, Liu S, Mo Q, Liu P, Xiao X, Ma H. Enhancing prime editing efficiency and flexibility with tethered and split pegRNAs. Protein Cell. 2023 Apr. 21; 14 (4): 304-308. doi: 10.1093/procel/pwac014
Gaudelli N M, Lam D K, Rees H A, Solá-Esteves N M, Barrera L A, Born D A, et al. Directed evolution of adenine base editors with increased activity and therapeutic application. Nat Biotechnol. 2020; 38:892-900. doi: 10.1038/s41587-020-0491-6
Kim D, Kim J, Hur J K, Been K W, Yoon S, Kim J-S. Genome-wide analysis reveals specificities of Cpf1 endonucleases in human cells. Nature Biotechnology. 2016; 34:863-868. doi: 10.1038/nbt.3609
Y. Bill Kim, Elizabeth B. Pierce, et al. A novel mechanistic framework for precise sequence replacement using reverse transcriptase and diverse CRISPR-Cas systems. BioRxiv (2022). 12.13.520319; doi: https://doi.org/10.1101/2022.12.13.520319
Li X, Zhou L, Gao B Q, Li G, Wang X, Wang Y, Wei J, Han W, Wang Z, Li J, Gao R, Zhu J, Xu W, Wu J, Yang B, Sun X, Yang L, Chen J. Highly efficient prime editing by introducing same-sense mutations in pegRNA or stabilizing its structure. Nat Commun. 2022 Mar. 29; 13 (1): 1669. doi: 10.1038/s41467-022-29339-9. PMID: 35351879; PMCID: PMC8964725
Lorenz, R. et al. ViennaRNA package 2.0. Algorithms Mol Biol 6, 26 (2011)
Nelson, James W et al. “Engineered pegRNAs improve prime editing efficiency.” Nature biotechnology vol. 40, 3 (2022): 402-410. doi: 10.1038/s41587-021-01039-7
Reed, Kelsey M, and Bastiaan O R Bargmann. “Protoplast Regeneration and Its Use in New Plant Breeding Technologies.” Frontiers in genome editing vol. 3 734951. 3 Sep. 2021, doi: 10.3389/fgeed.2021.734951
Tong Y, Jørgensen TS, Whitford C M, Weber T, Lee S Y. A versatile genetic engineering toolkit for E. coli based on CRISPR-prime editing. Nat Commun. 2021; 12:5206. doi: 10.1038/s41467-021-25541-3
Tóth E, Czene B C, Kulcsár PI, Krausz S L, Talas A, Nyeste A, et al. Mb- and FnCpf1 nucleases are active in mammalian cells: activities and PAM preferences of four wild-type Cpf1 nucleases and of their altered PAM specificity variants. Nucleic Acids Research. 2018; 46:10272-10285. doi: 10.1093/nar/gky815
Zheng C, Liang S Q, Liu B, Liu P, Kwan S Y, Wolfe S A, Xue W. A flexible split prime editor using truncated reverse transcriptase improves dual-AAV delivery in mouse liver. Mol Ther. 2022 Mar. 2; 30 (3): 1343-1351. doi: 10.1016/j.ymthe.2022.01.005. Epub 2022 Jan. 5. PMID: 34998953; PMCID: PMC8899602.

1-116. (canceled)

117. A prime editor complex comprising
(a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and
(b) at least one Cas12a prime editor comprising
(i) at least one Cas12a enzyme, preferably having nickase activity (nCas12a), more preferably having non-target strand (NTS) nickase activity, or an active fragment thereof;
(ii) at least one reverse transcriptase, or an active fragment thereof;
(iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and
(iv) optionally at least one organellar localization signal.

118. The prime editor complex of claim 117, further comprising in trans (c) a reverse transcriptase protein, preferably comprising at least one organellar localization signal and preferably comprising an MS2 coat protein (MCP); and/or wherein the at least one Cas12a enzyme, or active fragment thereof further comprises an RNA binding factor, preferably an N-terminal domain of La or a MCP.

119. The prime editor complex of claim 117, comprising the N-terminal domain of La, optionally comprising a reverse transcriptase protein comprising an MS2 coat protein (MCP), and a pegRNA system, and, optionally a nicking crRNA, wherein
a) the crRNA comprises in a 5′ to 3′ direction (i) a CRISPR-Cas class 2 type V scaffold sequence, (ii) a spacer sequence; and optionally (vi) a second scaffold sequence, optionally being the same as the first scaffold sequence; and/or wherein
b) the prime editing template RNA (petRNA) comprises in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence;
and an MS2 stem loop.

120. A prime editing guide RNA (pegRNA) system comprising the parts
(i) a CRISPR-Cas class 2 type V scaffold sequence;
(ii) a spacer sequence;
(iii) optionally a linker sequence;
(iv) a reverse transcriptase template having a length of 9 to 150 nucleotides;
(v) a primer binding site having a length of 5 to 50 nucleotides; and
(vi) optionally a second scaffold sequence, optionally being the same as the first scaffold sequence;
wherein the primer binding site is complementary to the non-target strand.

121. The pegRNA system of claim 120, comprising
a) a pegRNA comprising parts (i), (ii), optionally (iii), (iv), (v), and optionally (vi); or
b) a crispr RNA (crRNA) and a prime editing template RNA (petRNA), wherein
1) said crRNA comprises parts (i), (ii) and optionally (vi); and
2) said petRNA comprises optionally (iii), (iv) and (v), preferably, wherein the petRNA is linear.

122. The pegRNA system of claim 120, wherein the pegRNA further comprises
(vii) optionally at least one 3′ linker sequence; and
(viii) at least one structured motif, including at least one hairpin, including at least one MS2 stem loop, preferably at least two MS2 stem loops, and/or at least one pseudoknot sequence.

123. The pegRNA system of claim 120, wherein (iv) the reverse transcriptase template has a length of 25 to 140 nucleotides, or 30 to 120 nucleotides, or 40 to 100 nucleotides, or 50 to 90 nucleotides, or 60 to 90 nucleotides and/or wherein (v) the primer binding site has a length of 6 to 40 nucleotides, or 6 to 30 nucleotides, or 7 to 20 nucleotides, or 7 to 15, nucleotides, or 9 to 12 nucleotides.

124. The pegRNA system of claim 120, wherein
a) the pegRNA comprises the parts (i), (ii), optionally (iii), (iv), (v), optionally (vi), and, if present, optionally (vii) and (viii), in the following order in 5′ to 3′ direction:
1) (i), (ii), optionally (iii), (iv), (v) and optionally (vi); or
2) (iv), (v), (iii), (i), (ii), optionally (vi); or
3) (i), (ii), optionally (iii), (iv), (v), optionally (vii), (viii) and optionally (vi); or
b) the crRNA comprises in a 5′ to 3′ direction parts (i), (ii) and optionally (vi); and the petRNA comprises in a 5′ to 3′ direction parts (viii), optionally (iii), (iv), (v), optionally (iii), and (viii).

125. The pegRNA system of claim 120, further comprising a nicking crRNA, said nicking crRNA comprising a CRISPR-Cas class 2 type V scaffold sequence; a spacer sequence, and optionally a second scaffold sequence, wherein the target site of the spacer sequence of said nicking crRNA is in the vicinity of and at the opposite strand of the target site of the spacer sequence of said pegRNA.

126. A Cas12a prime editor comprising
(i) at least one Cas12a enzyme having nickase activity (nCas12a), preferably having non-target strand nickase activity, or an active fragment thereof;
(ii) at least one reverse transcriptase, or an active fragment thereof;
(iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof;
(iv) optionally at least one organellar localization signal;
wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or
wherein the at least one nCas12a is an RNase dead nCas12a, optionally comprising a mutation at position H759, including a H759A mutation; and/or
wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or
wherein the Cas12a prime editor comprises the reverse transcriptase protein in trans, preferably comprising at least one organellar localization signal and preferably comprising an MS2 coat protein (MCP).

127. The Cas12a prime editor of claim 126, wherein the Cas12a prime editor comprises the reverse transcriptase protein in trans, and further comprises an RNA binding factor, preferably the N-terminal domain of La or of MCP, more preferably wherein the Cas12a prime editor comprises the N-terminal domain of La, optionally comprising a reverse transcriptase protein comprising an MCP, and a pegRNA system, and, optionally a nicking crRNA, wherein
a) the crRNA comprises in a 5′ to 3′ direction (i) a CRISPR-Cas class 2 type V scaffold sequence, (ii) a spacer sequence and optionally (vi) a second scaffold sequence, optionally being the same as the first scaffold sequence; and
b) a prime editing template RNA the (petRNA) comprising in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

128. A nucleic acid molecule or more than one nucleic acid molecules encoding
the prime editor complex of claim 117; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP);
optionally wherein the nucleic acid molecule(s) is/are codon-optimized for a fungal cell, including a yeast cell, a prokaryotic cell, including a Gram-positive, Gram-negative or Gram-variable bacterial cell, or an archaea cell, preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile; and/or the prokaryotic cell or an archaeal cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

129. The nucleic acid molecule or more than one nucleic acid molecules of claim 128, wherein the nucleic acid molecule(s) comprise(s) or consist(s) of a sequence according to any one of SEQ ID NOs: 284 to 291 or 304 to 311, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%.

130. A fungal cell, including a yeast cell, a prokaryotic cell, including a Gram-positive, Gram-negative or Gram-variable bacterial cell, or an archaea cell comprising
a prime editor complex comprising (a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); and/or
the nucleic acid molecule or more than one nucleic acid molecules of claim 128;
preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile; and/or the prokaryotic cell or an archaeal cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

131. A method for modifying at least one nucleic acid sequence of interest in at least one nucleic acid molecule of at least one fungal cell, including a yeast cell, a prokaryotic cell, including a Gram-positive, Gram-negative or Gram-variable bacterial cell, or an archaea cell or at least one construct at or near at least one target site, the method comprising:
(a) providing at least one cell or construct comprising the nucleic acid sequence of interest to be modified;
(b) providing and/or introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal;
wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or
wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or
wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or providing and/or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) optionally, obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct;

132. A method for producing an edited one fungal, including yeast, prokaryotic, including a Gram-positive, Gram-negative or Gram-variable bacterial, or archaea cell, tissue, and/or organism, the method comprising:
(a) providing at least one cell, tissue, and/or organism comprising at least one nucleic acid sequence of interest in at least one nucleic acid molecule at or near at least one target site;
(b) introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal;
wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or
wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or
wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same, or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct.

133. The method of claim 131, wherein the cell or construct originates from a fungal cell, including the yeast cell, and is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile; or
wherein the cell or construct originates from a prokaryotic cell, including Gram-positive, Gram-negative and Gram-variable bacterial cells, preferably Gram-negative bacterial cells, or an archaeal cell, and is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

134. The method of claim 132, wherein the cell or construct originates from a fungal cell, including the yeast cell, and is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophile; or
wherein the cell or construct originates from a prokaryotic cell, including Gram-positive, Gram-negative and Gram-variable bacterial cells, preferably Gram-negative bacterial cells, or an archaeal cell, and is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

135. An edited fungal, including yeast, prokaryotic, including a Gram-positive, Gram-negative or Gram-variable bacterial, or archaea cell, tissue, organ, material or whole organism obtained by or obtainable by a method according to claim 131.

136. An edited fungal, including yeast, prokaryotic, including a Gram-positive, Gram-negative or Gram-variable bacterial, or archaea cell, tissue, organ, material or whole organism obtained by or obtainable by a method according to claim 132.

137. A method for introducing or modifying, preferably in vitro, in vivo and/or ex vivo modification, in a nucleic acid molecule, preferably in a genome fungal cell, including a yeast cell, a prokaryotic cell, including a Gram-positive, Gram-negative or Gram-variable bacterial cell, or an archaea cell, and/or for metabolic engineering in a fungal cell, including a yeast cell, a prokaryotic cell, including a Gram-positive, Gram-negative or Gram-variable bacterial cell, or an archaea cell, preferably a fungal cell, including a yeast cell, or a bacterial cell, comprising introducing a prime editor complex as defined in claim 117.

138. A method for introducing or modifying, preferably in vitro, in vivo and/or ex vivo modification, in a nucleic acid molecule, preferably in a genome fungal cell, including a yeast cell, a prokaryotic cell, including a Gram-positive, Gram-negative or Gram-variable bacterial cell, or an archaea cell, and/or for metabolic engineering in a fungal cell, including a yeast cell, a prokaryotic cell, including a Gram-positive, Gram-negative or Gram-variable bacterial cell, or an archaea cell, preferably a fungal cell, including a yeast cell, or a bacterial cell, comprising introducing a pegRNA system as defined in claim 120.

139. A method for introducing or modifying, preferably in vitro, in vivo and/or ex vivo modification, in a nucleic acid molecule, preferably in a genome fungal cell, including a yeast cell, a prokaryotic cell, including a Gram-positive, Gram-negative or Gram-variable bacterial cell, or an archaea cell, and/or for metabolic engineering in a fungal cell, including a yeast cell, a prokaryotic cell, including a Gram-positive, Gram-negative or Gram-variable bacterial cell, or an archaea cell, preferably a fungal cell, including a yeast cell, or a bacterial cell, comprising introducing Cas12a prime editor as defined in claim 126.

140. A method for introducing or modifying, preferably in vitro, in vivo and/or ex vivo modification, in a nucleic acid molecule, preferably in a genome fungal cell, including a yeast cell, a prokaryotic cell, including a Gram-positive, Gram-negative or Gram-variable bacterial cell, or an archaea cell, and/or for metabolic engineering in a fungal cell, including a yeast cell, a prokaryotic cell, including a Gram-positive, Gram-negative or Gram-variable bacterial cell, or an archaea cell, preferably a fungal cell, including a yeast cell, or a bacterial cell, comprising introducing a nucleic acid molecule or more than one nucleic acid molecules as defined in claim 128.

141. A method for introducing or modifying, preferably in vitro, in vivo and/or ex vivo modification, in a nucleic acid molecule, preferably in a genome fungal cell, including a yeast cell, a prokaryotic cell, including a Gram-positive, Gram-negative or Gram-variable bacterial cell, or an archaea cell, and/or for metabolic engineering in a fungal cell, including a yeast cell, a prokaryotic cell, including a Gram-positive, Gram-negative or Gram-variable bacterial cell, or an archaea cell, preferably a fungal cell, including a yeast cell, or a bacterial cell, comprising introducing a cell as defined in claim 130.

142. A method comprising using the prime editor complex of claim 117 in a plant cell, including an algal cell.

143. The method of claim 142, wherein the prime editor complex further comprises in trans (c) a reverse transcriptase protein, preferably comprising at least one organellar localization signal and preferably comprising an MS2 coat protein (MCP); and/or wherein the at least one Cas12a enzyme, or active fragment thereof further comprises an RNA binding factor, preferably an N-terminal domain of La or a MCP.

144. The method of claim 142, wherein the prime editor complex further comprises the N-terminal domain of La, optionally comprising a reverse transcriptase protein comprising an MCP, and a pegRNA system, and, optionally a nicking crRNA, wherein
a) the crRNA comprises in a 5′ to 3′ direction (i) a CRISPR-Cas class 2 type V scaffold sequence, (ii) a spacer sequence; and optionally (vi) a second scaffold sequence, optionally being the same as the first scaffold sequence; and/or wherein
b) the prime editing template RNA (petRNA) comprises in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

145. A method comprising using the prime editing guide RNA (pegRNA) system of claim 120 in a plant cell, including an algal cell.

146. The method of claim 145, wherein the pegRNA system comprises
a) a pegRNA comprising parts (i), (ii), optionally (iii), (iv), (v), and optionally (vi); or
b) a crispr RNA (crRNA) and a prime editing template RNA (petRNA), wherein
1) said crRNA comprises parts (i). (ii) and optionally (vi); and
2) said petRNA comprises optionally (iii), (iv) and (v), preferably, wherein the petRNA is linear.

147. The method of claim 145, wherein the pegRNA further comprises
(vii) optionally at least one 3′ linker sequence; and
(viii) at least one structured motif, including at least one hairpin, including at least one MS2 stem loop, preferably at least two MS2 stem loops, and/or at least one pseudoknot sequence.

148. The method of claim 145, wherein (iv) the reverse transcriptase template has a length of 25 to 140 nucleotides, or 30 to 120 nucleotides, or 40 to 100 nucleotides, or 50 to 90 nucleotides, or 60 to 90 nucleotides, and/or wherein (v) the primer binding site has a length of 6 to 40 nucleotides, or 6 to 30 nucleotides, or 7 to 20 nucleotides, or 7 to 15, nucleotides, or 9 to 12 nucleotides.

149. The method of claim 145, wherein
a) the pegRNA comprises the parts (i), (ii), optionally (iii), (iv), (v), optionally (vi), and, if present, optionally (vii) and (viii), in the following order in 5′ to 3′ direction:
1) (i), (ii), optionally (iii), (iv), (v) and optionally (vi); or
2) (iv), (v), (iii), (i), (ii), optionally (vi); or
3) (i), (ii), optionally (iii), (iv), (v), optionally (vii), (viii) and optionally (vi); or
b) the crRNA comprises in a 5′ to 3′ direction parts (i), (ii) and optionally (vi); and the petRNA comprises in a 5′ to 3′ direction parts (viii), optionally (iii), (iv), (v), optionally (iii), and (viii).

150. The method of claim 145, further comprising a nicking crRNA, said nicking crRNA comprising a CRISPR-Cas class 2 type V scaffold sequence; a spacer sequence, and optionally a second scaffold sequence, wherein the target site of the spacer sequence of said nicking crRNA is in the vicinity of and at the opposite strand of the target site of the spacer sequence of said pegRNA or said petRNA.

151. A method comprising using the Cas12a prime editor of claim 126 in a plant cell, including an algal cell.

152. The method of claim 151, wherein the Cas12a prime editor comprises the reverse transcriptase protein in trans, and further comprises an RNA binding factor, preferably the N-terminal domain of La or of MCP, more preferably wherein the Cas12a prime editor comprises the N-terminal domain of La, optionally comprising a reverse transcriptase protein comprising an MCP, and a pegRNA system, and, optionally a nicking crRNA, wherein
a) the crRNA comprises in a 5′ to 3′ direction (i) a CRISPR-Cas class 2 type V scaffold sequence, (ii) a spacer sequence and optionally (vi) a second scaffold sequence, optionally being the same as the first scaffold sequence;
b) a prime editing template RNA the (petRNA) comprising in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

153. A nucleic acid molecule or more than one nucleic acid molecules encoding
the prime editor complex of claim 117; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP);
wherein the nucleic acid molecule(s) is/are codon-optimized for a plant cell, including an algal cell, preferably wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

154. The nucleic acid molecule or more than one nucleic acid molecules of claim 153, wherein the nucleic acid molecule(s) comprise(s) or consist(s) of a sequence according to any one of SEQ ID NOs: 278 to 283 or 298 to 303, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%.

155. A plant cell, including an algal cell comprising
a prime editor complex comprising (a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); and/or
a nucleic acid molecule or more than one nucleic acid molecules of claim 153;
preferably wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

156. A method for modifying at least one nucleic acid sequence of interest in at least one nucleic acid molecule of at least one plant cell, including an algal cell, at or near at least one target site, the method comprising:
(a) providing at least one cell or construct comprising the nucleic acid sequence of interest to be modified;
(b) providing and/or introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or providing and/or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same, or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) optionally, obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct;

157. A method for producing an edited plant cell, tissue, and/or organism, including an algal cell, tissue, and/or organism, the method comprising:
(a) providing at least one cell, tissue, and/or organism comprising at least one nucleic acid sequence of interest in at least one nucleic acid molecule at or near at least one target site;
(b) introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same, or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct;

158. The method of claim 156, wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

159. An edited cell, tissue, organ, material or whole organism obtained by or obtainable by a method according to claim 156.

160. An edited cell, tissue, organ, material or whole organism obtained by or obtainable by a method according to claim 157.

161. A method for introducing or modifying, preferably in vitro, in vivo and/or ex vivo modification, in a nucleic acid molecule, preferably in a plant genome, including an algal genome, including uses for optimizing or modifying a trait in a plant, including the modification of a yield-related trait, or a disease-resistance related trait, and/or for metabolic engineering in a plant cell, an algal cell, comprising introducing a prime editor complex as defined in claim 117.

162. A method for introducing or modifying, preferably in vitro, in vivo and/or ex vivo modification, in a nucleic acid molecule, preferably in a plant genome, including an algal genome, including uses for optimizing or modifying a trait in a plant, including the modification of a yield-related trait, or a disease-resistance related trait, and/or for metabolic engineering in a plant cell, an algal cell, comprising introducing a pegRNA system as defined in claim 120.

163. A method for introducing or modifying, preferably in vitro, in vivo and/or ex vivo modification, in a nucleic acid molecule, preferably in a plant genome, including an algal genome, including uses for optimizing or modifying a trait in a plant, including the modification of a yield-related trait, or a disease-resistance related trait, and/or for metabolic engineering in a plant cell, an algal cell, comprising introducing a Cas12a prime editor as defined in claim 126.

164. A method for introducing or modifying, preferably in vitro, in vivo and/or ex vivo modification, in a nucleic acid molecule, preferably in a plant genome, including an algal genome, including uses for optimizing or modifying a trait in a plant, including the modification of a yield-related trait, or a disease-resistance related trait, and/or for metabolic engineering in a plant cell, an algal cell, comprising introducing a nucleic acid molecule or more than one nucleic acid molecules as defined in claim 153.

165. A method for introducing or modifying, preferably in vitro, in vivo and/or ex vivo modification, in a nucleic acid molecule, preferably in a plant genome, including an algal genome, including uses for optimizing or modifying a trait in a plant, including the modification of a yield-related trait, or a disease-resistance related trait, and/or for metabolic engineering in a plant cell, an algal cell, comprising introducing a cell as defined in claim 155.

166. A method comprising using the prime editor complex of claim 117 in an animal cell, including a human cell.

167. The method of claim 166, wherein the prime editor complex further comprises further comprising in trans (c) a reverse transcriptase protein, preferably comprising at least one organellar localization signal and preferably comprising an MS2 coat protein (MCP) and/or wherein the at least one Cas12a enzyme, or active fragment thereof further comprises an RNA binding factor, preferably an N-terminal domain of La or a MCP.

168. The method of claim 166, wherein the prime editor complex further comprises the N-terminal domain of La, optionally comprising a reverse transcriptase protein comprising an MCP, and a pegRNA system, and, optionally a nicking crRNA, wherein
a) the crRNA comprises in a 5′ to 3′ direction (i) a CRISPR-Cas class 2 type V scaffold sequence, (ii) a spacer sequence; and optionally (vi) a second scaffold sequence, optionally being the same as the first scaffold sequence; and/or wherein
b) the prime editing template RNA (petRNA) comprises in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence;
and an MS2 stem loop.

169. A method comprising using prime editing guide RNA (pegRNA) system of claim 120 in an animal cell, including a human cell.

170. The method of claim 169, wherein the pegRNA system comprises
a) a pegRNA comprising parts (i), (ii), optionally (iii), (iv), (v), and optionally (vi); or
b) a crispr RNA (crRNA) and a prime editing template RNA (petRNA), wherein
1) said crRNA comprises parts (1). (ii) and optionally (vi); and
2) said petRNA comprises optionally (iii), (iv) and (v), preferably, wherein the petRNA is linear.

171. The method of claim 169, wherein the pegRNA further comprises
(vii) optionally at least one 3′ linker sequence; and
(viii) at least one structured motif, including at least one hairpin, including at least one MS2 stem loop, preferably at least two MS2 stem loops, and/or at least one pseudoknot sequence.

172. The method of claim 169, wherein (iv) the reverse transcriptase template has a length of 25 to 140 nucleotides, or 30 to 120 nucleotides, or 40 to 100 nucleotides, or 50 to 90 nucleotides, or 60 to 90 nucleotides and/or wherein (v) the primer binding site has a length of 6 to 40 nucleotides, or 6 to 30 nucleotides, or 7 to 20 nucleotides, or 7 to 15, nucleotides, or 9 to 12 nucleotides.

173. The method of claim 169, wherein
a) the pegRNA comprises the parts (i), (ii), optionally (iii), (iv), (v), optionally (vi), and, if present, optionally (vii) and (viii), in the following order in 5′ to 3′ direction:
1) (i), (ii), optionally (iii), (iv), (v) and optionally (vi); or
2) (iv), (v), (iii), (1), (ii), optionally (vi); or
3) (i), (ii), optionally (iii), (iv), (v), optionally (vii), (viii) and optionally (vi); or
b) the crRNA comprises in a 5′ to 3′ direction parts (i), (ii) and optionally (vi); and the petRNA comprises in a 5′ to 3′ direction parts (viii), optionally (iii), (iv), (v), optionally (iii), and (viii).

174. The method of claim 169, further comprising a nicking crRNA, said nicking crRNA comprising a CRISPR-Cas class 2 type V scaffold sequence; a spacer sequence, and optionally a second scaffold sequence, wherein the target site of the spacer sequence of said nicking crRNA is in the vicinity of and at the opposite strand of the target site of the spacer sequence of said pegRNA or said petRNA.

175. A method comprising using the Cas12a prime editor of claim 120 in an animal cell, including a human cell.

176. The method of claim 175, wherein the Cas12a prime editor comprises the reverse transcriptase protein in trans, and further comprises an RNA binding factor, preferably the N-terminal domain of La or of MCP, more preferably wherein the Cas12a prime editor comprises the N-terminal domain of La, optionally comprising the reverse transcriptase protein comprising an MCP, and a pegRNA system, and, optionally a nicking crRNA, wherein
a) the crRNA comprises in a 5′ to 3′ direction (i) a CRISPR-Cas class 2 type V scaffold sequence, (ii) a spacer sequence and optionally (vi) a second scaffold sequence, optionally being the same as the first scaffold sequence;
b) a prime editing template RNA the (petRNA) comprising in a 5′ to 3′ direction an MS2 stem loop, optionally a linker sequence, a reverse transcriptase template having a length of 9 to 150 nucleotides; a primer binding site having a length of 5 to 50 nucleotides; optionally a linker sequence; and an MS2 stem loop.

177. A nucleic acid molecule or more than one nucleic acid molecules encoding
the prime editor complex of claim 117; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP);
wherein the nucleic acid molecule(s) is/are codon-optimized for an insect, poultry, fish or crustacea cell, or a mammalian cell, preferably wherein the cell is a mammalian cell being selected from a cell originating from a non-human primate, bovine, porcine, rodent, including mouse, or human cell.

178. The nucleic acid molecule or more than one nucleic acid molecules of claim 177, wherein the nucleic acid molecule(s) comprise(s) or consist(s) of a sequence according to any one of SEQ ID NOs: 292 to 297 or 312 to 317, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%.

179. A cell comprising
a prime editor complex comprising (a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; and/or
a pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); and/or
a nucleic acid molecule or more than one nucleic acid molecules of claim 177;
wherein the cell is an insect, poultry, fish or crustacea cell, or a mammalian cell, preferably wherein the cell is a mammalian cell being selected from a cell originating from a non-human primate, bovine, porcine, rodent, including mouse, or human cell.

180. A method for modifying at least one nucleic acid sequence of interest in at least one nucleic acid molecule of at least one cell at or near at least one target site, the method comprising:
(a) providing at least one cell or construct comprising the nucleic acid sequence of interest to be modified;
(b) providing and/or introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or providing and/or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same, or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) optionally, obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct;
wherein the cell originates from an insect, poultry, fish or crustacea cell, or a mammalian cell, preferably wherein the cell is a mammalian cell being selected from a cell originating from a non-human primate, bovine, porcine, rodent, including mouse, or human cell;
optionally wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes; further optionally, wherein the method does not include treatment of the human or animal body by surgery or therapy.

181. A method for producing an edited cell, tissue, and/or organism, the method comprising:
(a) providing at least one cell, tissue, and/or organism comprising at least one nucleic acid sequence of interest in at least one nucleic acid molecule at or near at least one target site;
(b) introducing
(b-i) at least one pegRNA system as defined in claim 120, or at least one nucleic acid molecule or expression vector or construct encoding the same, and
(b-ii) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); or at least one nucleic acid molecule or expression vector or construct encoding the same;
optionally allowing complex formation of (b-i) the at least one pegRNA or crRNA and (b-ii) the at least one Cas12 prime editor before the provision and/or introduction;
or introducing:
(b-iii) at least one prime editor complex comprising (a) at least one prime editing guide RNA (pegRNA) system as defined in claim 120 and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; or at least one nucleic acid molecule or expression vector or construct encoding the same, or at least one nucleic acid molecule or expression vector or construct encoding the same;
(c) allowing the modification of at least one nucleic acid sequence of interest by (b-i) the at least one pegRNA system and (b-ii) the at least one Cas12 prime editor; or by (b-iii) the at least one prime editor complex;
(d) obtaining at least one edited cell or construct comprising a modification at at least one nucleic acid sequence of interest at or near a target site;
optionally, where the method comprises the following step:
(e) regenerating at least one population of edited cells, tissues, organs, materials or whole organisms from the at least one edited cell or construct;
wherein the cell originates from an insect, poultry, fish or crustacea cell, or a mammalian cell, preferably wherein the cell is a mammalian cell being selected from a cell originating from a non-human primate, bovine, porcine, rodent, including mouse, or human cell.

182. An edited cell, tissue, organ, material or whole organism obtained by or obtainable by a method according to claim 180.

183. An edited cell, tissue, organ, material or whole organism obtained by or obtainable by a method according to claim 181.

184. A method for introducing or modifying, preferably in vitro, in vivo and/or ex vivo modification, in a nucleic acid molecule in a genome of a cell, and/or for metabolic engineering in cell, wherein the cell is an insect, poultry, fish or crustacea cell, or a mammalian cell, preferably wherein the cell is a mammalian cell being selected from a cell originating from a non-human primate, bovine, porcine, rodent, including mouse, or human cell, comprising introducing a prime editor complex as defined in claim 117.

185. A method for introducing or modifying, preferably in vitro, in vivo and/or ex vivo modification, in a nucleic acid molecule in a genome of a cell, and/or for metabolic engineering in cell, wherein the cell is an insect, poultry, fish or crustacea cell, or a mammalian cell, preferably wherein the cell is a mammalian cell being selected from a cell originating from a non-human primate, bovine, porcine, rodent, including mouse, or human cell, comprising introducing a pegRNA system as defined in claim 120.

186. A method for introducing or modifying, preferably in vitro, in vivo and/or ex vivo modification, in a nucleic acid molecule in a genome of a cell, and/or for metabolic engineering in cell, wherein the cell is an insect, poultry, fish or crustacea cell, or a mammalian cell, preferably wherein the cell is a mammalian cell being selected from a cell originating from a non-human primate, bovine, porcine, rodent, including mouse, or human cell, comprising introducing a Cas12a prime editor as defined in claim 126.

187. A method for introducing or modifying, preferably in vitro, in vivo and/or ex vivo modification, in a nucleic acid molecule in a genome of a cell, and/or for metabolic engineering in cell, wherein the cell is an insect, poultry, fish or crustacea cell, or a mammalian cell, preferably wherein the cell is a mammalian cell being selected from a cell originating from a non-human primate, bovine, porcine, rodent, including mouse, or human cell, comprising introducing a nucleic acid molecule or more than one nucleic acid molecules as defined in claim 177.

188. A method for introducing or modifying, preferably in vitro, in vivo and/or ex vivo modification, in a nucleic acid molecule in a genome of a cell, and/or for metabolic engineering in cell, wherein the cell is an insect, poultry, fish or crustacea cell, or a mammalian cell, preferably wherein the cell is a mammalian cell being selected from a cell originating from a non-human primate, bovine, porcine, rodent, including mouse, or human cell, comprising introducing a cell as defined in claim 179.

189. A method of treating or preventing a disease, the method comprising using
a prime editor complex comprising (a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; and/or
a pegRNA system prime editing guide RNA (pegRNA) system comprising the parts (i) a CRISPR-Cas class 2 type V scaffold sequence; (ii) a spacer sequence; (iii) optionally a linker sequence;
(iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); and/or
a nucleic acid molecule or more than one nucleic acid molecules of claim 177; and/or
a cell comprising
a prime editor complex comprising (a) at least one prime editing guide RNA (pegRNA) system comprising (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and (b) at least one Cas12a prime editor comprising (i) at least one Cas12a enzyme, preferably having nickase activity (nCas12a), more preferably having non-target strand (NTS) nickase activity, or an active fragment thereof; (ii) at least one reverse transcriptase, or an active fragment thereof; (iii) optionally: at least one linker covalently or non-covalently linking the at least one Cas12a enzyme, or active fragment thereof, and the at least one reverse transcriptase, or an active fragment thereof, preferably, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; and (iv) optionally at least one organellar localization signal; and/ora pegRNA system prime editing guide RNA (pegRNA) system comprising the parts (i) a CRISPR-Cas class 2 type V scaffold sequence; (ii) a spacer sequence; (iii) optionally a linker sequence; (iv) a reverse transcriptase template having a length of 9 to 150 nucleotides; (v) a primer binding site having a length of 5 to 50 nucleotides; and (vi) optionally a second scaffold sequence, optionally being the same as the first scaffold sequence; wherein the primer binding site is complementary to the non-target strand; and/or
a Cas12a prime editor comprising (i) at least one Cas12a enzyme having nickase activity (nCas12a), preferably having non-target strand nickase activity, or an active fragment thereof; (ii) at least one reverse transcriptase, or an active fragment thereof; (iii) at least one linker covalently or non-covalently linking the at least one nCas12a, or active fragment thereof, and the at least one reverse transcriptase, or active fragment thereof, optionally, wherein (ii) the at least one reverse transcriptase, or active fragment thereof, is linked, via (iii) the at least one linker, N-terminally or C-terminally, preferably N-terminally, to (i) the at least one Cas12a enzyme, or active fragment thereof; (iv) optionally at least one organellar localization signal; wherein (i) the at least one nCas12a is fused to (ii) the at least one reverse transcriptase and at least one organellar localization signal is located at the N-terminus of the Cas12a prime editor and at least one organellar localization signal is located at the C-terminus of the Cas12a prime editor; and/or wherein the at least one nCas12a is an RNase dead nCas12a, optionally comprising a mutation at position H759, including a H759A mutation; and/or wherein the Cas12a prime editor comprises at least one ssDNA-binding and/or ssDNA-stabilizing protein, domain, or active fragment thereof, preferably Brex27, RPA70-A, RPA70 B, RPA70-C, RPA32-D, BRCA2-OB2, BRCA2-OB3, HNRNPK KH, PUF60RRM, or Rad51DBD and/or wherein the Cas12a prime editor comprises the reverse transcriptase protein in trans, preferably comprising at least one organellar localization signal and preferably comprising an MS2 coat protein (MCP); and/or
a nucleic acid molecule or more than one nucleic acid molecules of claim 177;
wherein the cell is an insect, poultry, fish or crustacea cell, or a mammalian cell, preferably wherein the cell is a mammalian cell being selected from a cell originating from a non-human primate, bovine, porcine, rodent, including mouse, or human cell;
for introducing at least one modification in a genomic locus of interest of at least one cell of a subject in need thereof at or near at least one disease-state related target site, optionally, wherein the method comprises an ex vivo modification of the at least one disease-state related target site, wherein at least one cell of a subject is provided to perform an ex vivo modification of the at least one disease-state related target site to obtain at least one edited cell.

190. A method for cell therapy, comprising administering to a patient in need thereof said at least one edited cell of claim 189, wherein presence of said at least one edited cell remedies a disease in said patient.


SEQUENCE LISTING (415 sequences)

SEQ ID NO: 1 - protein, 1228 aa, unidentified
  source 1..1228: note = Lachnospiracea bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
  source 1..1307: note = BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1300 aa, Francisella tularensis
  source 1..1300: note = subsp. novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 4 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 5 - protein, 1262 aa, Porphyromonas crevioricanis
MPWIDLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYEPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 6 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNLFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPWVFLSSKKGMETYKPSAYILEGYKQNKHLKSSKDFDITFCHDLIDYFKNCIAIHPEWKNFGFEFSDTSTYEDISGFYREVELQGYKIDWTYISEKDIELLQEKGQLYLFQIYNKDFSKKSTGNDNLHTMYLKNLFSEENLKDIVLKLNGKAEIFFRKSSIKNPIIHKKGSILVNRTYEAEEKEQFGNIRIVRKNIPENIYQELYKYYNDKSDKELSDEAAKLKDIVGHHEATKDIVKDYRYTYDKYFLHMPITINFKANKTSFINDRILQYIATEKELHVIGIDRGERNLIYVSVIDTCGNIVEQKSFNIVNGYDYQIKLKQQEGARQTARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSNWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1227 aa, Methanomethylophilus alvus
  source 1..1227: note = Candidatus Methanomethylophilus alvus
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 8 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 9 - protein, 1206 aa, Butyrivibrio sp.
  source 1..1206: note = NC3005
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 10 - protein, 1298 aa, Thiomicrospira sp.
  source 1..1298: note = XS5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 11 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1222 aa, unidentified
  source 1..1222: note = Lachnospiracea bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLLKRPAATKKAGQAKKKK

SEQ ID NO: 13 - protein, 17 aa, synthetic construct
  REGION 1..17: note = Core lid domain consensus sequence or motif
  VARIANT 2..3: note = X can be any naturally occurring amino acid
  VARIANT 7..8: note = X can be any naturally occurring amino acid
  VARIANT 10..13: note = X can be any naturally occurring amino acid
  VARIANT 15..17: note = X can be any naturally occurring amino acid
LXXGFKXXRXXXXEXXX

SEQ ID NO: 14 - protein, 1228 aa, synthetic construct
  REGION 1..1228: note = Quadruple lid mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGAGVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 15 - protein, 1220 aa, synthetic construct
  REGION 1..1220: note = Lid deletion mutant RuvC Lid deletion 1
  VARIANT 1220: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 16 - protein, 1228 aa, synthetic construct
  REGION 1..1228: note = Lid mutant pRV026004.25836.C4
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGKFSFLVSSSKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 17 - protein, 1228 aa, synthetic construct
  REGION 1..1228: note = Lid mutant pRV26184.26182 rational
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNWSCNGGLQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 18 - protein, 1228 aa, synthetic construct
  REGION 1..1228: note = Lid mutant pRV26182.25836
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGLLFWSCNGGLQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 19 - protein, 1228 aa, synthetic construct
  REGION 1..1228: note = Lid mutant pRV26010.25836. rational
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNKWRYPEAQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 20 - protein, 1228 aa, synthetic construct
  REGION 1..1228: note = Lid mutant pRV026006.25836.C6
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGIIMLKHASGCQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 21 - protein, 1228 aa, synthetic construct
  REGION 1..1228: note = Lid mutant pRV026008.25836.C8
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGYWLHRQFTGRQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 22 - protein, 1220 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYESSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 23 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSCLRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 24 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYLGTVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 25 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSPITRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 26 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSIFRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 27 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSPRSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 28 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYIRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 29 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSTMFRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 30 - RNA, 128 nt, synthetic construct
  source 1..128: mol_type = other RNA
taatttctactaagtgtagatcgtcgccgtccagctcgaccagcgtgagcaagggcgaggagctgttcaccggggtggtgcccatcctggtcgagctggtaatttctactaagtgtagattttttttt

SEQ ID NO: 31 - RNA, 131 nt, synthetic construct
  source 1..131: mol_type = other RNA
taatttctactaagtgtagatcgtcgccgtccagctcgaccagcgtgagcaagggcgaggagctgttcaccggggtggtgcccatcctggtcgagctggacgtaatttctactaagtgtagattttttttt

SEQ ID NO: 32 - RNA, 139 nt, synthetic construct
  source 1..139: mol_type = other RNA
taatttctactaagtgtagatcgtcgccgtccagctcgaccagcgtgagcaagggcgaggagctgttcaccggggtggtgcccatcctggtcgagctggacggcgacgtataatttctactaagtgtagattttttttt

SEQ ID NO: 33 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtgcccatcctggtcgagctggtaatttctactaagtgtagattttttttt

SEQ ID NO: 34 - RNA, 151 nt, synthetic construct
  source 1..151: mol_type = other RNA
taatttctactaagtgtagatcgtcgccgtccagctcgaccagccttattaaaccaaaaccatggtgagcaagggcgaggagctgttcaccggggtggtgcccatcctggtcgagctggacgtaatttctactaagtgtagattttttttt

SEQ ID NO: 35 - RNA, 159 nt, synthetic construct
  source 1..159: mol_type = other RNA
taatttctactaagtgtagatcgtcgccgtccagctcgaccagccttattaaaccaaaaccatggtgagcaagggcgaggagctgttcaccggggtggtgcccatcctggtcgagctggacggcgacgtataatttctactaagtgtagattttttttt

SEQ ID NO: 36 - RNA, 168 nt, synthetic construct
  source 1..168: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggtaatttctactaagtgtagattttttttt

SEQ ID NO: 37 - RNA, 171 nt, synthetic construct
  source 1..171: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggacgtaatttctactaagtgtagattttttttt

SEQ ID NO: 38 - RNA, 179 nt, synthetic construct
  source 1..179: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggacggcgacgtataatttctactaagtgtagattttttttt

SEQ ID NO: 39 - RNA, 121 nt, synthetic construct
  source 1..121: mol_type = other RNA
ccatcctggtcgagctggaaattaacaaactaacatttgatcaggtcataatttctactaagtgtagatcgtcgccgtccagctcgaccagctaatttctactaagtgtagattttttttt

SEQ ID NO: 40 - RNA, 132 nt, synthetic construct
  source 1..132: mol_type = other RNA
ccatcctggtcgagctggacggcgacgtaaaattaacaaactaacatttgatcaggtcataatttctactaagtgtagatcgtcgccgtccagctcgaccagctaatttctactaagtgtagattttttttt

SEQ ID NO: 41 - RNA, 127 nt, synthetic construct
  source 1..127: mol_type = other RNA
tggtgcccatcctggtcgagctggaaattaacaaactaacatttgatcaggtcataatttctactaagtgtagatcgtcgccgtccagctcgaccagctaatttctactaagtgtagattttttttt

SEQ ID NO: 42 - RNA, 138 nt, synthetic construct
  source 1..138: mol_type = other RNA
tggtgcccatcctggtcgagctggacggcgacgtaaaattaacaaactaacatttgatcaggtcataatttctactaagtgtagatcgtcgccgtccagctcgaccagctaatttctactaagtgtagattttttttt

SEQ ID NO: 43 - RNA, 134 nt, synthetic construct
  source 1..134: mol_type = other RNA
accggggtggtgcccatcctggtcgagctggaaattaacaaactaacatttgatcaggtcataatttctactaagtgtagatcgtcgccgtccagctcgaccagctaatttctactaagtgtagattttttttt

SEQ ID NO: 44 - RNA, 145 nt, synthetic construct
  source 1..145: mol_type = other RNA
accggggtggtgcccatcctggtcgagctggacggcgacgtaaaattaacaaactaacatttgatcaggtcataatttctactaagtgtagatcgtcgccgtccagctcgaccagctaatttctactaagtgtagattttttttt

SEQ ID NO: 45 - RNA, 193 nt, synthetic construct
  source 1..193: mol_type = other RNA
taatttctactaagtgtagatcgtcgccgtccagctcgaccagccttattaaaccaaaaccatggtgagcaagggcgaggagctgttcaccggggtggtgcccatcctggtcgagctggtctctctccgcggttctatctagttacgcgttaaaccaactagaataatttctactaagtgtagattttttttt

SEQ ID NO: 46 - RNA, 196 nt, synthetic construct
  source 1..196: mol_type = other RNA
taatttctactaagtgtagatcgtcgccgtccagctcgaccagccttattaaaccaaaaccatggtgagcaagggcgaggagctgttcaccggggtggtgcccatcctggtcgagctggacgtctctctccgcggttctatctagttacgcgttaaaccaactagaataatttctactaagtgtagattttttttt

SEQ ID NO: 47 - RNA, 213 nt, synthetic construct
  source 1..213: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggtctctctccgcggttctatctagttacgcgttaaaccaactagaataatttctactaagtgtagattttttttt

SEQ ID NO: 48 - RNA, 216 nt, synthetic construct
  source 1..216: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggacgtctctctccgcggttctatctagttacgcgttaaaccaactagaataatttctactaagtgtagattttttttt

SEQ ID NO: 49 - RNA, 177 nt, synthetic construct
  source 1..177: mol_type = other RNA
taatttctactaagtgtagatcgtcgccgtccagctcgaccagccttattaaaccaaaaccatggtgagcaagggcgaggagctgttcaccggggtggtgcccatcctggtcgagctggccggagcagacgatatggcgtcgctccggtaatttctactaagtgtagattttttttt

SEQ ID NO: 50 - RNA, 180 nt, synthetic construct
  source 1..180: mol_type = other RNA
taatttctactaagtgtagatcgtcgccgtccagctcgaccagccttattaaaccaaaaccatggtgagcaagggcgaggagctgttcaccggggtggtgcccatcctggtcgagctggacgccggagcagacgatatggcgtcgctccggtaatttctactaagtgtagattttttttt

SEQ ID NO: 51 - RNA, 197 nt, synthetic construct
  source 1..197: mol_type = other RNA
taatttctactaagtgtagatcgtcgccgtccagctcgaccagccaaaagaactcttttctcttcttattaaaccaaaaccatggtgagcaagggcgaggagctgttcaccggggtggtgccctagctggtcgagctggccggagcagacgatatggcgtcgctccggtaatttctactaagtgtagattttttttt

SEQ ID NO: 52 - protein, 200 aa, synthetic construct
TAATTTCTACTAAGTGTAGATCGTCGCCGTCCAGCTCGACCAGCCAAAAGAACTCTTTTCTCTTCTTATTAAACCAAAACCATGGTGAGCAAGGGCGAGGAGCTGTTCACCGGGGTGGTGCCCTAGCTGGTCGAGCTGGACGCCGGAGCAGACGATATGGCGTCGCTCCGGTAATTTCTACTAAGTGTAGATTTTTTTTT

SEQ ID NO: 53 - RNA, 149 nt, synthetic construct
  source 1..149: mol_type = other RNA
taatttctactaagtgtagattcctgctagctttgacataatctcaagaccctcattgctagagctgttgctaatgaaacaggtgctttcttctttctgattaatggcattatgtcaaagtaatttctactaagtgtagattttttttt

SEQ ID NO: 54 - RNA, 152 nt, synthetic construct
  source 1..152: mol_type = other RNA
taatttctactaagtgtagattcctgctagctttgacataatctcaagaccctcattgctagagctgttgctaatgaaacaggtgctttcttctttctgattaatggcattatgtcaaagctataatttctactaagtgtagattttttttt

SEQ ID NO: 55 - RNA, 160 nt, synthetic construct
  source 1..160: mol_type = other RNA
taatttctactaagtgtagattcctgctagctttgacataatctcaagaccctcattgctagagctgttgctaatgaaacaggtgctttcttctttctgattaatggcattatgtcaaagctagcaggagataatttctactaagtgtagattttttttt

SEQ ID NO: 56 - RNA, 135 nt, synthetic construct
  source 1..135: mol_type = other RNA
taatttctactaagtgtagattcctgctagctttgacataatctctagagctgttgctaatgaaacaggtgctttcttctttctgattaatggcattatgtcaaagtaatttctactaagtgtagattttttttt

SEQ ID NO: 57 - RNA, 138 nt, synthetic construct
  source 1..138: mol_type = other RNA
taatttctactaagtgtagattcctgctagctttgacataatctctagagctgttgctaatgaaacaggtgctttcttctttctgattaatggcattatgtcaaagctataatttctactaagtgtagattttttttt

SEQ ID NO: 58 - RNA, 146 nt, synthetic construct
  source 1..146: mol_type = other RNA
taatttctactaagtgtagattcctgctagctttgacataatctctagagctgttgctaatgaaacaggtgctttcttctttctgattaatggcattatgtcaaagctagcaggagataatttctactaagtgtagattttttttt

SEQ ID NO: 59 - RNA, 163 nt, synthetic construct
  source 1..163: mol_type = other RNA
taatttctactaagtgtagattcctgctagctttgacataatctccccctggctctggaaagaccctcattgctagagctgttgctaatgaaacaggtgctttcttctttctgattaatggcattatgtcaaagtaatttctactaagtgtagattttttttt

SEQ ID NO: 60 - RNA, 166 nt, synthetic construct
  source 1..166: mol_type = other RNA
taatttctactaagtgtagattcctgctagctttgacataatctccccctggctctggaaagaccctcattgctagagctgttgctaatgaaacaggtgctttcttctttctgattaatggcattatgtcaaagctataatttctactaagtgtagattttttttt

SEQ ID NO: 61 - RNA, 174 nt, synthetic construct
  source 1..174: mol_type = other RNA
taatttctactaagtgtagattcctgctagctttgacataatctccccctggctctggaaagaccctcattgctagagctgttgctaatgaaacaggtgctttcttctttctgattaatggcattatgtcaaagctagcaggagataatttctactaagtgtagattttttttt

SEQ ID NO: 62 - RNA, 149 nt, synthetic construct
  source 1..149: mol_type = other RNA
taatttctactaagtgtagattcctgctagctttgacataatctcaaattaacaaactaacatttgatcaggtcagaaacaggtgctttcttctttctgattaatggcattatgtcaaagtaatttctactaagtgtagattttttttt

SEQ ID NO: 63 - RNA, 152 nt, synthetic construct
  source 1..152: mol_type = other RNA
taatttctactaagtgtagattcctgctagctttgacataatctcaaattaacaaactaacatttgatcaggtcagaaacaggtgctttcttctttctgattaatggcattatgtcaaagctataatttctactaagtgtagattttttttt

SEQ ID NO: 64 - RNA, 160 nt, synthetic construct
  source 1..160: mol_type = other RNA
taatttctactaagtgtagattcctgctagctttgacataatctcaaattaacaaactaacatttgatcaggtcagaaacaggtgctttcttctttctgattaatggcattatgtcaaagctagcaggagataatttctactaagtgtagattttttttt

SEQ ID NO: 65 - protein, 1975 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 66 - protein, 1975 aa, synthetic construct
MKRTADGSEFEPKKKRKVSGGSSGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYESSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGKRTADGSEFEPKKKRKV

SEQ ID NO: 67 - protein, 1992 aa, synthetic construct
MAPKKKRKVGIHGVPAAMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYESSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPKRPAATKKAGQAKKKKGSYPYDVPDYAYPYDVPDYAYPYDVPDYA

SEQ ID NO: 68 - protein, 2019 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKVSGGSSGGSALDFLSRLPLPPPVSPICTFVSPAAQKAFQPPRSCG

SEQ ID NO: 69 - protein, 2019 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVAPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKVSGGSSGGSALDFLSRLPLPPPVSPICTFVSPAAQKAFQPPRSCG

SEQ ID NO: 70 - DNA, 720 bp, synthetic construct
  source 1..720: mol_type = other DNA
atggtgagcaagggcgaggagctgttcaccggggtggtgccctagctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 71 - protein, 36 aa, synthetic construct
ALDFLSRLPLPPPVSPICTFVSPAAQKAFQPPRSCG

SEQ ID NO: 72 - protein, 110 aa, synthetic construct
QSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCE

SEQ ID NO: 73 - protein, 122 aa, synthetic construct
DFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQAL

SEQ ID NO: 74 - protein, 181 aa, synthetic construct
GGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 75 - protein, 129 aa, synthetic construct
AQHIVPCTISQLLSATLVDEVFRIGNVEISQVTIVGIIRHAEKAPTNIVYKIDDMTAAPMDVRQWVDTDDTSSENTVVPPETYVKVAGHLRSFQNKKSLVAFKIMPLEDMNEFTTHILEVINAHMVLSK

SEQ ID NO: 76 - protein, 250 aa, synthetic construct
LSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREP

SEQ ID NO: 77 - protein, 95 aa, synthetic construct
DLIGFVVSVVKKTGLAPFVYLSDECYNLLAIKFWIDLNEDIIKPHMLIAASNLQWRPESKSGLLTLFAGDFSVFSASPKEGHFQETFNKMKNTVE

SEQ ID NO: 78 - protein, 73 aa, synthetic construct
NTDEMVELRILLQSKNAGAVIGKGGKNIKALRTDYNASVSVPDSSGPERILSISADIETIGEILKKIIPTLEE

SEQ ID NO: 79 - protein, 79 aa, synthetic construct
CRVYVGSIYYELGEDTIRQAFAPFGPIKSIDMSWDSVTMKHKGFAFVEYEVPEAAQLALEQMNSVMLGGRNIKVGRPSN

SEQ ID NO: 80 - protein, 113 aa, synthetic construct
AMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQ

SEQ ID NO: 81 - DNA, 108 bp, synthetic construct
  source 1..108: mol_type = other DNA
gcgctcgatttcttatcccggcttccgctcccccccccggtgtcaccaatttgcaccttcgttagcccggccgcccaaaaggcgtttcagcctccgcggtcgtgcggc

SEQ ID NO: 82 - DNA, 330 bp, synthetic construct
  source 1..330: mol_type = other DNA
cagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgag

SEQ ID NO: 83 - DNA, 366 bp, synthetic construct
  source 1..366: mol_type = other DNA
gatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagcctta

SEQ ID NO: 84 - DNA, 543 bp, synthetic construct
  source 1..543: mol_type = other DNA
ggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatg

SEQ ID NO: 85 - DNA, 387 bp, synthetic construct
  source 1..387: mol_type = other DNA
gcccagcacattgtgccctgtactatatctcagctgctttctgccactttggttgatgaagtgttcagaattgggaatgttgagatttcacaggtcactattgtggggatcatcagacatgcagagaaggctccaaccaacattgtttacaaaatagatgacatgacagctgcacccatggacgttcgccagtgggttgacacagatgacaccagcagtgaaaacactgtggttcctccagaaacatatgtgaaagtggcaggccacctgagatcttttcagaacaaaaagagcctggtagcctttaagatcatgcccctggaggatatgaatgagttcaccacacatattctggaagtgatcaatgcacacatggtactaagcaaa

SEQ ID NO: 86 - DNA, 750 bp, synthetic construct
  source 1..750: mol_type = other DNA
ttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccc

SEQ ID NO: 87 - DNA, 285 bp, synthetic construct
  source 1..285: mol_type = other DNA
gacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgag

SEQ ID NO: 88 - DNA, 219 bp, synthetic construct
  source 1..219: mol_type = other DNA
aacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagag

SEQ ID NO: 89 - DNA, 237 bp, synthetic construct
  source 1..237: mol_type = other DNA
tgccgcgtctacgtgggctctatctactatgagctgggggaggacaccatccgccaggcctttgccccctttggccccatcaagagcatcgacatgtcctgggactccgtcaccatgaagcacaagggctttgccttcgtggagtatgaggtccccgaagctgcacagctggccttggagcagatgaactcggtgatgctggggggcaggaacatcaaggtgggcagacccagcaac

SEQ ID NO: 90 - DNA, 339 bp, synthetic construct
  source 1..339: mol_type = other DNA
gcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaa

SEQ ID NO: 91 - RNA, 21 nt, synthetic construct
  source 1..21: mol_type = other RNA
taatttctactaagtgtagat

SEQ ID NO: 92 - RNA, 37 nt, synthetic construct
  source 1..37: mol_type = other RNA
cgcggttctatctagttacgcgttaaaccaactagaa

SEQ ID NO: 93 - RNA, 29 nt, synthetic construct
  source 1..29: mol_type = other RNA
ccggagcagacgatatggcgtcgctccgg

SEQ ID NO: 94 - RNA, 30 nt, synthetic construct
  source 1..30: mol_type = other RNA
aaattaacaaactaacatttgatcaggtca

SEQ ID NO: 95 - RNA, 42 nt, synthetic construct
  source 1..42: mol_type = other RNA
ttgacgcggttctatctagttacgcgttaaaccaactagaaa

SEQ ID NO: 96 - RNA, 42 nt, synthetic construct
  source 1..42: mol_type = other RNA
ttgacgcggttctatctacttacgcgttaaaccaactagaaa

SEQ ID NO: 97 - RNA, 44 nt, synthetic construct
  source 1..44: mol_type = other RNA
cgcgagtctaggggataacgcgttaaacttcctagaaggcggtt

SEQ ID NO: 98 - RNA, 44 nt, synthetic construct
  source 1..44: mol_type = other RNA
cgcggatctagattgtaacgcgttaaaccatctagaaggcggtt

SEQ ID NO: 99 - RNA, 44 nt, synthetic construct
  source 1..44: mol_type = other RNA
cgcgtcgctaccgcccggcgcgttaaacacactagaaggcggtt

SEQ ID NO: 100 - RNA, 51 nt, synthetic construct
  source 1..51: mol_type = other RNA
gggtcaggagccccccccctgaacccaggataaccctcaaagtcggggggc

SEQ ID NO: 101 - RNA, 56 nt, synthetic construct
  source 1..56: mol_type = other RNA
gggtcaggagccccccccctgaacccaggataaccctcaaagtcggggggcaaccc

SEQ ID NO: 102 - RNA, 60 nt, synthetic construct
  source 1..60: mol_type = other RNA
gtcagggtcaggagccccccccctgaacccaggataaccctcaaagtcggggggcaaccc

SEQ ID NO: 103 - RNA, 56 nt, synthetic construct
  source 1..56: mol_type = other RNA
gggtcaggagccccccccctgaacccaggaaaaccctcaaagtcggggggcaaccc

SEQ ID NO: 104 - RNA, 56 nt, synthetic construct
  source 1..56: mol_type = other RNA
gggtcaggagccccccccctgcacccaggaaaaccctcaaagtcggggggcaaccc

SEQ ID NO: 105 - RNA, 56 nt, synthetic construct
  source 1..56: mol_type = other RNA
gggtcaggagccccccccctgcacccaggataaccctcaaagtcggggggcaaccc

SEQ ID NO: 106 - RNA, 60 nt, synthetic construct
  source 1..60: mol_type = other RNA
gtcagggtcaggagccccccccctgaacccaggaaaaccctcaaagtcggggggcaaccc

SEQ ID NO: 107 - RNA, 60 nt, synthetic construct
  source 1..60: mol_type = other RNA
gtcagggtcaggagccccccccctgcacccaggaaaaccctcaaagtcggggggcaaccc

SEQ ID NO: 108 - RNA, 63 nt, synthetic construct
  source 1..63: mol_type = other RNA
ggaggtcagggtcaggagcccccccctgaacccaggataaccctcaaagtcggggggcaaccc

SEQ ID NO: 109 - RNA, 55 nt, synthetic construct
  source 1..55: mol_type = other RNA
gggtcaggagcccccccctgaacccaggataaccctcaaagtcggggggcaaccc

SEQ ID NO: 110 - RNA, 13 nt, synthetic construct
  source 1..13: mol_type = other RNA
tggtggtggtggt

SEQ ID NO: 111 - RNA, 20 nt, synthetic construct
  source 1..20: mol_type = other RNA
gggacagggcagggacaggg

SEQ ID NO: 112 - RNA, 21 nt, synthetic construct
  source 1..21: mol_type = other RNA
gggtccgggtctgggtctggg

SEQ ID NO: 113 - RNA, 19 nt, synthetic construct
  source 1..19: mol_type = other RNA
gggcagggtctgggctggg

SEQ ID NO: 114 - RNA, 19 nt, synthetic construct
  source 1..19: mol_type = other RNA
gggctgggatgggaaaggg

SEQ ID NO: 115 - RNA, 19 nt, synthetic construct
  source 1..19: mol_type = other RNA
gggctctgggtgggccggg

SEQ ID NO: 116 - RNA, 18 nt, synthetic construct
  source 1..18: mol_type = other RNA
gggctgggctgggcaggg

SEQ ID NO: 117 - RNA, 17 nt, synthetic construct
  source 1..17: mol_type = other RNA
gggtgggctgggaaggg

SEQ ID NO: 118 - RNA, 17 nt, synthetic construct
  source 1..17: mol_type = other RNA
gggagggagggctaggg

SEQ ID NO: 119 - RNA, 17 nt, synthetic construct
  source 1..17: mol_type = other RNA
gggcagggctgggaggg

SEQ ID NO: 120 - RNA, 16 nt, synthetic construct
  source 1..16: mol_type = other RNA
gggtgggagggctggg

SEQ ID NO: 121 - RNA, 43 nt, synthetic construct
  source 1..43: mol_type = other RNA
gcgtaacctccatccgagttgcaagagagggaaacgcagtctc

SEQ ID NO: 122 - RNA, 160 nt, synthetic construct
  source 1..160: mol_type = other RNA
ggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttca

SEQ ID NO: 123 - RNA, 20 nt, synthetic construct
  source 1..20: mol_type = other RNA
taatttctactcttgtagat

SEQ ID NO: 124 - RNA, 19 nt, synthetic construct
  source 1..19: mol_type = other RNA
gggccctgaagaagggccc

SEQ ID NO: 125 - RNA, 21 nt, synthetic construct
  source 1..21: mol_type = other RNA
taagtctccatagaatggagg

SEQ ID NO: 126 - RNA, 34 nt, synthetic construct
  source 1..34: mol_type = other RNA
ggccaacatgaggatcacccatgtctgcagggcc

SEQ ID NO: 127 - RNA, 26 nt, synthetic construct
  source 1..26: mol_type = other RNA
ccaaaggctcttctcagagccaccca

SEQ ID NO: 128 - RNA, 31 nt, synthetic construct
  source 1..31: mol_type = other RNA
ggccagatctgagcctgggagctctctggcc

SEQ ID NO: 129 - RNA, 36 nt, synthetic construct
  source 1..36: mol_type = other RNA
atataaccttcaccattaggttcaaataatggtaat

SEQ ID NO: 130 - protein, 17 aa, synthetic construct
KRTADGSEFEPKKKRKV

SEQ ID NO: 131 - protein, 4 aa, synthetic construct
SGGS

SEQ ID NO: 132 - protein, 10 aa, synthetic construct
SGGSGGSGGS

SEQ ID NO: 133 - protein, 16 aa, synthetic construct
SGSETPGTSESATPES

SEQ ID NO: 134 - protein, 32 aa, synthetic construct
SGGSSGGSSGSETPGTSESATPESSGGSSGGS

SEQ ID NO: 135 - protein, 48 aa, synthetic construct
SGSETPGTSESATPESSGGSSGGSSGSETPGTSESATPESSGGSSGGS

SEQ ID NO: 136 - RNA, 27 nt, synthetic construct
  source 1..27: mol_type = other RNA
aacatgaggatcacccatgtctgcagg

SEQ ID NO: 137 - RNA, 30 nt, synthetic construct
  source 1..30: mol_type = other RNA
ggccaacatgaggatcacccatgtctgcag

SEQ ID NO: 138 - RNA, 21 nt, synthetic construct
  source 1..21: mol_type = other RNA
aacatgaggatcacccatgtc

SEQ ID NO: 139 - RNA, 26 nt, synthetic construct
  source 1..26: mol_type = other RNA
aacatgaggatcacccatgtctgcag

SEQ ID NO: 140 - RNA, 32 nt, synthetic construct
  source 1..32: mol_type = other RNA
ggcaacatgaggatcacccatgtctgcaggcc

SEQ ID NO: 141 - RNA, 34 nt, synthetic construct; identical to SEQ ID NO: 126
  source 1..34: mol_type = other RNA

SEQ ID NO: 142 - RNA, 68 nt, synthetic construct
  source 1..68: mol_type = other RNA
aacatgaggatcacccatgtctgcagggcctagaacttggccaacatgaggatcacccatgtctgcag

SEQ ID NO: 143 - RNA, 52 nt, synthetic construct
  source 1..52: mol_type = other RNA
aacatgaggatcacccatgtctgcagaacatgaggatcacccatgtctgcag

SEQ ID NO: 144 - RNA, 84 nt, synthetic construct
  source 1..84: mol_type = other RNA
ggccaacatgaggatcacccatgtctgcagggcctagcaagttaaaataaggccaacatgaggatcacccatgtctgcagggcc

SEQ ID NO: 145 - RNA, 91 nt, synthetic construct
  source 1..91: mol_type = other RNA
ggcaacatgaggatcacccatgtctgcaggcctagcaagttaataaggcttcaacttggccaacatgaggatcacccatgtctgcagggcc

SEQ ID NO: 146 - protein, 678 aa, Moloney murine leukemia virus
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 147 - protein, 675 aa, Cauliflower mosaic virus
MMDHLLQKTQIQNQTEQVMNITNPNSIYIKGRLYFKGYKKIELHCFVDTGASLCIASKFVIPEEHWINAERPIMVKIADGSSITINKVCRNIDLIIAGEIFHIPTVYQQESGIDFIIGNNFCQLYEPFIQFTDRVIFTKDRTYPVHIAKLTRAVRVGTEGFLESMKKRSKTHQPEPVNISTNKIAILSEGRRLSEEKLFITQQRMQKIEELLEKVCSENPLDPSKTKQWMKASIKLSDPSKAIKVKPMKYSPMDREEFDKQIKELLDLKVIKPSKSPHMAPAFLVNNEAEKRRGKKRMVVNYKAMNKATVGDAYNLPNKDELLTLIRGKKIFSSFDCKSGFWQVLLDQESRPLTAFTCPQGHYEWNVVPFGLKQAPSIFQRHMDEAFRVFRKFCCVYVDDILVFSNNEEDHLLHVAMILQKCNQHGIILSKKKAQLFKKKINFLGLEIDEGTHKPQGHILEHINKFPDTLEDKKQLQRFLGILTYASDYIPKLAQIRKPLQAKLKENVPWRWTKEDTLYMQKVKKNLQGFPPLHHPLPEEKLIIETDASDDYWGGMLKAIKINEGTNTELICRYASGSFKAAEKNYHSNDKETLAVINTIKKFSIYLTPVHFLIRTDNTHFKSFVNLNYKGDSKLGRNIRWQAWLSHYSFDVEHIKGTDNHFADFLSREFNRVNS

SEQ ID NO: 148 - protein, 333 aa, Escherichia coli
MGIHGVPAAMKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTSGGS

SEQ ID NO: 149 - DNA, 1307 bp, Arabidopsis thaliana
  source 1..1307: mol_type = genomic DNA
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatcaggtgttagtttctagtttgtgcgatcgaatttgtcgattaatctgagtttttctgattaacag

SEQ ID NO: 150 - DNA, 446 bp, Arabidopsis thaliana
  source 1..446: mol_type = genomic DNA
ttcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattg

SEQ ID NO: 151 - DNA, 528 bp, Cauliflower mosaic virus
  source 1..528: mol_type = genomic DNA
catggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 152 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaaattcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 153 - RNA, 23 nt, synthetic construct
  source 1..23: mol_type = other RNA
tcctacctcatctgggacatcat

SEQ ID NO: 154 - RNA, 24 nt, synthetic construct
  source 1..24: mol_type = other RNA
aaagtgccaccacttgggatcatc

SEQ ID NO: 155 - RNA, 23 nt, synthetic construct
  source 1..23: mol_type = other RNA
gtcccttatttctcatggaaaat

SEQ ID NO: 156 - RNA, 66 nt, Saccharomyces bayanus
  source 1..66: mol_type = other RNA
ttcttgtcgtacttatagatcgctacgttatttcaattttgaaaatctgagtcctgggagtgcgga

SEQ ID NO: 157 - protein, 609 aa, Homo sapiens [sequence omitted]

SEQ ID NO: 158 - protein, 482 aa, synthetic construct
  REGION 1..482: note = Synthetic polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 159 - RNA, 10 nt, Methanobacterium thermoautotrophicum
  source 1..10: mol_type = other RNA
aatttttgga

SEQ ID NO: 160 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 161 - RNA, 25 nt, Escherichia virus MS2
  source 1..25: mol_type = other RNA
gcgcacatgaggatcacccatgtgc

SEQ ID NO: 162 - protein, 116 aa, Escherichia virus MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 163 - RNA, 26 nt, Bacteriophage PP7
  source 1..26: mol_type = other RNA
ataaggagtttatatggaaaccctta

SEQ ID NO: 164 - protein, 127 aa, Bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 165 - RNA, 19 nt, unidentified
  source 1..19: mol_type = other RNA
ctgaatgcctgcgagcatc

SEQ ID NO: 166 - protein, 62 aa, unidentified
  REGION 1..62: note = Shigella virus
MKSIRCKNCNKLLFKADSFDHIEIRCPRCKRHIIMLNACEHPTEKHCGKREKITHSDETVRY

SEQ ID NO: 167 - protein, 1228 aa, synthetic construct
  REGION 1..1228: note = LbCas12a-RR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 168 - protein, 1228 aa, synthetic construct
  REGION 1..1228: note = LbCas12a-RVR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 169 - protein, 1228 aa, synthetic construct
  REGION 1..1228: note = enhanced LbCas12a
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 170 - protein, 1228 aa, synthetic construct
  REGION 1..1228: note = LbCas12a ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 171 - protein, 1228 aa, synthetic construct
  REGION 1..1228: note = LbCas12a-RR ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 172 - protein, 1228 aa, synthetic construct
  REGION 1..1228: note = LbCas12a-RVR ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 173 - protein, 1228 aa, synthetic construct
  REGION 1..1228: note = LbCas12a D156R
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 174 - DNA, 36 bp, synthetic construct
  source 1..36: mol_type = other DNA
ggcgatgcctgcttgccgaatatcatggtggaaaat

SEQ ID NO: 175 - DNA, 36 bp, synthetic construct
  source 1..36: mol_type = other DNA
ggctgatacctgcttgccgaatatcatggtggaaaa

SEQ ID NO: 176 - DNA, 36 bp, synthetic construct
  source 1..36: mol_type = other DNA
ggcgacgcctgcttgccgaatatcatggtggaaaat

SEQ ID NO: 177 - protein, 12 aa, synthetic construct
GDACLPNIMVEN

SEQ ID NO: 178 - protein, 10 aa, synthetic construct
YLLAEYHGGK

SEQ ID NO: 179 - RNA, 20 nt, synthetic construct
  source 1..20: mol_type = other RNA
ggaatcccttctgcagcacc

SEQ ID NO: 180 - RNA, 20 nt, synthetic construct
  source 1..20: mol_type = other RNA
gcggatgttccaatcagtac

SEQ ID NO: 181 - RNA, 20 nt, synthetic construct
  source 1..20: mol_type = other RNA
gattcctggtgccagaaaca

SEQ ID NO: 182 - RNA, 20 nt, synthetic construct
  source 1..20: mol_type = other RNA
ctgatggtccatgtctgtta

SEQ ID NO: 183 - RNA, 20 nt, synthetic construct
  source 1..20: mol_type = other RNA
gatgtctgcaggccagatga

SEQ ID NO: 184 - RNA, 20 nt, synthetic construct
  source 1..20: mol_type = other RNA
tgacctcccaaacagctaca

SEQ ID NO: 185 - RNA, 96 nt, synthetic construct
  source 1..96: mol_type = other RNA
ggaatcccttctgcagcaccgttttagagctagaaatagcaagttaaaataaggctagtccgttatcaacttgaaaaagtggcaccgagtcggtgc

SEQ ID NO: 186 - RNA, 126 nt, synthetic construct
  source 1..126: mol_type = other RNA
ggaatcccttctgcagcaccgttttagagctagaaatagcaagttaaaataaggctagtccgttatcaacttgaaaaagtggcaccgagtcggtgcggaaaagcgatcaaggtgctgcagaaggga

SEQ ID NO: 187 - RNA, 96 nt, synthetic construct
  source 1..96: mol_type = other RNA
gattcctggtgccagaaacagttttagagctagaaatagcaagttaaaataaggctagtccgttatcaacttgaaaaagtggcaccgagtcggtgc

SEQ ID NO: 188 - RNA, 124 nt, synthetic construct
  source 1..124: mol_type = other RNA
gattcctggtgccagaaacagttttagagctagaaatagcaagttaaaataaggctagtccgttatcaacttgaaaaagtggcaccgagtcggtgctcccgtcaccactgtttctggcaccagg

SEQ ID NO: 189 - RNA, 96 nt, synthetic construct
  source 1..96: mol_type = other RNA
gatgtctgcaggccagatgagttttagagctagaaatagcaagttaaaataaggctagtccgttatcaacttgaaaaagtggcaccgagtcggtgc

SEQ ID NO: 190 - RNA, 126 nt, synthetic construct
  source 1..126: mol_type = other RNA
gatgtctgcaggccagatgagttttagagctagaaatagcaagttaaaataaggctagtccgttatcaacttgaaaaagtggcaccgagtcggtgcgccatctggagcactcatctggcctgcaga

SEQ ID NO: 191 - RNA, 41 nt, synthetic construct
  source 1..41: mol_type = other RNA
taatttctactaagtgtagatgcggatgttccaatcagtac

SEQ ID NO: 192 - RNA, 94 nt, synthetic construct
  source 1..94: mol_type = other RNA
taatttctactaagtgtagatgcggatgttccaatcagtacggccctacttccgctttcaccttggagacggcgactctcagcgtactgattgg

SEQ ID NO: 193 - RNA, 41 nt, synthetic construct
  source 1..41: mol_type = other RNA
taatttctactaagtgtagatctgatggtccatgtctgtta

SEQ ID NO: 194 - RNA, 93 nt, synthetic construct
  source 1..93: mol_type = other RNA
taatttctactaagtgtagatctgatggtccatgtctgttatctggggaacacgcccggtgtcacgccacttgacaggccagtaacagacatg

SEQ ID NO: 195 - RNA, 41 nt, synthetic construct
  source 1..41: mol_type = other RNA
taatttctactaagtgtagattgacctcccaaacagctaca

SEQ ID NO: 196 - RNA, 94 nt, synthetic construct
  source 1..94: mol_type = other RNA
taatttctactaagtgtagattgacctcccaaacagctacagccctgggctctctgtacatgaagcaactccagtcccaattatgtagctgttt

SEQ ID NO: 197 - DNA, 27 bp, synthetic construct
  source 1..27: mol_type = other DNA
tcgagctcggtacctaatacgactcac

SEQ ID NO: 198 - DNA, 149 bp, synthetic construct
  source 1..149: mol_type = other DNA
  misc_feature 1..120: product = poly(A) Tail
ttttttttttttttttttttttttttttttttttttttttttttttttttttttttttttttttttttttttttttttttttttttttttttttttttttttttttttttttttttttttcttcctactcaggctttattcaaagacca

SEQ ID NO: 199 - protein, 1983 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 200 - protein, 853 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPKRPAATKKAGQAKKKK

SEQ ID NO: 201 - RNA, 73 nt, synthetic construct
  source 1..73: mol_type = other RNA
taatttctactaagtgtagattcttcttattaaaccaaaaccattaatttctactaagtgtagattttttttt

SEQ ID NO: 202 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtgccgatcctagtcgagctggtaatttctactaagtgtagattttttttt

SEQ ID NO: 203 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtacccatcctcgtcgagctggtaatttctactaagtgtagattttttttt

SEQ ID NO: 204 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtaccgatcctggtcgagctggtaatttctactaagtgtagattttttttt

SEQ ID NO: 205 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtgcccatcctcgtcgagctggtaatttctactaagtgtagattttttttt

SEQ ID NO: 206 - RNA, 148 nt, synthetic construct
  source 1..148: mol_type = other RNA
taatttctactaagtgtagatcgtcgccgtccagctcgaccagccttattaaaccaaaaccatggtgagcaagggcgaggagctgttcaccggggtggtgccgatcctggtcgagctggtaatttctactaagtgtagattttttttt

SEQ ID NO: 207 - RNA, 72 nt, synthetic construct
  source 1..72: mol_type = other RNA
gaggagctgttcaccggggtggtaccgatcctggtcgagctggtaatttctactaagtgtagattttttttt

SEQ ID NO: 208 - RNA, 72 nt, synthetic construct
  source 1..72: mol_type = other RNA
gaggagctgttcaccggggtggtacccatcctcgtcgagctggtaatttctactaagtgtagattttttttt

SEQ ID NO: 209 - RNA, 72 nt, synthetic construct
  source 1..72: mol_type = other RNA
gaggagctgttcaccggggtggtgccgatcctggtcgagctggtaatttctactaagtgtagattttttttt

SEQ ID NO: 210 - RNA, 72 nt, synthetic construct
  source 1..72: mol_type = other RNA
gaggagctgttcaccggggtggtgcccatcctcgtcgagctggtaatttctactaagtgtagattttttttt

SEQ ID NO: 211 - RNA, 72 nt, synthetic construct
  source 1..72: mol_type = other RNA
gaggagctgttcaccggggtggtgccgatcctagtcgagctggtaatttctactaagtgtagattttttttt

SEQ ID NO: 212 - RNA, 72 nt, synthetic construct
  source 1..72: mol_type = other RNA
gaggagctgttcaccggggtggtgcccatcctggtcgagctggtaatttctactaagtgtagattttttttt

SEQ ID NO: 213 - protein, 1900 aa, synthetic construct
MKRTADGSEFEPKKKRKVSGGSSGGSATVVSGQKQDRQGGERRRSQLDRDQCAYCKEKGHWAKDCPKKPRGPRGPRPQTSLLSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 214 - RNA, 188 nt, synthetic construct
  source 1..188: mol_type = other RNA
taatttctactaagtgtagatcgtcgccgtccagctcgaccagcgaactggtattccaaggaggcttattaaaccaaaaccatggtgagcaagggcgaggagctgttcaccggggtggtgcccatcctggtcgagctggcctccttggaataccagttctaatttctactaagtgtagattttttttt

SEQ ID NO: 215 - RNA, 168 nt, synthetic construct
  source 1..168: mol_type = other RNA
taatttctactaagtgtagatcgtcgccgtccagctcgaccagcgaactggtattccaaggagggtgagcaagggcgaggagctgttcaccggggtggtgcccatcctggtcgagctggcctccttggaataccagttctaatttctactaagtgtagattttttttt

SEQ ID NO: 216 - RNA, 148 nt, synthetic construct
  source 1..148: mol_type = other RNA
taatttctactaagtgtagatcgtcgccgtccagctcgaccagcgaactggtattccaaggagggttcaccggggtggtgcccatcctggtcgagctggcctccttggaataccagttctaatttctactaagtgtagattttttttt

SEQ ID NO: 217 - RNA, 188 nt, synthetic construct
  source 1..188: mol_type = other RNA
taatttctactaagtgtagatcgtcgccgtccagctcgaccagcggctagtccgttatcaacttcttattaaaccaaaaccatggtgagcaagggcgaggagctgttcaccggggtggtgcccatcctggtcgagctggaagttgataacggactagcctaatttctactaagtgtagattttttttt

SEQ ID NO: 218 - RNA, 168 nt, synthetic construct
  source 1..168: mol_type = other RNA
taatttctactaagtgtagatcgtcgccgtccagctcgaccagcggctagtccgttatcaacttgtgagcaagggcgaggagctgttcaccggggtggtgcccatcctggtcgagctggaagttgataacggactagcctaatttctactaagtgtagattttttttt

SEQ ID NO: 219 - RNA, 148 nt, synthetic construct
  source 1..148: mol_type = other RNA
taatttctactaagtgtagatcgtcgccgtccagctcgaccagcggctagtccgttatcaacttgttcaccggggtggtgcccatcctggtcgagctggaagttgataacggactagcctaatttctactaagtgtagattttttttt

SEQ ID NO: 220 - RNA, 133 nt, synthetic construct
  source 1..133: mol_type = other RNA
gcacatgaggatcacccatgtgccttattaaaccaaaaccatggtgagcaagggcgaggagctgttcaccggggtggtgcccatcctggtcgagctggcaaggcacatgaggatcacccatgtgctttttttt

SEQ ID NO: 221 - RNA, 306 nt, synthetic construct
  source 1..306: mol_type = other RNA
gccatcagtcgccggtcccaagcccggataaaatgggagggggcgggaaaccgcctaaccatgccgactgatggcagaactaagcacatgaggatcacccatgtgccttattaaaccaaaaccatggtgagcaagggcgaggagctgttcaccggggtggtgcccatcctggtcgagctggcaaggcacatgaggatcacccatgtgcaaattaacactgccatcagtcggcgtggactgtagaacactgccaatgccggtcccaagcccggataaaagtggagggtacagtccacgctttttttt

SEQ ID NO: 222 - RNA, 371 nt, synthetic construct
  source 1..371: mol_type = other RNA
gccatcagtcgccggtcccaagcccggataaaatgggagggggcgggaaaccgcctaaccatgccgactgatggcagaactaagcacatgaggatcacccatgtgccttattaaaccaaaaccatggtgagcaagggcgaggagctgttcaccggggtggtgcccatcctggtcgagctggcaaggcacatgaggatcacccatgtgcaaattaacactgccatcagtcggcgtggactgtagaacactgccaatgccggtcccaagcccggataaaagtggagggtacagtccacgctaatttctactaagtgtagatcgtcgccgtccagctcgaccagctaatttctactaagtgtagattttttttt

SEQ ID NO: 223 - RNA, 23 nt, synthetic construct
  source 1..23: mol_type = other RNA
cgtcgccgtccagctcgaccagc

SEQ ID NO: 224 - RNA, 149 nt, synthetic construct
  source 1..149: mol_type = other RNA
taatttctactaagtgtagatttgctggcgtttggtgttaggtttatgtgcacaatcttagccctgctcgcaaacgcctcgagctttcccgtgacgtggttatcaaacctaacaccaaactaatttctactaagtgtagattttttttt

SEQ ID NO: 225 - RNA, 150 nt, synthetic construct
  source 1..150: mol_type = other RNA
taatttctactaagtgtagatttgctggcgtttggtgttaggtttatgtgcacaatcttagccctgctcgcaaacgcctcgagctttcccgtgacgtggttatcaaacctaacaccaaacgtaatttctactaagtgtagattttttttt

SEQ ID NO: 226 - RNA, 151 nt, synthetic construct
  source 1..151: mol_type = other RNA
taatttctactaagtgtagatttgctggcgtttggtgttaggtttatgtgcacaatcttagccctgctcgcaaacgcctcgagctttcccgtgacgtggttatcaaacctaacaccaaacgctaatttctactaagtgtagattttttttt

SEQ ID NO: 227 - RNA, 127 nt, synthetic construct
  source 1..127: mol_type = other RNA
taatttctactaagtgtagatttgctggcgtttggtgttaggttctcgcaaacgcctcgagctttcccgtgacgtggttatcaaacctaacaccaaactaatttctactaagtgtagattttttttt

SEQ ID NO: 228 - RNA, 128 nt, synthetic construct
  source 1..128: mol_type = other RNA
taatttctactaagtgtagatttgctggcgtttggtgttaggttctcgcaaacgcctcgagctttcccgtgacgtggttatcaaacctaacaccaaacgtaatttctactaagtgtagattttttttt

SEQ ID NO: 229 - RNA, 129 nt, synthetic construct
  source 1..129: mol_type = other RNA
taatttctactaagtgtagatttgctggcgtttggtgttaggttctcgcaaacgcctcgagctttcccgtgacgtggttatcaaacctaacaccaaacgctaatttctactaagtgtagattttttttt

SEQ ID NO: 230 - RNA, 23 nt, synthetic construct
  source 1..23: mol_type = other RNA
catcaccacacacagacacgtga

SEQ ID NO: 231 - RNA, 23 nt, synthetic construct
  source 1..23: mol_type = other RNA
ttgctggcgtttggtgttaggtt

SEQ ID NO: 232 - RNA, 148 nt, synthetic construct
  source 1..148: mol_type = other RNA
taatttctactaagtgtagattcctacctcatctgggacatcatgagagggtgagggaggagagggaagtaagtggtggcgacgtagtagaagcaggaggctatgtgcatgtcccagattaatttctactaagtgtagattttttttt

SEQ ID NO: 233 - RNA, 193 nt, synthetic construct
  source 1..193: mol_type = other RNA
taatttctactaagtgtagattcctacctcatctgggacatcatgagagggtgagggaggagagggaagtaagtggtggcgacgtagtagaagcaggaggctatgtgcatgtcccagattctctctccgcggttctatctagttacgcgttaaaccaactagaataatttctactaagtgtagattttttttt

SEQ ID NO: 234 - RNA, 177 nt, synthetic construct
  source 1..177: mol_type = other RNA
taatttctactaagtgtagattcctacctcatctgggacatcatgagagggtgagggaggagagggaagtaagtggtggcgacgtagtagaagcaggaggctatgtgcatgtcccagatccggagcagacgatatggcgtcgctccggtaatttctactaagtgtagattttttttt

SEQ ID NO: 235 - RNA, 151 nt, synthetic construct
  source 1..151: mol_type = other RNA
taatttctactaagtgtagattcctacctcatctgggacatcatgagagggtgagggaggagagggaagtaagtggtggcgacgtagtagaagcaggaggctatgtgcatgtcccagatgagtaatttctactaagtgtagattttttttt

SEQ ID NO: 236 - RNA, 159 nt, synthetic construct
  source 1..159: mol_type = other RNA
taatttctactaagtgtagattcctacctcatctgggacatcatgagagggtgagggaggagagggaagtaagtggtggcgacgtagtagaagcaggaggctatgtgcatgtcccagatgaggtaggagataatttctactaagtgtagattttttttt

SEQ ID NO: 237 - RNA, 168 nt, synthetic construct
  source 1..168: mol_type = other RNA
taatttctactaagtgtagattcctacctcatctgggacatcatagaggccaggcgaagtaggagagagggtgagggaggagagggaagtaagtggtggcgacgtagtagaagcaggaggctatgtgcatgtcccagattaatttctactaagtgtagattttttttt

SEQ ID NO: 238 - RNA, 171 nt, synthetic construct
  source 1..171: mol_type = other RNA
taatttctactaagtgtagattcctacctcatctgggacatcatagaggccaggcgaagtaggagagagggtgagggaggagagggaagtaagtggtggcgacgtagtagaagcaggaggctatgtgcatgtcccagatgagtaatttctactaagtgtagattttttttt

SEQ ID NO: 239 - RNA, 179 nt, synthetic construct
  source 1..179: mol_type = other RNA
taatttctactaagtgtagattcctacctcatctgggacatcatagaggccaggcgaagtaggagagagggtgagggaggagagggaagtaagtggtggcgacgtagtagaagcaggaggctatgtgcatgtcccagatgaggtaggagataatttctactaagtgtagattttttttt

SEQ ID NO: 240 - RNA, 126 nt, synthetic construct
  source 1..126: mol_type = other RNA
taatttctactaagtgtagattcctacctcatctgggacatcataagtaagtggtggcgacgtagtagaagcaggaggctatgtgcatgtcccagattaatttctactaagtgtagattttttttt

SEQ ID NO: 241 - RNA, 129 nt, synthetic construct
  source 1..129: mol_type = other RNA
taatttctactaagtgtagattcctacctcatctgggacatcataagtaagtggtggcgacgtagtagaagcaggaggctatgtgcatgtcccagatgagtaatttctactaagtgtagattttttttt

SEQ ID NO: 242 - RNA, 137 nt, synthetic construct
  source 1..137: mol_type = other RNA
taatttctactaagtgtagattcctacctcatctgggacatcataagtaagtggtggcgacgtagtagaagcaggaggctatgtgcatgtcccagatgaggtaggagataatttctactaagtgtagattttttttt

SEQ ID NO: 243 - RNA, 127 nt, synthetic construct
  source 1..127: mol_type = other RNA
taatttctactaagtgtagattcctacctcatctgggacatcataagtaagtggtggcgacgtagtagaagcaggaggctatgtgcatgtcccagatgtaatttctactaagtgtagattttttttt

SEQ ID NO: 244 - RNA, 128 nt, synthetic construct
  source 1..128: mol_type = other RNA
taatttctactaagtgtagattcctacctcatctgggacatcataagtaagtggtggcgacgtagtagaagcaggaggctatgtgcatgtcccagatgataatttctactaagtgtagattttttttt

SEQ ID NO: 245 - RNA, 125 nt, synthetic construct
  source 1..125: mol_type = other RNA
taatttctactaagtgtagattcctacctcatctgggacatcataagtaagtggtggcgacgtagtagaagcaggaggctatgtgcatgtcccagataatttctactaagtgtagattttttttt

SEQ ID NO: 246 - RNA, 124 nt, synthetic construct
  source 1..124: mol_type = other RNA
taatttctactaagtgtagattcctacctcatctgggacatcataagtaagtggtggcgacgtagtagaagcaggaggctatgtgcatgtcccagtaatttctactaagtgtagattttttttt

SEQ ID NO: 247 - RNA, 190 nt, synthetic construct
  source 1..190: mol_type = other RNA
taatttctactaagtgtagatttgctggcgtttggtgttaggttgaactggtattccaaggaggtatgtgcacaatcttagccctgctcgcaaacgcctcgagctttcccgtgacgtggttatcaaacctaacaccaaacgcctccttggaataccagttctaatttctactaagtgtagattttttttt

SEQ ID NO: 248 - RNA, 190 nt, synthetic construct
  source 1..190: mol_type = other RNA
taatttctactaagtgtagatttgctggcgtttggtgttaggttggctagtccgttatcaactttatgtgcacaatcttagccctgctcgcaaacgcctcgagctttcccgtgacgtggttatcaaacctaacaccaaacgaagttgataacggactagcctaatttctactaagtgtagattttttttt

SEQ ID NO: 249 - RNA, 308 nt, synthetic construct
  source 1..308: mol_type = other RNA
gccatcagtcgccggtcccaagcccggataaaatgggagggggcgggaaaccgcctaaccatgccgactgatggcagaactaagcacatgaggatcacccatgtgctatgtgcacaatcttagccctgctcgcaaacgcctcgagctttcccgtgacgtggttatcaaacctaacaccaaacgcaaggcacatgaggatcacccatgtgcaaattaacactgccatcagtcggcgtggactgtagaacactgccaatgccggtcccaagcccggataaaagtggagggtacagtccacgctttttttt

SEQ ID NO: 250 - RNA, 135 nt, synthetic construct
  source 1..135: mol_type = other RNA
gcacatgaggatcacccatgtgctatgtgcacaatcttagccctgctcgcaaacgcctcgagctttcccgtgacgtggttatcaaacctaacaccaaacgcaaggcacatgaggatcacccatgtgctttttttt

SEQ ID NO: 251 - RNA, 135 nt, synthetic construct
  source 1..135: mol_type = other RNA
gcacatgaggatcacccatgtgctatgtgcacaatcttagccctgctcgcaaacgcctcgagctttcccgtgacgtggttatcgaatctaacaccaaacgcaaggcacatgaggatcacccatgtgctttttttt

SEQ ID NO: 252 - RNA, 150 nt, synthetic construct
  source 1..150: mol_type = other RNA
taatttctactaagtgtagatttgctggcgtttggtgttaggtttatgtgcacaatcttagccctgctcgcaaacgcctcgagctttcccgtgacgtggttatcgaatctaacaccaaacgtaatttctactaagtgtagattttttttt

SEQ ID NO: 253 - RNA, 188 nt, synthetic construct
  source 1..188: mol_type = other RNA
taatttctactaagtgtagattcctacctcatctgggacatcatgaactggtattccaaggagggagagggtgagggaggagagggaagtaagtggtggcgacgtagtagaagcaggaggctatgtgcatgtcccagatcctccttggaataccagttctaatttctactaagtgtagattttttttt

SEQ ID NO: 254 - RNA, 166 nt, synthetic construct
  source 1..166: mol_type = other RNA
taatttctactaagtgtagattcctacctcatctgggacatcatgaactggtattccaaggaggaagtaagtggtggcgacgtagtagaagcaggaggctatgtgcatgtcccagatcctccttggaataccagttctaatttctactaagtgtagattttttttt

SEQ ID NO: 255 - RNA, 188 nt, synthetic construct
  source 1..188: mol_type = other RNA
taatttctactaagtgtagattcctacctcatctgggacatcatggctagtccgttatcaacttgagagggtgagggaggagagggaagtaagtggtggcgacgtagtagaagcaggaggctatgtgcatgtcccagataagttgataacggactagcctaatttctactaagtgtagattttttttt

SEQ ID NO: 256 - RNA, 166 nt, synthetic construct
  source 1..166: mol_type = other RNA
taatttctactaagtgtagattcctacctcatctgggacatcatggctagtccgttatcaacttaagtaagtggtggcgacgtagtagaagcaggaggctatgtgcatgtcccagataagttgataacggactagcctaatttctactaagtgtagattttttttt

SEQ ID NO: 257 - RNA, 306 nt, synthetic construct
  source 1..306: mol_type = other RNA
gccatcagtcgccggtcccaagcccggataaaatgggagggggcgggaaaccgcctaaccatgccgactgatggcagaactaagcacatgaggatcacccatgtgcgagagggtgagggaggagagggaagtaagtggtggcgacgtagtagaagcaggaggctatgtgcatgtcccagatcaaggcacatgaggatcacccatgtgcaaattaacactgccatcagtcggcgtggactgtagaacactgccaatgccggtcccaagcccggataaaagtggagggtacagtccacgctttttttt

SEQ ID NO: 258 - RNA, 284 nt, synthetic construct
  source 1..284: mol_type = other RNA
gccatcagtcgccggtcccaagcccggataaaatgggagggggcgggaaaccgcctaaccatgccgactgatggcagaactaagcacatgaggatcacccatgtgcaagtaagtggtggcgacgtagtagaagcaggaggctatgtgcatgtcccagatcaaggcacatgaggatcacccatgtgcaaattaacactgccatcagtcggcgtggactgtagaacactgccaatgccggtcccaagcccggataaaagtggagggtacagtccacgctttttttt

SEQ ID NO: 259 - RNA, 133 nt, synthetic construct
  source 1..133: mol_type = other RNA
gcacatgaggatcacccatgtgcgagagggtgagggaggagagggaagtaagtggtggcgacgtagtagaagcaggaggctatgtgcatgtcccagatcaaggcacatgaggatcacccatgtgctttttttt

SEQ ID NO: 260 - RNA, 111 nt, synthetic construct
  source 1..111: mol_type = other RNA
gcacatgaggatcacccatgtgcaagtaagtggtggcgacgtagtagaagcaggaggctatgtgcatgtcccagatcaaggcacatgaggatcacccatgtgctttttttt

SEQ ID NO: 261 - RNA, 133 nt, synthetic construct
  source 1..133: mol_type = other RNA
gcacatgaggatcacccatgtgcgagagggtgagggaggagagggaagtaagtggtggcgacgtagtagaagcaggaggcgatatgcatgtcccagatcaaggcacatgaggatcacccatgtgctttttttt

SEQ ID NO: 262 - RNA, 111 nt, synthetic construct
  source 1..111: mol_type = other RNA
gcacatgaggatcacccatgtgcaagtaagtggtggcgacgtagtagaagcaggaggcgatatgcatgtcccagatcaaggcacatgaggatcacccatgtgctttttttt

SEQ ID NO: 263 - RNA, 148 nt, synthetic construct
  source 1..148: mol_type = other RNA
taatttctactaagtgtagattcctacctcatctgggacatcatgagagggtgagggaggagagggaagtaagtggtggcgacgtagtagaagcaggaggctatatgcatgtcccagattaatttctactaagtgtagattttttttt

SEQ ID NO: 264 - RNA, 148 nt, synthetic construct
  source 1..148: mol_type = other RNA
taatttctactaagtgtagattcctacctcatctgggacatcatgagagggtgagggaggagagggaagtaagtggtggcgacgtagtagaagcaggaggcgatatgcatgtcccagattaatttctactaagtgtagattttttttt

SEQ ID NO: 265 - RNA, 133 nt, synthetic construct
  source 1..133: mol_type = other RNA
gcacatgaggatcacccatgtgccttattaaaccaaaaccatggtgagcaagggcgaggagctgttcaccggggtggtacccatcctcgtcgagctggcaaggcacatgaggatcacccatgtgctttttttt

SEQ ID NO: 266 - protein, 1282 aa, synthetic construct
MAPKKKRKVGIHGVPAAMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 267 - protein, 1272 aa, synthetic construct
MKRTADGSEFEPKKKRKVSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 268 - protein, 1974 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPGGGGSGGGGSGGGGSGGGGSGGGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 269 - protein, 2145 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSSGSETPGTSESATPESSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKRTADGSEFEPKKKRKV

SEQ ID NO: 270 - protein, 2189 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFEPKKKRKV

SEQ ID NO: 271 - protein, 1426 aa, synthetic construct
MKRTADGSEFEPKKKRKVSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSSGSETPGTSESATPESSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKRTADGSEFEPKKKRKV

SEQ ID NO: 272 - protein, 1470 aa, synthetic construct
MKRTADGSEFEPKKKRKVSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFEPKKKRKV

SEQ ID NO: 273 - protein, 1634 aa, synthetic construct
MKRTADGSEFEPKKKRKVSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFEPKKKRKV

SEQ ID NO: 274 - protein, 1654 aa, synthetic construct
MKRTADGSEFEPKKKRKVSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSSGSETPGTSESATPESSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKRTADGSEFEPKKKRKV

SEQ ID NO: 275 - protein, 1632 aa, synthetic construct
MKRTADGSEFEPKKKRKVSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGSGGSSGGSSGSETPGTSESATPESSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFEPKKKRKV

SEQ ID NO: 276 - protein, 1264 aa, synthetic construct
MKRTADGSEFEPKKKRKVSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGKRTADGSEFEPKKKRKV

SEQ ID NO: 277 - protein, 2135 aa, synthetic construct
MKRTADGSEFEPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSSGGSSGSETPGTSESATPESSGGSSGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKRTADGSEFEPKKKRKV

SEQ ID NO: 278 - DNA, 4413 bp, synthetic construct
  source 1..4413: mol_type = other DNA
atgaaaagaaccgccgacggcagcgaattcgagcctaagaagaagaggaaggtttccggcggcagctcaggaggttccagcaagctggagaagttcaccaactgctactccctctcaaagaccctcaggttcaaggccatccctgtggggaagacccaggagaacattgacaacaagaggctgctggtggaggatgagaagagggctgaggactacaagggggtgaagaagctgctggacagatattacctctccttcatcaatgatgtgctgcactcaatcaagctgaagaacctcaacaactacatctccctcttcaggaagaagaccaggacagagaaggagaacaaggagctggagaacctggagatcaacctgaggaaggagattgccaaggccttcaagggcaatgaaggctacaagtcactcttcaagaaggacatcattgaaaccatcctcccagagttcctggatgacaaggatgagattgccctggtgaactccttcaatggcttcaccactgccttcactggcttctttagaaacagggagaacatgttctcagaggaggccaaatcaacaagcattgccttcaggtgcatcaatgagaacctcacccgctacatctccaacatggacatctttgagaaggtggatgccatctttgacaagcatgaggtgcaagagatcaaggagaagatcctgaactcagactatgatgtggaggacttctttgaaggagagttcttcaactttgtgctcacccaagaaggcattgatgtctacaatgccatcattggtggctttgtgacagagtcaggagagaagatcaagggcctcaatgagtacatcaacctctacaaccagaagaccaagcagaagctgcccaagttcaagcccctctacaagcaggtgctctctgacagggagagcttgagcttctatggagaaggctacacctctgatgaggaggtgctggaggtcttcaggaacaccctcaacaagaactcagagatcttctcctccatcaagaagctggagaagctcttcaagaactttgatgaatactcctctgctggcatctttgtgaagaatggccctgccatctccaccatctccaaggacatctttggagagtggaatgtcatcagggacaagtggaatgctgagtatgatgacatccacctaaagaagaaggctgtggtgacagagaagtatgaggatgacaggaggaagagcttcaagaagattggcagcttctccctggagcagctgcaagagtatgctgatgctgacctctctgtggtggagaagctgaaggagatcatcatccagaaggtggatgagatctacaaggtctatggctcctcagagaagctctttgatgctgactttgtgctggagaagagcctcaagaagaatgatgctgtggtggccatcatgaaggacctgctggactcagtgaagagctttgagaactacatcaaggccttctttggagaagggaaggaaaccaacagggatgagagcttctatggagactttgtgctggcctatgacatcctgctgaaggtggaccacatctatgatgccatcaggaactatgtcacccagaagccctactcaaaggacaagttcaagctctacttccagaacccccagttcatgggtggctgggacaaggacaaggagacagactacagggccaccatcctgaggtatggctcaaagtactacttggccatcatggacaagaagtatgccaagtgcctccagaagattgacaaggatgatgtcaatggcaactatgagaagataaactacaagctgctgcctggccccaacaagatgctgcccaaggtcttcttctccaagaagtggatggcctactacaacccctcagaggacatccagaagatctacaagaatggcaccttcaagaagggagacatgttcaacctcaatgactgccacaagctgattgacttcttcaaggactccatctccagataccccaagtggagcaatgcctatgacttcaacttctcagagacagagaagtacaaggacattgctggcttctacagggaggtggaggagcaaggctacaaggtgtcatttgagagtgcctccaagaaggaggtagacaagctggtggaggaagggaagctctacatgttccagatctacaacaaggacttctctgacaagagtcatggcacccccaacctccacaccatgtacttcaagctgctctttgatgagaacaatcatggccagatcaggctctcaggaggagcagagctcttcatgaggagggcctccctcaagaaggaggagctggtggtgcaccctgccaactcccccattgccaacaagaaccctgacaaccccaagaagaccaccaccctctcctatgatgtctacaaggacaagaggttctcagaggaccaatatgagctgcacatccccattgccatcaacaagtgccccaagaacatcttcaagatcaacacagaggtgagggtgctgctgaagcatgatgacaacccctatgtcattggcattgacagaggagagaggaacctcctatacattgtggtggtggatgggaagggcaacattgtggagcagtactccctgaatgagatcatcaacaacttcaatggcatcaggatcaagacagattaccactcactgctggacaagaaggagaaggagaggtttgaggcccgccaaaattggacaagcattgagaacatcaaggagctgaaggctggctacatctcccaggtggtgcacaagatctgtgagctggtggagaagtatgatgctgtgattgccctggaggacctcaactcttactgctcttccgtctaccagaagtttgagaagatgctgattgacaagctcaactacatggtggacaagaagagcaacccctgtgccactggtggtgccctcaagggctaccagatcaccaacaagtttgagagcttcaagagcatgagcacccagaatggcttcatcttctacatccctgcttggctcacctccaagattgacccctcaactggctttgtcaacctgctgaagaccaagtacacctccattgctgactccaagaagttcatctcctcctttgacaggatcatgtatgtgccagaggaggacctctttgagtttgccctggactacaagaacttctcaaggactgatgctgactacatcaagaagtggaagctctacagctatggcaacaggataaggatcttcaggaaccccaagaagaacaatgtctttgattgggaggaggtgtgcctcacctctgcctacaaggagctcttcaacaagtatggcatcaactaccagcaaggagacatcagagccctcctctgtgagcaatcagacaaggccttctactcctccttcatggccctgatgagcctgatgctgcagatgaggaactccatcactggaaggactgatgtggacttcctcatctccccagtgaagaactctgatggcatcttctatgactcaaggaactatgaggcccaggagaatgccatcctccccaagaatgctgatgccaatggagcctacaacattgcaaggaaggtgctgtgggccattggccagttcaagaaggctgaggatgagaagctggacaaggtgaagattgccatctccaacaaggagtggctggagtatgcccaaacctcagtgaagcactcaggtagtggggggagctccggcggcagcatggcggagaatggggacaacgagaagatggctgccctggaggccaagatctgccaccagattgaatactactttggggacttcaacctaccaagggacaagttcctcaaggagcagatcaagctggatgaggggtgggtgccactggagatcatgatcaagttcaaccgcctcaacaggctcaccacagacttcaacgtgatcgtggaggccctctccaagagcaaggcagagctgatggagatctcagaggacaagaccaagatcaggaggagcccctcaaagcccctgccggaggtgacggatgagtacaagaacgatgtgaagaacaggagtgtctacatcaagggcttccccaccgatgccaccctggatgacatcaaggagtggctggaggacaaggggcaggtgctgaacatccagatgaggaggaccctccacaaagcattcaaggggagcatcttcgtggtgtttgactcaattgagagtgccaagaagttcgtggagactcctggccagaagtacaaggagacagacctgctgatcctcttcaaggatgactactttgccaagaagaacgagagtggggggagcaaaagaaccgccgacggcagcgaattcgagcctaagaagaagaggaaggtttga

SEQ ID NO: 279 - DNA, 4413 bp, synthetic construct
  source 1..4413: mol_type = other DNA
atgaagcggaccgccgatggctcggagttcgagcccaagaagaagcggaaggtgtcgggggggagctccggggggagcagcaagctggagaagttcaccaactgctactctctctcaaagaccctccggttcaaggccatcccggtggggaagacccaggagaacattgacaacaagcggctgctggtggaggatgagaagcgggcggaggattacaagggggtgaagaagctgttggacagatattacctcagcttcatcaacgatgttttacactccatcaagctgaagaacctcaacaactacatctccctcttccggaagaagaccaggacggagaaggagaacaaggagctggagaacctggagatcaacctccggaaggagatcgccaaagcattcaaggggaatgagggctacaaaagtttgttcaagaaggatattattgaaaccatcctgccggagttcctagatgacaaggatgagattgccctggtcaattccttcaacggcttcaccaccgccttcaccggcttcttccggaaccgggagaacatgttctcagaggaagccaagagcaccagcattgccttccggtgcatcaacgagaacctgactcgttacatcagcaacatggacatcttcgagaaggtggacgccatcttcgacaagcatgaggttcaagagatcaaggagaagattctcaattcagactatgatgtggaggacttcttcgagggagagttcttcaacttcgtgctgacccaggaggggattgacgtctacaacgccatcatcgggggcttcgtgacagagtcaggggagaagatcaaggggctgaacgagtacatcaacctctacaaccagaagaccaagcagaagctgccaaagttcaagcccctctacaagcaggtgctctccgacagggaaagtttgagcttctatggggagggctacacttcagatgaggaggtgctggaggtgttccggaacaccctcaacaagaactcggagatcttctcctccatcaagaagctggaaaaacttttcaagaacttcgatgagtacagctccgccgggatcttcgtgaagaacggccctgccatctccaccatcagcaaggacatcttcggggagtggaacgtgatccgggacaagtggaacgccgagtatgatgatattcatttaaagaagaaggcggtggtgacggagaagtatgaggatgacagaaggaagagcttcaagaagattgggagcttctccctggagcagctccaggagtatgccgatgccgacctctcggtggtggagaagctgaaggagatcatcatccagaaggtggatgagatctacaaggtgtatgggagctcagagaagctgtttgatgccgacttcgtgctggagaaaagtttgaagaagaacgatgcggtggtggccatcatgaaggacctgctggactcggtgaaaagttttgagaactacatcaaagcattctttggggaggggaaggaaaccaaccgggatgaaagtttttatggggacttcgtgctggcctatgacattttattgaaggtggaccacatctatgatgccatccggaactatgtcacccagaagccctacagcaaggacaagttcaaactttacttccagaaccctcagttcatggggggctgggacaaggacaaggagacagactacagggccaccatcctaagatatgggagcaagtattacttggccatcatggacaagaagtatgccaagtgcctgcagaagatcgacaaggatgatgtgaatgggaactatgagaagatcaactacaagctgcttcctggacccaacaagatgctgcccaaggtgttcttctccaagaagtggatggcttactacaacccctcagaggacatccagaagatctacaagaacggcaccttcaagaagggggacatgttcaacctcaatgactgccacaagctgatcgacttcttcaaggacagcatctcaagataccccaagtggagcaacgcctatgacttcaacttctcagagacagagaagtacaaggacattgccggcttctacagggaggtggaggagcagggctacaaggtgagctttgagagtgcctcaaagaaggaggtggacaaattggtggaggaagggaaactttacatgttccagatctacaacaaggacttctccgacaaatctcatgggacccccaacctccacaccatgtacttcaagctgctgtttgatgagaacaatcatgggcagatccggctctcaggaggggcggagctcttcatgcggagggcctccctgaagaaggaggagctggtggtgcaccctgccaactctcccatcgccaacaagaaccctgacaaccccaagaagaccaccaccctcagctatgatgtctacaaggacaagcggttctcagaggaccagtatgagctgcacattcccatcgccatcaacaagtgccccaagaacatcttcaagatcaacaccgaggtgagggtgttgttgaagcatgatgataacccctatgtgatcgggatcgacaggggggagcggaacttgttatacattgtggtggtggacgggaaggggaacattgtggagcagtacagcttgaacgagatcatcaacaacttcaacgggatccggatcaagacagactaccattcattgttggacaagaaggagaaggagcggttcgaggccaggcagaactggacctccattgagaacatcaaggagctgaaggctggctacatcagccaggtggtgcacaagatctgcgagctggtggagaagtatgatgcggtgatcgccctggaggatctcaacagctattgctcttcagtttaccagaagttcgagaagatgctgatcgacaagctgaactacatggtggacaagaagagcaacccctgtgccaccgggggggcgctgaaaggataccaaatcaccaacaagtttgaaagtttcaagagcatgagcacccagaacggcttcatcttctacatccctgcctggctcaccagcaagatcgacccctcaactggcttcgtcaacctgctgaagaccaagtacacaagcatcgctgacagcaagaagttcatcagcagcttcgacaggatcatgtatgtgccggaggaggacctcttcgagttcgccctggactacaagaacttctcaaggaccgatgctgattacatcaagaagtggaagctctactcttatgggaaccggataaggattttccggaaccccaagaagaacaacgtcttcgactgggaggaggtgtgcctcacctccgcctacaaggagctcttcaacaagtatgggatcaactaccagcaaggggacatcagggcgctgctgtgtgagcaatcagacaaagcattttacagcagcttcatggccctgatgagcctgatgctgcagatgcggaactccatcaccgggaggaccgatgtggacttcctcatctccccggtgaagaactctgatgggatcttctatgactcaaggaactatgaggcccaggagaacgccatcctccccaagaacgccgatgccaatggggcctacaacattgccaggaaggtgctgtgggccatcggccagttcaagaaggccgaggatgagaagctggacaaggtgaagatcgccatcagcaacaaggagtggctggagtatgctcagacctcggtgaagcactcagggagtggggggagctccggggggagcatggcggagaacggggacaacgagaagatggctgccctggaggccaagatctgccaccagattgaatactacttcggggacttcaacctgccaagggacaagttcctgaaggagcagatcaagctagatgagggctgggtgcccttggagatcatgatcaagttcaaccggctgaaccggctcaccaccgacttcaacgtgatcgtggaggctctctccaaaagcaaggccgagctgatggagatctccgaggacaagaccaagatccggaggagcccctccaagcccctgccggaggtcaccgatgagtacaagaacgatgtgaagaacaggtcagtttacatcaagggcttccccaccgatgccaccctggatgacatcaaggagtggctggaggacaaggggcaggtgctgaacatccagatgcggaggaccctccacaaagcattcaaggggagcatcttcgtggtgttcgactcaatcgagagcgccaagaagttcgtggaaacccctgggcagaagtacaaggagacggacctgctgatcctcttcaaggatgattacttcgccaagaagaacgagagcggggggagcaagcggaccgccgatggctcggagttcgagcccaagaagaagcggaaggtgtga

SEQ ID NO: 280 - DNA, 4413 bp, synthetic construct
  source 1..4413: mol_type = other DNA
atgaagaggacggcggatggctcggagttcgagccgaagaagaagaggaaggtgtccggtgggagctccggtgggagctccaagctggagaagttcaccaactgctattcactctccaagaccctccggttcaaggccatcccggtggggaagacccaggagaacatcgacaacaagaggctgctggtggaggacgagaagcgggctgaggactacaagggggtgaagaagctcttggacagatactatctctccttcatcaacgatgttctacactccatcaagctgaagaacctcaacaactacatctccctcttccggaagaagacgaggacggagaaggagaacaaggagctggagaacctggagatcaacctccggaaggagatcgccaaggctttcaaggggaatgagggctacaagagcctcttcaagaaggacatcatcgagacgatcttaccggagttcctggatgacaaggacgagatcgcgctggtgaacagcttcaacggcttcaccaccgccttcaccggcttcttccggaaccgggagaacatgttctcagaggaagccaagagcacctccattgccttccggtgcatcaacgagaacctgactcgttacatctccaacatggacatcttcgagaaggtggacgccatcttcgacaagcatgaggttcaagagatcaaggagaagatcctcaactcagactacgatgtggaggacttcttcgagggggagttcttcaacttcgtgctgacccaagaggggattgacgtctacaacgccatcatcggaggcttcgtcacagagtcaggggagaagatcaaggggctcaacgagtacatcaacctctacaaccagaagaccaagcagaagctgccaaagttcaagccgctctacaagcaggtgctctccgaccgagagagcttgagcttctacggagagggctacacttcagatgaggaggtgctggaggtcttccggaacaccctcaacaagaactcggagatcttctcctccatcaagaagctggagaagctcttcaagaacttcgacgagtactcctccgctgggatcttcgtgaagaacggtccggcgatctccaccatctccaaggacatcttcggcgagtggaacgtgatccgggacaagtggaacgcggagtacgatgacattcatctaaagaagaaggcggtggtgacggagaagtacgaggacgaccggaggaagagcttcaagaagattgggagcttctccctggagcagctccaggagtacgctgatgctgacctctcggtggtggagaagctgaaggagatcatcatccagaaggtggacgagatctacaaggtctacgggagctcagagaagctcttcgacgctgacttcgtgctggagaagagcctcaagaagaacgatgcggtggtggccatcatgaaggacctgctggattcagtgaagagcttcgagaactacatcaaggctttcttcggagaggggaaggagacgaaccgagatgagagcttctacggagacttcgtgctggcttatgacatcctcctcaaggtggaccacatctatgatgccatccggaactatgtgacccagaagccttactcaaaggacaagttcaagctctacttccagaaccctcagttcatgggaggctgggacaaggacaaggagacagactacagagccaccatcctccggtatggatcaaagtactacctcgccatcatggacaagaagtatgccaagtgcctccagaagatcgacaaggatgatgtgaacgggaactacgagaagatcaactacaagctgcttcccggtcccaacaagatgctccccaaggtcttcttctccaagaagtggatggcttactacaacccctcagaggacatccagaagatctacaagaacgggaccttcaagaagggggacatgttcaacctcaatgattgtcacaagctgatcgacttcttcaaggactccatctcaaggtacccaaagtggagcaacgcttatgacttcaacttctcggagacggagaagtacaaggacatcgctggcttctaccgggaggtggaggagcagggctacaaggtgagctttgagagcgcttcaaagaaggaggtggacaagctggtggaggaggggaagctctacatgttccagatctacaacaaggacttctccgacaagagtcatgggacgccaaacctccacaccatgtacttcaaacttctcttcgatgagaacaaccacggccagatccggctctcaggaggagcggagctcttcatgaggagagcctccctcaagaaggaggagctggtggtccacccggccaactctccgatcgccaacaagaacccggacaaccccaagaagaccaccacgctgagctatgatgtctacaaggacaagaggttctcagaggaccagtacgagctccacatccccatcgccatcaacaagtgtccgaagaacatcttcaagatcaacacggaggtgagggtcctcctcaagcacgatgacaacccttatgtgatcgggatcgaccggggggagcggaacctcctctacatcgtggtggtggacgggaaggggaacatcgtggagcagtattcactcaacgagatcatcaacaacttcaatgggatcagaatcaagacagattaccactctctgctggacaagaaggagaaggagcggtttgaggcgaggcagaactggacctccatcgagaacatcaaggagctgaaggctggctacatctcacaagtggtccacaagatctgcgagctggtggagaagtatgatgcggtgatcgcgctggaggacctcaacagctactgctcttcagtctaccagaagttcgagaagatgctgatcgacaagctcaactacatggtggacaagaagagcaacccttgcgccaccggcggcgctctcaaaggttatcagatcaccaacaagtttgagagcttcaagagcatgagcacccagaacggcttcatcttctacatcccggcttggctcacctccaagatcgacccttcaaccggcttcgtcaacctcttgaagaccaagtacacctccattgctgactccaagaagttcatctcctccttcgaccggatcatgtacgttccggaggaggacctcttcgagttcgcgctggactacaagaacttctcaaggacagatgctgactacatcaagaagtggaagctctacagctatgggaaccggataagaatcttccggaaccccaagaagaacaacgtcttcgactgggaggaggtctgcctcacctccgcctacaaggagctcttcaacaagtatgggatcaactaccagcagggggacatccgagctctcctctgtgagcagagcgacaaggctttctactcaagtttcatggcgctgatgagcctgatgctgcagatgaggaactccatcaccgggaggacggatgtggacttcctcatctcgccggtgaagaactccgatgggatcttctacgactcaagaaactacgaggctcaggagaacgccatcctccccaagaacgctgatgccaacggagcctacaacatcgccaggaaggtgctctgggccatcggccagttcaagaaggctgaggacgagaaacttgacaaggtgaagatcgccatctcaaacaaggagtggctggagtatgctcagacctcggtgaagcactccggctccggtgggagctccggaggctcgatggcggagaacggcgacaacgagaagatggcggcgctggaggccaagatctgccaccagatcgagtactacttcggagacttcaacctaccgagagacaagttcctcaaggagcagatcaagctggatgagggctgggttccgctggagatcatgatcaagttcaaccggctcaaccggctcaccaccgacttcaacgtgatcgtggaggcgctctccaagagcaaggctgagctgatggagatctccgaggacaagaccaagatccggaggagcccttcaaagccgctgccggaggtcaccgacgagtacaagaacgatgtgaagaaccggagcgtctacatcaagggcttcccgacggatgccacgctggatgacatcaaggagtggctggaggacaagggccaggtgctcaacatccagatgaggaggaccctccacaaggctttcaaggggagcatcttcgtggtcttcgactccattgagtcagccaagaagttcgtggagactcccggccagaagtacaaggagacggacctcctcatcctcttcaaggatgattacttcgccaagaagaacgagagcggaggctccaagaggacggcggatggctcggagttcgagccgaagaagaagaggaaggtgtga

SEQ ID NO: 281 - DNA, 4413 bp, synthetic construct
  source 1..4413: mol_type = other DNA
atgaagcgcaccgccgacggcagcgagttcgagcccaagaagaagcgcaaggtgagcggcggcagcagcggcggcagcagcaagctggagaagttcaccaactgctacagcctctcaaagaccctccgcttcaaggccatcccggtggggaagacccaggagaacatcgacaacaagcggctgctggtggaggatgagaagcgcgccgaggactacaagggggtgaagaagctgctggaccgctactacctcagcttcatcaacgacgtgctgcattcaatcaagctgaagaacctcaacaactacatcagcctcttccggaagaagacccggacggagaaggagaacaaggagctggagaacctggagatcaacctccgcaaggagatcgccaaggccttcaagggcaatgagggctacaagagcctcttcaagaaggacatcatcgagacgatcctgccggagttcttggacgacaaggatgagatcgccctggtgaactcattcaacggcttcaccaccgccttcaccggcttcttccgcaaccgggagaacatgttctccgaggaggccaagagcaccagcatcgccttccgctgcatcaacgagaacctgacgcgctacatcagcaacatggacatcttcgagaaggtggacgccatcttcgacaagcatgaagttcaggagatcaaggagaagatcctcaattcagactacgacgtggaggacttcttcgagggagagttcttcaacttcgtgctgacccaggagggcattgatgtgtacaacgccatcatcggcggcttcgtcaccgagagcggcgagaagatcaagggcctcaacgagtacatcaacctctacaaccagaagaccaagcagaagctgcccaagttcaagccgctctacaagcaggtgctctcagaccgggagagcttgagcttctacggcgagggctacaccagcgatgaggaggtgctggaggtgttccgcaacaccctcaacaagaactcagagatcttcagcagcatcaagaagctggagaagctcttcaagaacttcgacgagtacagcagcgccggcatctttgtgaagaacgggccggccatctcaaccatcagcaaggacatcttcggcgagtggaacgtcatccgggacaagtggaacgccgagtacgatgacatccatctgaagaagaaggcggtggtgacggagaagtacgaggacgaccggcggaagagcttcaagaagatcggcagcttctcattggagcagctgcaagagtacgccgacgccgacctctccgtggtggagaagctcaaggagatcatcatccagaaggtggacgagatctacaaggtgtacggcagctcagagaagctcttcgacgctgacttcgtgctggagaagagcctgaagaagaacgacgccgtggtggccatcatgaaggacctgctggacagcgtcaagagcttcgagaactacatcaaggccttcttcggcgagggcaaggagacgaaccgggatgagtcattctacggcgacttcgtgctggcctacgacatcctcctcaaggtggaccacatctatgacgccatccgcaactatgtgacacagaagccctacagcaaggacaagttcaagctctacttccagaacccccagttcatgggcggctgggacaaggacaaggagacagactaccgggccaccatcctgaggtatggatcaaagtactacctggccatcatggacaagaagtacgccaagtgcctccagaagatcgacaaggacgacgtgaatggcaactatgagaagatcaactacaagctgctgccggggccgaacaagatgctgcccaaggtgttcttctcaaagaagtggatggcctactacaaccccagcgaggacatccagaagatctacaagaacggcaccttcaagaagggcgacatgttcaacctgaatgactgccacaagctcatcgacttcttcaaggacagcatcagcagataccccaagtggagcaacgcctatgacttcaacttctcagagacggagaagtacaaggacatcgccggcttctaccgggaggtggaggagcagggctacaaggtgagctttgagagcgccagcaagaaggaggtggacaagctggtggaggagggcaagctctacatgttccagatctacaacaaggacttctccgacaaatctcatggcacccccaacctccacaccatgtacttcaagctgctatttgatgagaacaatcatggccagatccggctctccggcggcgccgagctcttcatgcggcgagcatcattgaagaaggaggagctggtggtgcacccggccaacagccccatcgccaacaagaacccggacaaccccaagaagaccaccaccctcagctatgatgtgtacaaggacaagcgcttctcagaggaccagtacgagctgcacatccccatcgccatcaacaagtgccccaagaacatcttcaagatcaacaccgaggtgcgggtgctactgaagcacgatgacaacccatatgtcatcggcatcgaccgcggcgagcgcaacctcctctacatcgtggtggtggacggcaagggcaacatcgtggagcagtacagcctgaacgagatcatcaacaacttcaacggcatccgcatcaagaccgactaccattcattgctggacaagaaggagaaggagcgctttgaggcgcggcagaactggaccagcatcgagaacatcaaggagctgaaggccggctacatcagccaggtggtgcacaagatctgcgagctggtggagaagtatgacgccgtcatcgccctggaggacctcaacagctactgcagctcagtgtaccagaagttcgagaagatgctgatcgacaagctcaactacatggtggacaagaagagcaacccctgcgccaccggcggcgccctcaagggctaccagatcaccaacaagtttgagagcttcaagagcatgagcacccagaacggcttcatcttctacatcccggcgtggctcaccagcaagattgatccatcaaccggcttcgtcaacctgctgaagaccaagtacaccagcatcgccgacagcaagaagttcatcagcagcttcgaccgcatcatgtacgtgccggaggaggacctcttcgagttcgccctggactacaagaacttctcaaggaccgacgctgactacatcaagaagtggaagctctacagctacggcaaccggataaggatcttccgcaaccccaagaagaacaacgtcttcgactgggaggaggtgtgcctcacctcagcatacaaggagctcttcaacaagtacggcatcaactaccagcagggcgacatccgggcgctgctgtgtgagcagagcgacaaggccttctacagcagcttcatggcgctgatgagcctgatgctgcagatgcgcaacagcatcaccggccgcaccgacgtggacttcctcatctcgccggtgaagaactcagacggcatcttctatgacagccgcaactacgaggcgcaggagaacgccatcctccccaagaacgccgacgccaacggcgcctacaacatcgcccggaaggtgctatgggccatcggccagttcaagaaggcggaggatgagaagctggacaaggtgaagatcgccatcagcaacaaggagtggctggagtatgctcagacctccgtcaagcacagcggcagcggcggcagcagcggcggctcaatggcggagaacggcgacaacgagaagatggcggcgctggaggccaagatctgccaccagattgagtactacttcggcgacttcaacctgccgcgggacaagttcctcaaggagcagatcaagctagatgagggctgggtgccgctggagatcatgatcaagttcaaccggctcaaccggctcaccaccgacttcaacgtcatcgtggaggcgctcagcaagagcaaggcggagctgatggagatctcagaagacaagaccaagatccggcgctcgccgagcaagccgctgccggaggtcaccgacgagtacaagaatgatgtcaagaaccgctcagtgtacatcaagggcttccccaccgacgccaccctggacgacatcaaggagtggctggaggacaagggccaggtgctcaacatccagatgaggcgcaccctccacaaggccttcaagggcagcatcttcgtggtgttcgacagcattgaatcagccaagaagttcgtggagacgccggggcagaagtacaaggagacagacctcctcatcctcttcaaggatgactacttcgccaagaagaacgagagcggcggcagcaagcgcaccgccgacggcagcgagttcgagcccaagaagaagcgcaaggtgtga

SEQ ID NO: 282 - DNA, 4413 bp, synthetic construct
  source 1..4413: mol_type = other DNA
atgaagcgcaccgccgacggcagcgagttcgagcccaagaagaagcgcaaggtgtccggcggcagctccggcggctcaagcaagctggagaagttcaccaactgctacagcctctccaagaccctgaggttcaaggccatccccgtcggcaagacccaggagaacattgacaacaagcgcctcctggtggaggacgagaagcgcgccgaggactacaagggcgtcaagaagctactggaccggtactacctgagcttcatcaatgatgtcctccactccatcaagctcaagaacctcaacaactacatctccctcttccgcaagaagacccgcaccgagaaggagaacaaggagctggagaacctggagatcaacctccgcaaggagatagccaaggccttcaagggcaacgagggctacaagagcctcttcaagaaggacatcatcgagacgatcctaccggagttcctcgacgacaaggatgagatcgccctcgtcaacagcttcaacggcttcaccaccgccttcaccggcttcttccgcaaccgtgagaacatgttctccgaggaggccaagagcacctccatcgccttccgctgcatcaacgagaacctgacccgctacatctcaaacatggacatcttcgagaaggtggacgccatcttcgacaagcacgaggtccaggagatcaaggagaagatcctcaacagcgactatgatgtggaggacttcttcgagggcgagttcttcaacttcgtcctgacccaggagggcatcgacgtctacaacgccatcatcggcggcttcgtcaccgagtcaggagagaagatcaagggcctcaacgagtacatcaacctctacaaccagaagaccaagcagaagctccccaagttcaagcccctctacaagcaggtgctctcagaccgtgagagcctgagcttctacggcgagggctacacctccgacgaggaggtgctggaggtcttccgcaacaccctcaacaagaactccgagatcttctcctccatcaagaagctggagaagctcttcaagaacttcgacgagtactcaagcgccggcatcttcgtgaagaacggcccggccatctccaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggatgagatctacaaggtgtacggcagctcagagaagctcttcgacgccgacttcgtcctggagaagagcctcaagaagaacgacgccgtggtggccatcatgaaggacctcctcgacagcgtcaagagcttcgagaactacatcaaggccttcttcggcgagggcaaggagacaaaccgcgacgagtcattctacggcgacttcgtcctcgcctatgacatcctcctcaaggtggaccacatctacgacgccatccgcaactacgtgacccagaagccctacagcaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgacgtgaacggcaactacgagaagatcaactacaagctcctccccggccccaacaagatgctccccaaggtcttcttctcaaagaagtggatggcctactacaacccctccgaggacatccagaagatctacaagaacggcaccttcaagaagggtgacatgttcaacctcaacgactgccacaagctgatcgacttcttcaaggacagcatctcacggtaccccaagtggagcaacgcctacgacttcaacttctctgagacggagaagtacaaggacatcgccggcttctaccgggaggtggaggagcagggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccttgagctacgacgtctacaaggacaagcgcttctcagaagaccagtacgagctccacatccccatcgccatcaacaagtgccccaagaacatcttcaagatcaacaccgaggtgcgcgtcctactcaagcatgatgataacccctacgtgatcggcatcgaccgaggagagcgcaacctcctctacatcgtggtggtggacggcaagggcaacatcgtggagcagtacagcctcaacgagatcatcaacaacttcaacggcatccgcatcaagaccgactaccactcactccttgacaagaaggagaaggagcgcttcgaggcccggcagaactggacctccatcgagaacatcaaggagctgaaggccggctacatctcccaggtggtgcacaagatctgcgagctggtggagaagtacgacgccgtcatcgccctggaggacctcaacagctactgctcaagcgtctaccagaagtttgagaagatgctcatcgacaagctgaactacatggtggacaagaagagcaacccctgcgccaccggcggcgccctcaagggctaccagatcaccaacaagttcgagagcttcaagtcaatgtccacccagaacggcttcatcttctacatcccggcctggctcacctccaagattgacccctccaccggcttcgtcaacctcctgaagaccaagtacacctccatcgccgacagcaagaagttcatctcctccttcgaccgcatcatgtacgtgccggaggaggacctcttcgagttcgcccttgactacaagaacttctcacgcaccgacgccgactacatcaagaagtggaagctctacagctacggcaaccggataaggatcttccgcaaccccaagaagaacaacgtcttcgactgggaggaggtgtgcctcacctccgcctacaaggagctcttcaacaagtacggcatcaactaccagcaaggtgacatccgcgccctcctatgcgagcagagcgacaaggccttctactcctccttcatggccctcatgagcctcatgctccagatgcgcaacagcatcaccggccgcaccgacgtcgacttcctcatctccccggtgaagaactctgatggcatcttctacgactcacgcaactacgaggcccaggagaacgccatcctccccaagaacgccgacgccaacggcgcctacaacatcgcccgcaaggtgctctgggccatcggccagttcaagaaggctgaggacgagaagctggacaaggtcaagatcgccatctccaacaaggagtggctggagtacgctcagacctccgtgaagcactccggcagcggcggcagctcaggaggcagcatggccgagaacggcgacaacgagaagatggccgcccttgaggccaagatctgccaccagatcgagtactacttcggcgacttcaacctacctcgggacaagttcctcaaggagcagatcaagctggatgagggctgggtgccgctggagatcatgatcaagttcaaccgcctcaaccgcctcaccaccgacttcaacgtcatcgtggaggccctctccaagagcaaggccgagctcatggagatctccgaggacaagaccaagatccgccgctcaccctccaagcccctccctgaggtgaccgacgagtacaagaacgacgtcaagaaccgctccgtctacatcaagggcttccccaccgacgccaccctggatgacatcaaggagtggctggaggacaagggccaggtgctcaacatccagatgaggaggaccctccacaaggccttcaagggcagcatcttcgtcgtcttcgacagcatcgagtcagccaagaagttcgtggagacaccaggccagaagtacaaggagacggacctcctcatcctcttcaaggatgactacttcgccaagaagaacgagtccggcggcagcaagcgcaccgccgacggcagcgagttcgagcccaagaagaagcgcaaggtgtga

SEQ ID NO: 283 - DNA, 4413 bp, synthetic construct
  source 1..4413: mol_type = other DNA
atgaagcgcaccgccgacggcagcgagttcgagcccaagaagaagcgcaaggtgagcggcggcagcagcggcggcagcagcaagctggagaagttcaccaactgctacagcctctcaaagacgctgaggttcaaggccatccccgtcggcaagacccaggagaacatcgacaacaagcggctgctggtggaggatgagaagcgcgccgaggactacaagggcgtcaagaagctgctggaccgctactacctctcattcatcaacgacgtgctccacagcatcaagctgaagaacctcaacaactacatcagcctcttcagaaagaagacgcggacggagaaggagaacaaggagctggagaacctggagatcaacctaaggaaggagatcgccaaggccttcaagggcaatgagggctacaagagcctcttcaagaaggacatcatcgagaccatcctgccggagttcctggacgacaaggatgagatcgcgctggtgaacagcttcaacggcttcaccaccgccttcaccggcttcttcagaaaccgggagaacatgttctcagaggaggccaagagcaccagcatcgccttccgctgcatcaacgagaacctgacgcgctacatcagcaacatggacatcttcgagaaggtggacgccatcttcgacaagcatgaggtgcaagagatcaaggagaagatcctcaactcagactacgacgtggaggacttcttcgagggcgagttcttcaacttcgtgctgacccaggagggcattgatgtctacaacgccatcatcggcggcttcgtcaccgagtcaggggagaagatcaagggcctcaacgagtacatcaacctctacaaccagaagaccaagcagaagctgcccaagttcaagccgctctacaagcaggtgctctccgaccgggagagcttgagcttctacggcgagggctacaccagcgatgaggaggtgctggaggtgttcagaaacaccctcaacaagaactcagagatcttcagcagcatcaagaagctggagaagctcttcaagaacttcgacgagtacagcagcgccggcatcttcgtgaagaacggccccgccatctccaccatcagcaaggacatcttcggcgagtggaacgtcatccgcgacaagtggaacgccgagtacgatgacatccatctaaagaagaaggcggtggtgacggagaagtacgaggacgaccgccgcaagagcttcaagaagattggcagcttctccctggagcagctccaggagtacgccgacgccgacctctccgtggtggagaagctgaaggagatcatcatccagaaggtggacgagatctacaaggtgtacggcagctcagagaagctcttcgacgctgacttcgtgctggagaagagcctcaagaagaacgacgccgtggtggccatcatgaaggacctgctggattcagtgaagagcttcgagaactacatcaaggccttcttcggcgagggcaaggagaccaaccgcgacgagagcttctacggcgacttcgtgctggcctatgacatcctgctgaaggtggaccacatctatgacgccatccgcaactacgtcacccagaagccctactcaaaggacaagttcaagctctacttccagaacccgcagttcatgggcggctgggacaaggacaaggagacggactaccgcgccaccatcctgaggtatggatcaaagtactacctcgccatcatggacaagaagtacgccaagtgcctccagaagatcgacaaggatgacgtgaacggcaactacgagaagatcaactacaagctgctgcccggccccaacaagatgctgcccaaggtgttcttcagcaagaagtggatggcctactacaacccctcagaggacatccagaagatctacaagaacggcaccttcaagaagggcgacatgttcaacctcaatgactgccacaagctgatcgacttcttcaaggacagcatcagccgctaccccaagtggagcaacgcctatgacttcaacttctcagagacggagaagtacaaggacatcgccggcttctaccgcgaggtggaggagcagggctacaaggtgagcttcgagagcgcctcaaagaaggaggtggacaagctggtggaggagggcaagctctacatgttccagatctacaacaaggacttctccgacaagagccacggcacgcccaacctccacaccatgtacttcaagctgctgtttgacgagaacaatcatggccagatccgcctctcaggaggcgccgagctgttcatgcgccgcgccagcttgaagaaggaggagctggtggtgcaccccgccaacagccccatcgccaacaagaacccggacaaccccaagaagaccaccaccctcagctacgacgtctacaaggacaagcgcttctcagaggaccagtatgaactccacatccccatcgccatcaacaagtgccccaagaacatcttcaagatcaacaccgaggtgcgcgtgctactgaagcatgacgacaacccctacgtcatcggcatcgaccgcggcgagcgcaacctgctgtacatcgtggtggtggacggcaagggcaacatcgtggagcagtacagcttgaacgagatcatcaacaacttcaacggcatccgcatcaagacggactaccattcactgctggacaagaaggagaaggagcgctttgaggcgcggcagaactggacctccatcgagaacatcaaggagctgaaggccggctacatcagccaggtggtgcacaagatctgcgagctggtggagaagtacgacgccgtcatcgcgctggaggacctcaacagctactgctcttcagtgtaccagaagtttgagaagatgctgatcgacaagctgaactacatggtggacaagaagagcaacccctgcgccaccggcggcgcgctgaagggctaccagatcaccaacaagttcgagagcttcaagagcatgagcacccagaacggcttcatcttctacatccccgcctggctcaccagcaagattgatccctcaactggcttcgtcaacctgctgaagaccaagtacaccagcatcgccgacagcaagaagttcatcagcagcttcgaccgcatcatgtacgtgccggaggaggacctcttcgagttcgcgctggactacaagaacttctcaaggacggatgctgactacatcaagaagtggaagctctacagctacggcaaccggataaggatcttccgcaaccccaagaagaacaacgtctttgattgggaggaggtctgcctcacctccgcctacaaggagctgttcaacaagtacggcatcaactaccagcagggcgacatccgcgcgctgctctgcgagcaatcagacaaggccttctacagcagcttcatggcgctgatgagcctgatgctccagatgcgcaacagcatcaccggccgcactgatgtggacttcctcatctcgccggtgaagaacagcgacggcatcttctatgactcaaggaactacgaggcgcaggagaacgccatcctccccaagaacgctgacgccaacggcgcctacaacatcgcgcgcaaggtgctctgggccatcggccagttcaagaaggcggaggacgagaagctggacaaggtgaagatcgccatcagcaacaaggagtggctggagtacgctcagacctccgtgaagcactcaggcagcggcggcagcagcggcggcagcatggcggagaacggcgacaatgagaagatggcggcgctggaggccaagatctgccaccagattgaatactacttcggcgacttcaacctacctcgcgacaagttcctcaaggagcagatcaagctggatgagggctgggtgccgctggagatcatgatcaagttcaaccgcctcaaccgcctcaccaccgacttcaacgtcatcgtggaggcgctcagcaagagcaaggcggagctgatggagatctcagaggacaagaccaagatccgccgctcgccgagcaagccgctgccggaggtcaccgacgagtacaagaatgacgtcaagaaccgctcagtgtacatcaagggcttccccaccgacgccaccctggatgacatcaaggagtggctggaggacaagggccaggtgctgaacatccagatgcgccgcaccctccacaaggccttcaagggcagcatcttcgtcgtcttcgacagcatcgagtcagccaagaagttcgtggagacgccgggccagaagtacaaggagaccgacctgctgatcctcttcaaggacgactacttcgccaagaagaatgagagcggcggcagcaagcgcaccgccgacggcagcgagttcgagcccaagaagaagcgcaaggtgtga

SEQ ID NO: 284 - DNA, 4413 bp, synthetic construct
  source 1..4413: mol_type = other DNA
atgaaacgtacagccgacggctccgagtttgaacctaaaaagaagcgtaaagtaagcggtggttcgagcggaggatcctctaaactggaaaaattcacaaactgctatagcctaagcaagacactacggtttaaagcaattccggtcggcaagacacaagaaaatattgataataaaagattgttagtcgaggacgaaaaacgggctgaggattacaaaggagtgaaaaaacttttagacagatattatctgagttttattaatgacgtcctccattctattaagttaaaaaacttgaacaattatatttcattatttaggaaaaaaacaagaaccgaaaaagaaaacaaagaacttgaaaaccttgaaattaacctcagaaaagaaattgctaaagcatttaaaggtaacgagggctataagtcactgttcaaaaaagatatcatcgagacaatcctgccggagttcctggatgataaggacgaaatagcgcttgtgaatagcttcaatggatttacgacggcctttacgggtttttttagaaatagggaaaacatgtttagtgaagaagcaaaatcaacatccattgcttttcgctgtataaacgagaacctgacacgttatatcagcaacatggatatctttgagaaagtggatgctatctttgacaaacatgaggtgcaagaaataaaagaaaaaattttgaatagtgattatgatgtcgaagatttttttgagggcgaattttttaactttgttctgacacaagagggtattgatgtttacaatgccattattggaggattcgtgactgagtccggagaaaagatcaaaggcttaaatgaatatattaacttgtataaccagaagacgaaacaaaaacttcccaaattcaaaccgctttacaaacaagttctgagtgaccgggaatctctctccttttatggcgaaggctacacatcggatgaagaggtactggaagtttttcgtaatactctgaataaaaattccgaaatcttctcatccattaagaagttggaaaaattatttaagaacttcgacgagtacagttctgcaggaatatttgtgaaaaatggccctgctatatcgacaatcagcaaagacatctttggcgagtggaatgtgatccgtgataaatggaatgcggaatatgacgatattcatcttaagaaaaaagctgtagtaacagaaaagtacgaggatgaccgccgcaagtctttcaaaaaaatcggtagtttcagcctggaacagcttcaggaatatgcggatgcagatttgtcagtagtcgaaaaacttaaagaaataatcatccaaaaggtggatgaaatttataaagtttatggttcttcagagaaactgtttgacgcagactttgtcctcgagaaatcactgaaaaaaaatgatgcggtcgtagcaattatgaaagatttgctcgactcagtaaagtcatttgaaaattatattaaagccttctttggcgaaggcaaagagacgaatagagatgagtcattttacggagatttcgttctggcctatgacattcttcttaaggtagatcacatctatgatgctattaggaactatgtcacgcaaaaaccttattctaaagataaattcaagctgtattttcagaatccacagttcatgggtggctgggacaaagacaaagaaacggattatcgcgcaactattttacgatacggatctaagtactacttagcaattatggataaaaaatatgcgaagtgcttacagaagattgacaaagacgacgtcaatggaaattatgaaaaaatcaactataaactccttcctggacctaataaaatgttgcctaaggtattcttttcaaagaaatggatggcttactacaatccttcggaggatattcagaagatttacaagaatggaacattcaagaaaggcgatatgttcaatttgaacgattgccataagctgattgatttctttaaagattcgatttctagatatccgaaatggagcaatgcttacgattttaatttctccgaaacagaaaaatataaggacattgcaggattttatagggaagttgaagagcaaggatataaagtcagctttgaatcagcaagcaaaaaggaggtggataaacttgttgaagaaggaaaattgtacatgttccaaatttataacaaagatttctcagataagtcccacggcacgccgaacctccataccatgtattttaaattgctctttgatgaaaataatcacggacagatccgtttgtccggcggcgcggagctcttcatgagacgggcgtcactgaagaaagaagaacttgttgttcaccctgcgaactcaccgattgcgaataaaaacccagataacccaaaaaaaacaactacacttagctatgatgtatataaggataagagattctctgaagatcagtatgagttacacatcccgatcgctattaacaaatgtccaaaaaatatcttcaaaatcaatactgaggttagagttcttcttaagcatgatgacaacccgtatgtcattggcattgatcgtggcgaacgcaatttattatacatcgtggtggtggacggcaagggcaatattgtcgaacaatattcactgaatgagattatcaataattttaacggaatacggatcaaaactgattaccattccttactagacaaaaaagaaaaagaacgattcgaagctcgccagaattggactagtattgaaaatataaaggaattgaaagcaggttatatatcgcaagtagtccataaaatttgtgaactcgttgaaaaatacgatgcagtaatcgccctagaagatcttaattcatattgctcaagtgtttatcaaaaatttgaaaagatgcttattgacaaattaaattatatggtcgacaaaaagagcaatccgtgcgccaccggtggcgcgcttaaaggctaccagattacaaataaatttgaatcatttaaatctatgtcgacccaaaacggctttattttttacattccagcctggcttaccagtaagattgatccgagcacgggctttgtcaacttgctgaaaacaaagtacacttcgattgcagattctaaaaaatttatctcgagctttgataggattatgtatgtcccggaggaggacttgttcgaatttgccttagactacaaaaacttttctagaacggacgccgattatatcaagaaatggaagttgtactcatacggtaaccgcatacggatatttcgcaatccaaagaaaaataacgtatttgattgggaagaagtctgtctcacaagcgcatacaaagagctttttaataaatacggaattaattatcagcagggtgacatccgggctctgttgtgtgagcaatccgataaggcgttttattcgtcctttatggccctgatgagcttaatgcttcaaatgcgcaattcaattaccggacgaaccgatgtggatttccttatctctccggtgaaaaattctgatggtattttttatgactcaagaaactacgaagctcaagaaaacgctattttaccgaaaaacgccgatgctaacggcgcttataacatcgcacgcaaagtgctgtgggccataggccagtttaaaaaagcggaagacgaaaaattagataaagtaaaaatcgcgatatctaacaaggagtggcttgagtatgcgcaaacgtctgttaagcatagcggcagcggaggctctagtggcggaagcatggcggagaatggcgataatgaaaagatggcggctctcgaagcaaaaatctgccatcagatcgaatattatttcggcgactttaacctaccccgtgacaaatttttaaaagagcagattaagttagatgaaggctgggtgccgctggagattatgatcaaatttaaccgccttaacagattaacaacggattttaacgttatcgtcgaagcattgtcaaaatctaaggctgaactgatggagattagcgaagataaaacgaaaattcggagaagcccgagcaaaccactgcccgaagttacagatgaatataagaacgatgttaaaaatcggtctgtctatatcaaaggcttccctacggatgccacgttagacgacattaaagaatggctggaagataaaggccaggtgctcaacatacaaatgcgacgaaccctgcataaggcgtttaagggctccatcttcgttgtgtttgattctatcgaaagcgcgaaaaaatttgtggaaacacctggacaaaaatacaaagaaaccgacctattaatcctttttaaagatgattattttgcaaaaaaaaatgagtcgggtggatcaaaacgtacagcagacggctctgaatttgagccgaagaagaaacgtaaggtttaa

SEQ ID NO: 285 - DNA, 4413 bp, synthetic construct
  source 1..4413: mol_type = other DNA
atgaagcgcaccgcggacggctccgagtttgagccgaagaagaagcgcaaggtgtccgggggcagcagcgggggctcgtccaagctcgagaagttcaccaactgctactccctctcgaagaccctcagattcaaggcgattcctgtgggtaagacccaggagaacatcgacaacaagcgcctgctcgtcgaggacgagaagcgcgccgaggactacaagggtgttaagaaactgctcgaccgttactacctctcgttcatcaacgatgtcctgcacagcatcaagctcaagaacctgaacaactatatcagtctgttccgtaagaaaacccggacggagaaggagaacaaggaactggagaacctggagatcaacctgcggaaggagatcgccaaggcgttcaagggaaacgaaggctacaagtcgctcttcaagaaggacatcatcgagaccatcctcccggagttcctggacgacaaggatgagatcgccctcgtcaactcgttcaacggcttcaccaccgccttcaccggtttcttccgcaaccgcgaaaacatgttctcggaggaagcgaagtccacgtcgatcgcattccgctgcatcaacgagaacctcacccgctacatctcgaacatggacatcttcgagaaggtggacgcgatcttcgacaagcacgaggtgcaggagatcaaggagaagatcctcaacagcgactacgacgtggaagacttcttcgagggcgaattcttcaatttcgtcctcacccaggaaggcatcgatgtgtacaatgcgatcatcggcggattcgtgacggagtccggagagaagatcaagggcctcaacgagtacatcaacctgtacaaccaaaagaccaagcagaagttgccgaaattcaagcccctctacaagcaagtcctctccgaccgggaatcgctgtcgttctacggtgagggatacacgtcagacgaagaggtgctcgaggtcttccgcaacaccctgaacaagaactcggaaatcttcagctcgatcaagaagctcgagaagctgttcaagaacttcgacgagtactcctcagccggcatcttcgtcaagaacggtccggcaatcagcacgatcagcaaagatatcttcggagaatggaacgttatccgggacaaatggaacgcggagtacgacgacatccatctgaagaagaaagccgtggtcaccgagaagtacgaagacgaccggcggaagtccttcaagaagatcggcagtttctccctggaacagctccaggagtacgccgacgccgatctgtcggtggtcgagaagctgaaagagatcatcatccagaaggtcgacgaaatctataaggtctacggttcgagcgagaagctgttcgatgcggacttcgtgctggaaaagtccctgaagaagaatgacgcggtcgtcgcgatcatgaaggacctcctggactcggtcaagtccttcgagaactacatcaaggccttcttcggcgagggcaaggagacgaatcgcgacgaaagcttctacggggacttcgtgctggcctacgacatcctcctgaaggtggaccacatctacgacgcgatacggaattacgtcacgcagaagccctactccaaggacaagttcaagctctacttccagaacccccagttcatgggcgggtgggacaaggataaggaaacggactaccgagcgacgatcctgcgctacggctcgaagtactacctcgccatcatggacaagaagtacgcaaagtgccttcagaagatcgacaaggacgatgtgaacgggaattatgagaagatcaactataagttgctgccggggcccaacaagatgttgccgaaggtgttcttcagtaagaagtggatggcgtactacaacccgtcggaggacatccagaagatctacaagaacggtaccttcaagaagggtgacatgttcaacctgaacgactgccacaagctcatcgacttcttcaaggactccatctcgcgctaccccaagtggagcaacgcgtacgacttcaacttcagcgaaacggaaaagtacaaggacatcgcagggttctaccgtgaggtcgaggagcagggatacaaggtcagtttcgaatcggccagcaagaaagaagtcgacaagctcgtcgaagaaggtaagctctacatgttccagatctacaacaaggacttctccgacaaatcccacgggacgccgaacctgcacaccatgtatttcaagctcctgttcgatgagaacaaccacggccagatccggctgagcggcggcgccgagctgttcatgcgccgcgcctcgctcaagaaggaggagctggtcgtccacccggccaactctcccatcgcgaacaagaacccggacaatccgaagaagaccaccacgctgagctacgacgtctacaaggacaagcgcttctccgaggaccagtacgagctccacatccccatcgcgatcaacaagtgtcccaagaacatcttcaagatcaacacggaggtccgcgtcttgctcaagcacgacgataacccctacgtcatcggcatcgatcgaggcgaacgaaacctcctctacatcgtggtcgtcgacggtaagggcaacatcgtggaacagtactccttgaacgagatcatcaacaatttcaacggtatccgcattaagaccgattaccacagcctcctcgacaagaaggaaaaggagcgcttcgaggctcgccagaactggacctcgatcgagaacatcaaagagctgaaggccggttacatctcccaggtagtgcataagatctgcgaactggtggaaaagtacgacgccgtcatcgccctggaggacctcaactcgtattgctcctcggtgtaccagaagttcgagaagatgctcatcgacaagctgaactacatggtcgacaagaagtccaacccatgcgccacgggcggcgccctcaagggctaccaaatcaccaacaagttcgagtcgttcaagtccatgtccacgcagaacggtttcatcttctatatcccggcttggctgacctcgaagatcgacccgagcacgggcttcgtcaacctcctgaagaccaagtacacctccatcgcggattcgaagaagttcatctcgtcgttcgaccggatcatgtatgtgccggaggaggacctgttcgagttcgccctcgactacaagaacttctcgcggacagacgctgactacatcaagaaatggaagctctacagctacggcaaccgtatccgcatcttccgtaacccgaagaagaacaacgtgttcgactgggaggaggtctgcctgacctcggcgtacaaggagctcttcaacaagtacggtatcaattaccagcagggcgacatccgcgcgctgctctgcgagcagtccgacaaggcgttctacagcagcttcatggcgctcatgtccctgatgctccagatgcgtaactccattaccgggcgcaccgatgtcgacttcctcatcagcccggtcaagaactccgacggtatcttctatgactcgcgcaactacgaagcgcaggagaatgccatcctccccaagaacgccgacgcgaacggcgcctacaacatcgcccggaaggtcctctgggccatcggccagttcaaaaaggcagaggatgaaaagcttgacaaggtcaagatcgcgatctccaacaaggagtggctggagtatgctcagacctccgtgaagcacagcggctcgggcggcagctcgggaggctccatggccgaaaatggcgacaacgaaaagatggccgccctggaggcgaagatctgccaccagatcgagtactacttcggagacttcaacctgccgcgggacaaattcctcaaggagcagatcaagctcgacgagggctgggtgcccttggaaatcatgatcaagttcaaccgtctcaaccggctgaccacggatttcaacgtcatcgtcgaagccctgtcgaagtcgaaggcagagttgatggagatctcggaggacaagacaaagatccgccggtcgccctcgaaaccgctcccggaggtgacagatgaatacaagaacgatgtgaagaatcgctcggtgtacatcaagggattccccaccgacgccaccctggacgacatcaaggagtggctcgaggacaaagggcaggtcctcaacatccagatgcgccgaaccctccataaggcgttcaaaggttcgatcttcgtcgtcttcgactcgatcgagtcggccaaaaagttcgtcgagaccccggggcagaagtataaggagaccgacctgctcatcctcttcaaggacgactactttgcgaagaagaacgagtcgggaggctccaagcggaccgccgatggctcggagttcgagcccaagaagaagaggaaggtgtga

SEQ ID NO: 286 - DNA, 4413 bp, synthetic construct
  source 1..4413: mol_type = other DNA
atgaagcgaactgctgacggctctgagttcgagcccaagaagaagcgcaaggtctcgggagggtcctccggcggatcttcaaaactggaaaagtttacaaactgttactccctaagtaaaaccctgcgatttaaggccattcccgtcggcaagacccaggaaaatatcgataacaagagactgctggtggaggacgaaaaacgagccgaggactacaagggtgtgaaaaagttacttgaccggtactacctctctttcattaacgatgttctgcattccataaagctcaagaacctcaataactacatttcgctttttaggaagaagactcgcacggaaaaggaaaacaaggaactcgagaacctggagattaacctccggaaggaaatcgctaaggctttcaagggcaatgagggttataagtcgctctttaagaaggatatcattgaaacgattctccccgagttcttggatgataaggacgagatcgccctcgttaactcgtttaatggttttaccaccgcatttacaggtttcttccggaatcgagagaacatgttttctgaggaggctaagtccacgtctatcgcatttcgatgtatcaacgagaatctgactcgatatatttctaatatggacatctttgagaaggtcgacgccatctttgataaacacgaggtacaggagattaaggagaagatcctcaacagtgactacgacgtggaggatttcttcgagggggagttttttaacttcgtgctgacacaagagggcattgacgtgtacaacgcgatcatcggcggtttcgtgactgagagcggagagaagattaagggccttaacgaatatattaacttgtacaaccagaagaccaagcagaagctgcctaagttcaagccattgtacaagcaggtgctctccgaccgagagtctctgagtttctacggtgagggctacacctccgacgaagaggttctcgaagttttccgtaacactcttaacaagaactctgaaatcttctcgtcaattaagaagctcgagaagctgttcaagaatttcgatgagtactccagcgccggaattttcgttaaaaacggacctgcgatttctaccatctccaaggatattttcggtgagtggaacgtaattcgcgacaagtggaacgcggagtacgatgacatccacctcaagaaaaaggccgtcgtcaccgagaagtacgaagatgatcgacggaaatcttttaagaagatcggatctttctcgctggagcaactccaagagtatgccgatgcagacctgagtgtggtggagaagctcaaggagattattatccaaaaggtggacgaaatctataaggtttacggaagctctgagaagcttttcgacgccgactttgtccttgaaaagtccctgaagaagaacgacgccgtggtggcaatcatgaaggacctgttggactccgtaaaatcctttgagaactacatcaaagcattttttggcgagggtaaagagactaatcgggatgagtccttctacggagatttcgtcctggcctacgacatcctcctgaaggtggaccatatctatgacgccattcgtaactacgtcacgcagaaaccctactccaaggataagtttaagctgtacttccagaaccctcagttcatgggtggatgggacaaggacaaagagacagattaccgagctactatcctcagatacggctcgaagtactacctggccataatggacaagaagtacgccaagtgtcttcagaagattgacaaagacgacgttaatggcaactatgaaaaaattaattacaaattgctgcccggccctaacaaaatgctgccaaaagtgttcttctctaaaaagtggatggcctactataatccatcggaggacattcagaagatttacaagaatggcacatttaagaagggtgatatgtttaacctcaacgactgccacaagcttatcgatttcttcaaggattcgatctcacgttaccctaaatggtcgaatgcctacgattttaacttttcagagacggagaaatacaaggacattgccggcttttaccgagaggtcgaagagcagggttataaagtcagctttgagtccgcgtctaagaaggaagtcgacaagctagtcgaggaaggaaaactgtatatgtttcagatatacaacaaggatttctctgacaagtctcatgggactcctaaccttcacactatgtatttcaagctgctcttcgatgagaacaaccatgggcagattcgtctcagcggtggcgccgaactgttcatgcgacgtgcttcgctgaagaaggaggagctcgtcgttcacccggctaactcgccgatcgctaacaagaaccctgacaatcctaaaaaaacgaccaccctgtcctacgatgtttacaaggacaagcgattttcagaggaccagtatgaactgcacattcccatcgctatcaacaagtgccctaaaaacattttcaagatcaatacagaggtgcgggtcttgctgaagcatgacgacaatccttacgtcatcggaatcgaccggggagagagaaatctgctatacatagtggttgttgacggcaagggaaacatcgtagagcagtacagcctgaacgagattattaacaactttaacggaattcgaatcaaaaccgactaccactccttgctagataagaaggagaaggagcgatttgaggccagacaaaactggacttctattgagaacatcaaagagctgaaggccggctacatttcccaggttgtccacaagatctgcgagctagtggagaagtatgatgcggtcatcgcgctcgaggatcttaactcgtattgctcctccgtctatcagaagtttgagaagatgctcattgacaagctgaactacatggttgacaaaaagtccaatccctgtgctactggcggcgctttgaaaggctaccaaatcactaacaagttcgagtcatttaagtctatgagtacacagaacggtttcatcttttacatcccggcctggcttaccagcaagattgacccaagcacaggatttgtgaatttgctcaagaccaagtacacgtcgattgctgactcgaagaagttcattagcagcttcgatagaattatgtacgtccccgaagaggacctcttcgaatttgcacttgactacaaaaacttctctagaaccgacgctgactacatcaagaaatggaagctctactcttatggcaaccgaattcggatttttaggaacccaaagaaaaacaacgtttttgactgggaggaagtgtgcctgacttcagcatacaaggagcttttcaacaagtacggcattaactaccagcagggtgacatccgagctctgctgtgcgagcagtcggataaggctttctactcctccttcatggctctcatgagtctaatgcttcaaatgcgaaactccattaccggaagaaccgacgtggatttcctcatctcccccgttaagaactctgacggtatcttctacgattctcgcaactacgaggcccaggaaaacgccattctgcccaagaatgccgacgctaacggagcctacaacatcgctcgaaaagtgctgtgggccatcggacagtttaagaaagcagaggacgagaagctggataaagtgaaaatcgctatcagcaacaaagagtggctggagtacgcacagacctctgtgaagcactctggctcaggaggctcaagtgggggatcgatggctgaaaacggagacaacgagaagatggccgctttggaagcgaagatttgtcaccagatcgaatactactttggagatttcaacctgcccagagacaagttccttaaggaacagatcaagttggatgagggttgggtccccctcgagattatgattaagttcaaccgcttgaaccgactgacaacggacttcaacgtgatcgtggaggctttgagcaagtctaaggccgaactgatggagatctcggaagataagaccaagattcgacgatccccctctaagcccctgcctgaagtcaccgacgaatacaagaacgacgtcaagaacagaagcgtgtacattaagggtttccctactgacgccaccttggacgacattaaggaatggcttgaggataagggtcaagttcttaacatccagatgcgaaggaccctccataaggccttcaagggttcgatcttcgtcgtgttcgattctattgaatcggctaagaaattcgttgagactcccggccagaagtataaggagaccgacctactgatcttattcaaggatgactatttcgcgaagaagaacgagtctggcggatctaagcgtaccgcagacggaagtgagtttgagccgaagaagaagcgaaaggtgtaa

SEQ ID NO: 287 - DNA, 4413 bp, synthetic construct
  source 1..4413: mol_type = other DNA
atgaaacgcacggccgacggctcggagtttgagccgaagaaaaagcgcaaagtcagtggcggtagctcaggtggctcttcaaagctggagaaatttacaaactgttattcactttctaaaacgctgcgtttcaaagcaatccctgtggggaagactcaggaaaatattgacaacaaacggttactggttgaagacgaaaagcgtgcggaagattacaaaggcgtgaaaaaactgctggatcgctattatttaagcttcatcaacgacgtcttacactccattaaactgaaaaacttaaacaactatatctcactgttccgcaaaaaaacccgcacggagaaagaaaataaagaattagaaaacctcgaaattaacttacgcaaagagattgcgaaagcattcaaaggaaacgaaggttataaaagtctgttcaaaaaggacatcatcgagactattctaccggagttcctagacgacaaagatgagattgctctggttaattcgttcaacggctttacaacagcgtttacgggctttttccggaatcgtgaaaatatgttcagcgaagaggcaaaaagtaccagcattgcctttcgttgtatcaacgaaaatctcacgcgttatatctctaacatggatatatttgaaaaagttgacgctattttcgacaaacacgaagtgcaagaaataaaagaaaaaattctgaacagcgactatgatgtagaagatttcttcgaaggcgagttttttaattttgtgctgacacaggaaggaatcgatgtctataatgccatcatcggaggcttcgtgaccgagtccggcgaaaaaatcaaaggcttgaacgaatatattaatctctataaccaaaaaaccaaacaaaaactgccgaaatttaaacccctgtacaaacaagtgctttccgatcgcgaatccctgagcttctatggtgaagggtacacaagtgacgaagaagtattagaggtattccgtaataccctaaataagaacagcgaaattttctctagtattaaaaaactggaaaaattatttaaaaactttgatgagtactcttcggcgggtattttcgtgaaaaacggcccggcgatttcgactatttcaaaagatatttttggcgagtggaatgtgatccgtgataagtggaacgcggagtacgatgatattcatctgaaaaagaaagccgttgttacggaaaaatacgaagatgatcggcgcaaatcttttaaaaaaatcggctcattttccctggaacagctgcaggaatatgccgatgcggatctgtcggtggtggaaaaactgaaggaaataattatccagaaagttgatgaaatttataaggtctacggctcttctgaaaaactgttcgacgccgattttgtcttggaaaaaagcctcaaaaaaaatgatgccgtagtcgcgattatgaaagatttgcttgatagtgtgaaaagcttcgaaaattacattaaagcgttttttggtgaaggcaaagagaccaatcgtgatgaaagcttttacggtgactttgtcctggcctatgacatacttttgaaagttgatcatatttacgatgcgattcgcaactacgtcacgcaaaaaccatattccaaagataaatttaaattgtactttcaaaatccgcagtttatggggggttgggataaagataaagaaaccgactaccgtgccaccattctgcgctatggcagtaaatattacttggcgatcatggataagaaatatgccaaatgcctgcagaaaatcgataaagacgatgtgaacggtaactatgaaaaaatcaactacaaactgttaccgggccctaacaaaatgcttccaaaagtgtttttttcgaaaaaatggatggcttactataatccgagtgaagatatccaaaagatctataaaaacggtacgtttaagaaaggggatatgttcaatcttaacgactgtcacaagctgatcgatttcttcaaggactccatttctcgttaccccaaatggtctaacgcgtacgatttcaatttttcggaaacggaaaaatataaagatattgccggcttttatcgtgaagtcgaggagcaaggttataaagttagtttcgaaagcgcctcaaaaaaggaagtggataagctggtggaagaaggaaagctgtacatgttccagatttacaataaagatttttccgacaagagccatggcactcccaatttacataccatgtattttaaactgctgttcgatgagaacaaccatggtcagattcgtctcagtggaggcgcggagctattcatgcgccgtgcctccctgaaaaaagaggagctggttgtgcacccggcgaatagtccgattgcaaacaaaaacccggacaacccgaagaaaaccaccaccctgagttatgacgtttataaggacaaacgctttagcgaagaccagtacgaactgcacatcccgatagcaattaacaagtgcccgaaaaatatttttaaaattaacaccgaagtgcgtgttctgctgaagcatgacgataacccttacgtgatcggtattgatcgcggcgaacgcaatctgctgtacattgtggtcgtggatggtaaaggtaatatcgttgaacaatacagcttgaatgaaattattaataacttcaacggcattcgcattaaaactgattatcactcccttctcgacaaaaaagaaaaagaacgcttcgaggcgcgacaaaactggacgtctatcgaaaatattaaagaactgaaagcgggttacatctcccaggtagtgcataaaatctgtgaacttgtggaaaaatatgatgctgttattgctcttgaagatctgaatagctactgctcgtcagtgtatcagaagttcgaaaaaatgttgattgataaattgaactatatggtcgataaaaaatccaatccgtgcgcgaccggaggggccctgaaaggttatcagatcacgaacaaatttgagagttttaaatcgatgagcactcaaaatggttttatcttctatatcccagcatggctgacctcaaagattgacccctctaccggttttgttaacctgctgaaaacgaagtatacctcaattgctgacagcaagaaatttatttcatcgttcgacagaattatgtatgtccctgaagaagatctgttcgaatttgccctggattataagaatttctccaggaccgatgcggactatatcaaaaagtggaaattgtattcctatggtaaccggatccgcatttttcgtaatcctaaaaaaaacaatgttttcgactgggaagaggtatgtctgacgtctgcatacaaggaattatttaataaatatgggatcaattatcaacagggtgatattagagccttactgtgcgagcagagtgataaggcgttttatagctcgtttatggcgctgatgagcttgatgttgcagatgcgaaacagcattacgggccgcaccgatgtggattttctgatttcgccggtcaaaaattccgacggtatcttttacgattcgcgcaattatgaagcccaggagaacgcaattttgccgaagaacgcagacgcgaatggcgcatacaacatagctcgtaaagtcctctgggcgatcggacagttcaagaaagccgaggacgaaaaactggataaagtcaaaattgcaatcagcaataaagagtggttagaatatgcacagacatctgtcaagcatagcggaagtggcgggagcagcggcgggagcatggctgagaatggtgacaatgagaagatggccgcacttgaggcgaaaatatgccaccagatcgagtactactttggtgactttaatctgccgcgcgataaatttctgaaagaacagatcaaattagatgaaggctgggtgccgctcgaaatcatgatcaaatttaaccgccttaaccgtttgaccactgattttaacgttattgttgaagctttgagcaaatcaaaagcagagttaatggagatctctgaagacaaaaccaagatccggcgctctccatccaagccactgccagaggtaacagacgaatacaaaaatgatgtaaaaaaccgttcggtatacatcaaagggttcccaactgatgcgactctggacgatatcaaggagtggctggaagataaaggccaggtgctcaacattcagatgcgacgtaccctccataaagcttttaaaggctcgatcttcgtggtttttgatagcatcgaatcggctaaaaaatttgtagaaacccctggccaaaagtataaagaaacggatctactcattctgttcaaggatgattacttcgcaaagaaaaacgaaagcgggggtagcaaacgtaccgcggacgggagtgagtttgaaccgaaaaaaaaacgtaaggtttaa

SEQ ID NO: 288 - DNA, 4413 bp, synthetic construct
  source 1..4413: mol_type = other DNA
atgaaacggaccgcggatggtagcgaatttgagcctaagaagaagcgcaaggtcagcggaggttcgagtggcggctcctctaagctggagaagttcactaactgctactccctgtctaagaccctgcgtttcaaggcgatcccagtcgggaagactcaagaaaatatcgataacaagcgcctgctcgttgaggacgaaaagcgcgccgaggactataagggtgtcaaaaaacttcttgacaggtactacttgtctttcatcaacgacgtcctccactcgatcaagctgaagaatctcaacaattacatttctctcttccgtaagaagacccgaaccgaaaaggagaacaaggagctggaaaaccttgagatcaacctgcgcaaggagatcgctaaggcgttcaagggtaacgagggttacaagtcactgtttaagaaggacattatcgagaccattctccctgaatttttggacgataaggacgagatcgccctagtgaattcgttcaacggctttaccacggcctttacaggtttcttccggaaccgcgaaaatatgttcagcgaggaagctaagagcacctccatcgcttttcgttgcatcaacgagaatctcactcgctacatctccaacatggacatcttcgagaaggttgacgctattttcgataagcatgaggtccaagaaatcaaggagaagatcctcaatagcgactacgacgtcgaagatttcttcgagggtgagttcttcaactttgtcctcacgcaggagggcatcgacgtgtacaacgctatcatcggcggcttcgtcactgagtccggagaaaaaattaagggactcaatgaatacattaacctgtataatcagaagaccaagcagaagcttccaaaatttaagcccctgtacaagcaggtcttgtcggatagagaaagtctcagtttctacggcgagggctacacgtcagacgaagaagtgctcgaggtattccgaaacaccctgaataaaaactctgagattttcagcagcattaagaagctcgagaagttgtttaagaacttcgatgagtattcgtcggcggggatcttcgtcaagaacggccccgccatctcaacgatttcgaaggatatctttggtgagtggaacgtgatccgcgataagtggaacgccgaatacgatgatatacacctcaagaagaaggccgtggttaccgagaagtacgaggacgatcggcggaaatctttcaagaagatcggctcgttctcccttgagcagctgcaagaatacgccgacgccgatctatccgttgtcgagaaacttaaggagattataattcagaaggtcgacgaaatctacaaggtgtacgggagttccgagaagctcttcgacgcagatttcgtcctggagaagagtctgaagaaaaacgatgcagttgtcgcgatcatgaaagatctcttggactcagtcaagtccttcgagaattacatcaaggccttcttcggtgagggtaaggaaactaatcgcgacgaatccttctatggtgacttcgtcctggcctacgacatcctactgaaagtcgatcacatctatgacgctatcaggaattacgtcacccagaagccctactccaaggataagttcaagctctattttcagaacccgcagttcatgggcggctgggataaggacaaagagaccgactatcgcgccaccatcttgcgctacggaagcaagtactatttagctattatggataagaagtacgccaaatgcctacagaagattgataaggacgatgtcaatggtaactacgagaagattaactataaactcttgccgggccccaacaagatgttgcccaaggtctttttctccaagaagtggatggcatactataatccgtccgaagacattcagaagatctacaagaatggtacattcaagaagggcgatatgttcaacttgaacgattgccacaagctgatcgacttcttcaaggactcgatttcccgttatcccaagtggtccaatgcgtacgactttaacttctccgaaacggagaagtataaggatatcgcaggcttctatcgggaagtggaggaacaaggctacaaggtcagctttgaaagcgcctctaagaaggaggttgataagctggtcgaggagggtaagctctacatgttccagatctataacaaggatttttccgacaagtcccacggaaccccaaacctgcacacaatgtactttaagttgctttttgatgagaacaatcatggacaaatccggctatcaggcggtgccgagcttttcatgcgacgtgcgagcctcaaaaaggaggagcttgtagtccaccccgctaactctcccatcgccaacaaaaatccggacaaccctaaaaaaaccaccacgttgagctatgatgtgtacaaggacaagaggttctccgaagaccaatacgaactgcatatccccatcgccattaacaaatgtccgaagaacatcttcaagatcaatactgaggttcgcgtcctcctcaagcacgatgacaacccctacgtgatcggcattgaccgtggggagcgcaaccttttatacatcgttgtggttgacggaaaaggtaacatagttgagcagtactccctgaatgagatcattaacaacttcaacggtatccgtatcaagaccgactaccatagcctcttggataagaaggagaaggagagattcgaggcccggcagaactggacatctatcgagaacatcaaggaactaaaagcaggatacatctcccaggtcgtccataagatctgtgaacttgtcgagaagtacgacgccgtcattgcccttgaggacctcaactcctactgctcgagcgtctaccaaaagttcgagaagatgctcatcgataagctgaactacatggttgacaaaaagtcgaacccatgcgcgacggggggcgcgctgaagggatatcaaatcaccaacaaattcgagagtttcaagtccatgtctacccagaatggcttcatcttctacatccctgcgtggctcacgagtaagatcgaccctagtacgggtttcgtgaacttgctgaagacaaagtacacttccatcgctgactccaaaaagtttatctcgtctttcgaccgcatcatgtatgttccggaggaggacctgttcgagttcgctctcgactacaagaacttcagtcgcactgacgctgattatattaagaaatggaagctgtacagctacggtaaccgaattcgtattttcagaaaccccaagaagaataacgttttcgattgggaggaagtgtgtttgacctctgcctacaaggagctcttcaacaaatacggcattaactatcagcagggcgatatccgcgccctgctgtgtgagcagagcgacaaagccttctattcttctttcatggctctgatgagcctgatgctgcagatgcgtaactctattaccgggcgcactgacgtggactttttgatttctccagtgaagaactctgacggtattttctacgatagccgtaactacgaagctcaggagaacgcaatcctgcccaagaacgctgatgccaacggggcctataacattgctcgcaaggttctctgggccatcggacagttcaagaaggccgaagatgagaagctcgataaggtgaagatagctatctccaacaaggaatggttggagtacgctcagacttccgttaagcactcgggctcgggcggatcctcgggtggatccatggcggagaacggcgataacgaaaagatggccgcgctcgaggccaagatctgccatcagatcgagtactactttggcgatttcaaccttcctcgggataagtttttgaaagaacaaatcaagctcgacgagggatgggttcccctcgaaattatgatcaagttcaaccggctgaaccgtctgaccaccgacttcaatgtcattgttgaggcacttagcaagagcaaagctgagctgatggagatcagcgaggacaagaccaagatacgtcgttcccctagcaagcctctgcccgaggtgacagatgagtacaagaacgacgtgaagaacagatctgtctacattaagggcttcccgactgatgctaccctggacgacatcaaggaatggttggaggataagggtcaagtgcttaacatccagatgcgccgcactcttcacaaagcattcaagggatccatctttgtggtcttcgacagtattgagagtgccaagaagttcgtggagacgccgggccagaagtataaggagaccgacctcctgatccttttcaaggacgactactttgctaagaaaaacgagtccggtggttcgaaacgaaccgcagatggctcggaattcgagcctaagaagaagcgtaaggtataa

SEQ ID NO: 289 - DNA, 4413 bp, synthetic construct
  source 1..4413: mol_type = other DNA
atgaaaaggacggcagatggttcagaattcgaacctaaaaagaagaggaaagtgtcgggcggatcatctgggggttccagcaagttagagaagtttactaactgttactctttaagcaagacccttcgattcaaagccattccggtagggaagactcaggagaatatcgataacaagagacttcttgttgaggatgaaaagagagctgaagattacaaaggtgtgaagaaattgcttgacagatattacctgagttttattaatgatgtcctgcactccattaaattgaaaaacctaaataattatatctcgttatttagaaaaaaaaccagaacagaaaaggaaaataaagaattagagaatcttgaaataaatctgcgtaaagagatagctaaggcgtttaagggaaatgagggttataaaagcctattcaaaaaggatataatcgagacaatcctaccagaattcctcgacgataaggatgagattgcgttagtgaattctttcaatggtttcacaacagcatttacaggattttttagaaatagggagaatatgttttcggaagaagcaaaaagtacttctatcgcctttaggtgcatcaatgagaatttgacgagatatattagtaacatggatattttcgaaaaagttgacgccatatttgataagcatgaagtgcaagaaataaaagaaaagattttgaattcagattacgatgtcgaagatttctttgagggagagtttttcaacttcgtacttactcaagaaggaatcgatgtttataatgctataatagggggttttgttacagaatccggtgaaaaaataaaaggactaaacgaatacataaacttatataaccaaaaaaccaaacaaaagttgccaaaatttaagcccctatataaacaggtgctctccgaccgggaatctttatcattctatggcgaaggttacacctcagacgaggaggtccttgaagttttcaggaataccctaaacaagaacagtgaaatttttagtagtatcaaaaagctggaaaaattgtttaaaaattttgacgaatactcttccgcgggcatttttgtgaaaaatggtcccgcaatctcgaccataagcaaagatatttttggtgagtggaacgttatccgggataaatggaacgcggaatatgatgatatacacctcaagaaaaaagcagtagtaacagaaaaatatgaggatgaccgcaggaaaagctttaagaaaatcggctcattttctttggagcaactccaagaatacgcggacgccgacttgagcgttgtagaaaaattgaaggagataattattcaaaaggttgatgaaatatataaagtctatgggagctctgaaaaacttttcgatgctgattttgttttggaaaaaagcttgaaaaagaatgacgcagtggtagcaataatgaaggatctccttgatagtgtaaaatcgttcgaaaattatatcaaggcattttttggagaaggcaaagaaaccaaccgagacgagtccttttacggggattttgtgttagcctacgatatccttttgaaggtagaccatatctacgatgccattcgtaactatgtaacacaaaagccttactcaaaggataaatttaagctctactttcaaaatccacaatttatgggaggctgggataaagataaagagacagattatcgcgccacaatcctaaggtatggttctaaatattacttggcaatcatggataaaaaatatgctaagtgtctacaaaaaatcgataaagacgatgtcaacgggaactacgaaaagataaactacaaattacttccaggcccgaacaaaatgttgccaaaggtattcttttccaaaaaatggatggcatattataatcctagtgaggatattcagaaaatctataaaaatggaacatttaaaaaaggagatatgtttaacttaaatgattgtcataaattgatagatttttttaaggattccatttcgagataccctaaatggtctaacgcctatgatttcaacttttctgagactgaaaaatacaaagacatagcagggttctacagggaggtggaagaacaaggatataaagtatcgttcgaaagcgcgtcaaaaaaagaagtggataaattagttgaggaaggaaaactgtatatgtttcagatatacaacaaagatttttcagataagtcccatggaacacccaatcttcatacaatgtatttcaagttgctctttgatgagaataatcatggtcagattcgtttatctggtggagcagaattatttatgcgaagagcctcgttgaagaaagaggaactagtagtccacccagcaaatagtccgattgcaaataaaaacccggataatccaaaaaagacaaccacgctaagttatgatgtgtacaaagataaaaggttctccgaggatcaatacgagctgcacattccaattgctataaataagtgtccgaaaaacatatttaagataaatacagaggtgagagttctcttgaagcacgacgataatccctatgtgatcggtatcgaccggggggaacgtaatctgttatacattgtcgttgtagatggcaaaggaaatatagttgagcaatacagtttaaatgagattatcaataattttaatggcattagaataaaaacggactatcactctctactagataaaaaagaaaaggaaaggtttgaggcccgacagaactggacgtctattgagaatattaaagaattgaaagctggttatatctcacaggttgttcataaaatttgtgagttagtagaaaagtatgatgcggtgattgctctggaggatctcaattcatattgctcttctgtttatcagaagtttgaaaaaatgttaatcgataaactgaattatatggttgacaagaaatcaaatccatgtgctactggaggtgctctgaaaggttatcaaattaccaataaattcgaatcttttaagagtatgtccacacaaaatggatttattttctacattcctgcatggcttacgtcaaagatagatcctagtacgggattcgttaaccttttaaaaacgaaatatacttcaatagccgactccaagaagtttattagtagtttcgacagaattatgtatgtccctgaagaagatttgtttgagttcgcgcttgattataaaaattttagtagaactgatgctgattatattaaaaagtggaaactctattcgtacggaaatagaattcgtattttccggaatcctaaaaaaaacaatgttttcgactgggaagaggtctgcctcacatctgcctataaggaattgttcaataagtatgggataaactaccaacaaggcgatatacgtgctcttctttgcgaacagtcagataaagctttctacagctccttcatggcattgatgtcactcatgttgcaaatgcgaaattcaatcacaggacgcactgatgtcgattttttaattagtccggttaaaaactcggacggaattttctatgactctagaaattatgaagcgcaagaaaacgctattttgcccaagaatgcagacgctaatggagcatataacatagctagaaaagtgctgtgggcaatcggccaattcaaaaaggccgaagacgagaagttagacaaagtcaaaatagcgatttcaaacaaagagtggcttgagtatgctcagacctctgttaaacattctgggtcaggtggatcttcaggggggtccatggccgaaaatggcgataacgaaaagatggccgctcttgaggctaagatctgccatcaaattgagtattattttggcgactttaaccttccaagagacaaatttcttaaagaacagattaagctcgacgaagggtgggtgcccctcgaaatcatgattaaattcaatcgactcaacagactgactactgactttaatgtgatagtagaggcactatccaaaagcaaagcagaactaatggaaatatctgaagataaaactaagattcgccggtcgccctcaaagcctttaccagaggttaccgatgaatataagaacgacgtaaaaaaccggagcgtttatataaaaggtttcccaactgatgctacattggacgacattaaggagtggctcgaagacaaaggtcaagtattaaatatacagatgagacgaactcttcataaagcttttaaagggagcatttttgtcgtttttgactcaattgaatccgcgaagaagttcgtcgaaactccaggtcaaaaatataaagaaacagatcttttaatattgttcaaggatgattattttgctaagaaaaacgagtcaggagggtctaaaagaaccgcagatggcagcgagtttgaacctaaaaagaaacgaaaggtttga

SEQ ID NO: 290 - DNA, 4413 bp, synthetic construct
  source 1..4413: mol_type = other DNA
atgaaacgcaccgctgatggttctgaattcgagccgaaaaagaagcgaaaggtctctggcggctcttccggaggttcatccaaactcgaaaagttcaccaattgctacagtttgtccaagaccctgcgcttcaaggccattccagttggtaagacgcaagaaaatatcgacaataagcgattgctggtggaagacgagaaacgtgcagaggattacaagggagttaaaaagttgctcgaccgatactacttgagctttatcaacgatgttcttcactcaattaaacttaagaatctcaacaactatatttccctctttcgtaagaagacccgtactgaaaaggaaaacaaagagctggaaaacctggaaattaatctccgcaaggaaatcgccaaggcctttaagggtaacgagggctacaagtctctttttaagaaagatattatcgaaaccatccttccagagttcttggacgataaggacgagattgccttggtgaactcgttcaatggtttcacgaccgcatttactgggttcttccggaatcgcgaaaacatgttctcggaagaggcgaagtctacttcgatcgccttccgctgcattaatgagaacctgacacgttacattagcaacatggatattttcgagaaggtggacgctatcttcgacaaacacgaggttcaggagattaaagaaaaaatccttaactcagactatgatgtggaagacttttttgagggcgaatttttcaactttgtcctcacccaagaaggcatcgatgtgtataatgccattatcggcggctttgtaaccgaatccggtgagaagatcaaaggtctgaatgagtacatcaatttgtataaccaaaagacgaagcagaagctgccgaaattcaaacccttgtacaagcaggtgctcagtgaccgcgaatcgctgtccttctatggagagggctacactagcgacgaagaggtcttggaggtcttcaggaacaccttgaacaagaactccgaaattttctcctccattaaaaaacttgagaaactcttcaaaaatttcgacgaatactcgtcagccggcattttcgtgaaaaatggccctgctatttccaccatcagcaaggacattttcggtgagtggaatgtgatccgagacaagtggaatgcggaatacgacgatatccacttgaagaagaaggccgttgtgacagaaaagtacgaggatgatcgccgcaagtccttcaagaagatcggatcgttttccttggagcagctccaggagtatgctgatgccgatctctccgtcgttgagaagcttaaggaaatcatcatccaaaaagttgatgaaatctacaaagtttacggtagcagcgagaaactcttcgacgcagacttcgttttggaaaagtccttaaagaaaaatgacgccgttgtggcaattatgaaagacctcttggattctgtaaagagctttgagaattatatcaaggcattcttcggcgaaggcaaggagaccaatcgtgacgaatcattctacggtgatttcgtgctggcgtacgatatcctgctaaaggtggaccatatttacgatgctatccgtaactacgtaacgcaaaagccatacagcaaggataaatttaagttgtattttcagaacccccagttcatgggagggtgggacaaagataaagagaccgattaccgtgcaaccatccttcgatacggctcgaagtactacctagctatcatggacaaaaaatacgcgaagtgcctccagaagattgataaggatgacgtcaacggaaactacgagaaaattaactacaaattgctgccaggccctaataagatgctcccaaaagtctttttttctaagaaatggatggcgtattacaacccgtctgaagacatccagaaaatctataagaacggcaccttcaaaaaaggcgacatgtttaaccttaacgattgccataaattaattgacttttttaaggattcaatctcccgctaccccaagtggtcaaacgcatacgatttcaacttctcggagaccgagaaatacaaggacatcgcaggtttttaccgcgaagtggaagaacaaggatataaggtgagttttgagagcgcctctaaaaaggaggtggacaaactcgtcgaagaaggtaagctgtacatgttccaaatttacaacaaggacttctctgataagtcacacggcactcctaacctgcacaccatgtacttcaagctgctttttgatgagaacaaccacggccagatccgcctgagtggcggcgctgaacttttcatgagacgcgcgtctctaaaaaaggaagagctggtggtgcaccccgctaactcccccatcgccaacaagaacccagataaccccaagaaaactaccaccttatcgtacgatgtgtacaaagataagcgcttctccgaagaccagtacgagctgcacattccaatcgccattaacaagtgtcctaaaaacatttttaagattaacaccgaggtccgtgtcctactgaaacacgatgataacccatacgtcatcggtatcgaccgcggagagcgtaatttactgtacattgtcgttgttgacggaaaaggaaacattgtggaacagtattcccttaacgagatcattaataacttcaatggcattcgcatcaaaacagactaccactccctcctggataagaaggagaaagagcggttcgaggcaaggcagaactggacgtctattgagaatatcaaagaactaaaagcggggtatatatcccaagtagttcacaagatttgcgaactggtggaaaagtatgatgcagttatcgctctggaagatcttaacagctactgcagttcagtctatcaaaagttcgaaaaaatgctcattgataagcttaactatatggtcgataagaaatcaaacccctgtgcaaccggtggtgcactcaagggttaccagattacaaacaagtttgaatcttttaagtcaatgtccactcagaacggcttcattttctatatcccagcctggctgacctccaaaatcgatccgtccactggcttcgttaacctgctgaagactaaatataccagcatcgcagactctaaaaagtttatcagcagcttcgatcgcattatgtacgtgccagaagaagatctttttgaattcgccttggactacaagaatttctcacgcaccgatgcggattatatcaaaaagtggaagctctactcttacggcaaccgtatccgcatctttagaaatccgaagaagaataatgtgtttgattgggaagaggtgtgcctgacgtccgcatataaggagctgttcaataagtatggaatcaactaccaacagggcgacattcgtgctcttttatgtgaacagtccgataaagctttttacagttccttcatggcgctgatgtcattgatgctgcagatgcgcaactccattacaggtcgtaccgatgttgactttctcatcagtccagtcaaaaactccgatggaatattctatgattctcggaattatgaggcccaggaaaatgcgatcctgccgaagaacgcagacgctaacggcgcatataacatcgctcgcaaggtattgtgggcgatcggacagttcaagaaggccgaagacgagaagttagataaggtaaagattgctatcagcaacaaggaatggttggaatatgcacaaacctccgtcaaacattcaggcagcggtgggtccagcggtggttcgatggcagaaaacggcgacaacgaaaagatggcagcccttgaagcgaaaatctgtcatcaaatcgaatattacttcggcgattttaacctaccacgggataagtttctgaaggagcagatcaaattggatgagggctgggttccgctcgagataatgatcaaattcaacaggcttaaccgcctcaccaccgacttcaacgttatcgtcgaagctctttctaaatccaaggcggaactcatggaaatctcggaagataaaaccaagatccgtcggtccccttccaaacctctcccagaagtgacagacgagtacaagaatgatgtcaagaaccgatcggtgtatatcaaggggttcccgaccgacgcgactttggacgacattaaggaatggctcgaagataaagggcaggtactcaacatccaaatgcgtcgcactttgcataaggctttcaagggttcgatcttcgtcgtcttcgattccatcgaaagcgcaaagaaattcgtggagacacctggccaaaaatacaaagaaacggatctactgatcctgtttaaggatgactactttgctaaaaaaaatgaatctggtggatccaaacgcacggcggatggctccgaattcgagcctaaaaagaagcgcaaagtttaa

SEQ ID NO: 291 - DNA, 4413 bp, synthetic construct
  source 1..4413: mol_type = other DNA
atgaagcgaactgctgatggttcggaattcgagccaaagaaaaagcgtaaagtaagcggcggttccagcgggggcagctcgaaacttgaaaagttcactaactgttattctctctctaagaccctccgcttcaaggcgattccagttggcaagacccaggagaatatcgataacaaacgcctgctcgtcgaggacgagaagcgagccgaggattacaagggtgtcaagaagcttctagaccgttactaccttagcttcattaatgacgtgttgcattccattaagctcaaaaacctgaacaactacatctcgttgtttcgcaagaagactcgtacagaaaaagaaaataaggagctggagaacttggaaatcaacttgaggaaggaaattgctaaggcgttcaaaggtaacgaaggatataaaagtctcttcaagaaggacatcatcgaaacgattttgcccgaattcctggatgacaaggatgaaatcgcgctggtgaactcttttaacggctttacaactgctttcaccggattcttcaggaacagggagaacatgttctcggaggaggctaagtcgacaagtatcgcgtttcgctgcattaacgagaatctcacgcgctacatctcaaacatggatatatttgaaaaggtcgacgctatcttcgacaagcacgaagttcaggagatcaaggaaaagatcctcaacagcgactatgacgtagaagacttcttcgagggtgaattcttcaacttcgtgttaacccaagagggaattgatgtttacaacgccattatcggcggatttgtcaccgagtccggggaaaagattaagggtctcaacgagtacatcaacctttacaatcagaagactaagcagaagctccccaagttcaagcccctttataaacaagtgcttagcgaccgcgagtccctgtcgttctacggagaaggatacacttcggatgaggaggttttggaggtcttccgaaatactctcaataagaattcagaaatctttagcagtatcaaaaaacttgaaaagctgtttaagaacttcgacgaatacagcagcgccggaatcttcgtcaagaatggcccagcgatctcaactatcagcaaggatatctttggcgaatggaacgtgatccgagacaaatggaatgccgaatacgatgacatccatctcaagaagaaagcggtcgtcaccgaaaagtacgaggacgaccgccgaaagtccttcaagaagatcggctcgttctcattggaacagctgcaagaatacgctgatgccgaccttagcgttgtagagaagctcaaggagatcattatccagaaggtggacgagatctacaaagtctacggttcaagcgaaaagctctttgacgcggacttcgtgctggagaagtcgttgaagaaaaacgacgccgttgtggccatcatgaaggatctcctggactcggttaagtcgtttgagaactacatcaaggcattcttcggagagggcaaggaaacgaaccgcgatgagtctttctacggcgactttgtgctcgcctacgacatcctccttaaggttgaccacatatatgatgcgatccgtaactacgtaacacagaagccatactccaaagataagttcaagctttacttccagaacccgcagttcatgggtggctgggacaaggataaagaaacggactacagagctacaattcttcgctacggcagtaagtactaccttgccatcatggacaagaagtacgctaaatgtcttcagaagattgacaaggatgatgtcaacggtaactatgaaaagattaattacaagctgctccccggtccgaataagatgttgcccaaggtttttttctcaaagaaatggatggcttattacaaccccagcgaagatatccagaaaatctacaagaacggcaccttcaaaaaaggagacatgttcaacctcaacgactgccacaaactcatcgactttttcaaggattccatcagcagatatcctaagtggtcgaacgcatacgatttcaacttctccgagacggaaaagtacaaggacatcgctggtttttatcgcgaggtcgaagagcaaggctacaaggtctcgttcgagtcggcgtccaaaaaagaggtcgataaactggttgaggaaggtaaactctacatgttccaaatttataataaggactttagtgataagtcccacggtacacctaatctacataccatgtatttcaagttgttgttcgacgaaaacaatcacggtcaaatcagactcagtggaggtgctgagttgttcatgcgtcgagcttctctcaaaaaggaagagctcgttgtccacccagctaattcgccaatcgcgaataagaatcccgataacccaaagaaaaccacaacgctgtcgtacgatgtgtataaagataaaagattctccgaggatcagtacgaactccatatccctatcgcaatcaacaagtgccctaaaaacatcttcaaaatcaacacagaagtgcgtgtcctgttgaagcacgacgataatccttacgtgatcggcattgaccgcggtgaaaggaatctcctctatattgtagtcgtggatgggaagggaaacattgttgagcaatattctctgaacgaaatcatcaacaacttcaacgggattcggattaaaacggattaccactcgcttctagataaaaaggagaaagagcgattcgaggcacgacagaactggacctcaatagagaatatcaaagaactcaaggcggggtatatctctcaagtcgtccacaaaatatgcgagctcgttgagaagtacgatgctgtcatcgcattggaggacctcaattcctactgctcctctgtttaccaaaagtttgagaagatgctaattgacaagcttaactacatggtcgataagaagtccaatccgtgcgcaacaggcggcgccctcaagggataccagattacgaacaagttcgagagcttcaaatcgatgtcgacacagaacggttttattttttacatccctgcctggttgactagcaagatcgatccctcaaccggattcgtcaacttgcttaagacaaagtacacctcgattgccgactcaaagaagttcatctcctcgtttgatcgaatcatgtacgtccccgaagaagatttgttcgagttcgccttggactataaaaacttttctaggaccgatgccgactatatcaagaagtggaagctgtactcgtacggtaacagaattcgtattttcagaaacccgaaaaagaacaatgtctttgactgggaagaggtctgccttacgagcgcttacaaggagctcttcaataagtacggtatcaattaccaacagggtgatatccgagccctgttgtgtgagcagtctgataaggcattttatagttcatttatggcactcatgtcactcatgcttcagatgcgcaactcgatcactggtcgtaccgacgtcgacttcctcatctccccggtgaaaaactcagacggcatcttttacgactcacgtaattacgaggcccaagaaaatgcgattctcccaaaaaacgctgatgccaacggggcatacaacatcgcacgtaaggtcctgtgggcaatcggccagttcaagaaagcagaagacgagaagttggataaggtgaagatcgcaatcagcaacaaggagtggttggagtatgcccaaacctcggtaaaacattctggttccggaggtagtagcggcggatccatggccgagaacggcgataacgagaagatggctgcattggaggctaaaatctgccatcaaatcgaatactacttcggcgatttcaacctccctcgcgacaagttcctaaaagagcaaatcaagctcgacgagggctgggtccctctagaaatcatgatcaaattcaaccgacttaaccggctcacgaccgacttcaacgtcatcgtagaagctttgagcaagtctaaagctgagttgatggagatttcggaagacaagaccaagatccgtcgttctccatctaagccgcttcctgaggtaacggatgagtataagaacgatgttaaaaacaggtcggtctacattaagggcttccctacagacgctacgctggatgacatcaaggaatggcttgaggacaagggacaagtcttaaacatccagatgcgcaggactctccacaaagccttcaagggctccatcttcgtcgtcttcgacagcattgagagtgcaaagaagtttgtggagacccctgggcagaagtataaagaaaccgatctccttatcctcttcaaggacgactactttgcaaagaagaacgaatctggcggctccaaaaggacggcagatggttctgaatttgagcccaaaaagaagcgcaaggtatga

SEQ ID NO: 292 - DNA, 4410 bp, synthetic construct
  source 1..4410: mol_type = other DNA
atgaaaagaacagccgatggtagcgagttcgaaccaaaaaaaaaaagaaaggtctcgggagggtcttccggtggcagttctaaactggaaaagtttaccaactgctattcattgtccaagaccctacgctttaaagcaattccagtcggtaagacccaagaaaacattgataacaagcgactcctggtcgaagacgagaagagagctgaagattataagggagtgaagaaactcttagacaggtactacctgtcattcatcaatgatgtgctccattcaattaagctaaaaaatctcaacaactatatctccttgtttcgcaagaaaacccggaccgagaaagaaaacaaagagctggagaacctggagataaacttacggaaggaaattgccaaggcattcaagggaaatgagggctataagtctctgtttaagaaggacataattgagacaattctgccagagtttcttgacgataaggatgagattgcattagtgaactctttcaacgggtttacaactgctttcaccggattcttccggaatcgggaaaatatgttcagcgaagaggccaagtctaccagcattgccttcagatgcatcaatgagaacctgaccaggtacatatctaacatggacattttcgagaaagtcgacgcaatcttcgataagcatgaggtgcaggaaataaaagagaaaatcctcaactcagactacgacgtcgaggacttcttcgaaggcgagtttttcaacttcgttttaacccaggagggcattgatgtttataacgcaatcatcggtggctttgttaccgaaagcggcgagaagatcaaaggactgaatgagtacatcaacctctacaatcagaaaacaaagcagaaactgccgaaatttaaacccctatacaagcaggttctgtcagaccgggagagcttgtctttctacggcgagggttacacgtcagatgaagaggtacttgaggtgttcaggaacacattgaacaagaattctgagatcttctcatcaataaaaaagctggagaagctcttcaagaatttcgatgagtacagtagtgcaggcatttttgtaaaaaacggaccagccatctcaacaatttccaaagatatttttggtgaatggaatgtcattcgggataagtggaatgccgagtatgatgacatccacctcaaaaagaaagccgttgtaaccgagaagtatgaggacgaccggaggaaaagctttaagaagatcggctccttcagtcttgagcagctgcaggagtacgccgacgctgacctgtctgtggtagagaagctcaaggaaattattatccaaaaggtggacgagatatataaggtctacggatccagcgagaaacttttcgacgctgatttcgtcctagagaaaagtcttaagaaaaatgatgcggtcgtggccatcatgaaagaccttttggattctgttaagagtttcgaaaattacattaaagccttttttggggagggaaaagagacaaacagggacgaatcattttatggggatttcgtgctggcctacgatatactacttaaggtagatcatatctatgatgctatccgcaattacgttactcagaaaccttacagcaaggacaagtttaaactctactttcagaatccccagttcatgggcggatgggacaaagataaagaaacagactatagagctactattctgcggtacggctcaaagtactacctcgctataatggacaaaaagtacgccaagtgcttgcagaaaattgataaggacgatgtgaatggaaattacgagaagatcaactacaaactgctaccgggacctaataagatgctaccgaaagtcttcttcagcaagaagtggatggcatactacaacccatccgaggatatacagaagatctacaagaacgggacatttaagaagggtgacatgtttaacttgaatgattgccacaaactcatagactttttcaaggatagtatcagtagataccccaagtggtcaaacgcatacgacttcaattttagtgagacagagaagtacaaagatatcgccggcttttatagggaggtggaggaacaagggtataaagttagttttgagagcgcttccaaaaaggaagttgataaactggtggaagagggaaaactgtacatgttccagatatacaataaagacttttctgataagtcacacggaacccccaatttgcataccatgtactttaagctactgtttgacgaaaacaaccacgggcagatacgtctctctgggggagctgagttgtttatgcggcgcgctagtctgaaaaaggaagagctggtggtacacccagccaacagtccaattgccaataagaacccagacaaccctaaaaagaccacaacactaagctacgatgtttacaaggacaaaagatttagtgaagaccagtacgaactacatattccaatcgcaatcaacaagtgtcccaaaaatatttttaagataaatactgaggtccgcgtgcttttgaaacatgatgataacccgtacgtcatcggcattgaccgcggagagcgcaatttgctttacattgtggtggtggatgggaaggggaatatcgtggagcagtattccttgaatgagattataaacaacttcaacgggattcgcatcaagaccgattatcattcactactggataagaaggaaaaagaaagatttgaagctcggcagaactggacgagcatcgagaacattaaagaactgaaagccggctacattagtcaggtggtccataaaatctgtgaactggtagagaagtacgatgctgtgattgcactcgaagacttgaattcctactgctcttcagtttaccagaagttcgagaagatgcttatcgacaaactgaactacatggtcgacaaaaagtccaatccatgtgccacaggcggggccctgaaaggttaccaaatcactaacaaattcgagagcttcaagtccatgtccactcagaatgggttcatattctacatccccgcgtggctgacttccaagatcgatccttccactggctttgtgaacctgctgaagacaaagtatacaagtattgcggactccaaaaagttcatttcttcatttgaccggattatgtacgtccctgaagaggacctgttcgaatttgctctcgattataagaatttttccaggactgacgcagattatataaagaagtggaagctttactcgtacggcaatagaattaggattttcagaaatcccaagaaaaataatgtgtttgactgggaggaggtctgcctcacaagtgcatataaggaattattcaacaagtacggaattaattaccagcagggggatattagggccctgctgtgtgaacaatccgataaagctttttactcctctttcatggccttgatgtcattaatgctacagatgcgtaattctataacaggaaggaccgatgtagactttttgatcagccccgtgaagaactctgatggcatcttctatgattccaggaactacgaagctcaggagaacgccatactgcctaaaaatgccgacgctaacggcgcttataacattgctcgaaaggtgctgtgggccataggacagttcaaaaaggccgaggacgagaagctagacaaagttaagatcgctatctccaacaaggagtggttagagtacgcgcagactagtgtgaagcacagcgggtccgggggctcctccggggggtccatggctgaaaacggtgacaatgagaaaatggccgctttggaagcaaagatttgccaccagatagagtactattttggagactttaatcttccacgtgataaattcctcaaggagcagatcaaacttgatgagggttgggtgccactagaaataatgattaagttcaatcggttaaacaggttgacaactgacttcaatgtcatcgtggaggctctaagcaagtcgaaggcagagctcatggagatctcagaggacaagaccaagattcggagaagcccttccaaacctttgccggaggttactgatgagtataaaaatgacgtcaaaaatcggtccgtctacatcaagggcttccccaccgacgccacattagatgatataaaggaatggttggaagataaaggccaagtgctgaatatccagatgaggaggacgctgcacaaagccttcaagggctcaatctttgtggtttttgacagcattgagtccgcaaagaagttcgtggagacgccaggccagaagtacaaagagacagacctgctgatactatttaaagacgattacttcgcaaaaaagaatgaatctggggggtcaaaacgcacggcagacggctcagagttcgaaccaaagaaaaaaagaaaagtg

SEQ ID NO: 293 - DNA, 4410 bp, synthetic construct
  source 1..4410: mol_type = other DNA
atgaaaagaactgccgacggatcagagtttgagcccaagaaaaaacgtaaggtctccgggggcagcagtggaggatctagtaaactcgaaaagttcaccaattgttactccctgtcaaagactctgcggttcaaggctattcctgttgggaagacacaggagaatatcgataacaaaagacttctggtggaagacgagaaacgggctgaggactacaaaggagtcaagaagctgcttgaccgctactacctgagcttcattaatgacgtcctacacagtatcaaattaaagaacctaaataactatatctccctgtttagaaagaaaacaagaactgaaaaagagaataaggagttagagaatttggagatcaacttgaggaaggaaatcgccaaggccttcaaaggtaacgagggctacaagtccctttttaagaaggacattattgagactattctgccggagttcctggacgacaaagatgaaatagccttagtgaactccttcaacggatttactaccgcgtttactggcttctttaggaatagggagaatatgtttagcgaggaagcaaagagtacttcaatcgcattccgatgcattaatgagaacctgacgcgatatattagcaacatggacatttttgagaaagtagacgctatcttcgacaaacacgaggttcaagagattaaggaaaaaatactgaattctgactatgatgttgaggattttttcgaaggggaattttttaattttgtactgacccaggagggcattgatgtgtacaacgccatcatcggtggattcgtcacggagagcggggagaaaatcaaaggtctgaacgaatacatcaacctgtacaatcagaagactaagcagaaactgcccaagtttaaacccctgtacaaacaggtactgtcagatcgagagtcattgtccttttacggagaaggatatacgagcgatgaggaggtactcgaggtcttcagaaatacactgaataagaactctgagatattcagctcgatcaagaagctggagaagctgttcaaaaacttcgatgaatacagtagtgccggcatcttcgtgaaaaacggtcccgccatcagtaccatttccaaggatatatttggggagtggaatgtcattagagacaagtggaacgccgagtacgatgacatccacctgaaaaagaaagctgtagtgacagaaaagtacgaagacgatagaaggaagagcttcaagaaaatcggatccttctcactggagcagctccaggagtacgcggacgccgacctctcagtcgtagagaagctgaaggagattataatccaaaaagtggatgaaatttacaaggtatatggctcatccgagaagcttttcgacgctgattttgtactggaaaagtcactgaagaaaaatgatgctgtcgtggccattatgaaggatctgctggacagtgtgaaatcgttcgaaaactacataaaagccttcttcggagaaggtaaggaaaccaacagagacgaatccttctatggggatttcgtgttagcttacgacatattgctgaaggtggatcacatctacgatgccatccgcaattacgtgacccagaaaccctactcaaaggacaaattcaaattatacttccagaacccccagttcatgggcggctgggataaggacaaggagacggactacagggccaccattctgcgatacgggagcaagtattacctggccatcatggacaagaagtatgcaaaatgtctacagaaaatcgataaggacgacgtgaatgggaactatgagaaaatcaactacaagctcctgccaggccccaacaaaatgttgcctaaagtgtttttcagtaaaaaatggatggcgtattacaatccttcagaagacatacaaaagatatacaagaacgggacttttaaaaagggggatatgtttaacttgaatgactgtcacaaactgattgattttttcaaggactccattagcagatacccaaaatggagcaatgcatatgattttaacttttcagagaccgaaaagtataaggacatcgctggattctacagggaagttgaggaacagggttacaaggtgagtttcgaatcagccagcaagaaggaggtggataagctcgtcgaggaagggaagctctacatgtttcagatttacaacaaggattttagtgataagtcacatggaacaccaaatctccacaccatgtacttcaagctgttattcgatgagaacaatcacggacagatcagactgtctggaggagcggaattattcatgagacgggcgagcctgaaaaaggaggaattggtggtgcacccagcgaattctccaatcgcaaacaaaaatccagacaaccctaagaaaaccactactctcagttacgatgtgtataaagataagcggttcagcgaagatcagtacgagctgcatatcccgattgccattaacaagtgccccaaaaatattttcaagatcaacacagaggtcagggtgctgcttaagcacgatgacaacccatacgttatcggtattgacagaggggaaagaaacctgctctacatcgttgttgtagacggaaaagggaacatcgtcgagcagtactccctgaatgagattatcaataatttcaacggcatcaggatcaaaacagattaccatagcctcctggataaaaaggaaaaggaaagattcgaggcccggcaaaactggacctcgattgaaaacatcaaggagcttaaggcgggctacatttcccaggtggttcacaagatttgcgagttggtagaaaaatacgatgcggttatcgcactggaggacctgaactcctactgcagctcagtgtaccaaaagttcgagaaaatgctgatagataaactaaattatatggttgataaaaagtctaatccatgtgcaacgggaggtgcgctcaagggataccagatcaccaacaagttcgaatccttcaagtcaatgtctacacagaatggcttcattttctacatcccagcctggctgacatccaaaatcgacccttctacggggttcgtgaatttgctaaagacaaaatatacctccatcgctgactccaagaagttcattagcagctttgataggattatgtacgtgccagaagaagatctgttcgaattcgccctggattacaagaatttcagtcgaacagatgcagattacataaaaaagtggaagctctactcttacggtaaccggatccgcatcttcaggaatcccaagaaaaataatgtcttcgactgggaggaggtttgcctgacttcagcctacaaagaattgtttaacaagtacggcatcaattatcagcagggagacatcagggctctgctctgtgagcaaagtgacaaggccttttactcctcctttatggctctgatgtctctgatgctgcagatgagaaactcaatcactggacgaactgacgtcgacttcctgataagccctgtgaaaaattccgatggtattttctatgatagcaggaactatgaagctcaggagaatgcaatactgccaaagaacgccgatgcgaatggcgcttacaacatcgcgcggaaggtcctctgggccattggtcagttcaaaaaagctgaagatgagaagttagataaggtgaaaatcgctattagtaataaggaatggctggagtacgcccagacaagcgttaaacacagcgggagcggcggctcatcaggaggatctatggccgaaaatggcgacaacgagaagatggctgcactggaggcgaaaatctgccatcagatagagtactactttggtgacttcaacctgccacgggacaagttcctgaaagagcagatcaagctcgacgagggatgggttccgctcgagattatgattaagttcaaccgcctgaaccgcttaactaccgacttcaacgtaattgtggaggctctgtcaaagagcaaggcagaactgatggagatctcagaggacaagactaagatccgtcggagcccctccaaacccctgcctgaggtgactgacgaatataaaaacgatgttaagaaccggagcgtctatatcaaaggtttccctacggacgctacactggatgacataaaggaatggctcgaggataaagggcaagtgcttaatatccagatgcgacggacgttgcataaggcgttcaagggatccatattcgtggtctttgacagcattgagtcagctaaaaaattcgtggagactcccggacaaaaatacaaggaaacagacctgctaattctgtttaaggacgactatttcgccaagaagaatgagagtgggggatcaaagaggaccgccgatgggagcgaatttgagccgaagaagaaacgcaaagtt

SEQ ID NO: 294 - DNA, 4410 bp, synthetic construct
  source 1..4410: mol_type = other DNA
atgaaaagaaccgctgatggatctgagttcgagccaaagaagaagcgcaaggtgagcgggggaagttcgggtgggtcttccaagctggagaaattcaccaattgctactcactctcaaagacattaagattcaaagcgatccccgtgggcaaaacacaggagaatattgacaacaagcgtctactggtcgaagatgagaagcgcgcggaagattataagggtgtcaagaagctgctggacagatattatctgtcatttattaacgacgttctgcatagcatcaagttgaagaacctcaacaactacattagccttttccgaaaaaagactagaacagaaaaggaaaataaggagctcgagaacctggagatcaaccttcgaaaggagatagccaaagctttcaaaggcaatgagggctacaaatccctgtttaaaaaggacattatcgaaacgatcctccccgagttcctagacgataaggatgagattgctttggtgaattccttcaacggttttactacagcctttaccggattttttcggaaccgtgaaaacatgtttagcgaggaagcaaagtctacatcaatagcattccgctgcattaatgagaacctcactcggtatatttcaaatatggacatttttgagaaagtggacgctatcttcgataaacacgaggtccaggaaatcaaagagaagattttgaatagcgattacgatgtcgaggatttcttcgaaggtgagtttttcaacttcgtactgacacaggagggaatcgacgtttacaacgctattatcggcggcttcgtaacggagagtggcgagaagatcaagggactcaacgaatacatcaacctttacaatcaaaagacgaaacaaaagcttcccaaattcaagcccctgtacaaacaggtgctctcagaccgcgagtcattatctttttacggtgaggggtatacaagcgatgaggaggttttagaggtgttccgcaacacactcaacaagaactccgagatcttttcctcaattaaaaagctcgaaaagctcttcaaaaacttcgatgagtatagttcggccgggattttcgtgaaaaatgggcccgccatttctaccatttccaaagacatctttggcgaatggaacgtgatccgagacaagtggaacgcagagtatgacgacatacaccttaaaaaaaaagctgttgttacggagaagtacgaggatgaccgtcgtaagtcctttaaaaagatcgggagcttttccctcgaacagctgcaggaatacgcagatgctgatctgtcagtggtggagaaattgaaggagatcatcatccagaaggtggatgagatttacaaggtttacggttccagcgaaaagctattcgacgctgactttgtcctggagaaatccctcaaaaaaaacgacgccgtcgtagctatcatgaaggacctgctggattctgtcaagtcatttgagaactacatcaaggccttctttggagagggaaaggaaactaacagggacgagtctttctacggcgactttgtcctagcctacgatatccttttgaaagtcgaccatatatacgacgctattcgcaactatgtgacccagaaaccttacagcaaagacaaatttaaactttatttccagaatccccagttcatgggaggctgggacaaagacaaggagaccgactaccgcgccacaatcttgcgctatggaagtaagtactatttggcgattatggacaagaagtacgcaaagtgcctccaaaaaatcgataaagatgatgtgaacggcaattatgaaaagatcaactacaagcttctgcctgggcccaacaaaatgttaccaaaagtcttttttagcaaaaaatggatggcctattacaacccctctgaggacatccaaaagatttataagaatggtacattcaaaaaaggcgatatgtttaatctgaatgattgtcataagctgattgatttttttaaggactctatctccaggtaccctaagtggtctaacgcatacgattttaacttttcggagactgaaaagtataaggatatcgctggcttctaccgtgaagtggaggagcaaggctacaaagtgagtttcgaatcagcgagcaagaaagaggtcgataagctggtcgaggagggcaaactttatatgttccagatctataataaagatttctcggataagtctcacggcacccccaatcttcataccatgtatttcaagctcctgtttgacgagaataatcatggtcagatccggctgagcgggggagccgagttgttcatgcggagggcctctctgaaaaaggaggagctggtggtccatcccgctaactctccaatcgctaataagaatcccgacaaccccaagaaaaccacaacactgtcttacgatgtgtacaaagacaagcgcttctcagaggatcaatatgaactgcatatcccaatcgctattaacaaatgccccaagaatattttcaaaatcaatacagaggttagagtgctcctgaagcatgatgacaatccttacgtgataggtatcgatagaggcgaacgcaacctgctgtacatcgtcgtggtagacggtaagggaaacatcgtggaacagtactcactgaacgaaataatcaacaatttcaacggcatccggatcaaaactgattaccacagcctcctggacaagaaggagaaggaaagattcgaggctagacagaattggacaagcatagagaatatcaaagagctgaaagcagggtatatttctcaggtcgttcacaagatatgtgagctggtcgaaaagtatgatgccgtgattgccctggaggatttgaactcctactgcagctctgtctatcagaagtttgaaaaaatgctgatcgataagctcaattatatggtggataagaagtctaacccctgtgcaacaggaggcgcactaaagggctaccagattaccaataaatttgagagtttcaagtccatgagcacccaaaacggatttatcttttacattcctgcctggctgacgagtaagatcgatcccagcaccgggttcgtgaatctcctcaaaaccaagtacacgagtatcgcagacagcaagaagttcatctctagcttcgataggattatgtacgtgcccgaggaggatctgtttgaattcgccctggactataagaattttagtcgcactgatgccgactatataaagaaatggaaattgtattcatacggaaatagaattaggatttttagaaaccccaaaaaaaacaatgtcttcgattgggaagaagtgtgcctgacgagcgcctacaaggagctatttaataagtacggcattaactaccagcagggagacatcagagctcttctttgtgaacagtcagacaaggcattctatagtagctttatggctctgatgagtctgatgctgcagatgcgcaattccatcacaggtcggacggacgtggacttcctgatatcccccgttaaaaactcagacggaatcttctacgatagccgtaattatgaagcacaggagaacgccatactgcctaaaaacgccgacgccaacggagcctacaacatagccagaaaggtcttgtgggccatcggccagttcaagaaggctgaagatgaaaaattggataaggtgaagattgcaatttccaacaaagagtggctggagtacgcccagacgtccgtgaagcacagcggaagtggggggtcctccgggggctccatggccgagaacggcgacaacgagaaaatggccgccttggaggccaagatatgccatcagatcgagtattattttggcgacttcaaccttccaagagataaattcctgaaggaacagattaagctcgatgaaggctgggtgccactcgagattatgatcaaatttaatagactgaatcggttgacaacagatttcaatgtgatcgttgaggcattatcaaagtccaaggcagaactaatggagattagtgaagataagaccaagattagacggtcaccatctaagcccctccctgaggttacagacgagtataagaacgatgtgaaaaaccgtagcgtttacattaaggggttccctacagacgctactctggacgacattaaagagtggctggaagacaaaggtcaagtactcaatattcagatgcggagaactttgcacaaggcttttaaaggctctattttcgtggttttcgattccattgagtccgccaagaaattcgttgagaccccgggccagaagtacaaggagacagacttgctcatactcttcaaggacgattatttcgcaaagaagaacgagtcaggcggctcaaagagaaccgccgacgggagcgaatttgagcccaagaaaaaaagaaaggtg

SEQ ID NO: 295 - DNA, 4410 bp, synthetic construct
  source 1..4410: mol_type = other DNA
atgaaacgcacagcagacggcagtgaatttgagcccaagaagaaacgtaaggtgtctggcgggtcttcgggcgggagtagcaagcttgaaaaattcactaactgctactctcttagtaaaactctgcgttttaaagcaatccccgtgggtaaaacccaagagaacatagacaacaaaagactgttagtcgaggacgagaaacgagctgaagattacaagggagtgaaaaagctacttgacagatactatctgagttttatcaatgacgtgctgcacagcatcaagctaaagaacctcaacaattacatatctctcttcaggaagaagactagaaccgagaaagagaacaaggaactcgaaaatctcgagattaatttacgcaaagaaatcgccaaggcgtttaagggcaatgagggatacaaatccttgtttaagaaggatatcatcgagactattctgcccgaattcctggacgataaggacgaaatcgctttagtaaacagctttaatggattcacaaccgcatttacggggttcttccgaaatagggaaaatatgttttctgaggaggcaaaatctaccagtatcgctttcaggtgcattaacgaaaaccttacacgatacatctctaacatggatatctttgaaaaagtggacgctattttcgacaagcacgaggtacaggagatcaaggagaagattttaaattcagattacgatgttgaggacttcttcgagggggagttcttcaatttcgtcctcacccaggagggaatcgatgtgtataacgcaattatcggtgggtttgtaacggagtctggcgagaagatcaaggggttgaatgagtacattaacttgtataaccagaagaccaagcagaagctgccaaagttcaagcccctatacaagcaagtgctgagtgacagggagtcattgtctttctatggcgaaggctacaccagtgacgaagaggtgcttgaggtttttcggaatacgttgaataagaactccgaaattttctcatccatcaaaaaattggagaaactgtttaaaaactttgacgaatacagcagcgccggcatcttcgttaagaacgggcccgccatctccacaattagtaaggatatctttggggaatggaacgtgatcagagataagtggaacgccgagtacgacgatattcacctgaagaaaaaagccgtagtgactgagaagtacgaagacgacaggcgaaaatcattcaagaaaattggcagtttctccctcgaacagctccaagagtacgcagacgcagacctgtcagtagtggaaaaactgaaagagattatcattcagaaggtcgatgaaatctataaggtatacggctcatctgaaaaattgtttgacgccgactttgtccttgagaaaagcttaaagaagaacgacgccgtggtggctatcatgaaggacttacttgacagcgtaaagagtttcgagaactacataaaggcgtttttcggcgaggggaaggagacgaacagggatgaatcattttacggggacttcgtacttgcctacgatatcctgttgaaggtcgaccacatttacgacgcaatccgtaactacgtgactcaaaagccctatagcaaggacaagttcaagctgtatttccaaaacccccagttcatgggcgggtgggacaaggataaagaaactgattacagggctacaatcttacggtatggttctaagtattatcttgccattatggataagaagtatgcaaagtgccttcaaaagatagacaaggatgacgtgaacggcaactatgaaaaaattaattacaagttgctgcccggcccaaacaaaatgcttccaaaagtgttcttctctaaaaagtggatggcttattacaacccaagtgaggacatccagaagatatacaagaatgggacctttaaaaaaggcgacatgttcaacttgaatgactgtcacaagctgatcgatttcttcaaagactctatttccagataccctaagtggtcaaacgcatacgactttaacttcagcgagaccgaaaagtacaaagacatagctggtttctaccgcgaagtggaggagcaaggctacaaggttagctttgaatctgcatcgaagaaagaagtagacaagctggtggaggagggcaagctttacatgtttcagatctataacaaggattttagtgataaaagccacggtacccctaatctacacacaatgtacttcaaactgctgtttgatgaaaataatcatgggcagattcggctatccgggggagccgaactatttatgaggcgggcatcacttaagaaagaagaacttgtcgtgcatcccgcaaattctccaatcgccaacaagaaccctgacaatcctaagaaaacaactaccctgtcttacgacgtgtacaaagataagagattcagtgaagaccagtatgaattgcacatccctatagcgatcaataaatgcccaaagaacatattcaagatcaatacggaagtccgagtcctgctgaagcacgatgacaatccttacgtaatcggcatcgacagaggcgagcgtaacttgctgtatatcgttgtggtcgacggtaagggtaacattgtagaacagtattcactcaatgaaatcattaacaacttcaacggcattcgcattaagacagactatcattctcttttggacaagaaggaaaaggagagattcgaggcgcggcagaactggacgagtatcgagaatatcaaggaactgaaagcaggatatattagtcaagtggttcacaaaatctgtgagttagtcgagaagtacgacgctgtcatcgcactggaggacctgaatagttactgcagtagcgtgtaccagaagtttgagaagatgctgatcgataagctgaattacatggttgacaagaaaagcaacccatgtgcaaccggaggggcactcaagggctatcagataactaacaaatttgaatcttttaagtccatgtccacccaaaatggtttcattttctacatccctgcctggcttacaagtaagatagatccatctaccgggttcgtgaacctgttaaagaccaagtatacctccatcgcagatagcaagaaatttatcagctctttcgatcgcatcatgtatgtgccagaagaagacctgtttgagtttgccttggactacaagaatttctctcggacagacgcggattatattaagaagtggaagctctacagttatggcaaccggatcaggatttttcgcaatcccaagaagaataacgtctttgactgggaggaagtttgccttacttcagcttataaagagctatttaataaatatggcattaattatcagcagggagacattagagcactcctatgtgagcagtcagacaaagccttctattcttctttcatggcactgatgagtcttatgctgcagatgaggaacagtataactgggcggaccgacgtggatttcctcatcagccccgttaagaacagtgacgggatcttctacgattctcgcaactacgaagcacaggaaaacgccattctcccaaaaaacgcggacgccaatggcgcgtacaacatcgcaagaaaagtactctgggccattggacagttcaaaaaggcggaggacgaaaagttagacaaggtgaaaatcgctataagcaataaagaatggctggagtatgcacaaacttctgtcaagcattccgggtcgggcggatcatccggggggagtatggccgaaaatggtgacaacgaaaaaatggctgcacttgaggccaaaatatgtcatcaaatcgagtattattttggggacttcaatcttccacgggacaagttcctgaaggaacagatcaaattggacgaaggatgggtacctttggagattatgattaaatttaaccgacttaacaggttgacaacagactttaatgtgatagtcgaggcgctctctaagtctaaggctgagctaatggaaatcagtgaagataagaccaagatccggaggtctccgagcaagccactgccggaggtgactgatgagtataagaatgatgtgaaaaataggagcgtgtacattaagggctttccaaccgatgcaacgctcgacgatattaaagagtggttggaggacaaagggcaagtactgaacattcagatgagaaggactctgcataaggcctttaaaggatctatttttgtcgtatttgacagcatagaatcagccaagaagtttgtcgaaacgcccggtcagaaatacaaagaaactgatctacttattctctttaaggatgattacttcgccaaaaaaaacgagtctggaggatctaaacgcacagctgatggatctgaattcgagcctaagaagaaacggaaagtt

SEQ ID NO: 296 - DNA, 4410 bp, synthetic construct
  source 1..4410: mol_type = other DNA
atgaagcgcacagctgacggatcggagtttgaacccaagaaaaagcgaaaggtgtctggaggctcatcaggtgggtcatcaaaactggagaagtttactaattgctattcccttagtaagaccctgagattcaaggcaatacccgtgggcaaaactcaggagaatattgacaataaaagactgctcgtggaagatgagaaacgcgctgaagattataaaggcgttaaaaaactgttggaccgatactacctgtcttttattaatgatgtcctccactccatcaagctgaaaaaccttaacaactacattagtctctttcggaagaagacgcgcacagaaaaagaaaacaaggaactggagaacctcgagattaatctgcggaaggagatcgcgaaggcgttcaaggggaatgagggctacaaatctctctttaaaaaggatatcattgagaccatactccctgaattcctcgacgataaagatgaaattgccctcgtgaatagtttcaacgggttcaccacggcctttactgggttcttcaggaatcgtgaaaatatgttttcagaggaggccaagagcacctcaatcgccttcaggtgcatcaatgagaacctgaccaggtacatcagcaacatggacattttcgaaaaggtcgacgccatcttcgacaagcacgaggtccaggagatcaaagaaaagattctgaattccgattacgacgtggaggactttttcgagggcgaatttttcaactttgttctcacccaggagggtattgatgtgtataatgccataataggcggcttcgtcaccgaatctggagaaaaaatcaaggggcttaatgaatacattaacctttacaaccagaagaccaagcaaaagctgcccaagttcaagcccctctataaacaggtgctcagcgatagagaatctctcagcttttacggagaggggtacacatctgatgaggaagtgctggaggtgtttagaaatacactcaacaagaacagcgagatcttctctagcatcaaaaagctggaaaagctgtttaagaatttcgacgagtactcgagcgcaggtatcttcgttaagaacggcccagcgatcagcaccatcagcaaggacattttcggggagtggaatgtgatcagggataagtggaacgccgagtatgatgatatacatctgaagaaaaaggccgtggtcaccgaaaagtatgaagacgacaggagaaaaagtttcaagaaaatcgggagcttcagtctggagcaactgcaggagtacgctgatgctgacctttccgtggtggaaaagcttaaggaaataattatccagaaggtcgacgagatctacaaagtctatgggagctccgagaaactgttcgacgccgatttcgtccttgagaagtctcttaaaaagaatgatgcagtggtggccatcatgaaagacctcctagacagcgtgaagtccttcgaaaattacatcaaagcgttttttggcgaaggcaaagaaacaaacagagatgaaagcttttatggagacttcgttctcgcctacgacatcctgcttaaagtggaccacatctatgacgccatacggaactacgtgactcagaagccctattctaaggacaagtttaagttatattttcagaaccctcaattcatgggaggatgggacaaggacaaagagacggactatagagctacgatcctgcgttacgggagcaaatactacctcgccatcatggacaagaagtatgctaagtgtctgcagaaaatcgacaaagacgatgtcaacggtaactatgagaagattaattataagctgctcccaggacccaacaagatgctccccaaagtgttcttcagcaaaaagtggatggcgtattataatccatcagaggacatacagaagatctataagaacgggacatttaaaaagggcgatatgttcaacctgaacgattgtcacaaactgatagatttctttaaggactccatctctcgctatcccaagtggtctaacgcttatgacttcaacttctccgagactgagaaatacaaggatattgcaggattctatcgcgaagtggaggagcagggatacaaggttagcttcgaatctgcctctaaaaaggaggtggacaaactggtggaggaaggaaaactctacatgttccagatatataataaggacttttcagacaagtcccacggaactccaaatcttcatacgatgtacttcaaactgctgtttgacgagaataaccacggtcaaattcgtctttccggaggagccgaactgtttatgagacgtgcaagtctgaagaaggaagagctggtggttcacccagccaactccccgattgccaacaagaacccagacaatcctaagaagactacgaccctgagctacgacgtctacaaagacaagcggttttccgaggatcagtacgagctgcatatcccaatcgccatcaataaatgtccaaagaacatctttaagatcaacaccgaagttcgcgtgctcttgaaacacgacgacaacccatacgtcatcggcattgacaggggagagcgtaaccttctttacatcgtggtggttgacgggaagggtaatattgtggagcagtatagtctcaacgaaattattaataacttcaacggcatcaggatcaagaccgactaccattccctcctcgacaagaaggagaaagagcgcttcgaggcccggcagaattggacatctatcgaaaacatcaaagaactgaaagccggctacatctctcaggtggttcataagatttgtgagctagtggaaaagtacgatgccgtcatagctttggaagacctgaacagctactgttcttccgtctaccagaagtttgaaaaaatgttgattgataagctgaattacatggtagataaaaagtcaaacccctgtgccaccggaggagccctgaaaggctatcaaataaccaacaagttcgagagctttaagagcatgagcacacaaaatggattcattttttatattcccgcctggttaaccagcaagatcgatccttcgactggcttcgtcaacctgcttaaaacaaagtacacatccattgccgactccaagaagttcatatctagctttgatcggataatgtatgttcccgaggaggacctgtttgaatttgcgctcgactacaagaatttttctagaacggacgccgactacatcaagaagtggaagctctattcctacggtaacagaattcggatcttcaggaatcctaaaaagaacaacgtgttcgactgggaagaagtttgcctcacgagcgcttataaggaactctttaacaaatatggcattaattaccagcagggggacatccgtgccctgctgtgtgaacagtctgataaggctttctactctagcttcatggccctgatgtcactcatgctgcagatgaggaacagtataacaggccgcacagacgtggacttcctgatctccccagtcaaaaatagcgatggtatattttacgatagtcgcaactatgaggcccaagaaaacgctattttgcctaagaatgccgatgccaacggtgcctataacattgctcggaaagtactatgggcaattggccagttcaagaaggcggaagacgaaaagctggataaggtgaaaatcgccataagcaataaggaatggctggagtatgcccagacttccgtgaagcacagtgggtctggcggttccagcggcggctccatggccgagaacggcgacaacgagaagatggctgccctggaggcgaaaatctgtcaccagattgagtattacttcggtgattttaacctcccgagagataaattcctgaaggagcaaattaaactggatgaggggtgggtccctctggaaatcatgatcaaatttaatcgactgaatcggctgaccacagattttaacgtgattgtggaggccctcagtaagtcaaaggccgagcttatggagatttcggaggacaagactaagatcagacgctcaccttctaagcctttgccagaggtcaccgacgaatacaagaacgacgtgaagaaccgctctgtttatataaaggggtttccaaccgatgctactttagatgacatcaaagagtggctggaagataaagggcaggtgctgaatattcagatgagaaggacgctgcataaggcttttaagggctccatttttgtcgtgttcgacagcattgagagcgctaagaaattcgtggagacgcccgggcagaagtacaaggagaccgacctcctgatcctcttcaaagacgactactttgctaagaagaatgagagcggcggttctaagcggaccgctgatggcagtgagttcgaacccaaaaaaaagcgcaaagtt

SEQ ID NO: 297 - DNA, 4410 bp, synthetic construct
  source 1..4410: mol_type = other DNA
atgaaaagaaccgccgacgggtccgaatttgaacccaagaagaagcgaaaagtgtcagggggctcctcaggaggtagctccaagcttgaaaagtttaccaactgctactccctgagcaagaccttacggttcaaagccatcccagtgggcaaaacacaggaaaacatcgataacaaacgactactcgttgaagacgagaagagagcagaagactataaaggtgtcaagaagctcttggataggtattatctgtcatttatcaatgacgtgcttcacagtataaagctgaaaaatctgaacaattatatttccttgtttaggaagaagactaggacggagaaggagaataaagagctcgagaaccttgaaattaatttaaggaaggagatcgcaaaagcctttaaggggaatgagggctacaagtcacttttcaaaaaagacataatcgagacgatcctgcctgagttcctagacgacaaggacgaaatcgctctcgtgaattcctttaatggatttactactgcattcacgggattcttccgcaacagagagaatatgttttcagaagaagcaaaaagcacatcgatagcttttaggtgtattaatgaaaacttaacacgctacatctctaatatggatatctttgagaaagtggatgcaatttttgacaagcacgaggtgcaggagattaaagagaaaattcttaactcggattatgatgtggaggacttcttcgaaggggagttctttaattttgtgcttacgcaggagggaattgacgtgtacaacgcaatcatcggtggcttcgtgactgaatccggagagaagatcaaaggactgaatgaatacattaatctgtataaccagaagacaaaacaaaaactgccgaagttcaaaccactttacaaacaggtcctcagcgaccgggaatcactgagtttttacggggagggatacacatcagacgaagaggtcttagaggtgtttaggaatacgctgaacaaaaactctgaaatcttctcttcgatcaaaaagttggagaagctgtttaagaactttgatgaatactcttcagcgggcatcttcgtgaagaacggcccagcaatttcgacaataagtaaggatattttcggtgagtggaacgtgataagagacaaatggaatgctgaatacgacgacatccacctgaagaagaaagctgtcgttacggaaaaatatgaggacgaccgacgaaaaagttttaaaaaaatcgggagcttttctttggaacagctacaggaatatgccgacgctgatttgagcgttgttgagaaactgaaagagataatcatccagaaggtagacgagatatacaaagtttatggcagctctgaaaaattatttgacgctgacttcgtactggagaagagcttgaaaaagaacgatgcagtggtggcgattatgaaggatctgctggatagtgtcaaaagcttcgagaactatatcaaggcctttttcggggagggcaaggagacaaacagggacgaaagcttttatggagatttcgttctcgcttacgatatcctcctaaaggtggaccacatttatgacgccatacggaattatgtgacacagaaaccctacagtaaagacaagttcaagctgtactttcagaatccacaattcatgggaggctgggacaaggataaggagactgactacagagctaccatcctccggtatggttcgaagtactacctggcaatcatggacaaaaagtacgcgaagtgcctccaaaaaattgacaaagatgacgtgaacggaaactacgaaaagatcaactataagctgttaccagggcctaacaagatgttgcctaaggtgttctttagcaaaaagtggatggcgtattacaacccatctgaagacatacagaagatttacaaaaatggtacctttaagaaaggcgacatgttcaacctgaacgattgtcacaagttaatagacttctttaaggattctatctcgagataccccaaatggagtaacgcatatgactttaacttttcagaaacagaaaaatacaaggatatcgcagggttctacagggaagtcgaggagcagggctacaaggtgtccttcgagagcgccagcaaaaaagaagtggacaagctggtcgaggagggcaagctatatatgtttcagatttacaacaaagatttttcagacaagagccatgggacacccaacctgcatacgatgtactttaaacttttgtttgatgagaacaaccacggtcagattcgtctgagcggaggtgctgaactcttcatgagacgcgcttccctgaaaaaggaagagctcgttgttcaccctgctaacagtcctattgccaacaaaaacccagacaatcctaaaaagacaacgactttgtcttatgacgtttacaaagataaaagatttagcgaagaccagtacgagctccacatacctatcgcaatcaataagtgccccaaaaacatattcaaaatcaatactgaggtgcgcgtgctcctgaagcatgacgataacccctacgtaatcggaatagatcgaggtgagcgcaatcttctgtacatcgtggtagtcgacgggaagggtaacatagtggagcaatatagtctcaacgagattatcaataatttcaatggaattaggataaagaccgactatcactcactgctggataagaaggaaaaggagagatttgaggcgaggcagaactggactagtattgagaatatcaaggaactgaaggcgggctacatctctcaggttgtccacaaaatatgcgagctggtggaaaagtacgatgctgtgatagcccttgaggaccttaactcgtattgctcttcagtctaccagaagttcgagaagatgttaatcgacaagcttaattacatggttgataagaaaagcaacccatgcgcaacaggcggagccctcaaaggctatcagatcactaacaagttcgaatctttcaaatcgatgagtactcaaaacgggttcatcttttatatcccagcctggctaacctcgaagatagaccctagcacagggtttgtcaacctcctcaaaacgaagtacacatccatcgcagattctaagaaattcatttcctccttcgatcgtatcatgtatgtccccgaggaagatcttttcgaattcgcactggattataaaaacttctcaagaacagacgctgactatatcaagaagtggaaactgtattcgtacgggaacagaattcgaatcttcaggaaccctaaaaagaacaatgtgtttgactgggaagaggtatgcctgacttctgcctataaggaattgttcaataagtacggtataaactatcagcagggagatattagagctctactttgcgaacagtcagataaggcattttacagtagctttatggctctgatgagcctgatgctgcagatgcgcaacagcatcaccgggcggacagatgtggatttcctgatatcacctgtaaagaacagtgatgggatattctatgattcgagaaactatgaggctcaggagaatgccattcttcctaagaacgccgatgctaacggcgcatacaacattgcccggaaagtgctgtgggcaatcggccagttcaaaaaggctgaggacgagaagctggataaggtcaagatagctattagcaataaggaatggttagagtacgcgcagacgtcagttaaacactctgggtcaggaggcagctctggcggatctatggcagaaaacggcgataatgagaagatggcagccctcgaggcgaagatttgtcatcagattgagtactatttcggagactttaacttgccccgggacaaattcctgaaggagcagattaaactggatgagggctgggtgcctctggagattatgattaagtttaatcggcttaaccgcctcactacggatttcaacgtgatcgtcgaggccctgagcaagtctaaagccgaactcatggaaatcagcgaggacaagactaaaatccggcgttctccatccaagccgcttcctgaggtgacagatgaatacaaaaatgacgtgaaaaacagaagcgtatatatcaaagggttccccaccgatgccacactggatgatataaaagagtggttggaggacaaaggacaggtcttgaacattcaaatgaggcgcactttacacaaggccttcaagggtagcatctttgtagtttttgattccattgagtcagctaagaaatttgtggagactcctggtcagaagtacaaagagaccgatctgctgatactatttaaagatgattacttcgctaagaagaacgaaagtggcggtagtaaaagaacagctgatggaagcgagttcgagccaaaaaaaaaacggaaagtg

SEQ ID NO: 298 - DNA, 6570 bp, synthetic construct
  source 1..6570: mol_type = other DNA
atgaaaagaaccgccgacggcagcgaattcgagcctaagaagaagaggaaggtttccggcggcagctcaggaggttccaccctcaacatcgaggatgagtaccgtcttcatgaaacaagcaaggaaccagatgtgagcttgggctcaacatggttgtcagacttccctcaagcatgggctgagactggtggcatgggcctcgccgtccgtcaagctcctctcatcatccctctcaaggccacctccacccccgtctccataaagcagtaccccatgagccaagaagcaaggcttggcatcaagcctcacatccaaaggctccttgatcaaggcatcttggtgccatgtcaatcaccttggaacactcctctcctccccgtcaagaagcctggcaccaacgactaccgtccagttcaagatcttcgagaagtgaacaagagggtggaggacatccatcccaccgtccccaacccttacaacctcctctcaggccttccaccatctcatcaatggtacaccgtccttgacctcaaggatgccttcttctgcctccgcctccatccaacaagccaacctctcttcgccttcgagtggagagatccagagatgggcatctcaggccagctgacatggacaaggcttcctcaaggcttcaagaactcaccaaccctcttcaacgaggctcttcatcgagatctggccgacttcaggatccaacatccagatttgatcctcctccagtacgtggatgacctcctcctcgccgccacctcagagcttgattgtcaacaaggaacaagagctctcctccagacccttggcaatcttggctacagggcctccgccaagaaggctcaaatttgccaaaagcaagtcaagtaccttggctacctcctcaaggaaggccaaagatggttgaccgaggcaaggaaggagacggtgatgggacaaccaacacccaagacaccaagacaactacgtgagttccttggcaaggctggcttctgccgcctcttcatcccaggcttcgccgagatggcagctcctctctaccctttgaccaagcctggcaccctcttcaactggggaccagatcagcagaaggcctaccaagagatcaagcaagctctactcaccgctccagctctcggccttccagatctgaccaagccatttgagctcttcgtggatgagaagcaaggctatgccaagggagtcctcacccagaagctgggaccttggagaaggccggtggcctacctctccaagaagcttgatccagtggccgccggatggccaccatgcttaaggatggtggcagccatcgccgtcctcaccaaggatgctggcaagctcaccatgggacaacccttggtgatccttgctcctcatgctgtggaggccttggtgaagcaacctccagatcgatggttgtcaaatgcaaggatgacccactaccaagctctcctccttgacaccgaccgagtccagtttggaccggtggtggccctcaacccagcaaccctccttcctcttccagaagaaggcctccaacacaactgccttgacatcttggccgaggcccatggaacaagaccagatttgacagatcaacctcttccagatgctgaccacacctggtacactgatggatcaagccttcttcaagaaggacaaaggaaggccggcgccgccgtcaccaccgagactgaggtgatctgggccaaggcccttccagctggcacctccgctcaaagagctgagctcatcgccttgacccaagctctcaagatggcagaaggcaagaagctcaatgtctacaccgactcaaggtatgccttcgccaccgctcacatccatggtgagatctaccgccggcgaggatggttgacaagcgagggcaaggagatcaagaacaaggatgagatcttggccctcctcaaggctctcttccttcccaagaggttgagcatcatccattgtccaggccaccagaagggccattcagctgaagcaagaggcaacaggatggcagatcaagcagcaaggaaggccgccatcaccgagacaccagacacctccaccctcctcatcgagaactcatcaccctccggaggatctagcggaggctcctctggctctgagacacctggcacaagcgagagcgcaacacctgaaagcagcgggggcagcagcggggggtcaggctccagcaagctggagaagttcaccaactgctactccctctcaaagaccctcaggttcaaggccatccctgtggggaagacccaggagaacattgacaacaagaggctgctggtggaggatgagaagagggctgaggactacaagggggtgaagaagctgctggacagatattacctctccttcatcaatgatgtgctgcactcaatcaagctgaagaacctcaacaactacatctccctcttcaggaagaagaccaggacagagaaggagaacaaggagctggagaacctggagatcaacctgaggaaggagattgccaaggccttcaagggcaatgaaggctacaagtcactcttcaagaaggacatcattgaaaccatcctcccagagttcctggatgacaaggatgagattgccctggtgaactccttcaatggcttcaccactgccttcactggcttctttagaaacagggagaacatgttctcagaggaggccaaatcaacaagcattgccttcaggtgcatcaatgagaacctcacccgctacatctccaacatggacatctttgagaaggtggatgccatctttgacaagcatgaggtgcaagagatcaaggagaagatcctgaactcagactatgatgtggaggacttctttgaaggagagttcttcaactttgtgctcacccaagaaggcattgatgtctacaatgccatcattggtggctttgtgacagagtcaggagagaagatcaagggcctcaatgagtacatcaacctctacaaccagaagaccaagcagaagctgcccaagttcaagcccctctacaagcaggtgctctctgacagggagagcttgagcttctatggagaaggctacacctctgatgaggaggtgctggaggtcttcaggaacaccctcaacaagaactcagagatcttctcctccatcaagaagctggagaagctcttcaagaactttgatgaatactcctctgctggcatctttgtgaagaatggccctgccatctccaccatctccaaggacatctttggagagtggaatgtcatcagggacaagtggaatgctgagtatgatgacatccacctaaagaagaaggctgtggtgacagagaagtatgaggatgacaggaggaagagcttcaagaagattggcagcttctccctggagcagctgcaagagtatgctgatgctgacctctctgtggtggagaagctgaaggagatcatcatccagaaggtggatgagatctacaaggtctatggctcctcagagaagctctttgatgctgactttgtgctggagaagagcctcaagaagaatgatgctgtggtggccatcatgaaggacctgctggactcagtgaagagctttgagaactacatcaaggccttctttggagaagggaaggaaaccaacagggatgagagcttctatggagactttgtgctggcctatgacatcctgctgaaggtggaccacatctatgatgccatcaggaactatgtcacccagaagccctactcaaaggacaagttcaagctctacttccagaacccccagttcatgggtggctgggacaaggacaaggagacagactacagggccaccatcctgaggtatggctcaaagtactacttggccatcatggacaagaagtatgccaagtgcctccagaagattgacaaggatgatgtcaatggcaactatgagaagataaactacaagctgctgcctggccccaacaagatgctgcccaaggtcttcttctccaagaagtggatggcctactacaacccctcagaggacatccagaagatctacaagaatggcaccttcaagaagggagacatgttcaacctcaatgactgccacaagctgattgacttcttcaaggactccatctccagataccccaagtggagcaatgcctatgacttcaacttctcagagacagagaagtacaaggacattgctggcttctacagggaggtggaggagcaaggctacaaggtgtcatttgagagtgcctccaagaaggaggtagacaagctggtggaggaagggaagctctacatgttccagatctacaacaaggacttctctgacaagagtcatggcacccccaacctccacaccatgtacttcaagctgctctttgatgagaacaatcatggccagatcaggctctcaggaggagcagagctcttcatgaggagggcctccctcaagaaggaggagctggtggtgcaccctgccaactcccccattgccaacaagaaccctgacaaccccaagaagaccaccaccctctcctatgatgtctacaaggacaagaggttctcagaggaccaatatgagctgcacatccccattgccatcaacaagtgccccaagaacatcttcaagatcaacacagaggtgagggtgctgctgaagcatgatgacaacccctatgtcattggcattgacagaggagagaggaacctcctatacattgtggtggtggatgggaagggcaacattgtggagcagtactccctgaatgagatcatcaacaacttcaatggcatcaggatcaagacagattaccactcactgctggacaagaaggagaaggagaggtttgaggcccgccaaaattggacaagcattgagaacatcaaggagctgaaggctggctacatctcccaggtggtgcacaagatctgtgagctggtggagaagtatgatgctgtgattgccctggaggacctcaactctggcttcaagaactcaagggtgaaggtggagaagcaagtctaccagaagtttgagaagatgctgattgacaagctcaactacatggtggacaagaagagcaacccctgtgccactggtggtgccctcaagggctaccagatcaccaacaagtttgagagcttcaagagcatgagcacccagaatggcttcatcttctacatccctgcttggctcacctccaagattgacccctcaactggctttgtcaacctgctgaagaccaagtacacctccattgctgactccaagaagttcatctcctcctttgacaggatcatgtatgtgccagaggaggacctctttgagtttgccctggactacaagaacttctcaaggactgatgctgactacatcaagaagtggaagctctacagctatggcaacaggataaggatcttcaggaaccccaagaagaacaatgtctttgattgggaggaggtgtgcctcacctctgcctacaaggagctcttcaacaagtatggcatcaactaccagcaaggagacatcagagccctcctctgtgagcaatcagacaaggccttctactcctccttcatggccctgatgagcctgatgctgcagatggccaactccatcactggaaggactgatgtggacttcctcatctccccagtgaagaactctgatggcatcttctatgactcaaggaactatgaggcccaggagaatgccatcctccccaagaatgctgatgccaatggagcctacaacattgcaaggaaggtgctgtgggccattggccagttcaagaaggctgaggatgagaagctggacaaggtgaagattgccatctccaacaaggagtggctggagtatgcccaaacctcagtgaagcactcaggtagtggggggagctccggcggcagcatggcggagaatggggacaacgagaagatggctgccctggaggccaagatctgccaccagattgaatactactttggggacttcaacctaccaagggacaagttcctcaaggagcagatcaagctggatgaggggtgggtgccactggagatcatgatcaagttcaaccgcctcaacaggctcaccacagacttcaacgtgatcgtggaggccctctccaagagcaaggcagagctgatggagatctcagaggacaagaccaagatcaggaggagcccctcaaagcccctgccggaggtgacggatgagtacaagaacgatgtgaagaacaggagtgtctacatcaagggcttccccaccgatgccaccctggatgacatcaaggagtggctggaggacaaggggcaggtgctgaacatccagatgaggaggaccctccacaaagcattcaaggggagcatcttcgtggtgtttgactcaattgagagtgccaagaagttcgtggagactcctggccagaagtacaaggagacagacctgctgatcctcttcaaggatgactactttgccaagaagaacgagagtggggggagcaaaagaaccgccgacggcagcgaattcgagcctaagaagaagaggaaggtttga

SEQ ID NO: 299 - DNA, 6570 bp, synthetic construct
  source 1..6570: mol_type = other DNA
atgaagaggactgctgatggctcagagttcgagcccaagaagaagaggaaggtgagtggggggagctctggggggagcaccctcaacattgaggatgagtacaggctgcatgaaaccagcaaggagccggatgtttcattggggagcacttggctctccgacttccctcaagcatgggctgagactggggggatggggctggcggtgaggcaggctcccctgatcatccccctgaaggccacctccacccctgtttcaatcaagcagtacccaatgagccaggaggcaaggctggggatcaagcctcacatccagaggctgctggaccaggggatcctggtgccttgccaatcaccctggaacactcctctgctgccggtgaagaagcctggcaccaatgattacagaccagtgcaagatctcagggaggtgaacaagagggtggaggacatccacccaacggtgcccaacccttacaacctgctgagtgggctgcctccttcacaccagtggtacactgtgctggacctcaaggatgccttcttctgcctgaggctgcaccccaccagccagcctctctttgcctttgagtggagggatccggagatggggatctctgggcagctaacatggaccaggctgcctcagggcttcaagaacagccccaccctcttcaatgaagctctccacagggatctggctgacttcaggattcaacatccagacctgatcctgctgcagtatgtggatgatctgctgctggctgccacctcagagctggactgccagcagggcaccagggctctgctgcagaccctggggaacctgggctacagggcttcagccaagaaggcccagatctgccagaagcaggtgaagtaccttggctacttgctgaaggaggggcagaggtggctcactgaggcaaggaaggagacggtgatggggcagcccacccccaagacccccaggcagctgagggagttcctggggaaggctggcttctgcagattattcatccctggctttgctgagatggctgctcccctctaccccctcaccaagcctgggaccctcttcaactgggggccagaccagcagaaagcataccaagagatcaagcaagctcttttgactgctcctgccttggggctgcctgatctcaccaagcccttcgagctatttgtggatgagaagcagggctatgccaagggggtgctgacccaaaaacttggtccttggaggaggccggtggcttatctctccaagaaacttgacccggtggctgctggctggcctccctgcctgaggatggtggctgccattgctgtgctcaccaaggatgctgggaagctaacaatggggcagcccctggtgatcctggctccccatgctgtggaagcattggtgaagcagccaccagaccggtggctcagcaatgccaggatgacccattaccaagctcttttgctggacactgacagggtgcagtttgggccggtggtggctttaaaccctgccaccctgctgcccttgccagaggaggggctgcagcacaactgcctggacatcctggctgaggctcatggcaccaggcctgatctcaccgaccagcccctgccggatgctgaccacacctggtacaccgatggaagctcattgctgcaagaggggcagaggaaggctggggctgctgtgaccacagagacagaggtcatttgggccaaggccttgccggctgggacctctgctcagagggctgagctgattgctttgactcaagcattgaagatggctgaggggaagaagctgaatgtctacaccgactcaaggtatgcctttgccactgctcacattcatggggagatctacaggaggaggggatggctcaccagtgaagggaaggagatcaagaacaaggatgagatcctggctctgctgaaagctctcttcctcccaaagaggctctccatcatccactgccctggccaccagaagggccattcagctgaggcaagggggaacaggatggcggatcaggctgccaggaaggctgccatcaccgaaaccccagacacctccaccctgctgattgagaacagcagcccctccggtgggagctctggggggagcagtggctcagaaacccctgggacctcagagagtgccaccccggagagctctggggggagctctggggggagtgggagcagcaagctggagaagttcaccaactgctactctctctcaaagaccctgaggttcaaggccatcccggtggggaagacccaggagaacattgacaacaagaggctgctggtggaggatgagaagagggctgaggattacaagggggtgaagaagctgttggacagatattacctcagcttcatcaacgatgtgctgcattcaatcaagctgaagaacctcaacaactacatctccctcttccggaagaagaccaggacggagaaggagaacaaggagctggagaacctggagatcaacctgaggaaggagattgcaaaggctttcaaggggaatgagggctacaaaagtttgttcaagaaggatattattgaaaccatcctgccggagttcctggatgacaaggatgagattgctttagtgaacagcttcaatggcttcaccactgccttcaccggcttcttccggaaccgggagaacatgttctccgaggaggccaagagcaccagcattgccttcaggtgcatcaacgagaacctgactcgttacatcagcaacatggacatcttcgagaaggtggatgccatcttcgacaagcatgaagttcaagagatcaaggagaagattctcaattcagactatgatgtggaggacttcttcgagggagagttcttcaactttgtgctgacccaggaggggattgatgtctacaatgccatcatcgggggctttgtgacagagagtggggagaagatcaaggggctaaatgagtacatcaacctctacaaccagaagaccaagcagaagctgccaaagttcaagcccctctacaagcaggtgctctccgacagggaaagtttgagcttctatggggaaggctacacttcagatgaggaggtgctggaggtgttcaggaacaccctcaacaagaactcggagatcttctcctccatcaagaagctggaaaaacttttcaagaacttcgatgagtacagctcggctgggatcttcgtgaagaatggccctgccatctccaccatcagcaaggacatctttggggagtggaatgtgatccgggacaagtggaatgccgagtatgatgatattcatttaaagaagaaggctgtggtgacggagaagtatgaggatgacagaaggaagagcttcaagaagattgggagcttctccctggagcagctccaggagtatgctgatgccgacctctcggtggtggagaagctgaaggagatcatcatccagaaggtggatgagatctacaaggtgtatgggagctcagagaagctgtttgatgccgactttgtgctggagaaaagtttgaagaagaatgatgctgtggtggccatcatgaaggatctgctggactcggtgaaaagttttgagaactacatcaaagcattctttggggaggggaaggaaaccaaccgggatgaaagtttttatggggactttgtgctggcctatgacattttattgaaggtggaccacatctatgatgccatcaggaactatgtcacccagaagccctacagcaaggacaagttcaaactttacttccagaaccctcagttcatggggggctgggacaaggacaaggagacagactacagggccaccatcctgaggtatggttcaaagtattacttggccatcatggacaagaagtatgccaagtgcctgcagaagatcgacaaggatgatgtgaatgggaactatgagaagatcaactacaagctgcttcctggacccaacaagatgctgcccaaggtgttcttctccaagaagtggatggcttactacaacccctcagaggacatccagaagatctacaagaatggcaccttcaagaagggggacatgttcaacctcaatgactgccacaagctgatcgacttcttcaaggacagcatctcaagataccccaagtggagcaatgcctatgacttcaacttctcagagacagagaagtacaaggacattgctggcttctacagggaggtggaggagcagggctacaaggtgagctttgagagtgcttcaaagaaggaggtggacaaattggtggaggaagggaaactttacatgttccagatctacaacaaggacttctccgacaaatctcatgggacccccaacctccacaccatgtacttcaagctgctgtttgatgagaacaatcatgggcagatcaggctctccggtggggcggagctcttcatgaggagggcctcattgaagaaggaggagctggtggtgcaccctgccaactctcccattgccaacaagaaccctgacaaccccaagaagaccaccaccctcagctatgatgtctacaaggacaagaggttctcagaggaccaatatgagctgcacattcccattgccatcaacaagtgccccaagaacatcttcaagatcaacaccgaggtgagggtgttgttgaagcatgatgataacccctatgtgattgggatcgacaggggggagaggaacttgttatacattgtggtggtggatgggaaggggaacattgtggagcagtacagcttgaacgagatcatcaacaacttcaatgggatcaggatcaagacagactaccattcattgttggacaagaaggagaaggagaggttcgaggcaaggcagaactggacctccattgagaacatcaaggagctgaaggctggctacatcagccaggtggtgcacaagatctgtgagctggtggagaagtatgatgcggtgattgccctggaggatctcaacagtggcttcaaaaattcaagggtgaaggtggagaagcaggtgtaccagaagttcgagaagatgctgatcgacaagctgaactacatggtggacaagaagagcaacccttgtgccaccggtggagccctgaaaggataccaaatcaccaacaagtttgaaagtttcaagagcatgagcacccagaatggcttcatcttctacatccctgcctggctcaccagcaagatcgacccctcaaccggctttgtcaacctgctgaagaccaagtacacaagcattgctgacagcaagaagttcatcagcagcttcgacaggatcatgtatgtgccagaggaggacctcttcgagtttgccctggactacaagaacttctcaaggactgatgctgattacatcaagaagtggaagctctactcttatgggaacaggataaggattttccggaaccccaagaagaacaatgtcttcgactgggaggaggtgtgcctcacctcagcttacaaggagctcttcaacaagtatgggatcaactaccagcaaggggacatcagggctctgctgtgtgagcagagtgacaaagcattttacagcagcttcatggccctgatgagcctgatgctgcagatggccaacagcatcaccgggaggaccgatgtggacttcctcatctccccggtgaagaactctgatgggatcttctatgactcaaggaactatgaggcccaggagaatgccatcctccccaagaatgctgatgccaatggagcctacaacattgccaggaaggtgctgtgggccattgggcagttcaagaaggctgaggatgagaagctggacaaggtgaagattgccatcagcaacaaggagtggctggagtatgctcagacctcggtgaagcactctgggagtggggggagctctggtggctcaatggctgagaatggggacaacgagaagatggctgccctggaggccaagatctgccaccagattgaatactactttggggacttcaacctgccaagggacaagttcctgaaggagcagatcaaattggatgagggctgggtgcccttggagatcatgatcaagttcaaccggctgaaccggctaacaacagacttcaatgtgattgtggaggccctctccaagagcaaggctgagctgatggagatctccgaggacaagaccaagatccggaggagcccctcaaagcccctgccggaggtcaccgatgagtacaagaatgatgtgaagaaccggagtgtttacatcaagggcttccccaccgatgccaccctggatgacatcaaggagtggctggaggacaaggggcaggtgctgaacatccagatgaggaggaccctccacaaagcattcaaggggagcatctttgtggtgttcgactcaatcgagagtgccaagaagtttgtggaaacccctgggcagaagtacaaggagacagatctgctgatcctcttcaaggatgattactttgccaagaagaatgagagtggggggagcaagaggactgctgatggctcagagttcgagcccaagaagaagaggaaggtgtga

SEQ ID NO: 300 - DNA, 6570 bp, synthetic construct
  source 1..6570: mol_type = other DNA
atgaagaggacggctgatggctcggagttcgagccgaagaagaagaggaaggtgtccggaggaagctccggaggctccaccctcaacatcgaggacgagtaccgtctccacgagacgagcaaggagccggatgtctctcttggctcaacatggctctccgacttccctcaagcatgggctgagactggagggatgggcttggcggtgaggcaagctcctctcatcatccctctcaaagccacctcaactccggtgagcatcaagcagtatccgatgagccaagaggcgcggctggggatcaagcctcacatccagaggctgctggaccaagggatcctggttccctgccaatcaccgtggaacactcctctcctcccggtgaagaagccgggaaccaatgactacagaccggtccaggatctcagagaggtgaacaagagggtggaggacatccatcccaccgtccccaacccctacaacctcctctcaggtcttcctccgagccaccagtggtacacggtgctggatctcaaggatgccttcttctgcttgaggctccatccaacgagccaacctctcttcgcctttgagtggagagatccggagatggggatctcaggacagctcacctggacgaggcttcctcaaggcttcaagaactctccgaccctcttcaacgaagctctccaccgagatctggctgacttccggatccagcatccggatctgatcctcctccagtatgtggatgatctgctgctggctgccacctcggagctggactgccagcaagggacgagagctctcctccagacgctgggaaaccttggctacagagcttcagccaagaaggctcagatctgccagaagcaggtgaagtaccttggttatctcctcaaggaaggccagaggtggctgacggaggcaaggaaggagacggtgatgggacagccaactcctaagacgccaaggcagctgagagagttcctggggaaggctggcttctgcagactcttcatccctggctttgctgagatggctgctcctctctaccctctaacaaagccggggaccctcttcaactggggaccggaccagcagaaggcttaccaggagatcaagcaagctctcctcaccgctccggctctggggcttccggatctcaccaagcccttcgagctcttcgtggatgagaagcaaggctacgccaagggagttttgactcagaagctgggtccttggagaaggccggtggcttacctctcaaagaaacttgacccggtggctgctggctggcctccctgcctccggatggtggctgccatcgccgttctcaccaaggatgctgggaagctgacgatgggacagccgctggtgatcctggctcctcacgctgtggaagctcttgtgaagcagcctccagaccggtggctctcaaacgcgaggatgactcactaccaagctctcctccttgacaccgacagagtgcagtttgggccggtggtggctctcaacccagccactctccttcctctcccggaggaaggactccaacacaactgcttggacatcctagctgaggctcatgggacgaggccggatctcaccgatcagcctctcccggatgctgaccacacctggtacaccgatgggagctctctcctccaggaaggccagaggaaggctggagctgcggtgacgacggagacggaggtgatctgggccaaggctctcccggctgggacctcagctcagagagctgagctgatcgctctcacccaagctctcaagatggcggaagggaagaagctcaacgtctacacagactctcgctatgctttcgccacagctcacattcatggagagatctacaggaggagaggctggctgacctcagaggggaaggagatcaagaacaaggatgagatcctggctctcctcaaggctctcttcctccccaagaggctctccatcatccactgccctggccaccagaagggacactctgctgaggcgagagggaaccggatggcggatcaagctgcaaggaaggctgccatcacggagactccggacacctcaacgctgctgattgagaacagctctccctccggaggaagctccggaggaagctccggctcggagactccgggaacctcagagagtgccacgccggagagctccggaggaagctccggaggctccggaagctccaagctggagaagttcaccaactgctactctctctcaaagacgctgaggttcaaggccatcccggtggggaagacccaggagaacattgacaacaagaggctgctggtggaggacgagaagagagctgaggactacaagggagtgaagaagctcttggacagatactatctctccttcatcaacgatgttctacactccatcaagctgaagaacctcaacaactacatctctctcttccggaagaagacgaggacggagaaggagaacaaggagctggagaacctggagatcaacctgaggaaggagatcgccaaggctttcaaggggaatgaaggctacaagagcctcttcaagaaggacatcatcgagacgatcttaccggagttcctggatgacaaggacgagatcgctctggtgaacagcttcaacggcttcaccacagccttcaccggcttcttcaggaaccgagagaacatgttctcagaagaagccaagagcacctccattgccttcaggtgcatcaacgagaacctgactcgttacatctccaacatggacatcttcgagaaggtggatgccatcttcgacaagcatgaggttcaggagatcaaggagaagatcctcaactcagactacgatgtggaggacttcttcgaaggagagttcttcaacttcgtcctcacccaagaggggattgatgtctacaacgccatcatcggaggcttcgtgacagagtcaggagagaagatcaaggggctcaacgagtacatcaacctctacaaccagaagaccaagcagaagctgccaaagttcaagcctctctacaagcaggtgctctccgacagagagagcttgagcttctacggagaaggctacacttcagatgaggaggtgctggaggtcttcaggaacaccctcaacaagaactcggagatcttctcctccatcaagaagctggagaagctcttcaagaacttcgatgagtacagctctgctgggatcttcgtgaagaacggtccggcgatctccaccatctccaaggacatcttcggagagtggaacgtgatcagagacaagtggaacgcggagtacgatgacattcatctaaagaagaaggcggtggtgacggagaagtacgaggatgaccggaggaagagcttcaagaagattgggagcttctccctggagcagctccaggagtatgctgatgctgatctctcggtggtggagaagctgaaggagatcatcatccagaaggtggacgagatctacaaggtctatgggagctcagagaagctcttcgatgctgacttcgtgctggagaagagcttgaagaagaacgatgctgtggtggccatcatgaaggatctgctggattcagtgaagagcttcgagaactacatcaaggctttcttcggagaagggaaggagacgaacagagatgagagcttctacggagacttcgtcttggcttatgacatcctcctcaaggtggaccacatctatgatgccatcaggaactatgtgacccagaagccttactcaaaggacaagttcaagctctacttccagaaccctcagttcatgggaggctgggacaaggacaaggagacagactacagagccaccattctccggtatggatcaaagtactacttggccatcatggacaagaagtatgccaagtgcctccagaagatcgacaaggatgatgtgaacgggaactacgagaagatcaactacaagctgcttcccggtcccaacaagatgctccccaaggtcttcttctccaagaagtggatggcttactacaacccctcagaggacatccagaagatctacaagaacgggaccttcaagaagggagacatgttcaacctcaatgattgtcacaagctgatcgacttcttcaaggactccatctcaaggtacccaaagtggagcaacgcttatgacttcaacttctcggagacggagaagtacaaggacatcgctggcttctaccgagaggtggaggagcaaggctacaaggtgagctttgagagtgcttcaaagaaggaggtggacaagctggtggaggaagggaagctctacatgttccagatctacaacaaggacttctccgacaagagtcatgggacgccaaacctccacaccatgtacttcaaacttctcttcgatgagaacaatcatggccagatccggctctcaggaggagctgagctcttcatgaggagagcttctctcaagaaggaggagctggtggtccacccagccaactctccgatcgccaacaagaacccggacaaccccaagaagaccaccacgctgagctatgatgtctacaaggacaagaggttctcagaggaccagtacgagctccacatccccatcgccatcaacaagtgtccgaagaacatcttcaagatcaacacggaggtgagggttctcctcaagcatgatgacaacccttatgtgatcgggatcgaccgaggagagaggaatctactctacatcgtggtggtggatgggaaggggaacatcgtggagcagtactctctcaacgagatcatcaacaacttcaatgggatcagaatcaagacagattaccactcactcttggacaagaaggagaaggagaggtttgaggcgaggcagaactggacctccatcgagaacatcaaggagctgaaggctggctacatctcccaggtggtccacaagatctgcgagctggtggagaagtatgatgcggtgatcgctctggaggatctcaactccggcttcaagaactcaagggtgaaggtggagaagcaggtctaccagaagttcgagaagatgctgatcgacaagctcaactacatggtggacaagaagagcaacccttgtgccaccggaggagctctcaaaggttatcagatcaccaacaagtttgagagcttcaagagcatgagcacccagaacggcttcatcttctacatcccggcttggctcacctccaagatcgatccttcaaccggcttcgtcaacctcttgaagaccaagtacacctccattgctgactccaagaagttcatctcctccttcgaccggatcatgtatgttccggaggaggatctcttcgagtttgctctggactacaagaacttctcaaggacagatgctgactacatcaagaagtggaagctctacagctatgggaaccggataagaatcttccggaaccccaagaagaacaacgtcttcgactgggaggaggtctgcctcacctcagcttacaaggagctcttcaacaagtatgggatcaactaccagcaaggagacatcagagctctcctctgtgagcagagcgacaaggctttctactcttctttcatggctctgatgagcttgatgctgcagatggccaactccatcaccgggaggacggatgtggacttcctcatctctccggtgaagaactccgatgggatcttctacgactcaaggaactacgaagctcaggagaacgccatcctccccaagaacgctgatgccaacggagcttacaacatcgcaaggaaggtgctctgggccatcggccagttcaagaaggctgaggacgagaaacttgacaaggtgaagatcgccatctccaacaaggagtggctggagtatgctcagacctcggtgaagcactctggctcaggaggaagctccggaggaagcatggcggagaacggagacaacgagaagatggctgctctggaggccaagatctgccaccagatcgagtactacttcggagacttcaacctaccaagagacaagttcctcaaggagcagatcaagctggatgaaggctgggttccgctggagatcatgatcaagttcaacaggctcaacaggctcaccaccgacttcaacgtgatcgtggaggctctctcaaagagcaaggctgagctgatggagatctccgaggacaagaccaagatcaggagaagcccctccaagcctctcccggaggtcaccgacgagtacaagaacgatgtgaagaacagatcagtctacatcaagggcttcccaacggatgcaacgctggatgacatcaaggagtggctggaggacaagggccaggtgctcaacatccagatgaggaggactctccacaaggctttcaaggggagcatcttcgtggtcttcgactccatcgagtcagccaagaagttcgtggagactcccggccagaagtacaaggagacggatctcctcatcctcttcaaggatgattacttcgccaagaagaacgagagcggaggaagcaagaggacggctgatggctcggagttcgagccgaagaagaagaggaaggtgtga

SEQ ID NO: 301 - DNA, 6570 bp, synthetic construct
  source 1..6570: mol_type = other DNA
atgaagaggacagcagatggctcagagttcgagcccaagaagaagaggaaggtgtcaggaggcagctctggaggcagcacattgaacatcgaggatgagtaccggctgcatgagacaagcaaggagccagatgtcagcctcggcagcacctggctctcagacttccctcaagcatgggcagagacaggaggcatggggctggctgtgcggcaagctcccctcatcatccctcttaaggccaccagcacgccggtgagcatcaagcagtacccaatgagccaagaagcaaggctgggcatcaagccacacatccagaggctgctggatcaaggcatcctggtgccatgccagtcaccttggaacacgccgctgctgccggtgaagaagccgggcaccaatgactaccggccggtgcaagacctcagagaggtgaacaagagggtggaggacatccacccaacagtgcccaacccctacaacctcctctccgggctgccgccaagccaccagtggtacaccgtgctggacctcaaggacgccttcttctgcctgaggcttcatccaacatcacagcctctcttcgcctttgagtggagggatccagagatgggcatctccggccagctcacctggacaaggctgccacaaggcttcaagaactctccaaccctcttcaatgaagctctccaccgggatctggcagacttcaggatccagcacccagatctgatcctccttcaatatgtggatgatctgctgctggcggcaacatcagagctggactgccagcaaggcacaagggcgctgctgcagacactgggcaaccttggctacagggcatcagcaaagaaggcgcagatctgccagaagcaggtgaagtacctgggatatttgctgaaggagggccagaggtggctcactgaagcaaggaaggagacagtgatgggtcagccaacacccaagacaccaaggcagctccgggagttccttggtaaggccggcttctgcagattattcatccccggctttgcagagatggcggcgccgctgtaccccctcaccaagccgggcaccctcttcaactgggggccggaccagcagaaggcatatcaggagatcaagcaggcgctgctgacggcgccggcgctggggctgccagacctcaccaagccattcgagctctttgtggatgagaagcaaggctatgccaagggggtgctaacacagaagctggggccttggaggaggccggtggcatacctcagcaagaaacttgatccagtggctgctggctggccgccgtgcctgaggatggtggccgccatcgccgtgctcaccaaggatgccggcaagctgacaatggggcagccactggtgatcctggctcctcatgctgtggaggcgctggtgaagcagcctcctgacaggtggctctcaaatgcaaggatgacacactaccaagctctgctgctggacaccgaccgggtgcagttcggcccggtggtggctctcaacccggccaccttgctgccgctgccagaagaagggctgcagcacaactgcctggacatcctggcagaagctcatggcaccaggccagacctgacagaccagccactaccagatgctgaccacacctggtacacagatggcagcagcctgctgcaagaaggccagaggaaggctggagctgctgtcaccacagagacagaggtgatctgggccaaggctctgccagctggcacatcagctcaaagagctgagctcatcgccctcacccaagctctcaagatggcagaagggaagaagctcaacgtctacacagacagcagatatgccttcgccacagcacacattcatggggagatctacaggaggagaggctggctcacctcagaaggcaaggagatcaagaacaaggatgagatcctggcgctgctgaaggcgctcttcctccccaagaggctctccatcatccactgccccggccaccagaagggccacagcgccgaggccagaggcaacaggatggcagatcaagcagcaaggaaggccgccatcacagagacgccggacaccagcaccctgctgattgagaacagctcgccatcaggaggcagctctggaggcagcagcggctcagagacgccgggcacatcagaatcggcgacgccggagagctctggaggcagctctggaggcagcggcagcagcaagctggagaagttcaccaactgctacagcctctcaaagaccctgaggttcaaggccatcccagtggggaagacacaggagaacattgacaacaagaggctgctggtggaggatgagaagagggcagaggactacaagggggtgaagaagctgctggacagatactacctctcattcatcaatgatgtgctgcacagcatcaagctgaagaacctcaacaactacatcagcctcttcaggaagaagacaaggacagagaaggagaacaaggagctggagaacctggagatcaacctgaggaaggagatcgccaaggccttcaagggcaatgaaggctacaaatcactcttcaagaaggacatcattgaaaccatcctcccagagttcttggacgacaaggatgagattgctctggtgaacagcttcaatggcttcaccaccgccttcaccggcttcttcaggaaccgggagaacatgttctcagaagaggccaagagcaccagcatcgccttcaggtgcatcaacgagaacctgacacgctacatcagcaacatggacatcttcgagaaggtggacgccatcttcgacaagcatgaagttcaagagatcaaggagaagatcctcaactcagattatgatgtggaggacttctttgaaggagagttcttcaacttcgtgctgacccaagaaggcattgatgtgtacaacgccatcattggaggcttcgtcacagagagtggagagaagatcaagggcctcaacgagtacatcaacctctacaaccagaagaccaagcagaagctgcccaagttcaagccgctctacaagcaggtgctctcagacagagagagcttgagcttctatggagaaggctacacctcagatgaggaggtgctggaggtgttcaggaacaccctcaacaagaactcagagatcttcagcagcatcaagaagctggagaagctcttcaagaactttgatgagtacagcagcgccggcatctttgtgaagaatgggccggccatcagcaccatcagcaaggacatctttggagaatggaatgtcatcagggacaagtggaatgcagagtatgatgacatccatctaaagaagaaggcggtggtgacggagaagtatgaggatgacaggaggaagagcttcaagaagatcggcagcttctccctggagcagctgcaagaatatgctgatgctgacctctccgtggtggagaagctcaaggagatcatcatccagaaggtggatgagatctacaaggtgtatggcagctcagagaagctctttgatgctgactttgtgctggagaagagcttgaagaagaatgatgctgtggtggccatcatgaaggacctgctggacagcgtcaagagcttcgagaactacatcaaggccttctttggagaaggcaaggagacaaacagggatgaatcattctatggagactttgtgctggcatatgacatcctcctcaaggtggaccacatctatgatgccatcaggaactatgtgacacagaagccctacagcaaggacaagttcaagctctacttccagaacccccagttcatgggaggctgggacaaggacaaggagacagactacagggccaccatcctcagatatggatcaaagtactacctggccatcatggacaagaagtatgccaagtgcctccagaagatcgacaaggatgatgtcaatggcaactatgagaagataaactacaagctgctgccggggccaaacaagatgctgcccaaggtgttcttcagcaagaagtggatggcctactacaacccatcagaggacatccagaagatctacaagaatggcaccttcaagaagggcgacatgttcaacctcaatgactgccacaagctgatcgacttcttcaaggacagcatcagcagataccccaaatggagcaatgcatatgacttcaacttctcagagacagagaagtacaaggacatcgccggcttctaccgggaggtggaggagcaaggctacaaggtgagctttgaatcagcaagcaagaaggaggtggacaagctggtggaggagggcaagctctacatgttccagatctacaacaaggacttctccgacaaatctcatggcacacccaacctccacaccatgtacttcaagctgctgtttgatgagaacaatcatggccagatcaggctctctggaggagcagagctcttcatgaggagggcatcattgaagaaggaggagctggtggtgcatccagccaacagccccatcgccaacaagaacccggacaaccccaagaagaccaccaccttgagctatgatgtgtacaaggacaagaggttctcagaggaccaatatgagctgcacatccccatcgccatcaacaagtgccccaagaacatcttcaagatcaacaccgaggtgagggtgctactgaagcatgatgacaacccatatgtcatcggcatcgacagaggagagaggaacctcctctacatcgtggtggtggatggcaagggcaacatcgtggagcagtacagcctgaacgagatcatcaacaacttcaatggcatcaggatcaagacagactaccattcattgctggacaagaaggagaaggagaggtttgaggccaggcagaactggaccagcatcgagaacatcaaggagctgaaggctggctacatcagccaggtggtgcacaagatctgcgagctggtggagaagtatgatgctgtcattgctctggaggacctcaattcaggcttcaagaacagcagggtgaaggtggagaagcaggtgtaccagaagttcgagaagatgctcatcgacaagctcaactacatggtggacaagaaatcaaacccatgcgccacaggaggagctctcaagggctaccagatcaccaacaagtttgagagcttcaagagcatgagcacccagaatggcttcatcttctacatcccggcatggctcaccagcaagattgatccaagcacaggcttcgtcaacctgctgaagaccaagtacacatcaatcgccgacagcaagaagttcatcagcagcttcgacaggatcatgtatgtgccagaagaagacctcttcgagtttgctctggactacaagaacttcagcaggacagatgctgactacatcaagaagtggaagctctactcatatggcaacaggataaggatcttcaggaaccccaagaagaacaatgtgttcgactgggaggaggtgtgcctcacctcggcctacaaggagctcttcaacaagtatggcatcaactaccagcaaggagacatcagggcgctgctgtgtgagcaatcagacaaggccttctacagcagcttcatggcgctgatgagcttgatgctgcagatggccaacagcatcaccgggaggacagatgtggacttcctcatctcgccggtgaagaacagtgatggcatcttctatgacagcaggaactatgaggcacaggagaacgccatcctccccaagaatgctgatgccaatggagcctacaacattgcaaggaaggtgctatgggccatcggccagttcaagaaggcagaggatgagaagctggacaaggtgaagatcgccatcagcaacaaggagtggctggagtatgctcagacatcggtgaagcacagtggctctggaggcagctctggaggctcaatggcggagaatggcgacaacgagaagatggcggcgctggaggccaagatctgccaccagattgagtactactttggagacttcaacctaccaagggacaagttcctcaaggagcagatcaagctggatgaaggctgggtgccgctggagatcatgatcaagttcaacaggctcaacaggctcaccacagacttcaacgtcatcgtggaggcgctcagcaagagcaaggcggagctgatggagatctcagaagacaagaccaagatcaggagaagcccaagcaagccgctgccggaggtgacagatgagtacaagaatgatgtcaagaacagatcagtgtacatcaagggcttccccacagatgccaccctggacgacatcaaggagtggctggaggacaagggccaggtgctcaacatccagatgaggaggacgctgcacaaggccttcaagggcagcatcttcgtggtgttcgacagcattgaatcagccaagaagttcgtggagacgccggggcagaagtacaaggagacagacctcctcatcctcttcaaggatgattacttcgccaagaagaacgagagtggaggcagcaagaggacagcagatggctcagagttcgagcccaagaagaagaggaaggtgtga

SEQ ID NO: 302 - DNA, 6570 bp, synthetic construct
  source 1..6570: mol_type = other DNA
atgaagaggaccgccgacggcagcgagttcgagcccaagaagaagaggaaggtgtccggcggcagctcaggaggctccaccctcaacatcgaggacgagtaccgcctccatgagacaagcaaggagccagatgtgagcctcggcagcacctggctctccgacttccctcaagcatgggcagagactggtggcatgggcctcgccgtccgtcaagctcccctcatcatccccctcaaggccacctcaacccccgtctccatcaagcagtaccccatgagccaagaagcaaggctgggcatcaagccccacatccagcgcctccttgaccaaggcatcctggtgccatgccaatcaccctggaacacccccctcctccccgtcaagaagcccggcaccaacgactaccggccggtgcaagacctccgcgaggtgaacaagagggtggaggacatccaccccaccgtccccaacccctacaacctcctctcaggcctcccaccaagccaccagtggtacaccgtccttgacctcaaggatgccttcttctgcctccgcctccaccccaccagccaacccctcttcgccttcgagtggagagatccagagatgggcatctcaggccagctgacctggacaaggcttcctcaaggcttcaagaacagccccaccctcttcaacgaggccctccaccgcgacctcgccgacttcaggatccagcaccctgacctgatcctcctccagtacgtcgacgacctcctcctcgccgccacctccgagctggactgccaacaaggaacaagagctctactccagaccctgggtaacctcggctaccgcgcctccgccaagaaggcccagatctgccagaagcaggtcaagtacctcggctacctcctcaaggaaggccagaggtggctcaccgaggcaaggaaggagacggtgatgggccagcccacccccaagaccccaaggcaactacgtgagttcctcggcaaggccggcttctgccgcctcttcatccccggcttcgccgagatggcggcgccgctctaccctctaacaaagcccggcaccctcttcaactgggggccggaccagcagaaggcctaccaggagatcaagcaagctctcctcaccgcgccggccctcggcctccccgacctcaccaagccctttgagctcttcgtcgacgagaagcaaggctacgccaagggcgtcctcacccagaagctaggaccctggaggaggccggtggcctacctctccaagaagcttgaccccgtcgccgccggctggccaccatgcttgaggatggtggccgccatcgccgtcctcaccaaggacgccggcaagctgacaatgggccaacccctcgtcatcctagctcctcatgccgtggaggccctcgtcaagcagcctcctgaccggtggctctccaatgcaaggatgacccactaccaagctctcctcctcgacaccgaccgcgtccagttcggcccggtggtggccctcaacccggccaccctcctacccctcccagaagaaggcctccagcacaactgcctcgacatcctcgccgaggctcatggcaccaggccagacctgaccgaccagcccctcccagatgctgaccacacctggtacaccgacggctcaagcctccttcaagaaggccagaggaaggccggcgccgccgtcaccaccgagacggaggtgatctgggccaaggccctccccgccggcacctccgcccagcgagctgagctcatcgccctcacccaagctctcaagatggccgagggcaagaagctcaacgtctacaccgactcaaggtacgccttcgccaccgcccacatccacggtgagatctaccgccggcgaggatggctgacctccgagggcaaggagatcaagaacaaggacgagatcctcgccctcctcaaggccctcttcctccccaagaggctctccatcatccactgccccggccaccagaagggccactcagctgaggcaagaggcaacaggatggcagatcaagcagcaaggaaggccgccatcaccgagacgccggacacctccaccctcctgatcgagaacagctcaccctccggcggcagctccggcggcagctccggcagcgagacaccaggcacctccgagtccgccacccctgagagctccggcggcagctccggcggcagcggctcaagcaagctggagaagttcaccaactgctactccttgagcaagaccctccgcttcaaggccatccccgtcggcaagacccaggagaacatcgacaacaagaggctgctggtggaggatgagaagagggccgaggactacaagggcgtcaagaagctactggaccggtactacctgagcttcatcaacgacgtcctccactccatcaagctcaagaacctcaacaactacatctccctcttcaggaagaagacaaggactgagaaggagaacaaggagctggagaacctggagatcaacctaaggaaggagatcgccaaggccttcaagggcaacgagggctacaagagcctcttcaagaaggacatcatcgagacgatcctccccgagttcctcgacgacaaggatgagatcgccctggtgaacagcttcaacggcttcaccaccgccttcaccggcttcttcaggaacagggagaacatgttctccgaggaggccaagagcacctcaatcgccttcaggtgcatcaacgagaacctgacccgctacatctccaacatggacatcttcgagaaggtggacgccatcttcgacaagcatgaggttcaagagatcaaggagaagatcctcaactcagactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgggagagcttgagcttctacggcgagggctacacctcagatgaggaggtgctggaggtcttcaggaacaccctcaacaagaactccgagatcttctcaagcatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccatctcaagaagaaggccgtggtgaccgagaagtacgaggatgacaggaggaagagcttcaagaagatcggcagcttctcactggagcagctccaggagtacgccgacgccgacctctccgtggtggagaagctgaaggagatcatcatccagaaggtggatgagatctacaaggtgtacggcagctccgagaagctcttcgacgccgacttcgtcctggagaaatcactcaagaagaacgacgccgtggtggccatcatgaaggacctccttgacagcgtcaagagcttcgagaactacatcaaggccttcttcggcgagggcaaggagacaaaccgcgacgagagcttctacggcgacttcgtcctcgcctacgacatcctcctcaaggtggaccacatctatgatgccatcaggaactacgtgacccagaagccctactcaaaggacaagttcaagctctacttccagaaccctcagttcatgggaggctgggacaaggacaaggagacggactaccgcgccaccatcctccgctacggcagcaagtactacctcgccatcatggacaagaagtacgccaagtgcctacagaagatcgacaaggatgatgtgaacggcaactacgagaagatcaactacaagctcctccccggccccaacaagatgctccccaaggtcttcttctccaagaagtggatggcctactacaacccttcagaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcaagcctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggttctactcaagcatgatgacaacccctacgtcatcggcatcgaccgaggagagaggaacctcctctacatcgtggtggtggatggcaagggcaacatcgtggagcagtactccttgaacgagatcatcaacaacttcaacggcatcaggatcaagaccgactaccactcactccttgacaagaaggagaaggagaggttcgaggcaaggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccttgcgccaccggcggcgccctcaagggctaccagatcaccaacaagtttgagagcttcaagagcatgtcaacccagaacggcttcatcttctacatcccggcatggctcacctccaagatcgacccctccaccggcttcgtcaacctcctcaagaccaagtacacctccatagctgacagcaagaagttcatctcctccttcgaccgcatcatgtatgttccagaagaagatctcttcgagttcgccctcgactacaagaacttctcaaggacagatgctgactacatcaagaagtggaagctctacagctacggcaacaggatcaggatcttcaggaaccccaagaagaacaacgtcttcgactgggaggaggtgtgcttgacctccgcctacaaggagctcttcaacaagtacggcatcaactaccagcaaggagacatcagggccttgctatgtgagcagagcgacaaggccttctactcctccttcatggccttgatgagcttgatgctccagatggccaacagcatcaccggccgcaccgacgtcgacttcctcatctccccggtgaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctccaacaaggagtggctggagtacgctcagacctccgtcaagcactccggcagcggcggcagctcaggaggaagcatggctgagaacggcgacaacgagaagatggccgccctagaggccaagatctgccaccagatcgagtactacttcggcgacttcaacctaccaagggacaagttcctcaaggagcagatcaagctggatgaaggctgggtgccgctggagatcatgatcaagttcaaccgcctcaaccgcctcaccaccgacttcaacgtcatcgtggaggccctctccaagagcaaggccgagctcatggagatctccgaggacaagaccaagatccgccgctcaccctccaagcccctccctgaggtgaccgacgagtacaagaacgacgtcaagaacaggagcgtctacatcaagggcttccccaccgacgccaccctggatgacatcaaggagtggctggaggacaagggccaagtcctcaacatccagatgaggaggaccctccacaaggccttcaagggcagcatcttcgtcgtcttcgacagcatcgagtcagccaagaagttcgtggagacaccaggccagaagtacaaggagacggacctcctcatcctcttcaaggatgactacttcgccaagaagaacgagtccggcggcagcaagaggaccgccgacggcagcgagttcgagcccaagaagaagaggaaggtgtga

SEQ ID NO: 303 - DNA, 6570 bp, synthetic construct
  source 1..6570: mol_type = other DNA
atgaagaggacggcggatggcagcgagttcgagcccaagaagaagaggaaggtgagcggcggcagcagcggcggcagcaccctcaacatcgaggacgagtaccgcctccacgagacgagcaaggagcctgatgtcagcctcggcagcacctggctctcagatttccctcaggcctgggcggagactggcggcatgggcctcgccgtccgccaggcgccgctcatcatcccgctgaaggccaccagcacgcctgttagcatcaagcagtaccccatgagccaggaggcgcggctgggcatcaagccgcacatccagcggctgctggatcagggcatcctggtgccctgccagtcaccttggaacacgccgctgctgccggtgaagaagcccggcaccaatgactaccggccggtgcaagacctcagggaggtgaacaagagggtggaggacatccaccccaccgtgcccaacccctacaacctgctctcagggctgccgccgagccaccagtggtacaccgtgctggacctcaaggacgccttcttctgcctccgcctccaccccaccagccagccgctcttcgcctttgagtggcgcgacccggagatgggcatctcagggcagctgacatggacgcggctgcctcagggcttcaagaactcgccgacgctcttcaacgaggcgctgcaccgcgacctcgccgacttcaggatccagcaccccgacctgatcctgctgcagtatgtggatgacctgctgctggccgccacctcagagctggactgccagcagggcacgcgcgcgctgctgcagacgctgggcaacctgggctaccgcgcctccgccaagaaggctcaaatttgccaaaagcaggtgaagtacctgggctacctgctgaaggagggccagcgctggctcactgaggcaaggaaggagacggtgatgggccagccgacgcccaagacgccgcggcagctacgagagttcctcggcaaggccggcttctgccgcctcttcatccccggcttcgctgagatggcggcgccgctctacccgctcaccaagcccggcaccctcttcaactgggggccggaccagcagaaggcctaccaggagatcaagcaggcgctgctgacggcgccggcgctggggctgccggacctcaccaagcccttcgagctatttgttgatgagaagcagggctatgccaagggcgtgctcacccagaagctggggccgtggcggcggccggtggcctacctctccaagaaacttgatccggtggctgctggctggccgccgtgcctgaggatggtggccgccatcgccgtgctcaccaaggacgccggcaagctgacaatgggccagccgctggtgatcctagctccccacgccgtggaggcgctggtgaagcagccgccggaccgctggctgagcaatgccaggatgacccactaccaggcgctgctgctggacactgacagggtgcagttcggcccggtggtggcgctcaacccggcgacgctgctgccgctgccggaggaggggctgcagcacaactgcctggacatcctagcagaggctcatggcaccaggccggacctcaccgaccagccgctgcctgatgccgaccacacctggtacaccgacggcagcagcctcctccaggaaggccagaggaaggccggcgccgccgtcaccaccgagacggaggtgatctgggccaaggcgctgcccgccggcacctcagctcagcgcgccgagctcatcgcgctcacccaggcgctgaagatggctgaaggcaagaagctcaacgtctacaccgacagcagatatgccttcgccaccgcccacatccacggcgagatctacaggaggagaggctggctcacctcagagggcaaggagatcaagaacaaggacgagatcctggcgctgctgaaggcgctcttcctccccaagcgcctctccatcatccactgccccggccaccagaagggccattcagctgaggcgcgcggcaacaggatggcggatcaggcggcaaggaaggccgccatcaccgagacgccggacaccagcacgctgctgatcgagaacagctcgccaagcggcggcagcagcggcggcagcagcggctcagagacgccgggcacctcggagagcgccacgccggagagcagcggcggcagcagcggcggcagcggcagcagcaaacttgagaagttcaccaactgctacagcctctcaaagacgctgaggttcaaggccatccccgtcggcaagacccaggagaacatcgacaacaagcggctgctggtggaggacgagaagcgcgccgaggactacaagggcgtcaagaagctgctggaccgctactacctctccttcatcaatgatgtgctgcattcaatcaagctgaagaacctcaacaactacatcagcctcttcaggaagaagacgaggacggagaaggagaacaaggagctggagaacctggagatcaacctccgcaaggagatcgccaaggccttcaagggcaatgagggctacaagagcctcttcaagaaggacatcattgaaaccatcctaccagagttcctggacgacaaggatgagatcgcgctggtgaacagcttcaacggcttcaccaccgccttcaccggcttcttcaggaacagggagaacatgttctcagaggaggccaagagcaccagcatcgccttccgctgcatcaacgagaacctgacgcgctacatcagcaacatggacatcttcgagaaggtggacgccatcttcgacaagcatgaagttcaggagatcaaggagaagatcctcaactcagactacgacgtggaggacttcttcgagggcgagttcttcaacttcgtgctgacccaggagggcattgatgtctacaacgccatcatcggcggcttcgtcaccgagtcaggggagaagatcaagggcctcaacgagtacatcaacctctacaaccagaagaccaagcagaagctgcccaagttcaagccgctctacaagcaggtgctctccgaccgggagagcttgagcttctatggagagggctacaccagcgatgaggaggtgctggaggtgttcaggaacaccctcaacaagaactcagagatcttcagcagcatcaagaagctggagaagctcttcaagaacttcgacgagtacagcagcgccggcatcttcgtgaagaacggccccgccatctccaccatcagcaaggacatcttcggcgagtggaacgtcatccgcgacaagtggaatgctgagtatgatgacatccatctaaagaagaaggcggtggtgacggagaagtacgaggacgacaggaggaagagcttcaagaagattggcagcttctccctggagcagctccaggagtatgctgatgccgacctctccgtggtggagaagctgaaggagatcatcatccagaaggtggacgagatctacaaggtgtacggcagctcagagaagctcttcgacgccgacttcgtgctggagaagagcctcaagaagaatgatgccgtggtggccatcatgaaggacctgctggattcagtgaagagcttcgagaactacatcaaggccttcttcggcgagggcaaggagacaaacagggatgagagcttctatggagacttcgtgctggcctatgacatcctgctgaaggtggaccacatctatgacgccatcaggaactacgtcacccagaagccctactcaaaggacaagttcaagctctacttccagaacccgcagttcatgggcggctgggacaaggacaaggagacggactaccgcgccaccatcctgagatatggatcaaagtactacctcgccatcatggacaagaagtacgccaagtgcctccagaagatcgacaaggatgatgtgaacggcaactacgagaagatcaactacaagctgctgcccggccccaacaagatgctgcccaaggtgttcttcagcaagaagtggatggcctactacaacccctcagaggacatccagaagatctacaagaacggcaccttcaagaagggcgacatgttcaacctcaatgactgccacaagctgatcgacttcttcaaggacagcatcagcagataccccaagtggagcaacgcctatgacttcaacttctcagagacggagaagtacaaggacatcgccggcttctaccgcgaggtggaggagcagggctacaaggtgagcttcgagagcgcctcaaagaaggaggtggacaagctggtggaggagggcaagctctacatgttccagatctacaacaaggacttctccgacaagagccacggcacgcccaacctccacaccatgtacttcaagctgctgtttgatgagaacaatcatggccagatccgcctctcaggaggcgccgagctcttcatgaggagggccagcttgaagaaggaggagctggtggtgcatcccgccaacagccccatcgccaacaagaacccggacaaccccaagaagaccaccaccctcagctacgacgtctacaaggacaagcgcttctcagaggaccagtatgagctgcacatccccatcgccatcaacaagtgccccaagaacatcttcaagatcaacaccgaggtgcgcgtgctactgaagcatgatgacaacccatatgtcatcggcatcgaccgcggcgagaggaacctgctctacatcgtggtggtggacggcaagggcaacatcgtggagcagtacagcttgaacgagatcatcaacaacttcaacggcatccgcatcaagacggactaccattcactgctggacaagaaggagaaggagcgctttgaggcgcggcagaactggacctccatcgagaacatcaaggagctgaaggccggctacatcagccaggtggtgcacaagatctgcgagctggtggagaagtatgatgccgtcatcgcgctggaggacctcaactcaggcttcaagaacagccgcgtcaaggtggagaagcaggtgtaccagaagtttgagaagatgctgatcgacaagctgaactacatggtggacaagaaatcaaacccctgcgccaccggcggcgcgctgaagggctaccagatcaccaacaagttcgagagcttcaagagcatgagcacccagaacggcttcatcttctacatccccgcctggctcaccagcaagattgatccctcaactggcttcgtcaacctgctgaagaccaagtacaccagcatcgccgacagcaagaagttcatcagcagcttcgaccgcatcatgtacgtgccagaggaggacctcttcgagttcgcgctggactacaagaacttctcaaggacggatgctgactacatcaagaagtggaagctctacagctacggcaacaggataaggatcttcaggaaccccaagaagaacaacgtctttgattgggaggaggtctgcctcacctccgcctacaaggagctcttcaacaagtatggcatcaactaccagcagggcgacatccgcgcgctgctctgcgagcaatcagacaaggccttctacagcagcttcatggcgctgatgagcctgatgctgcagatggccaacagcatcaccggcagaactgatgtggacttcctcatctcgccggtgaagaacagcgacggcatcttctatgactcaaggaactacgaggcgcaggagaacgccatcctccccaagaatgctgatgccaacggcgcctacaacatcgccaggaaggtgctctgggccatcggccagttcaagaaggcggaggatgagaagctggacaaggtgaagatcgccatcagcaacaaggagtggctggagtatgctcagacctccgtgaagcattcaggcagcggcggcagcagcggcggcagcatggcggagaatggcgacaatgagaagatggcggcgctggaggccaagatctgccaccagatcgagtactacttcggcgacttcaacctacctcgcgacaagttcctcaaggagcagatcaagctggatgagggctgggtgccgctggagatcatgatcaagttcaaccgcctcaaccgcctcaccaccgacttcaacgtcatcgtggaggcgctcagcaagagcaaggcggagctgatggagatctcagaggacaagaccaagatcaggaggagccccagcaagccgctgccggaggtcaccgacgagtacaagaatgatgtgaagaaccgctcagtgtacatcaagggcttccccaccgacgccaccctggatgacatcaaggagtggctggaggacaagggccaggtgctgaacatccagatgaggaggacgctgcacaaggccttcaagggcagcatcttcgtcgtcttcgacagcatcgagtcagccaagaagttcgtggagacgccgggccagaagtacaaggagacggacctgctgatcctcttcaaggacgactacttcgccaagaagaatgagagcggcggcagcaagaggacggcggatggcagcgagttcgagcccaagaagaagaggaaggtgtga

SEQ ID NO: 304 - DNA, 6570 bp, synthetic construct
  source 1..6570: mol_type = other DNA
atgaaacgtacggcggatggttcagaatttgaaccgaaaaaaaaacgtaaggttagcggaggatcctcgggaggatcaaccctaaacattgaggacgaatatcgcttgcatgaaacgtctaaagaacctgacgtttcacttggctcgacctggttaagcgactttccgcaggcgtgggctgagacaggaggaatgggactcgcggtgcgtcaagctccgcttatcattcctcttaaagcaacaagcacaccagtatcaattaagcaatatccgatgtcacaagaagctagactcggcatcaaacctcatattcaacgacttttggatcagggcatcctggttccctgccagagcccttggaatacgccacttcttcctgtaaaaaaacctggcacaaacgattaccgcccggtgcaggatctcagggaagtgaataaacgcgttgaagatattcatccgacagttccgaacccttataatttattaagcggtttgccgccgtcccatcaatggtacacagttcttgatttaaaagatgccttcttttgcctacgtctgcatccgactagtcagcctttgtttgcgttcgagtggagggaccccgaaatgggcatctcgggccagttaacgtggacccgtctgcctcagggatttaaaaactcacccacgctgtttaacgaggccctgcatcgagatctcgcagatttcagaatccagcatcctgatcttatccttcttcagtacgtcgacgacttattattggccgcaacgtcggaattagattgtcaacagggaactagagcgcttttacaaactttaggaaatcttggctatcgcgcgtctgcgaagaaagcgcaaatttgtcaaaagcaggtcaaatatctgggctacttattaaaagaaggacagagatggctgacagaggcgcggaaagaaacggttatgggtcaaccgacaccgaaaactccgcgccaattgagggaattcctgggtaaagctggcttttgtcgtctgtttattccaggctttgcagagatggcagcgcccctttatccgttgaccaagccgggcacattatttaactggggcccggatcaacaaaaggcttatcaagaaattaaacaagccttgttaaccgcacctgcgttgggattgccggacctgacgaaaccatttgagctttttgttgatgaaaaacaaggctacgcaaaaggagttcttacccaaaaacttggcccatggcgccggcccgtagcctacttgtccaagaaactggatccggtcgcggctggctggccaccttgtctccgaatggtagcagctattgcggttttgactaaagatgccggcaaactcacgatgggccagcctctcgtcatcttagcgccgcacgccgtggaggctctcgtcaagcagccgccagatcggtggcttagcaacgccaggatgacccattaccaggcgcttctgctcgatacagaccgcgtccaatttggcccagtagtggcacttaatcccgcgacgctgctgccacttccagaagagggcttacaacacaattgcctggatattttggcagaagcacatggaacccggccagatctgacagaccagcctttgcctgatgctgatcatacctggtacacagatggatcttcccttctccaagaaggccaaaggaaggctggtgctgcggtaacaacagaaacagaagtgatatgggcaaaagcactcccggcaggaacgagcgcgcagagggcagaacttatcgcactcacacaggcactgaagatggccgagggcaaaaaacttaatgtgtatacggattcacggtatgcctttgctacagctcatatccacggtgaaatctatcggcgtagaggttggctgacatccgaaggcaaggaaattaaaaacaaagatgaaattctggccttactgaaagcattgtttctaccgaaacggctgtcgattatacactgcccgggacatcaaaagggccactctgcggaggcaagaggtaaccgaatggcggatcaagcggctagaaaagctgcaatcacagagacaccggatactagtacacttttgattgaaaactcttcaccgagcggaggatcaagcggcggctcgtctggcagtgagacaccgggaacgtctgaatcagcaacaccagagtcgtctggtggctcatcaggtggctcgggatcttcaaagcttgagaaattcacgaactgttacagtttgtctaaaacattaagatttaaagccatccctgtaggcaaaacccaggaaaacattgataataagcgtctgttagtggaagatgagaaacgcgccgaagattataaaggcgtgaaaaaacttttagatcgctattatttgagctttattaatgacgtcctgcatagcataaaactaaaaaatttgaacaactatatctcgttatttcggaaaaaaacgcgcacggaaaaagaaaataaagaattagaaaacttggaaattaaccttcgaaaagaaatcgcgaaagcatttaaaggcaacgaaggttataagagcttgtttaaaaaggacataattgaaacaattttacctgaatttttagacgataaggatgagattgcgttagtgaactcctttaacggttttactactgccttcactggctttttccgcaatcgcgagaacatgttttctgaggaggctaaaagcacttccattgcttttcgctgtattaatgaaaatcttaccagatatatttccaacatggatatttttgaaaaagtggatgcgatttttgataaacatgaggtgcaagagatcaaagaaaaaattctgaattcagattacgatgtggaagatttctttgaaggcgaattctttaatttcgtacttacgcaagaaggaattgacgtctataatgctattattggcggtttcgtgacagaatccggtgagaaaattaaaggtctcaacgaatatatcaatctgtacaaccaaaaaacaaaacaaaagcttcctaagtttaagccgttgtataagcaggtactgtctgatagggaatcgctgtctttctacggcgaaggatatacctctgacgaggaagttttagaagtctttcgtaatacgcttaataaaaatagtgagattttcagcagcatcaagaagctggaaaaactcttcaaaaattttgatgaatattcttcagcgggaatctttgtaaaaaacggtccggcaatctcaactatctccaaagacatatttggcgagtggaatgtcataagggataaatggaatgctgaatatgatgacatccatttgaaaaaaaaggctgtagttacagaaaaatacgaagacgaccgaagaaagtcatttaagaaaatcggttcattcagcttagagcaattacaagagtacgcagacgccgatcttagtgtcgttgaaaaacttaaagagattataatccagaaagtagatgagatctataaagtatatggatcctcagagaagctttttgatgcggatttcgtgctggaaaagtcgctcaagaagaatgacgcggttgttgctatcatgaaagatctgctggacagtgtaaagtcttttgagaactatatcaaggccttttttggcgaaggtaaagagacaaacagagacgaatctttttatggcgacttcgtgctggcatacgatattcttcttaaagtcgatcatatttatgatgccatacggaactacgtcacacaaaaaccatattcaaaagataaatttaaattgtattttcaaaacccgcaattcatgggcggctgggataaagataaggagacggactacagagctacaattttaagatacggcagcaagtattatctcgccatcatggataaaaagtacgcgaaatgccttcagaaaatcgacaaggacgacgttaatggaaattacgagaaaattaactacaaactcctgcctggcccgaataaaatgttgccgaaggtctttttctccaagaaatggatggcctattataatccgtctgaagacatccaaaagatttataaaaatggcacatttaaaaagggagacatgtttaatcttaatgactgccacaaacttattgacttttttaaagactctatatctagatatccgaaatggagcaatgcttacgactttaacttttctgaaacagagaagtataaggatattgctggtttttatcgcgaagtcgaagaacagggctacaaagtctcctttgaatccgccagtaaaaaggaagtggataaattggtagaggaaggaaaactttacatgttccagatctataacaaagatttttcagataaatcacatggcactcctaatttacatactatgtatttcaagcttttattcgatgaaaataaccacggacaaattcgtcttagcggaggcgcagaattattcatgcgtagagcgtccctgaaaaaggaggaactggttgtacaccctgctaactcaccgattgccaataaaaatcctgacaaccctaaaaaaacgacaacattaagctatgatgtatataaagacaagcgttttagcgaagaccaatatgagctgcacattccaatagcaattaataagtgtccgaagaacatcttcaaaattaatactgaagtccgcgtattacttaagcatgacgataatccttatgtgatcggcatcgatcgaggtgagagaaacttgctgtatatcgtggtggttgatggaaaaggcaatatcgtcgagcagtactcccttaatgaaataattaataattttaacggcatcagaattaaaacagattaccattcattgctcgataaaaaagaaaaggaaagattcgaagcgcgacaaaattggacatcaattgaaaatattaaggaattgaaagccggttatattagtcaagtggttcataaaatttgtgaacttgtagaaaaatatgatgctgtcattgcactcgaagatctgaattccggatttaaaaatagtagagtcaaagttgaaaaacaggtgtatcaaaagtttgaaaagatgttaattgacaaattaaactacatggtcgacaaaaagtcgaatccgtgcgcaactggaggcgctcttaaaggctatcaaattacaaataagtttgagtcattcaagtcgatgagcacgcagaatggatttattttttacatcccggcgtggcttacttctaaaatcgatccatctacgggcttcgtcaatttattaaaaacaaaatatacaagcattgctgactctaaaaaatttattagtagttttgacagaatcatgtacgtgccagaggaagatttgtttgaattcgcacttgattataaaaacttttctagaaccgatgctgactacataaagaaatggaaattgtattcatacggtaaccggatacggatctttcggaacccgaaaaaaaataatgtctttgattgggaagaagtctgcctcacgagcgcttacaaggaactatttaacaagtatggtatcaattatcaacagggcgacatccgcgctctactgtgcgaacaaagtgacaaggcattttatagttcgttcatggcactaatgtccctgatgttacagatggcaaattctattacaggccggactgatgttgatttcctaattagccctgttaaaaatagcgatggaatcttctatgattctcgaaactacgaagcacaggagaatgcaattcttccaaaaaatgcagacgcgaatggcgcctataacattgctcgtaaggtgctgtgggccataggtcagtttaaaaaagccgaggatgaaaaattagacaaagtcaaaatagcaatctcaaacaaagagtggctggaatacgcccagaccagcgttaaacacagcggctctggaggcagcagtggcggctccatggcggagaacggcgacaatgaaaagatggcagcgctggaagcgaaaatttgccatcagattgaatattatttcggagattttaatctacctcgcgataagttcttgaaggaacagatcaaactggacgagggttgggttcccctggaaatcatgatcaaatttaaccgtctcaatagactgacgaccgattttaacgtgatcgtcgaagccctgtcaaaaagcaaagctgaactaatggagatatctgaagataaaaccaaaatacgccggtcaccgagcaagccacttcctgaagttacggatgagtataaaaacgacgtcaaaaacagaagcgtttatatcaaaggctttccgacggatgcgacactggatgacattaaagaatggttggaagataaaggacaggttctgaatattcagatgcgtcgtaccctccacaaagcatttaagggaagcattttcgtggttttcgattcgattgaatcagctaaaaaattcgtggaaacgcccggacagaagtataaagagacggatctcctgattttatttaaagatgattattttgctaaaaaaaacgagtctggcggctcaaaaagaacagccgatggatcagaatttgaaccgaagaagaaacgcaaagtttaa

SEQ ID NO: 305 - DNA, 6570 bp, synthetic construct
  source 1..6570: mol_type = other DNA
atgaagcggaccgcagacgggtcggaattcgaaccgaagaagaagcgcaaggtctcgggcggctcctcgggtggatcgaccctcaacatcgaagatgagtaccgcctgcacgagacgtcgaaggaaccggatgtctccctcggctcgacctggctctcggacttcccgcaggcgtgggcggaaacgggcgggatgggtttggcagtgcgccaggcgccgctcatcatcccgcttaaggcgacctcgaccccggttagcatcaagcagtatcccatgtcccaggaggcgcggctcggcatcaagccgcacatccagcgactcctggatcagggtatcctggtcccctgccagtcgccttggaacacccccctcttgcccgtgaagaagccgggcacgaatgattaccgccccgttcaagatctccgggaggtcaataagcgggtggaagacatccaccccaccgtgccgaatccgtacaacctgctcagtggcctgccgccctcgcaccagtggtacacagtcctcgatctgaaggacgctttcttctgcctgcgcctccacccgacctcgcagcccctcttcgccttcgaatggcgtgaccccgagatgggaatctcgggacagctcacctggacgcggctcccccagggcttcaagaactccccgaccctgttcaatgaggcgctccatagggacctagccgacttccgcatccagcaccccgacctcatcctcctgcagtacgtcgacgacctgctgctggccgcgaccagcgagctcgactgccagcagggcacgcgcgcgctgctgcagaccctgggcaacctgggataccgcgcatcggccaagaaggcccagatctgccagaagcaggtgaagtacttggggtacctgctcaaggagggccagcgctggctcaccgaggcgcgcaaggagacggtcatgggacagcctaccccgaagaccccgcgccagctccgcgagttcctgggcaaggccggcttctgccggctgttcatcccgggcttcgccgaaatggcggccccgctgtacccgttgaccaagcccgggactctcttcaactggggcccggaccagcaaaaggcctaccaggagatcaaacaggctctcctcaccgcccccgccctcggcctcccggacctcacgaaaccgttcgagctcttcgtcgacgagaagcagggctacgcaaaaggggtgctgacccagaagctgggcccgtggcggcggccggtggcctacctctctaagaagctggacccggtggcggccggatggccgccgtgtctccggatggtggccgccatagcggtgctcaccaaggacgccggcaagctcaccatgggtcagccgctggtcatcctggccccccacgcggtcgaagccctcgtcaagcagccccctgaccgctggctctcgaacgcacgaatgacccactaccaagccctgctgctcgacacggaccgtgtccaattcggcccggtcgtcgcactgaatcccgccaccctgctcccgctgcccgaggagggcctgcagcacaattgcctcgacatcctggccgaggcacacggcacgcggccggacctgacagaccaacccctgcccgacgccgaccacacttggtataccgacggttcctcgctcctccaggaggggcaacgcaaggccggcgccgccgtcaccacggagacggaggtcatctgggcgaaggcgctgcccgccgggacgtccgcccagcgcgcggagctgatcgccctcacgcaggcgctgaagatggcggagggtaagaagctcaatgtctataccgactcgcgctacgccttcgccaccgcgcacatccacggcgagatctaccggcgtcgcggttggcttacctcggaggggaaggaaatcaagaacaaggacgagatcctcgcactcctcaaggccctgttcctccccaagcgcctgagcatcatccattgtccaggccaccagaagggccactcggcggaggcccgcggcaaccgaatggcggaccaggccgcgcgcaaggcggccatcaccgagaccccggacacgtccacgctgctcatcgagaactcctcgccgagcggcgggtcctccggcggttcgtcgggctcggaaacaccgggcacctccgagtccgccaccccggaatcgtccggaggctcgtccgggggctcgggctcctcgaaactcgagaagttcacgaactgctacagcctctcgaagaccctgcgcttcaaagccatccccgtcggtaagacgcaggaaaacatcgacaacaagcgactgctggtggaagacgagaagcgggcggaagactacaagggtgtcaagaagctgctcgaccgctactacctgtccttcatcaacgatgtcctccactcaatcaagctcaagaatctgaacaactacatcagcctcttccgcaagaagacgcgcaccgagaaggagaacaaggagttggagaacctcgagatcaacctgcgcaaggagatcgcaaaggcgttcaagggcaacgagggctataagtccctcttcaagaaggacatcatcgaaaccatcctcccggagttcctcgatgacaaggacgagatcgcgctcgtcaactccttcaacggtttcaccaccgcgttcaccggcttcttccgcaaccgcgagaacatgttcagcgaggaagccaagtccacctccatcgcgttccgttgcatcaacgaaaatttgacgcgctacatcagcaacatggacatcttcgagaaggtcgacgccatcttcgacaagcacgaagtgcaggagatcaaggagaaaatcctgaattccgattacgacgtagaagacttcttcgagggtgagttcttcaacttcgtcctcacccaggagggcatcgatgtgtacaacgccattatcggcggcttcgtcacggagagcggtgagaagatcaagggactgaacgaatacatcaacctgtacaaccagaaaaccaagcagaagctcccaaagttcaagccgctgtacaagcaggtcctctcggaccgcgagtcactctcgttctacggcgagggatacacctcggacgaagaagtgctggaagtgttccggaacaccctcaacaagaactcggagatcttcagctccatcaaaaagctggaaaagctcttcaagaacttcgacgagtactcgagcgccggcatcttcgtcaagaacggccccgcgatcagcaccatctcgaaggacatcttcggcgagtggaacgtgatccgggataagtggaacgcggaatacgacgacatccacctcaagaagaaggcggtggtcaccgagaagtacgaagacgaccgccgtaagtcgttcaagaagatcggctccttctccctcgagcagctgcaggagtacgccgacgccgacctgtcggtggtcgagaagctcaaggagatcatcatccagaaagtggacgagatctacaaggtctacggcagttcggagaagctgtttgacgccgacttcgtcctggagaagtcgctgaagaagaatgacgcggtggtggcgatcatgaaagacctccttgactcggtcaaaagcttcgagaactacatcaaggcattcttcggtgaaggtaaggagaccaaccgggatgaatcgttctatggcgatttcgtcctggcgtacgacatcttgcttaaggtggaccacatctacgacgcgatccggaattatgtgacgcaaaagccctactcgaaggacaagttcaagctgtacttccagaacccgcagttcatgggtggttgggataaggacaaggagacggactaccgcgccacgatcctgcgatacggatcgaagtactacctggccatcatggacaagaagtacgccaagtgcctccagaagatcgacaaggacgacgtcaacggtaactacgagaagattaactataagctcctgccggggcccaacaagatgttgccgaaggtcttcttcagtaagaagtggatggcctactacaacccgtcggaggacatccagaagatctacaagaacggtaccttcaaaaagggtgacatgttcaacctgaacgattgccacaagctgatcgatttcttcaaggattcgatctcgagataccccaagtggtcgaacgcgtatgacttcaacttctccgaaaccgagaagtacaaggacatcgccggattctacagggaggtcgaggagcagggttacaaggtcagcttcgagtcggccagcaagaaggaagtcgacaagctcgtggaggagggaaagctgtacatgttccagatttacaacaaggacttcagcgacaagtcccatggcacaccgaacctccataccatgtacttcaagctcctcttcgacgaaaacaaccacgggcagatccgcctgtcgggaggcgccgagctcttcatgcggcgtgcaagcctgaagaaggaggagctcgtcgtgcacccggcgaattcgcccatcgccaacaagaaccccgacaaccccaagaagaccaccaccttgagctacgacgtctacaaggataagcggtttagcgaggaccagtacgaactccacatcccgatcgccatcaacaagtgcccgaaaaacatcttcaagatcaacaccgaggtccgcgtcctgctgaagcacgatgacaacccctatgtgatcgggatcgaccgcggcgagcgtaacctgctgtatatcgtggtggtcgacgggaagggtaatatcgtcgaacagtacagcctcaacgagatcatcaacaacttcaacggtatccggatcaagaccgactaccactcgctgctcgacaagaaagagaaggagcgtttcgaggctcgtcagaactggacgtcgatcgaaaacatcaaggagctgaaggcgggttacatctcccaggtggtccacaagatctgcgagctcgtcgagaagtacgatgccgtcatcgccctggaggatctgaacagcggattcaaaaactcgcgcgtcaaggtggaaaaacaggtctaccagaagttcgaaaagatgctcatcgacaagctcaactacatggtagacaagaagtccaacccgtgcgccaccggcggcgcgctcaagggttaccagatcaccaacaagttcgagagcttcaagagcatgtcgacgcagaacggattcatcttctacatccccgcgtggctcacgagcaaaatcgatccttccacgggattcgtcaacctgctgaagaccaagtacacgtccatcgccgatagcaagaaattcatctcctccttcgaccggatcatgtatgtgccggaggaggacctcttcgagttcgcgctcgactacaagaacttctcgcggaccgacgcggactacatcaagaagtggaagttgtactcctacgggaaccgtatccgtatcttccggaaccccaagaaaaacaacgtcttcgactgggaggaggtgtgcctcaccagtgcctacaaggagctcttcaacaaatacggtatcaactaccagcagggcgacatccgggctctgctctgcgagcagtccgacaaggcgttctacagctccttcatggccctgatgtcgttgatgctccagatggccaactcgatcaccggccggaccgacgtcgacttcctgatcagtcccgtgaagaacagcgacggaatcttctacgactcacgcaactacgaagcgcaggagaacgcgatcctccccaagaacgcggacgctaacggcgcttacaacatcgcgcgcaaggtcctctgggccatcggacagttcaagaaggctgaggacgagaagctcgacaaggtgaagatcgccatcagcaacaaggagtggctggagtacgcccagacgtcggtgaagcacagcggttccggtggctcgtccggcgggtcgatggccgagaacggcgacaacgagaagatggcggccctcgaggcgaagatctgccatcagatcgagtattacttcggcgatttcaacctcccgcgcgacaagttcctcaaggaacagatcaagctggacgaaggctgggtcccgctggaaatcatgatcaagttcaaccgcctcaaccgcctgaccacagacttcaacgtgatcgtcgaggcactgtccaagtcgaaggcggaactgatggagatcagcgaggacaagaccaagatccgtcgatcgccgtccaagccgctccccgaagtgaccgacgagtacaagaacgacgtgaaaaaccggtccgtgtacatcaagggcttcccgacggacgcgacgctcgacgacattaaggagtggctcgaagataaggggcaggtgctgaacatccagatgcgccgcaccttgcacaaggccttcaagggctccatcttcgtcgtcttcgactccatcgaaagtgcgaagaagttcgttgagacgccgggccagaagtataaggaaaccgacctgctcatcctcttcaaggacgactacttcgcgaagaagaacgagtccggggggtcgaagcgcaccgcggacggctccgagttcgaacccaagaagaagcgcaaggtctga

SEQ ID NO: 306 - DNA, 6570 bp, synthetic construct
  source 1..6570: mol_type = other DNA
atgaagcgcaccgccgacggttcagaatttgagcccaagaaaaagcgaaaggtgtctggtggctcctccggtggatccaccttaaacatcgaggacgagtaccgactccacgagacgagcaaagaaccggacgtcagccttggttcgacgtggctgtcagactttccccaggcctgggctgagacaggagggatgggtctggccgtgagacaggcccccctgatcattccactcaaggctacttccacccccgtctccattaagcagtatccgatgtctcaagaagctcgactgggtatcaaaccccatatccagcgactcttggaccagggtattcttgttccctgtcaatctccgtggaacacccccctcctgcctgtgaagaagcctggtacaaacgattaccgacccgttcaggacctacgagaagtaaataagcgggtggaagacattcatcccaccgttccgaacccctacaacttactgagtggcctcccgccatcgcaccagtggtatactgtcctggacctcaaggacgcattcttctgcctgcgattgcaccccacctctcagcctttgttcgccttcgagtggcgagaccctgagatgggaatctcaggtcagctcacctggacacggctgcctcaaggctttaaaaactctcccacccttttcaatgaagccctccaccgcgatctggcagatttcagaatacaacaccccgacctgatcctccttcaatacgtggatgacctgctccttgcggccacctctgagcttgactgccagcaaggaaccagagccttgcttcagacgctcggaaacctgggataccgagcttccgctaagaaagctcagatctgtcagaaacaggtcaaatatctaggttacctcctgaaagaaggacagagatggctgactgaagcccgcaaggagacggtgatgggccagcccactcctaagactccgcggcaacttcgagaattccttggcaaggctggtttctgccgattattcatccccggcttcgcggagatggccgctcccctgtaccctcttaccaaaccaggtaccctcttcaactggggccccgaccagcaaaaggcttaccaagaaatcaagcaggccctgctgaccgctcctgctctgggactgcccgacctgacaaagccctttgagttgttcgttgatgagaagcagggctacgcaaagggtgtactgactcagaagctcggcccctggcgacgtccagtcgcatacctctctaagaagttggatcccgtggctgcgggatggcctccatgtttgagaatggttgccgccattgccgtgctgaccaaggacgcaggaaagctcacaatgggtcagcctcttgtgatccttgccccccacgccgtggaggccctcgtcaagcagcctcccgacaggtggctgtctaatgcccgtatgacgcactaccaggcattgctgcttgataccgatcgagttcaattcgggcctgtggtggcactgaatccagccacactgctgccattgccagaggaggggttgcagcataactgtttggacattctggcagaggcccatggtacgcgcccggatttgaccgaccaacccctgcctgacgccgatcatacttggtacacagacggatcctctctgcttcaagagggacagcgtaaggcgggtgctgccgttaccactgagaccgaggtgatctgggccaaggccctgcccgctggtactagcgcccaaagggctgagctgatcgccctgacccaggctctgaagatggccgagggcaagaaattgaatgtgtacacggatagtcggtacgctttcgctacagcacacattcacggggagatctaccgacgacgcggctggcttacttctgagggaaaagaaatcaagaacaaggatgagattcttgctctgctgaaagcccttttccttcccaagcgtctctctattattcactgccctggtcatcagaagggccactcagcggaggcacgaggcaacagaatggcagatcaggccgcccgaaaggctgctatcactgagacgccagacacatcgactctcctgatcgagaactcctcgcccagcggtggcagtagcggtgggtccagcggatccgagactccgggcacaagtgaatctgctactcccgagagttcgggaggcagttcaggaggatctggatccagcaagctggaaaaattcaccaactgctacagcctgtctaagacgttgcgattcaaggcgatacccgttggcaaaacgcaggagaacattgacaacaagcggctcctggtagaggacgagaagcgggctgaagactacaagggagttaagaaactgctggacagatactacctatcttttattaacgatgttctgcattctattaagttgaagaacctgaacaactacatctccttgtttcgtaagaagactcgaacggaaaaagagaacaaggaactggaaaatctagaaatcaacctccgaaaagagattgccaaggccttcaaggggaacgagggctacaaatccttgtttaagaaggacatcatcgagacaatcctccccgaatttcttgacgacaaggatgagatcgccctcgttaacagtttcaacggatttaccactgccttcacgggcttcttccgaaaccgcgagaacatgttctcggaagaggccaagagcacttcaattgctttccgatgtatcaacgagaatctgacccgatacatttcgaatatggacatttttgaaaaggtcgacgcgatttttgacaagcacgaggtgcaggagatcaaggagaagattctgaactctgactatgacgtcgaagactttttcgaaggtgagtttttcaatttcgtgttgacccaggagggtatcgacgtgtacaacgctattatcggaggatttgtgaccgagtccggagaaaaaattaagggactcaacgagtacatcaacctttataaccagaagacgaagcagaagctgcctaagtttaagcccctgtacaagcaggtcctatccgaccgagagtccctgagcttctacggagagggctacacctcggacgaagaggtccttgaagtgtttcgtaacaccctgaacaaaaacagcgagattttttcctcgattaagaagctcgagaagctttttaagaacttcgacgagtactcgtccgctgggatttttgttaagaacggtcctgctatctctacaatcagcaaggacatcttcggagagtggaatgttattagggacaagtggaacgcggaatacgacgacatccacctcaagaagaaggcggtggtcaccgagaagtatgaggacgaccgtcgtaaatctttcaagaaaatcggctcattcagtctcgaacagctacaggagtacgccgatgccgacctcagtgtcgtggagaagcttaaagaaatcattattcaaaaggtggacgagatctacaaagtgtacggatcttccgagaagctctttgacgccgacttcgtgctcgagaagtctctgaagaagaatgacgctgtcgtggccattatgaaggatctgctagactctgtgaagtcattcgaaaattatattaaagccttttttggtgagggaaaggagaccaacagagatgagtcgttctacggagacttcgtactcgcttatgacatccttctgaaggtcgatcatatttacgacgccatccgtaactacgtgacacagaagccctactctaaggacaagttcaaactgtactttcagaatccccagtttatggggggctgggataaggataaagagacagattatcgagctacaatcctgcgatatggctccaagtactatttggccattatggataagaagtacgctaagtgtcttcagaagattgataaggacgacgtcaacggcaactacgagaagattaactacaagcttctaccgggccctaacaagatgctgcccaaggttttcttctccaagaagtggatggcctactacaacccttcggaggacattcagaagatatacaagaacggaaccttcaaaaagggagacatgtttaacctcaacgattgccataagttgattgacttctttaaggattcgatctctcgatatcctaagtggtccaacgcctacgactttaacttctcggagactgagaagtataaggatatcgcgggcttttaccgggaggtggaagagcagggatacaaggtcagtttcgaatcagcttccaaaaaggaggttgataagctggtggaggaaggcaagctctacatgtttcagatctacaacaaggacttctcggataagtcgcacggaacccccaacctccatactatgtatttcaagctcttgtttgacgagaacaaccacggccagattagactctcaggtggcgccgagctcttcatgcgacgcgcatccctgaagaaggaggagctcgtggttcaccctgctaactctccgattgctaacaagaatccagacaatccaaagaagaccactaccctgtcttacgatgtctacaaagataaacggttctctgaggaccagtacgagttgcacatccctattgccattaacaagtgcccgaagaacatcttcaagatcaacacagaggtacgggttctcctcaaacatgatgacaatccttacgtcataggaatcgacagaggcgagagaaacctgctttacatcgtagttgtcgacggtaagggaaatattgtggagcagtattccctcaacgagattattaacaacttcaacggcatccgaatcaagaccgactaccactccctgttggacaagaaggagaaagaacgatttgaggcgcgacagaactggactagcatcgaaaacatcaaagagctcaaggcgggatatatttcccaagtcgtccacaagatttgtgagctggttgagaaatacgatgccgttatcgccctcgaggatttgaactcgggcttcaagaactcgagagtcaaggtcgagaagcaagtttaccagaaatttgagaagatgcttatcgacaagctgaattacatggtcgataaaaagtctaacccttgtgctaccggcggtgcactcaagggctaccagattaccaacaagttcgaatcgtttaagtcaatgtctacccagaatggcttcatcttctatatccctgcttggctcactagcaagattgatccttcgacaggcttcgtcaacctgctcaagacaaagtacacctccatcgcagactctaagaagtttatctcttcgttcgaccggatcatgtacgtcccagaggaggacctcttcgagtttgcactagactataagaactttagtcgaactgatgcagactacattaagaaatggaagctctattcgtacggcaaccgaatccgaattttccgaaaccctaaaaagaacaacgtctttgactgggaggaagtgtgcctgacctcggcttacaaagaactgttcaacaagtatggaattaactatcagcagggtgacatccgagcgctgctgtgcgagcagtcggacaaggctttctattcctcgtttatggccttaatgtctctgatgctgcagatggctaacagcatcacgggccgaaccgacgtggattttctgatctctcccgtcaaaaactccgatggaattttctacgactcgcgcaactacgaggctcaagagaatgctattctccctaagaatgctgacgcaaatggagcctacaacatcgcccgaaaggttctctgggccattggacagtttaagaaggcggaggacgagaagctggacaaggtgaagattgccatctccaacaaagagtggctcgaatacgcacaaacttccgttaagcattctggttcaggcggttccagtggaggctcgatggctgagaacggtgataacgagaagatggctgccctcgaggccaagatctgtcaccagattgagtactacttcggagatttcaaccttcctcgggataaatttctgaaggaacagataaagctagatgaaggatgggtccccctggagatcatgattaagttcaatcggctgaaccgactaactaccgactttaacgtgattgtcgaggcattgtccaaatctaaggctgagctaatggagatttctgaggataagaccaagattagacggtctccgtccaaacccctgcctgaggtgaccgacgagtacaagaatgatgtcaagaaccgttctgtttacattaagggctttcccaccgatgccacccttgacgatattaaagaatggctcgaggacaagggtcaggtcctgaatatccagatgagacggacgcttcataaggcgttcaagggcagcatttttgtggtgtttgattcaatcgagtccgccaagaagtttgtcgaaactcccggccagaagtacaaggaaactgacctgctcatcctgttcaaagatgattacttcgctaagaagaacgaatcgggcggttctaagagaactgccgacggtagcgaattcgaacctaagaagaaacgtaaggtgtaa

SEQ ID NO: 307 - DNA, 6570 bp, synthetic construct
  source 1..6570: mol_type = other DNA
atgaaacgcactgccgacggtagcgaattcgagccgaaaaaaaagcgtaaagtgtctgggggttctagcggtggcagcacgctgaacatcgaggatgaatatcgcctgcatgagaccagtaaagaaccggatgtttctctcggtagcacctggttgtccgacttcccacaggcgtgggctgagactggtggtatgggtctggcagttcggcaggcgccgctgatcattccacttaaagcaacctcaacccctgtgagtattaaacaatacccaatgtcccaggaagctcggttagggatcaaaccgcatattcagaggctgttagatcaaggcattttagtgccatgccaatcgccgtggaacactccactgttgccagttaaaaaaccgggaaccaatgattaccgaccggttcaagacttgcgggaagtgaataaacgcgtcgaagatattcatccgacggtcccgaacccgtataatctgcttagcggactcccgccttcgcatcagtggtatactgtattagatttgaaagacgccttcttctgtctgcgcctgcatccgacctcgcagccgctgtttgcattcgaatggcgtgacccggagatgggcatctccggccaattgacatggacgcggcttccccagggcttcaaaaactcccccacgctgtttaatgaagccttacatcgcgacttggcggactttcgtatccagcaccctgacctgatcctgttacagtatgttgatgatttactgcttgcggcaacgagtgaattagattgccagcaagggacccgggccctgctgcaaaccctggggaatctgggttatcgcgcgtcagcgaaaaaagcacagatttgccaaaaacaggtcaaatacttaggctacctcctgaaagagggccaacgctggttgaccgaggcccgcaaagaaaccgttatgggccagcccaccccgaaaacgccgcgtcagctgcgtgaattcctgggtaaagccggcttctgccgcctgttcatccccgggtttgccgagatggccgcgccgctgtacccgcttaccaaaccgggaaccctgttcaactggggtccagatcaacaaaaagcgtaccaggaaatcaagcaagcgttgttaaccgcgccagcactgggtctgcccgatctaacgaaaccgttcgaattattcgtggatgagaagcaaggctacgccaaaggcgtgctgacacaaaaactcggcccttggcggcgcccagtagcttatctcagcaaaaaactggacccggtagccgcagggtggccgccgtgcttacggatggttgctgcaatcgcggtgctgaccaaagatgcaggtaaactgacaatggggcaaccgcttgtgatcctcgcgccccatgcggttgaggctctggtgaaacaaccacctgatcgatggttgtctaatgcgcgcatgacccactaccaggcactgctgcttgacacggatcgcgtgcagttcggtccggtggtcgctctgaacccggcaacgctcctgccgctaccggaagaaggcctgcagcataattgcctcgacattctggccgaagcccatgggacccgcccggatctgactgatcagcctctgccggacgcggaccacacgtggtacaccgatggttcgagtctgctgcaggaaggccagcgtaaagctggcgcggcggtgaccacagagacggaagtaatttgggcgaaagcattgccggccggcacttcggcccagcgtgcagaactgatagcgttgactcaagcgctgaaaatggccgaaggcaagaaattgaacgtgtacacggatagccgttatgcattcgcaactgcgcatatccatggggaaatctatcgtagacgcggctggctgacatccgaaggcaaagagatcaagaacaaagatgaaatcctggcgctgctgaaggcgttgtttctccccaaacgtctgagcatcattcattgtccaggtcaccagaaaggacacagtgcggaagcccgcggtaaccgtatggcggaccaagcagcccgtaaggctgcgattacggagacgcctgataccagtaccctgttgatcgaaaacagttcgccatcagggggctcctcgggaggcagcagtggcagcgaaacccctggtactagcgaaagcgcgaccccggaatcctcaggtggctcttcgggcggcagtggttcttccaaattagagaaatttacgaattgttattctctgtctaagacgttgcgttttaaggccatcccggtcggcaagacccaggaaaacattgataataagcgccttctggttgaagatgagaagcgggcggaagattacaaaggtgttaaaaaattgctggatcgttactatttaagctttatcaacgacgtgctgcatagcatcaaactgaaaaatttaaataactatatttctctgttccgcaaaaaaacccggacggagaaagaaaacaaagaattggagaacttagaaattaacctacgcaaagaaattgcgaaagcgttcaagggcaacgagggctacaaatcgttgttcaagaaagacataatcgaaaccattttgcctgaatttctggacgataaagatgaaatcgccttggtgaattcgtttaacggttttaccaccgccttcacgggattttttcgaaatcgagaaaatatgtttagcgaagaggccaagtccaccagtattgcatttcgctgcatcaatgaaaacctgacccgctatattagcaatatggatatttttgaaaaagtcgatgcgatttttgataaacacgaagtgcaggaaattaaggaaaaaattctgaattccgattacgacgtcgaagacttttttgagggtgaattctttaacttcgtcttaactcaggaaggtattgacgtgtataatgcaattataggtggcttcgtaacggaaagtggtgagaagatcaagggtttgaacgagtacatcaacctgtacaatcagaaaacgaaacagaaattacccaagttcaaaccgttgtataaacaagtcctgagtgatcgcgaatctctgtccttttacggtgaaggctatacctcggacgaggaggtcctagaagttttccgtaacacgttaaacaaaaacagcgaaatcttctcctcaattaagaaattagaaaagctgtttaagaacttcgatgaatacagctcagctggtattttcgttaaaaacggtccggctatctccaccatttcaaaagatatcttcggcgaatggaatgtaatccgtgataagtggaacgcagagtatgacgatattcatttgaagaaaaaggccgtcgtaaccgaaaaatatgaagacgacagacgcaaaagtttcaaaaaaataggctcttttagtcttgaacaactgcaagaatatgcggacgcagacctttccgttgtggagaaactgaaagaaattattatccagaaagtggatgaaatctacaaagtctatggcagctccgagaagctgtttgatgcagattttgttcttgaaaaatcactgaaaaaaaatgacgctgtggtagcgatcatgaaagatcttctggatagtgtaaaaagcttcgaaaattacattaaagcttttttcggcgaagggaaagaaacaaatcgcgatgagtcgttttacggtgattttgtgctggcatatgatatcctgctcaaagtagatcacatctatgatgcgatccgtaactatgtcacccagaaaccctattcaaaagataagtttaaactgtactttcagaatccccagtttatgggcgggtgggacaaagataaagaaaccgattatcgcgctaccattctgcgttacggatcaaaatactacctcgctattatggataaaaaatatgcgaagtgcctgcaaaagattgacaaagatgatgtgaacggcaactatgagaagattaactataaactgttaccgggtcccaataagatgctgcctaaagtgttcttcagcaaaaaatggatggcgtattacaatccatctgaggatatacagaaaatttataaaaatgggacatttaaaaaaggggatatgttcaaccttaacgattgccataaattaattgacttcttcaaggactcaatttcaagatatccgaaatggtccaacgcgtatgatttcaactttagtgaaaccgagaaatacaaagatatcgcgggcttctatcgcgaagtcgaggagcagggttacaaagtgtcctttgaaagcgcgagtaaaaaggaggtggataagctggtagaagaaggcaaactctatatgtttcagatatataataaggactttagtgataaaagccacggcacgccgaacctccatacgatgtatttcaaacttctgtttgatgaaaataatcacggccaaattcgtctgtcgggtggcgccgagctgtttatgcgtcgtgcctcactgaagaaggaagaactggtcgtgcatcctgcgaattcgcctatcgcaaataaaaacccggacaatccgaaaaaaacaacgacgctgagctatgacgtttataaggacaaacgattttccgaggaccagtatgagctgcacataccgatcgcaattaacaaatgtcctaagaacatctttaagattaacacggaagtgcgtgttctgctgaaacacgatgataacccgtacgtcatcggtattgaccgtggcgagcgtaatctgttgtacattgtggttgtagatggaaaaggcaacatcgtagagcaatatagcctgaacgagatcattaacaacttcaatggtattcgcatcaaaactgattatcacagcctattagataaaaaggaaaaggaacgttttgaagctcgccagaactggacatcgatcgaaaatattaaggaactcaaagccgggtacatctcgcaggtcgtgcacaaaatttgtgaacttgtggaaaaatacgatgctgtaattgcactggaagatttgaacagcggcttcaaaaatagtcgtgttaaggtggaaaagcaggtttatcagaaatttgaaaagatgcttattgacaaattaaactatatggttgacaaaaaatcaaatccatgtgccacagggggtgcgctgaagggctaccagattacaaacaaatttgaaagctttaagagcatgagtactcagaatggattcattttttatatcccggcgtggctgacctcgaaaatcgatccttccactggctttgtgaacctgcttaagacaaaatacacctctattgccgacagtaaaaaatttattagcagcttcgatagaatcatgtacgttccagaagaagatctatttgaattcgcgcttgactataaaaatttctctcgtaccgatgcagattacattaagaagtggaaactctacagctacggtaaccgcattcgtatattccgtaatccgaaaaaaaacaatgtgtttgattgggaagaggtctgtcttacctcagcgtacaaagagctctttaataaatatggtatcaattatcaacagggcgatattcgcgccctgctgtgcgaacagagcgataaagccttttactcgagcttcatggcattaatgtctctcatgttgcagatggcaaattccattactggacgtacggatgtggattttctgattagcccggttaaaaactcggacggtatcttttatgattcacgcaactatgaggcccaggaaaatgctatccttccaaaaaacgccgacgccaacggagcctataatattgcgaggaaagtcctgtgggcgattggtcagttcaaaaaagccgaggatgagaaactggacaaagtgaaaattgccatttctaacaaagaatggctcgagtatgcccaaacctctgtgaaacattctggcagcgggggatcgtctgggggttctatggcagagaacggcgataatgaaaaaatggctgcgctcgaagcgaaaatttgtcatcagattgaatattatttcggagactttaacctcccgcgcgataaatttctaaaagagcaaatcaagctggatgaaggttgggtcccgctggaaatcatgatcaaatttaatcgtctgaaccgcttaaccaccgactttaatgtgatcgtggaagccctgagcaaatccaaagctgaactcatggaaataagcgaagataaaacaaaaattcgccgcagtccgtcgaaaccgctgcctgaagtcacggatgaatataaaaacgacgttaaaaatcgctctgtttacattaaaggatttccaactgacgctactctggatgatatcaaagagtggctggaagataaaggtcaggttctgaatatccagatgcgtcgtactcttcacaaagctttcaaaggcagcatttttgtcgtttttgacagcattgaatcggcgaaaaaatttgtagaaaccccgggtcagaaatataaagaaactgacctgttgattttatttaaagatgactattttgctaaaaaaaacgaatctggcgggagcaaacgtaccgcggacggctccgaatttgagccaaaaaaaaagcgcaaagtttaa

SEQ ID NO: 308 - DNA, 6570 bp, synthetic construct
  source 1..6570: mol_type = other DNA
atgaagcgtacagcagatggttccgagttcgaaccgaagaagaagcgcaaggtgtcgggtggttcttcggggggatcgaccttgaacattgaggacgaatatcggctccatgagacgtccaaagagcccgacgtatcgctgggctccacctggctcagcgatttcccccaggcctgggctgaaactggtggcatgggtctggctgtgaggcaagctcccctcatcattcctctgaaggcaactagcacccctgtgtcgattaaacagtaccctatgtcccaggaagcccgccttggaatcaagccccacatccagcgtctgttggatcagggtattctagttccgtgccagtctccctggaacacacctctgctccccgttaagaagcctggtacgaatgactaccgccctgtgcaggatctgcgtgaggtgaacaagcgtgtcgaagacatacacccaaccgtacccaacccttacaacctcctgtccggcctgcctcccagccatcagtggtacacggtgctggacctgaaggacgcgtttttctgtcttcgcctgcacccgacatcccaaccgcttttcgctttcgagtggcgagaccccgagatgggaatctctggtcagctcacttggactcggctccctcagggttttaagaactctccgaccctattcaacgaggcattgcaccgtgacctcgccgattttcgcatccaacaccccgacctgatccttctccagtacgtggacgacctactgctggccgctacctcggagctcgactgtcagcaggggacccgtgccctgctccagaccctcggtaacttgggttaccgcgcgtctgctaagaaggctcagatatgccagaagcaggtcaagtatttgggttatttgctgaaggaaggtcagcggtggctgaccgaggcacggaaggagaccgttatgggtcaacctaccccgaagacaccgcgacagctgcgtgagttcttaggtaaggcaggtttctgccggctgttcattcccggcttcgccgagatggctgctccactctacccgctcaccaagcctggaactctgttcaactggggtcccgatcaacagaaggcgtaccaggagatcaagcaggcgttgctcaccgcccccgctctgggcctacccgatcttaccaagcctttcgagctgtttgtggatgagaagcagggctacgcgaaaggcgtcctcacacagaagctgggcccatggcgccgccccgtggcctacctgagcaagaaactagatcccgtcgccgccggctggcctccttgcctgcgtatggtcgctgccatcgccgtcttgaccaaagatgccggcaagcttaccatgggccaaccgctggttatcctcgctccgcatgcggtcgaagctcttgttaagcagccgccagaccgctggctgtctaacgctagaatgacgcactaccaagccctgctgcttgacacagaccgcgtgcagttcgggccggtagtagccctgaatcccgcgactctgctgcccctgccggaggagggcctccaacataactgtctcgatattcttgctgaagcacacggcactcgtccggacctgaccgatcaacccctgccagacgcagatcacacgtggtatactgacggatctagcctccttcaggaaggtcaacgaaaggccggtgccgccgtaacgaccgaaaccgaggtcatctgggctaaggctctcccagctgggacctccgcacagcgcgcggagctcattgccctcacgcaagcgctcaaaatggccgaaggaaagaagcttaacgtctacaccgatagtcgctatgcattcgctactgcccatattcacggggagatctatcgccggcggggttggttgacctcggaaggtaaggagattaagaataaggacgagatactggccctcctgaaggccctgttcttgccaaagcgtctttccattattcattgtcctggtcaccagaagggccatagtgccgaagcgaggggtaaccgcatggccgaccaggccgcccgaaaggccgccatcaccgagactcctgatactagcaccttgttgattgagaactcctctccctccggtggaagcagtggtggatccagcggctcggagactcccggcacttctgaatcagcgactcccgaatccagcgggggtagcagtggaggctccggctcatcgaagctggaaaagttcacaaattgctacagtctgagtaagacacttcgtttcaaggctatcccggtcggcaagacccaagagaacatcgacaacaaacgtctcctcgtcgaggacgaaaagcgcgccgaggattacaagggagttaagaagctgctggatcgctactacttgtctttcatcaacgacgtcttgcattctattaagctgaaaaaccttaacaactatatatccctgttccgcaagaaaactaggaccgagaaggagaataaggaactggagaaccttgagatcaacctccgaaaggaaatcgctaaggcttttaagggcaacgagggctataagtcccttttcaagaaggatatcattgaaacaatcctccccgagttcctagacgacaaggatgaaatcgctctggttaatagcttcaatggcttcaccacagctttcacgggtttctttcgaaatcgcgagaatatgtttagcgaagaggctaaatcgaccagcattgctttcagatgcattaacgagaacctcacgcgctatatctcgaacatggacatcttcgaaaaggtggacgccatctttgacaagcacgaggttcaagaaatcaaggagaagatcctcaacagcgactacgacgtcgaggactttttcgagggagagttctttaacttcgtgttaacccaggaggggatcgatgtgtataatgctatcatcggtggcttcgtcaccgagtcgggcgagaaaatcaaaggtttgaatgagtatatcaacctgtacaaccagaagaccaagcagaagttgcctaagttcaagccactatacaagcaggttctttcggaccgcgagagtctttcattctatggcgagggatatacctccgatgaagaggtcctcgaggtgtttaggaatacgctcaacaagaattccgagatcttctcttctatcaagaagctggagaagttgtttaagaatttcgacgaatactcttccgccggtatttttgtgaagaatggccccgcgatctccaccatctcaaaggacatcttcggcgagtggaatgtgatccgcgataagtggaacgcggagtacgatgacatccaccttaagaagaaggctgtcgttaccgagaaatacgaagatgacagacgtaagagcttcaagaaaatcggttccttctcactcgagcagttgcaggagtacgcggatgcagatctcagcgtggtggagaagctgaaagaaataatcatccagaaagtcgacgagatttacaaagtctacggtagcagtgagaagctcttcgatgctgacttcgtgcttgagaagtcgctcaagaagaacgacgctgtcgttgccattatgaaagatttgctggacagtgtgaagtctttcgagaactacattaaggccttcttcggagagggcaaggaaaccaaccgcgacgagtccttctacggcgatttcgtgctggcgtacgatatcctcctcaaggtcgaccatatttacgacgccatccggaattacgttacacagaagccatattccaaggacaagttcaaactctactttcaaaacccgcagtttatgggtgggtgggataaggataaggagaccgactaccgggccaccattttgcgttacggctccaagtactacttggccatcatggataaaaaatacgccaagtgtctccagaagattgacaaggacgacgttaatggtaactacgaaaagattaactacaagctcctacccggtcccaacaagatgctccccaaggtgttcttcagtaagaagtggatggcctactacaatccctctgaggacatacagaagatttacaagaacggcacctttaagaagggtgacatgttcaacctaaacgactgccacaaactgatcgacttcttcaaggactctatctcacgctatcccaagtggtccaacgcctacgacttcaatttctccgagactgagaagtacaaggatatcgcgggcttctaccgagaggtcgaggagcagggctacaaagtttccttcgagtccgcctcgaagaaggaagtcgacaagttggtcgaggagggtaagctgtacatgttccagatctataacaaagacttctcggacaagtctcacggtactcccaacttgcataccatgtacttcaagctgctgttcgacgagaacaaccacgggcaaatccgtctttccggcggcgcggagcttttcatgagacgtgccagtctcaagaaggaagagctggtcgttcaccccgcaaactcgcctattgctaacaaaaaccccgacaaccccaagaagaccactactctttcatatgatgtctataaagataagcgcttctccgaggatcaatacgaactccacatccccatcgcgatcaacaagtgcccaaaaaacatatttaagatcaataccgaagtccgcgtgttactcaagcacgacgacaacccgtacgtgattggaatcgatcgcggagagcggaaccttctctacattgtggtcgttgatggcaagggcaacatcgttgaacagtacagcttgaacgagattatcaacaacttcaatggcatccgtatcaagaccgattaccattcgttgctcgataaaaaagagaaggaacggttcgaggccagacagaactggacctccatcgagaacatcaaggagctcaaggctggctacatcagccaggtcgttcataagatttgcgaactggtcgaaaagtatgatgcggtcattgcacttgaggaccttaattccggttttaaaaactcgcgcgtcaaagttgagaagcaagtctatcagaagttcgagaagatgctcatcgacaagttaaactacatggtcgacaagaagagcaacccgtgcgcgacgggaggtgccttgaaaggataccagatcaccaacaagtttgaatcctttaagagtatgtctacgcagaatggctttattttttacatccctgcatggctgacttcgaagatcgaccctagtactggatttgtcaacctgctgaagacaaagtatacgtctatcgcggattccaagaagttcatctcgtctttcgaccgtatcatgtatgtccccgaggaggatttgttcgagttcgccttggactacaaaaacttctcgcgtaccgacgcagactacatcaagaaatggaagttgtacagctatggaaaccggatccggatcttcagaaacccaaagaagaacaacgtcttcgattgggaggaggtgtgtttgacgtccgcatacaaagaactgtttaacaagtacgggatcaactaccaacagggcgacatcagagccctcctgtgcgagcaatccgacaaggcattctactcctccttcatggccctcatgagcctgatgctccagatggcgaatagcatcacgggacggactgatgtcgatttcttgatttcccccgtcaagaacagcgacggaattttctacgattcccgcaattacgaggcccaggagaacgccatccttcctaagaacgctgacgctaacggagcttacaacattgctcgcaaggttctatgggctatcggccaattcaagaaggccgaggatgagaaactggacaaggtcaagatcgcaatctccaataaggaatggttggagtatgctcagacctctgtcaagcactccggaagcggcggcagctctggtggttcaatggccgagaacggagataacgagaagatggccgctctcgaggccaagatctgccaccagatcgagtattacttcggtgactttaacctcccaagggataagttcctcaaggaacagattaagctggacgagggctgggttcccctggaaattatgattaagtttaacaggctcaaccgtctcacgaccgatttcaacgtgatcgttgaggccctgtctaaaagcaaggccgaactgatggagatctccgaagacaagaccaagatccgccgttccccgagtaagcccctcccggaggtgacggatgaatacaagaatgatgtcaagaaccgaagcgtctatatcaagggatttcctaccgatgccacgcttgatgacatcaaagagtggcttgaagacaagggccaggtcctcaacatccaaatgcgccgcaccctgcacaaggctttcaaggggtcgattttcgtggtctttgatagcatcgagagcgccaagaagttcgtcgagacccctggccaaaagtacaaggaaacggatcttcttattttgttcaaggatgactatttcgcgaagaagaatgagtccggcggcagtaaacgtactgccgatggctctgaattcgaacctaagaagaagcgtaaggtttaa

SEQ ID NO: 309 - DNA, 6570 bp, synthetic construct
  source 1..6570: mol_type = other DNA
atgaaacgtaccgcagatggatctgagttcgagcccaaaaaaaaaagaaaagtcagcggcgggtcttctgggggttcgaccctcaatattgaggatgaatatcggttgcatgagacgagcaaagaaccagacgtatctcttggatctacctggctcagtgatttccctcaagcatgggccgaaaccggaggaatggggcttgcagtaagacaagctcctctcatcattccccttaaagcgacatcgactccggtatccattaagcaatatcctatgtcccaagaagcacggcttggtattaagccacacatacaacggttgttagatcagggcattttagtgccctgtcaatcaccttggaatactcctttattgccagtgaaaaaaccgggaacaaatgactataggccagtgcaagacttaagagaggttaataagcgggttgaagatatccaccctacagttccaaatccgtataatttgttaagtggattgccaccatcccaccagtggtataccgtcttagatttgaaggatgcgttcttctgtctacggttgcacccgacatctcaacctctgtttgctttcgaatggagagatcccgaaatggggatctcgggacagttgacttggactaggcttccacaaggttttaaaaatagccccaccctttttaatgaggcattacacagggacttggctgactttcgtatccagcacccagacctcattctcttgcaatatgttgatgacctgttgcttgccgcaacctcagaattggattgtcaacaaggcacgagagctttacttcagacgttaggtaacctcggttatagggcgagtgccaagaaggcccaaatatgccagaaacaggtaaagtatttgggctatcttctaaaagaaggccagaggtggttgaccgaagcacgaaaagaaactgtaatggggcaacccaccccaaagacacctcggcaattgcgggaatttctgggtaaagcaggcttttgccgtttgttcattccaggttttgctgaaatggcagcaccactatatcccttgacgaagcccggaacactatttaactggggtccagatcagcaaaaagcgtaccaggagataaaacaagctctcttaactgcgccggcccttggtttgcccgacttgaccaagccatttgaactttttgttgatgaaaaacagggatacgctaaaggcgttcttacacaaaaacttggtccgtggagacgacctgtcgcatatttgagcaaaaaactggacccagtggctgctggatggccgccatgtctaaggatggtcgctgctattgctgtactcactaaggatgcaggtaaacttactatgggacaaccgttagtcattcttgctccgcatgcagttgaagccctcgttaagcagccccctgataggtggttaagcaacgcaagaatgacacattatcaagcattactcttggataccgacagagtacaatttgggcctgttgttgctttaaaccctgcgacgcttctgcccctcccggaagagggactacagcataactgtcttgatatactcgcggaggctcacggcacaagacctgacctaacggatcaacctcttcccgatgctgatcatacttggtacacagatgggtcttcattactccaagagggccaacgaaaagcaggagcggcggtgactactgaaaccgaagttatatgggccaaagcattgccggctggcacatccgcgcaacgcgccgagttaatcgcattaacacaagcactaaaaatggccgaagggaagaaattgaatgtatatactgattcccggtatgcctttgctactgctcatatacacggcgaaatctaccgccgcagaggatggttaactagtgagggcaaggagattaaaaacaaagacgagatacttgcattattgaaggcccttttcctcccgaaacgattgtccattatacactgtccaggacatcagaagggccattctgccgaggctcgagggaatcggatggcagatcaggcagctcgtaaagccgctataacagaaacacctgatactagtacattacttattgagaattcctcaccgtctggcggatcttctggggggtcttcaggatccgagacacctggaacgtcagaatccgcgacaccagaatcatcgggaggatctagcgggggatcaggttcctcaaaattagaaaaatttaccaattgttattcactgagcaaaactctccgattcaaggcaatccccgtgggcaagacacaagagaatattgataataagaggcttttggttgaagatgagaaaagggcggaggattacaaaggtgtcaaaaaactgttggatcgttattatttaagttttattaatgatgttttgcattctattaaattgaaaaacctgaataactatatttcattgtttaggaagaaaacaaggactgaaaaagagaataaagaacttgaaaatctcgagattaacctgagaaaagaaatagctaaagcatttaaaggaaacgaagggtataaatccctctttaagaaggacattatagaaaccatactacctgagtttctagacgataaggatgaaatcgcgcttgtaaactcttttaatggtttcaccacagcatttactggattcttccgcaaccgcgagaatatgttcagtgaggaagcaaagagtacatcaatagccttcagatgcataaacgagaatttaacaagatacatttctaatatggacattttcgaaaaagtagacgccattttcgacaagcacgaggtacaagaaatcaaggaaaagattttgaattcggattatgatgtagaggatttcttcgagggagaattctttaattttgttttgactcaagaaggtattgatgtttataatgctataattgggggattcgtgacagaatcgggagagaaaatcaaaggcttgaatgaatacattaacctgtacaatcaaaagacaaagcagaaactcccaaagtttaagccactatataaacaggtgctatctgatagggagtcattgtccttttacggagaaggttacacttcagatgaagaagtgttggaagtattcagaaatacattaaataaaaatagcgaaattttttcctccatcaagaaactcgaaaaactgtttaagaactttgacgaatattcatctgcaggtatcttcgtcaaaaacgggcctgcaatatcaaccatcagcaaggacatctttggtgaatggaacgttatacgcgacaaatggaacgctgaatatgacgacatccacttaaaaaagaaagccgtagtgacggaaaagtacgaagatgacagacgtaagtctttcaaaaaaatagggtcattcagtttagagcaactacaagagtatgcagacgcagacttgtccgtggtcgagaaactcaaggaaattattatacagaaagttgatgaaatctacaaagtatatggttctagcgagaagctgtttgatgcagattttgtattggagaaatcattgaaaaaaaatgatgctgttgttgcgattatgaaggacctgctagattcggttaaatcgtttgaaaactatattaaagcattttttggcgaagggaaagagaccaatcgtgatgaatcgttttacggtgattttgtgctagcttacgacatccttttaaaagtcgatcatatctatgatgccatcagaaattacgtaacccagaaaccatacagcaaggacaagtttaaactttactttcaaaacccacaattcatgggtggctgggataaagataaagaaacagattatagagccacgatactcaggtatggaagtaagtattatcttgccataatggacaagaaatatgccaagtgcctacaaaaaatagataaggacgatgtgaatggcaactatgaaaagataaattataaacttcttcccggtccgaacaaaatgttaccaaaagtgtttttcagtaagaagtggatggcctactacaacccatccgaagacatccaaaaaatttacaaaaacgggacctttaaaaaaggggatatgtttaaccttaacgactgtcataaacttatagactttttcaaagattcaatttcaagatatcctaagtggagtaatgcttacgactttaattttagcgagactgaaaagtataaagacattgcaggcttttatagagaagttgaagaacaagggtataaagtttccttcgagtcggcgagtaaaaaggaggtagataaactagtcgaggaggggaaactctacatgttccaaatttacaacaaggacttcagtgacaagtctcacggtacccccaacctacataccatgtattttaaactattgttcgatgagaataatcatggacaaattagactcagtgggggtgctgagttatttatgcgaagagcatcactcaaaaaagaggagctcgttgtccatcctgctaactcaccaatagcaaataagaatccggataatcctaaaaaaacaacaactctttcctacgatgtctacaaagataaacggttttcagaagatcaatatgaattacatatcccaattgccattaataagtgcccaaaaaacatattcaagataaatacggaagttagagttcttttgaaacatgatgataatccatatgtcataggaattgatcgtggtgagcgaaacctgttatacatagttgttgttgacggtaagggaaacatagttgagcagtactcgttgaacgaaattatcaataattttaatggtattcgtataaagacagactatcattcactgttagacaaaaaagagaaggagagattcgaagctcgacaaaactggacaagtattgagaatattaaagagctcaaggctggttacatttctcaagtggtccataaaatttgtgaattagtagaaaagtatgatgccgtgattgcgttagaagatctaaactccggcttcaaaaattcaagagttaaagtagagaagcaagtataccagaaattcgagaaaatgctcattgataaattgaattatatggttgacaaaaagagcaatccttgtgctacgggcggagcactaaaaggatatcagataacaaataagtttgaatctttcaagtcgatgagcacccaaaacgggtttattttttatatccctgcttggttaacttcaaaaatcgatccgtccacgggatttgtaaatcttttaaaaacaaaatatacatctattgctgacagcaagaaattcatctcttcgtttgatcgaatcatgtacgttcccgaggaagatctgtttgaattcgccttggattacaaaaattttagcaggacagatgcggattatataaaaaaatggaagctctatagttatgggaaccgaatcaggatatttagaaatccaaaaaaaaacaatgttttcgattgggaagaagtctgccttacatcagcatataaagaattatttaataaatatggaataaactaccaacagggtgatattagagctttgctgtgtgaacaatccgataaagccttctattcaagttttatggcactgatgtctctgatgttgcaaatggcaaatagtataactgggagaaccgatgttgattttttaatctctccagtgaaaaatagtgatggcatattttatgattcacgaaattacgaagcccaagaaaacgctatcctgcccaaaaatgccgatgcaaacggagcatataacattgctagaaaggtcctttgggctatcggtcaattcaagaaagcagaggacgaaaagcttgacaaggtgaaaatagccataagcaataaagagtggttagaatatgcgcaaactagtgtaaaacatagtggatctggtggctcgtcgggaggtagtatggctgaaaatggtgataatgaaaaaatggctgcgcttgaggccaaaatttgccatcagatagagtattatttcggagattttaatttgccgagggataagtttcttaaagaacaaattaagctggatgaaggttgggttcctttagaaattatgatcaaattcaatcggcttaatcgtctaacaactgattttaacgtgatcgtagaagcactgtctaaaagcaaggcggaattgatggaaatatctgaagataagactaaaattagaagatctccatcgaagcccctacctgaagtgacggatgagtataaaaacgatgtcaaaaatcgttcagtgtacatcaagggttttccaactgatgcgacgttagatgacattaaagagtggctggaagataaaggacaggtgcttaacatacagatgcgacgaactctacacaaggcatttaaggggagcatatttgttgtttttgattcaattgagtctgctaaaaaattcgtcgaaacaccaggacagaaatataaagaaacggatttgctcatcctatttaaggatgactactttgctaaaaagaatgaatcaggaggttccaaaaggacggcagatggatctgaatttgagcctaagaagaagagaaaagtgtga

SEQ ID NO: 310 - DNA, 6570 bp, synthetic construct
  source 1..6570: mol_type = other DNA
atgaaacgtacggcggacggatctgaattcgaaccaaagaagaaacgcaaggtttctggaggctcctcgggcggcagcaccctgaatattgaagacgagtataggcttcatgagaccagcaaagaaccagatgtctcgctgggatccacgtggttgtctgattttccacaagcatgggcagaaactggtggtatggggctggcggtgcgccaagcgccattaatcatccccctcaaggcaactagtaccccggtctccattaagcagtacccgatgtcccaggaagcccgcctgggcatcaagccgcacatccagcgtttgttggatcaaggtatcctggtaccctgccagagcccctggaacacccccctcctgcccgtgaaaaagcctggcactaacgattaccgcccggtccaggatttgcgtgaagtgaacaaacgcgtggaagatattcaccctaccgtgccaaacccgtacaatcttctctctggtcttcctccgtctcatcaatggtataccgtcctggacctcaaggacgcgtttttttgcctccgacttcacccaacctcccagcccctgtttgccttcgaatggcgcgatcctgagatgggcatttccggccagttgacctggacccgccttccacagggtttcaagaactcccctacccttttcaacgaggcgctccaccgcgatctggctgacttccgtattcaacaccctgatctaatcctattgcagtacgtggatgatctcttgcttgcggcaaccagtgaattggattgtcaacagggaacccgagcactgctgcagacactgggtaacttgggctatcgcgccagtgctaagaaagcacagatctgccaaaagcaagtgaagtacttgggctatctgcttaaggaaggacaacgttggttgaccgaagcgcgaaaggagactgtgatgggccaaccgaccccaaagacgccacgtcaattgcgagagtttctgggcaaagctggtttctgtcggctcttcatccccggctttgcagaaatggccgcgcccctttacccgttgacaaagccaggaaccctgtttaactggggacccgaccagcagaaggcgtaccaggaaatcaaacaggcgctacttactgcgcctgcacttggactaccagacctgaccaagccattcgagctgttcgtcgacgaaaagcaaggttacgcaaagggcgtgctgacgcaaaagctgggcccttggcgtcgcccagtggcatatctgagcaaaaagctggacccagttgcagccggttggccaccctgcctacgtatggtggccgctatcgcggttcttaccaaagacgctggcaagctcactatgggtcaaccactggtcattctggcacctcacgcggtggaggctctggtcaagcaaccgccagatcgatggttaagcaatgcacgcatgacccactaccaggcactcctccttgatactgatcgtgttcagttcggaccagttgttgccctcaatcctgcaacgctccttcctctgcccgaggaaggattacagcataactgcctggacatcttagctgaagctcatggcacccgcccggatctaacagatcaacccttgcccgatgcggatcacacttggtatactgacggatcatcccttctacaggaaggccagcgaaaggccggcgctgccgtgacgaccgagaccgaggttatttgggccaaagccttgccagctggcacaagcgcccagcgagctgaacttattgcccttactcaagcgcttaaaatggcagagggaaaaaagctgaatgtctacactgatagtcgctacgcgttcgcaaccgcgcacatccatggtgagatctaccgtaggcgtggctggctcacaagcgagggcaaagaaatcaaaaacaaagatgaaattttggcgttgttaaaagcattgtttctgccaaagcgtttgtcgattattcattgcccaggacaccagaagggccacagcgcagaggcgcgcggtaataggatggcagaccaagctgcgagaaaggcagccattacggaaactccagatacttccacgctactcatcgagaactcctcgccctccgggggctcgtcggggggttcatcaggcagcgaaacacctggcaccagcgaaagcgccactccagaatcctccggtgggagctccgggggctccggttcatcaaagctcgagaagtttaccaactgctactctctcagtaaaactctgcgttttaaagccataccggtagggaagacccaggaaaatattgacaacaagaggttgctcgtcgaggatgaaaagcgcgccgaagactataagggcgtcaagaagctgctggaccgctactatctctccttcatcaacgatgtactacactccatcaagctcaaaaacctcaataactacatctctctcttccgcaaaaagacccgcactgagaaagaaaataaagaactcgaaaatcttgagatcaatttgcgcaaagaaatcgcaaaagccttcaagggcaatgaaggttataaatctttgttcaagaaggacattatcgaaaccatcctcccggagttcctggacgataaggatgagatcgctctggtcaacagtttcaacggtttcaccactgccttcacgggatttttccgcaatcgcgaaaacatgttctcagaggaggcgaaaagtaccagtattgcattccgatgcatcaacgaaaaccttacccgttatattagcaacatggatatcttcgaaaaggtggacgccatttttgacaagcacgaagtccaggagataaaggagaagatccttaactctgattacgacgttgaggatttctttgaaggtgagttcttcaacttcgtactgacccaggagggcatagatgtgtacaatgcaatcatcggaggcttcgtgaccgaatccggtgagaaaatcaaggggctgaatgagtacataaacctgtacaatcaaaagaccaagcaaaaactgccgaagttcaagcctctctataaacaagttttgtccgatcgagagtccctctcattttacggagaaggatacacctccgatgaagaggtgttggaagttttccgtaacacgctcaacaagaacagtgagatcttttcctcgattaagaaattggaaaagttatttaaaaacttcgatgagtactcatctgctggtatcttcgttaagaacggcccggcgattagcaccatctctaaggatatttttggggagtggaacgtaatccgtgataagtggaatgctgaatatgacgacatccacttgaagaaaaaggccgtggtcacggaaaaatacgaggatgatcgcagaaaatctttcaaaaaaatcggatccttctccctcgagcaacttcaggagtatgccgatgcggatctctccgttgtcgaaaagcttaaggaaatcattatccagaaagtcgacgagatctataaggtgtatggctcttccgaaaagctgtttgatgctgatttcgtgctagaaaagtccctgaagaagaacgatgcagtcgtcgcaatcatgaaggatctgcttgattctgtgaaatcattcgagaactacatcaaagcctttttcggtgaaggcaaagaaacaaatcgggacgagtcattttacggcgattttgttttggcctacgacatcctgctcaaagtcgaccacatctacgacgcaatccgcaactatgttacacagaaaccttactccaaggataaattcaagttgtactttcagaacccacagttcatgggcggttgggataaggacaaggagactgactaccgggctacgatcctgcgttacggatcgaaatactatctcgcaattatggacaaaaaatacgcaaagtgcctgcagaaaattgacaaagacgacgtgaacgggaactatgaaaaaattaattacaaattactgcctggaccaaacaaaatgctccctaaggtatttttttctaaaaaatggatggcgtattataatccttcagaagatattcaaaagatttataaaaacggtactttcaagaaaggcgatatgttcaatctaaacgattgccacaaactcatcgacttcttcaaggattcaatctctcgctacccgaaatggtccaatgcgtacgactttaatttttcggagactgagaagtacaaggacattgcaggattctatcgagaagtagaagagcagggttacaaagtgtcattcgagtccgcctccaagaaagaagtggacaagctcgttgaagaaggtaagctctacatgtttcaaatctacaacaaggatttctcggataagagtcacggtaccccaaatctgcacaccatgtatttcaagctcttgttcgacgaaaataatcatggccagattcgcctttccggcggtgcagagctcttcatgcgccgcgcctccttgaaaaaggaagaactcgtggttcaccctgcaaattccccgattgcgaacaaaaacccagacaacccaaagaaaacgaccaccttatcctacgatgtatacaaagacaagcgcttctccgaagaccagtatgaacttcatatcccaatcgctattaacaaatgtcctaagaacattttcaaaatcaataccgaagttcgtgtcttgctcaagcatgatgataacccctacgtgattggcattgatcgcggcgaacgcaatctgttgtacattgttgtcgtggacggtaagggcaatattgtggaacaatactccctgaatgaaattatcaacaactttaacggcatccgaatcaaaaccgattaccactcattgttggacaagaaggagaaggaacgtttcgaagctcgccagaactggacatctattgagaacatcaaggaacttaaggctggctacatctcacaggtcgttcataaaatctgcgagcttgtggagaaatatgatgcggttatcgctctggaagacttgaattctggatttaagaacagccgcgtcaaagttgagaagcaggtttatcaaaaattcgaaaaaatgctgattgacaagctcaactacatggtcgacaagaaatccaacccgtgtgcaacaggtggtgcactgaagggttaccagattaccaacaagttcgaatctttcaagtctatgtccacccagaacggctttattttttacatcccagcttggctgacctcaaagatcgacccatccaccggtttcgttaacctgcttaaaaccaagtacacctctattgccgatagtaagaaatttatctcctccttcgatcgtatcatgtatgtccccgaagaagacctgtttgagtttgccctcgattacaaaaatttttcccggacggatgcagattacatcaaaaaatggaagctttactcttacggaaatcggatccgcattttccggaacccgaagaagaacaacgttttcgattgggaggaggtttgtctgacctcagcatacaaggagctgttcaacaaatacggcatcaactaccagcagggagatatccgcgctcttctctgtgagcagtctgacaaggcattctactcctcctttatggctttgatgagcttaatgctgcagatggccaatagcatcaccggtcgcaccgacgtcgatttcctgatctctcccgtgaaaaactcagatggcatcttttacgactcacggaactatgaagcccaagaaaacgccatcctgccgaagaacgctgacgccaacggtgcgtataatattgctcggaaggtgctttgggcaatcggtcagttcaagaaagctgaagatgaaaagctggacaaggtgaaaatcgctatctccaacaaagagtggttggaatatgcgcagaccagcgtgaaacactcgggatctggcggttcgtcgggcggctcgatggctgaaaacggcgacaacgaaaaaatggcagccctcgaggcaaagatttgccatcaaattgagtattattttggtgacttcaacctccctcgcgataagtttttgaaggagcaaatcaagttggatgagggttgggtgccgttggagattatgatcaagttcaatcgcctgaaccgcctcacaacggatttcaacgtaattgtggaggccctaagcaaatcgaaggctgaacttatggagatctcggaggacaagacaaagattcgtcgctcgccaagcaaaccgttgcctgaggtcaccgacgaatacaaaaacgatgtcaagaataggagcgtttacattaagggcttccccactgacgccaccctcgatgacatcaaggagtggctggaagataaaggccaggttttaaacatccaaatgcgccgcacactccacaaggctttcaaaggcagcatttttgtcgtgttcgacagtattgaaagcgctaagaagtttgtcgaaaccccaggtcagaagtataaggagaccgatcttcttatcctctttaaggatgactacttcgcgaaaaagaacgagtccggcgggtccaagcgtaccgctgatggctccgaattcgaacctaaaaagaagcgtaaggtgtaa

SEQ ID NO: 311 - DNA, 6570 bp, synthetic construct
  source 1..6570: mol_type = other DNA
atgaaacgaaccgctgacggatccgaattcgagcctaagaaaaaaaggaaggtctcaggtggatcgtcgggcggaagtacgctgaacatcgaagatgagtatcgactccacgaaacatctaaggagccggacgtctctctcgggagcacttggctctctgactttccacaggcctgggctgaaacgggcgggatgggtctcgctgtccgtcaggctccgcttatcatcccgttgaaggctacgtctacaccagtttcgatcaagcaataccccatgagtcaggaggctcgcctgggcattaagcctcacatccagcgcctcctggatcagggtatcctcgtcccgtgccagtctccttggaacactccactcctgccagtgaagaaacctggcaccaatgactacaggccggtccaggacctgcgggaagtaaataagcgtgttgaggacatccaccctacagtccccaacccctacaacttgctctccgggctgcctccttcgcatcagtggtacacagtgcttgatttgaaggatgcctttttttgcctccgtcttcatcctacatcccaaccgcttttcgctttcgaatggcgtgatccggaaatgggtatctctggtcaactcacttggacacgtttgcctcagggcttcaagaacagccctactttattcaacgaggcactccatcgtgatctagccgatttccgcatccaacatcccgacctcatcctgcttcagtatgtggacgacttgttgctggctgccacctcagagttggactgtcagcaaggcacacgcgcgctgttgcaaaccctcggaaacctcggctatcgagcatcggctaagaaggctcaaatctgccaaaagcaggttaaatatctcggatatctgctcaaagagggtcaaagatggcttactgaagcacgaaaagagactgtcatgggccaacctacccccaagacgccaagacagctcagagagtttctcggcaaggcgggtttctgcagactctttatccccggtttcgccgaaatggctgccccactctaccctctgaccaagcccggaaccttgttcaactggggtcctgatcagcaaaaagcgtaccaggaaatcaagcaggcgcttcttacagctccagccctcggcctaccagatctcacaaagccgttcgagttgttcgtcgatgagaagcagggttacgctaagggcgtgttgacccagaagctcggaccctggagacgtccagttgcatatttgtccaagaaactcgatcccgtagctgctggctggcccccttgcctccgcatggtagcagcaatcgcggtcctcacaaaggacgcaggcaaattgacgatgggtcagccgcttgtgattctcgcaccgcatgcagtcgaggcattggtcaagcaaccgccagatagatggctttctaacgcacgaatgacccactatcaagcgttattgctcgacactgatcgcgtacagtttggtcccgttgtggccctaaacccagcgacgttacttccactgccggaagaaggccttcagcacaattgcctcgacatccttgccgaggcgcacggtacgcgacctgaccttaccgatcagccattgcccgatgccgatcacacgtggtacactgacggatcatctctgctccaagagggccagcgcaaggccggagcggcggtcaccactgagacggaggtgatctgggccaaagcgctccctgctggcacctcagcacaaagggccgagctcattgcccttacgcaggcgctcaaaatggccgaaggtaagaaactgaacgtctacaccgattcgcgttacgctttcgccaccgctcatatccacggtgaaatctatcggagacgcggctggctgacctcggaaggaaaggagataaaaaacaaggacgaaattctggccttgctcaaagcactcttcttgccaaagcgcctttctatcatccattgccccggccaccagaagggacactcggcggaggctcgcggtaaccgaatggcggatcaagctgcccgcaaagccgccattacggaaaccccagacacaagtactctgctcatcgaaaactcgagcccttcaggtggatcgtctggcgggtcgtcgggctctgagactcccggtacttccgagagcgcaacccctgagagttctggcggatcgagcgggggcagtggaagttctaaactcgaaaagttcacaaactgttactccctatcaaagaccttgcgcttcaaagcgatcccggtcggaaagacgcaagagaacatcgacaataagcgcttgctggttgaagatgagaagagggctgaggactataagggtgtaaagaagcttcttgaccgttactatctctcattcatcaatgatgtgttgcactcgatcaaactcaagaacctaaacaactacatcagtctcttccgcaagaaaaccaggacagaaaaagaaaacaaagagctagagaacctggagatcaacctgcgaaaggaaattgcgaaggcctttaagggaaacgagggatacaaaagcctcttcaagaaagacatcatcgagaccatcttgccagagttcctcgatgataaggacgagatcgcattagttaacagttttaacgggttcacaaccgcattcaccggtttctttaggaaccgtgagaacatgtttagcgaggaggcaaagagcaccagcatcgcctttcgttgtatcaacgaaaatctgactcgttacatcagcaacatggacatcttcgagaaggttgatgcgatcttcgataagcatgaagtccaagaaatcaaggaaaagatcctaaatagcgattacgatgttgaagacttcttcgagggtgagttcttcaacttcgtgcttacccaagaaggcatagatgtttacaacgccattattggtggattcgtaaccgaatctggcgagaagattaagggcctcaacgagtacattaacctctacaaccaaaaaacgaagcaaaagttgcccaagttcaagcccctgtacaagcaagtcttgagcgaccgcgagtccctctccttctacggagagggatatacgtcagacgaagaagtcctcgaggtcttccgcaatacccttaataagaattccgagatcttctcatctataaagaagctcgagaaactcttcaaaaacttcgacgagtactcctcggccggcatctttgtcaagaatggcccggcgatctcgacaattagcaaggacatctttggagagtggaacgttatccgcgataagtggaacgcagagtacgatgatattcaccttaagaagaaagctgtggttacagaaaaatacgaagacgacaggcgaaagagcttcaagaagatcggttccttctcgttggaacagttgcaggaatacgcggacgcagaccttagcgtcgtcgagaagctcaaggagatcatcatccaaaaggtcgatgagatctacaaggtgtacggcagttcggagaaacttttcgacgcagactttgttcttgagaagtccttgaagaagaatgacgcagttgtagccattatgaaggacctcttggatagcgtgaagtcgttcgagaattacatcaaggccttcttcggtgaaggtaaagagactaacagggacgaatctttctacggtgacttcgtgctcgcctacgacatcctcctcaaagtggatcacatttacgacgccatcaggaattacgtcactcaaaagccatactccaaagacaagtttaagctctacttccaaaacccgcagttcatggggggttgggataaggacaaggaaaccgattaccgtgccactatccttaggtatggctccaaatactacctagcaattatggacaaaaaatacgccaagtgcctgcagaagatcgacaaggacgacgttaacggtaattatgagaagatcaactacaaactgttgccaggccctaacaaaatgctccctaaagtcttcttctctaagaaatggatggcttattacaaccccagcgaggacatccagaagatctataagaacgggactttcaaaaagggagatatgtttaacctcaatgattgccataagctgatcgacttttttaaggactcgatctccaggtacccaaagtggtctaacgcatacgacttcaatttcagcgagacagagaaatacaaggatatcgctgggttttatcgcgaggtggaagagcagggttacaaggtgtcgttcgagtcggcgtcgaagaaagaggtggacaagcttgtagaagaggggaagctctacatgttccagatctacaacaaagatttctccgataagtcgcacggcacccccaacctacacaccatgtactttaagttgctgttcgatgaaaacaatcacggtcagatcagactgtccggcggtgccgaattgtttatgcgtagggcctccctcaaaaaggaagagcttgtcgtccaccccgctaactccccaatcgcaaacaagaacccagataatcccaagaagaccacgaccctctcgtatgatgtctacaaagacaagcgattcagcgaagaccaatacgaactccacattcctattgctattaataagtgtcctaagaatatttttaagatcaacacggaagtccgcgtattgctcaagcacgacgataatccctatgtcattggtatcgatcgaggcgaacgcaacttgctctacattgtagttgtcgacggcaaaggcaacatcgtcgagcaatacagcttgaatgaaatcatcaacaactttaacggtattcggatcaagactgattaccactccttgctcgataagaaggaaaaggagcgattcgaagcccgacagaattggacgtcaatcgaaaatatcaaggaactcaaagcaggatacatttcacaagttgtccataagatttgcgagctggttgagaaatatgacgccgtcatcgccctcgaagacctcaacagtggttttaagaactcgcgagtcaaagttgagaagcaagtgtatcagaaattcgagaaaatgctgatcgataagctgaactacatggtcgataagaagagtaacccttgtgcaaccggtggcgctctcaagggataccagatcacaaacaagtttgaatcgttcaagtcgatgagcacacagaatggcttcatcttttacatcccggcttggctcacctccaagatcgaccccagcaccggtttcgtcaatctccttaagacaaaatatacgtcaattgcagactcgaagaagttcatctcttctttcgaccgtatcatgtacgtccccgaagaggaccttttcgagttcgctcttgactacaagaacttctcacgtaccgacgctgattacatcaaaaagtggaagctctactcatacggcaaccgtattcgaatttttcgaaaccctaagaagaacaatgtgttcgattgggaagaggtgtgcttgaccagcgcttacaaggaattgttcaataagtacggtattaattaccagcagggtgacatccgcgccctcctttgcgaacagtctgacaaggctttctacagctccttcatggcactcatgtccctgatgcttcaaatggcgaacagcatcacgggcaggacggacgtagacttccttatttcgccggtcaagaactccgatggcatcttctacgacagccgtaattacgaagcacaggagaacgccatccttccaaagaacgctgatgctaacggtgcgtacaacatcgctagaaaggtcttgtgggcaataggtcaatttaagaaggctgaggacgagaagctcgataaggttaagatcgccatcagtaacaaggaatggctcgaatacgcccaaacgagcgtgaagcatagcggatcaggcggcagctccggcggcagtatggcagagaacggcgataacgagaagatggcagccctggaggctaagatctgtcatcaaattgagtattatttcggtgactttaaccttccacgcgacaaatttctgaaagagcagatcaaactagatgaggggtgggttcctctggaaattatgattaagtttaatcggcttaacagacttactacggacttcaacgtcattgtggaggctttgtcaaagtcgaaggctgaacttatggagatttccgaagataaaaccaagatacgtcgatctccctcgaagcctttgcctgaagtcacagacgaatacaagaacgatgtaaaaaaccgctcggtctatattaaaggatttcccacagatgcgacacttgatgatatcaaagaatggttggaggacaagggtcaggtcttgaacatccaaatgcgtcgcacgctgcataaggctttcaagggctcgatttttgtcgtcttcgattccatcgagtcagctaaaaaattcgtcgaaactcctggtcagaaatataaggaaacggacttgctcatcctcttcaaagacgattacttcgctaagaagaatgagtccggagggtcgaagcgcactgcagacggatcggagtttgaaccgaaaaaaaagcgaaaggtctga

SEQ ID NO: 312 - DNA, 6567 bp, synthetic construct
  source 1..6567: mol_type = other DNA
atgaaacgcacggcagatggttccgagtttgaacctaaaaaaaaacgtaaggtcagtgggggaagcagcgggggatccacgctgaatatagaagatgagtaccgcctccatgagactagcaaagagcctgatgtttcccttggcagcacatggctttccgacttccctcaggcctgggcagagactgggggcatgggcctagctgtcaggcaggctccactgatcattcctctgaaggcaacctcaacccccgtctctattaaacagtatccgatgtcccaggaggcacggctgggcattaaacctcatatccagagacttttagaccaaggcattttagtaccatgccagtccccatggaacacgccacttcttcctgtcaagaaacccgggaccaatgattacaggccagtccaggacttgcgtgaggtcaataagcgggtggaagacatccatcccacagtgccgaacccgtataacctgttatcaggccttccaccatcgcatcagtggtacacagtcctcgatctgaaggatgcttttttctgtctgaggcttcatccaacatcccagcctctcttcgcattcgagtggcgggacccagaaatgggaatttcagggcagctgacttggacgaggttaccacaaggcttcaagaattctccaacattgtttaatgaggcgctacatcgggacctggccgattttcgaatccagcacccagatttgattcttttacagtatgttgacgacttactcctagccgccacctctgaactggattgtcagcagggcaccagagcccttctgcaaactcttgggaatctgggttatagagcctccgcaaagaaggcccagatttgccagaagcaggtgaagtatcttggatacctgctgaaagaggggcaacgctggttgaccgaagcaagaaaagagaccgtgatgggccaacccactccaaagaccccgcgacagcttcgggagttcttagggaaagccggattctgtagactctttatccctggatttgcagaaatggccgccccgctctatccccttactaaacccgggaccctgtttaattggggcccagaccagcaaaaagcataccaagagataaaacaggcactcctcacagcacccgccttaggcctccctgacttaaccaagccctttgaactctttgtggacgaaaagcagggatacgccaagggagtgctgacacagaagctcggaccctggcgacgtcctgttgcctatctgtccaagaagctcgatcccgtggccgctggttggccaccatgcctgaggatggtcgcagccattgctgtcttaaccaaagatgccgggaaactgaccatgggtcagcccctggtgatccttgccccacacgcggtggaagcactcgttaaacagccacctgatagatggcttagcaatgcccggatgacacactatcaagccttactgctcgacacagacagagtgcagtttggcccagtcgtggctctcaatcccgcaacactccttcctctgcccgaggagggtctgcagcacaactgcctggacatacttgccgaggcccatggcactcggccagatttgacagatcagccccttccagatgcagaccatacctggtacaccgatggaagttccttgctccaggaaggtcagcgtaaagctggggcggcagtgaccaccgagacggaggttatatgggctaaggcactgccggctggcacttctgcgcagcgggcagaactgatcgccctgacccaagcattgaaaatggccgagggcaagaagcttaacgtatatactgattcccgttatgcgttcgccacggcccatattcacggtgagatctataggcggcgaggatggttgacaagtgaaggtaaggaaatcaaaaacaaggacgagatcctcgcattgttgaaggcactgtttttacccaagcggctctcaatcatccactgcccggggcaccaaaaggggcattcagccgaggctcgcggcaaccgcatggcagaccaggctgcaagaaaagccgccatcaccgaaacaccagataccagcacactgctgatcgaaaattcttcaccaagcgggggctcgagcggaggttcaagcggcagtgagacacccggcacatccgagtccgctaccccagagagtagcggtggatcttctggaggcagcgggtcctccaagctggagaagttcaccaattgctactccttatctaagactctgcgcttcaaagccatccctgtgggaaaaactcaggaaaacatcgacaataaacgacttctggtggaagacgagaagcgagctgaagattataaaggggttaaaaagctgttggaccgctactacctgtctttcatcaatgacgtgcttcacagcataaagctgaagaacctgaacaactacattagcttatttaggaaaaaaacgcgcacagaaaaggagaataaggagctggaaaatttagaaattaatctgaggaaagagatcgcaaaagccttcaaaggaaacgagggatataaaagtctgttcaagaaagacatcatcgagaccattctacctgaatttctggacgacaaagacgagatcgctctcgtgaacagttttaacggctttacgactgccttcaccggctttttcaggaaccgcgagaacatgttctctgaggaggccaagtccacttctatagcatttcgttgcatcaacgaaaatttgaccagatatatttctaatatggatatctttgaaaaagtcgacgcgattttcgacaagcacgaggtgcaagagattaaagaaaagattctcaattctgattacgatgtggaagactttttcgagggtgaattttttaacttcgtgctcacccaggagggcatcgatgtgtacaatgccatcataggcgggtttgtaacagagtccggagagaagatcaaaggtttgaacgaatacataaacctgtataaccagaaaaccaaacagaagctacccaagtttaaacctctgtataaacaggtcctgtcagatcgcgaatcacttagcttctacggagagggctacaccagcgatgaggaagttctcgaggtatttcgcaacaccctgaataagaacagcgaaattttcagcagcattaaaaaattggagaaactgttcaaaaatttcgacgagtattcaagcgccggaatctttgtgaagaacggccccgcgatttcaaccatctccaaagacatctttggagaatggaacgttatacgggacaagtggaacgcggaatacgacgacatacatctgaaaaaaaaggcggtggtgaccgagaagtatgaggacgatcgcagaaagtcattcaaaaagattggctcgttctctttggaacagttgcaggagtatgcggacgctgatttatctgtcgtggaaaagttgaaggagatcattattcagaaagttgacgagatatacaaagtgtatggatcctccgaaaagctgtttgatgctgacttcgtgctggagaaaagtctgaagaagaatgacgccgtcgtagcaatcatgaaagatcttctggattccgtgaagtcgttcgagaactatatcaaagcattctttggtgagggcaaagaaacgaaccgcgatgaatccttttatggtgacttcgtgttggcttatgacatcctgctgaaggttgaccatatctacgatgccattagaaactacgtgactcaaaaaccatatagcaaagacaaattcaaactctactttcagaatccacaatttatgggtggctgggataaggataaagaaaccgattacagggccacaattttgaggtatggatcaaagtactacctagctattatggataagaaatacgcaaagtgcctccaaaaaatcgacaaagatgatgtgaacgggaattatgaaaagattaattacaaattactgcctggacccaataagatgctgccaaaggtattcttctcaaaaaaatggatggcctactataatcctagcgaggatatccagaagatctacaaaaatggtacattcaagaagggagatatgttcaatctgaatgattgtcacaaacttattgactttttcaaagactcgatttcccgctatcccaagtggtctaacgcctacgatttcaacttcagcgaaacggagaagtacaaggatatcgctgggttctaccgcgaagtggaagagcagggttacaaggtgtcttttgagagtgcctccaagaaagaagttgacaagctggtggaagaagggaaattatacatgttccaaatttacaacaaggacttctccgataagtcacacgggactccgaacctgcacactatgtactttaagctgttattcgacgagaataatcatggccagatccggttgagcggcggggccgaactgtttatgcgcagagcttcactgaagaaagaagagctggtggttcaccccgccaacagccccatagccaataaaaacccagacaaccccaagaagaccaccacactgagctacgatgtctataaggataaacgcttctccgaggatcagtacgagctccacatccctatcgctatcaacaagtgccccaagaacatattcaaaatcaatactgaagtcagagtcttgctgaagcacgacgacaatccttatgtcataggtattgatcggggcgaacgcaacctgctgtatatcgttgttgtcgatggaaaaggaaacattgtggaacagtatagtctgaacgagatcattaataactttaatggaatcagaataaagacagactatcactccctgctcgacaagaaggagaaagaacggtttgaggcccggcaaaattggactagtatcgagaatatcaaagaactgaaagccggctatatttcccaggtggtgcacaagatttgcgagctcgtagagaaatatgatgccgtgatcgctctcgaggacttgaactcgggattcaagaatagtcgcgtaaaggttgagaagcaggtctaccagaagttcgagaagatgctcattgacaaactcaattacatggtggataagaagagtaatccatgtgcaacagggggggccctgaaagggtatcaaatcacgaataagtttgagtcctttaagtccatgtccactcagaatggatttattttctatatccctgcatggctgacctccaagatagatcccagcaccgggtttgttaacctcctgaagacaaagtacacatccatagcagacagcaagaagttcattagtagctttgatcgaatcatgtatgtgccagaggaggatcttttcgaattcgccctcgactataaaaatttcagccgcacagacgcagactacattaagaaatggaagctttattcctacggcaataggatccggatttttcgcaatccaaaaaagaataatgtcttcgattgggaggaagtgtgtttaacctcagcctataaggagttgttcaacaagtatgggatcaattatcagcaaggcgacattcgtgctctactgtgtgaacagagcgacaaggcattttactccagttttatggctttaatgagcctgatgctgcaaatggccaactccataaccggccgcaccgatgtggacttcctgatcagccccgttaaaaacagtgatggtatcttttacgactctcgtaactatgaagctcaggaaaatgccattttgcctaaaaacgccgacgctaatggagcatataacatagcccgaaaggtgttgtgggccatcggtcaattcaaaaaggcagaggatgagaaattggataaagtgaaaatcgcaatttccaataaggagtggctcgaatatgcgcagacgtctgtcaaacattcaggctccgggggcagctccgggggctcaatggcagagaacggagataatgagaaaatggccgccctcgaggctaagatttgccaccaaattgagtactacttcggtgattttaatctcccccgcgacaaatttctgaaagaacagataaaactggacgaagggtgggtgccactagaaataatgataaaattcaatcgactgaatcggcttactaccgatttcaatgtgattgtcgaggctctgagcaagtcaaaagctgagctgatggagatcagtgaagacaaaacaaagatcaggcgttcaccgtcaaaacccttacccgaagttactgatgagtacaagaatgacgttaaaaacagatcggtctatataaagggattccctactgatgccacccttgacgacattaaagaatggttggaggacaaaggtcaggttcttaatatccagatgaggcgaacgcttcataaggcattcaaaggatctatcttcgtggttttcgactcaattgagagtgctaaaaaatttgtggaaaccccgggacaaaaatataaggagactgatttgctgatcctgttcaaggatgattacttcgccaagaagaatgagagcgggggttcaaagcgaacagcagacgggtcagagttcgagcctaagaaaaaaaggaaagtg

SEQ ID NO: 313 - DNA, 6567 bp, synthetic construct
  source 1..6567: mol_type = other DNA
atgaagagaacagccgacggcagtgaattcgagccgaaaaagaagagaaaagtttcaggcggttcatctggtgggagtacactgaatattgaagacgagtataggctacacgaaacgtcaaaggagccagacgtctcattgggcagcacctggttgagtgacttcccgcaggcctgggcagagacaggcggaatgggattggccgtgcgccaggcacccctcatcatccccctgaaggctaccagcactcctgtttcgattaagcagtatcctatgtcccaagaagcccgcctgggaatcaagccccacattcagcgtctcctggatcagggcatcttggtaccatgtcaaagcccttggaacacacccctactgccggtcaagaagcctggcactaacgactaccggcccgtccaggatctgagagaggtgaacaagagggtggaagatatccacccaaccgtaccaaacccttacaacctgctgagcggtctaccaccatctcaccagtggtacactgtcctggatctgaaggatgctttcttctgtctgcgactgcatcccacctcacagcccttgttcgccttcgaatggagggatcccgaaatgggcatctccggacaactgacttggactaggctgccacagggcttcaagaatagccccaccttattcaacgaggctctccaccgcgatctggctgattttcggattcaacatcctgatctgattctacttcagtacgtagacgatctgttactcgcggcaacctcagagctggattgccagcagggcacccgggcattgctccagacgttgggaaacctggggtaccgggcaagtgccaagaaggcgcagatctgccagaagcaggtgaaatatctgggatacctactcaaggaggggcagcgctggctcaccgaagcccgcaaggagacagtcatgggacagccgacacctaaaaccccgagacagctgagagagtttctggggaaggctgggttctgcaggttattcatccccggcttcgctgaaatggcagccccattatatcccctcacaaaacccggcactctgttcaattggggtcccgaccaacagaaggcataccaagaaatcaagcaagccttgctgacagctcccgcactgggtctgccagatctcaccaagcccttcgaactgtttgtagacgagaagcaggggtacgccaaaggggtactcacacagaagctgggtccttggcggcgtcccgtggcctacctgagcaagaagttggacccggtggcggccggctggcccccctgtctgagaatggtcgccgccattgccgttctcacaaaagacgcagggaagctgacaatgggccagcccttagttatccttgctccccatgcagtcgaggctctggtgaagcagcctcccgatcgctggctctcaaacgcgaggatgacacattaccaggcactactgctggacaccgacagagtccagttcggtccggttgtggcgcttaaccctgcaactctgcttcccctgccggaggagggccttcagcacaactgcctggacatactggcagaggcacatgggacccgccccgacctcacagatcaaccactgccagacgcggatcatacatggtacaccgatggttcatcattgctgcaggagggtcaacgcaaagccggagctgccgtgacaaccgagacagaagtcatctgggctaaggctctgcccgctgggacgtcggcgcagagggcagagctcattgctctgacacaggcactgaagatggccgagggaaagaaactcaacgtctatacggactccagatacgctttcgccacagcccacatccacggagagatttacaggcgtcgggggtggttgacttccgaaggcaaggagatcaagaataaagacgagatccttgcacttctgaaggccctcttcctccccaagcggctttccatcatacattgtccaggacatcagaagggtcattccgccgaggccaggggcaaccggatggcggatcaagctgctcgcaaagctgccataacggagacacccgacaccagcacactgctgattgaaaatagtagcccgagcggtgggtcaagcggaggttcaagcgggagcgagactccaggaaccagtgagtcagccacccccgagtcgtcgggcggatcaagcggcggtagtggatcgtctaaattggaaaagttcaccaactgctattctctgtcaaagaccctccgttttaaagccattccggtgggaaagacccaggaaaacattgataacaagcggctacttgttgaggacgagaagagagcggaggactataagggtgttaaaaagttgttagatcgatactacctgtctttcatcaacgatgtcttacactcaatcaagttaaaaaatctgaacaactatatctctctgttcagaaaaaagacccgaactgagaaggagaacaaggagctggagaatcttgaaatcaaccttagaaaggagatcgccaaagcttttaaaggaaacgaggggtacaagtctctcttcaaaaaagatattatcgagactatactgcccgaatttttggatgacaaggacgagattgcccttgtgaatagctttaacggatttaccacagcgttcactggctttttccggaaccgtgaaaatatgtttagcgaggaggcgaagtccacatccatcgcctttcgctgtatcaacgagaacctgactcggtacatcagtaacatggatatatttgagaaggtcgacgctatctttgataagcacgaggtgcaggagatcaaggagaagatcctgaatagtgattacgacgtagaggacttcttcgagggagagttttttaacttcgttctgacacaggaaggcattgatgtttacaatgcgattatcgggggcttcgtcaccgagagtggggagaagatcaagggactgaacgagtacatcaacctctacaaccagaagacaaagcaaaagctgcccaagttcaagccactgtataagcaggtgctgagcgatcgtgagagtttgtccttctacggagaagggtatacttcagacgaagaagtattagaagtgttcaggaacaccctgaacaagaactctgagatcttcagtagcataaaaaaactggaaaaactttttaagaattttgatgagtatagttccgctggcattttcgtgaagaacgggccagcaatctctaccatcagtaaggacatcttcggcgagtggaatgtgattcgggacaagtggaacgctgagtacgacgatatccaccttaagaaaaaagctgtagtcacagagaagtacgaggacgatcgacggaaatccttcaagaagattggctctttctccctcgagcagctgcaggaatacgcagacgctgacctgtccgtggtagagaagctgaaagagattatcatccaaaaggtggacgaaatctacaaagtctacggctcttctgagaagctgttcgacgctgacttcgtgctggagaagtccttaaaaaagaacgacgcagtggtagcaataatgaaagatttgctggactcggtgaaaagcttcgaaaactatatcaaggccttctttggagagggtaaggagactaacagagacgagtcattttacggcgacttcgtcctggcctatgacatccttctaaaagttgatcacatctatgatgcaattcgaaactatgtcacgcagaaaccctacagcaaagacaaattcaagctctattttcaaaaccctcagttcatgggagggtgggataaggacaaggagaccgattatagagccaccattctgcgctacggttccaaatattacctggccatcatggataagaagtacgccaaatgcctccagaagattgacaaggacgatgtcaacggtaactatgaaaagatcaactacaagctgctgcccggacctaacaagatgctcccaaaagtgttctttagcaaaaaatggatggcctactacaatccgagcgaggacatccaaaaaatttataagaacggaacatttaagaaaggagatatgttcaatttgaatgattgccacaagctcatcgatttcttcaaggattccatctctcgttaccctaagtggagcaatgcctatgactttaatttcagcgagactgagaagtacaaggacatcgcaggcttctacagggaggtggaggagcaagggtataaggtgtcttttgaatctgcgagcaagaaggaggttgacaaactggtggaggagggcaagttatatatgtttcagatctacaataaggacttcagcgacaagagccacggtactcccaatctgcatacgatgtactttaagttactctttgacgagaataaccatggacagattcgattgagtggtggcgcagagctcttcatgcgccgggctagcctgaaaaaggaagagctggtagtacatccagctaactcgcccatcgcgaacaagaatcccgacaatcccaagaaaacgacaacactatcttatgatgtctacaaggacaaacggtttagtgaggaccaatatgaactgcatatacccattgccatcaacaagtgcccaaaaaatattttcaagattaacaccgaagtgcgtgttctcttgaaacacgacgataacccatatgtgataggcattgacaggggggaaaggaatctgctgtacatcgtcgtggttgacggcaagggcaacatcgtggagcagtactctcttaacgagattattaataacttcaacgggattcggatcaaaacagattaccactccttgcttgacaagaaggagaaagaacgtttcgaggcccggcagaactggacaagcatcgagaacattaaagaactgaaagccggttatatttctcaagtggtgcataagatatgcgagctggtggagaagtatgacgctgtgattgccctggaggaccttaatagtggcttcaagaatagtagggtgaaggtggagaaacaggtgtatcagaaattcgagaagatgttaatcgacaaactgaactacatggttgacaagaagtcaaacccctgcgccactggcggcgcgttgaagggttaccaaatcacaaacaagttcgagtcatttaaaagtatgtctacccagaatggattcatcttctatatccccgcttggttaacctctaaaattgacccctctaccggctttgtgaacctactcaaaacaaagtatacttctatcgctgacagtaagaagttcatctcatccttcgatcggatcatgtacgttcccgaggaagatctgtttgagtttgcccttgactataaaaacttttctagaacagacgccgattacattaagaagtggaaactgtattcctatggaaatagaatcaggatctttcggaacccgaagaagaataacgtgtttgactgggaggaagtctgcttgacttcagcatacaaagaactgttcaacaagtacggaatcaattaccagcagggggacattcgggctttactgtgcgaacagagtgacaaagctttctacagctcatttatggcgctgatgtcactcatgctccagatggccaactctattactgggcggacagacgttgatttcctgatctctccagtgaagaattctgatggcatcttctacgactctcgaaactatgaagcccaggagaatgcgattctccctaagaacgccgatgccaacggagcatacaatatcgcacgcaaagtgttatgggctatcggtcagtttaagaaggctgaagacgaaaaactagataaagttaaaatagcaataagcaataaggagtggctggagtacgcacagacaagcgtaaagcacagcggttcaggtggcagctcgggtgggtccatggcggagaatggcgacaacgagaagatggcagcactcgaggcaaagatctgtcaccagatcgaatactacttcggggattttaatctgcctcgggataagttccttaaagagcagatcaaattagacgaaggctgggtgcctttggaaatcatgattaaattcaatagactgaataggcttacaacagactttaacgtgattgtggaagccttgtccaagtctaaggccgaattgatggaaatatcagaagacaagactaagatcagaagatccccatctaaacccttaccggaggtaacagacgaatacaagaacgatgtcaagaatcggagcgtatacatcaaagggttccctacagatgccactttggacgatatcaaggaatggctcgaagataaaggccaggtactgaacatacagatgagacggactctgcataaagccttcaagggctcaatcttcgtcgtgttcgattctatagagagcgccaaaaaatttgtcgaaactcctggtcaaaagtacaaagaaaccgacctgctgatcctctttaaggacgactactttgcaaagaagaatgaaagcggcggttctaagcgcaccgccgatggtagtgaatttgagcctaagaagaagcgcaaggtc

SEQ ID NO: 314 - DNA, 6567 bp, synthetic construct
  source 1..6567: mol_type = other DNA
atgaagcgcacggcagacggctctgaattcgaacccaagaaaaagcggaaggtcagtggcggttcaagcggaggatcaacgctgaacatcgaggatgagtacaggctccacgagacatccaaggagccagacgtgagtctggggtcaacctggctatcagacttcccccaagcatgggcagaaacaggcggaatgggtctggccgtgcgccaggcgcccttgataattcctttgaaggccactagtaccccagtgagcataaagcagtatccaatgtcacaagaggctcggctggggataaaacctcacatacaacgcctgctggatcaagggatcctggttccctgtcaatctccttggaacactccgctcctgccggtaaagaagcccgggactaacgattatcggcccgtccaggacctcagggaagtgaataagcgggttgaggatatccaccctactgtgccaaatccatacaacctgctctctggcctgcccccttcgcaccagtggtatactgtgttagacctgaaggacgcgttcttttgtctacgcttacatcctacctcacaacctctcttcgccttcgagtggagagaccctgaaatggggatttctggacagctgacctggacccggctcccccaaggcttcaagaactctcccacccttttcaatgaggcgctgcatcgcgatctggccgacttccggatccagcaccctgatctgatcctacttcaatacgtggatgacctgcttttggccgccacatccgaactggactgccagcagggcacaagggcgctactgcagaccttagggaatctggggtaccgggcttcggcaaaaaaggctcagatatgccaaaaacaggtgaaatatctgggctatcttcttaaggagggccagcgatggctgacagaggcccggaaggaaactgtgatgggccagccaactcccaaaacaccgaggcagctcagggaatttctggggaaagcaggattctgcaggctgttcatccccggttttgcagagatggccgcccccctctatccactcaccaagcctggcaccctgttcaattggggccccgaccagcagaaggcctatcaggaaatcaagcaggcgttacttaccgctccagctctaggcctgcctgatctgaccaaaccgtttgagctcttcgtcgacgaaaagcagggttacgccaagggcgtcctgactcagaagctgggaccatggcgcagaccagtagcatatctgtcgaagaaactagaccctgttgccgcaggttggccaccctgcctacgcatggtggcagcgatcgccgtgctgaccaaagacgccggcaaactgactatgggccaaccccttgtgatcctcgccccccacgcggtcgaggcgctggtcaaacagcctccagatcggtggctgagcaatgctcgcatgacacactaccaggctctactgcttgacacggaccgcgtccagtttggccctgtcgttgccctgaaccctgccactctcctacccctccctgaggaaggactgcagcataattgccttgacatactggctgaagcccatgggacacgtcccgacctgaccgatcagcccctgccggatgccgaccatacatggtacacagacggctcttcccttctccaggagggacagaggaaggccggagctgccgtgacaactgagaccgaagtcatctgggcaaaggcactccctgccggaaccagcgcgcagagggccgagctcatcgcacttacacaggccctgaagatggcggaaggaaaaaaacttaatgtctatacggattctcggtacgccttcgccacggcccacatccacggcgagatctacagacgaaggggatggctaacaagcgaaggaaaggagatcaagaataaagacgagattctcgccctgttgaaagccctgtttctgcctaagcgtcttagtattattcattgtcccggtcaccaaaagggacacagcgccgaggctagaggcaaccgcatggccgatcaggctgcacgcaaagccgctatcactgaaacccccgacacatctacactcttgatagagaactcctcacccagcgggggctctagcggcggcagcagcggatctgagacgcctgggacatctgagagcgctacaccagaaagtagcggaggcagctctggcggatctggcagcagtaagctcgagaagtttactaactgttacagtctgagcaaaactctgcgttttaaggcaatcccagtgggcaagactcaagaaaacatcgataacaagaggctgctggtggaggacgagaaacgcgcagaggactacaaaggcgtgaagaagctgttggatcggtattacttgtctttcattaatgacgtcttacactctatcaagcttaaaaacctgaacaactacatatctctattcagaaaaaagacacgtaccgagaaggaaaataaggagctagagaacctggaaatcaatctgcgtaaggaaattgccaaagcattcaaggggaacgaaggctataagtcattgttcaagaaagacatcatcgaaacaattctgcctgagttcttggacgacaaagatgagattgccctggtaaattcttttaatggatttacgactgcatttacgggtttcttcaggaatcgggagaacatgttcagcgaggaagctaaatccactagtattgcattcagatgtatcaatgaaaacttgactcgctatatttctaacatggatatttttgagaaggtcgacgctatatttgacaagcatgaggtacaggaaatcaaagagaagattctgaatagcgactatgatgtggaggacttcttcgaaggcgagttctttaattttgtactgacccaggaagggatcgatgtttacaatgcgataattggaggcttcgtcactgaatcaggcgagaagatcaaagggttgaacgagtacattaatctttacaaccagaagaccaagcaaaagctgcccaagtttaaacctctgtacaagcaagttctgtcggatagggaatcactctctttctacggcgaaggatacaccagcgatgaagaggtcctggaggtgttcaggaacactctgaacaagaactcagagatcttctccagcataaaaaagttggagaaactcttcaaaaactttgacgagtacagcagtgccggcatcttcgtgaagaacgggcctgcgattagcaccatttcaaaagatatcttcggtgaatggaacgtgattcgtgacaagtggaacgcggaatacgacgacatccacctgaaaaagaaagccgtcgttacagaaaaatacgaggacgatagacggaaatcattcaagaagataggtagtttttccctcgagcagttgcaagagtatgccgacgctgatctgagcgttgtcgagaaactaaaggagatcataatacagaaggtggacgagatatataaggtctatggaagcagcgagaaattattcgacgcggatttcgtattggagaaatcactgaagaagaatgacgcagtcgtggcaattatgaaggacctactcgactcagttaagagcttcgagaattatattaaggccttctttggagaaggaaaggaaacaaacagggacgagagtttctatggagacttcgtcctcgcatacgacattctgctcaaggttgaccatatttacgacgcgatccggaactatgtcactcagaagccctacagcaaagacaagttcaagctatacttccagaaccctcagttcatgggcggatgggacaaggataaggaaaccgattaccgggctaccattctaagatacgggtcaaaatactatctggccattatggataagaaatacgcaaagtgtttacagaagattgacaaggacgacgtgaacggcaactacgaaaaaatcaattataaactgttgcctggtccgaacaagatgctgcccaaagtctttttcagcaagaaatggatggcgtattacaatccgagcgaggacatccagaaaatctacaagaacggaacgttcaaaaaaggagacatgttcaacctaaatgattgccacaaactaatcgatttttttaaagatagcattagccgctaccccaagtggtccaatgcctatgactttaacttttctgaaaccgaaaaatataaggacatagccgggttttacagggaggtcgaggagcagggctacaaggtgtcctttgagtcagcgtcaaagaaagaggtggacaagttggtggaagaagggaagctctacatgtttcagatctataataaagacttttctgacaagagtcacggcacccctaatctccacaccatgtacttcaaattgctttttgatgagaacaaccacggccagataagactcagtggaggtgccgaattatttatgcggagggctagtttgaagaaagaggagctcgtcgtgcatcccgctaactctccaattgctaacaagaacccggacaatccaaagaaaaccaccaccctgagctatgatgtgtacaaagacaagcggttctccgaagaccagtacgaactgcacatcccgatagccatcaacaaatgccctaaaaatatcttcaagataaacactgaagtgcgcgtactgctcaagcatgatgataacccctatgtcatcggcattgaccggggcgagcggaacctgctgtacattgtcgtggtggatgggaagggaaacattgtggagcaatatagtctcaatgagatcattaacaactttaatggaatacggatcaaaacggactaccatagcctcttggataagaaggagaaagagcgtttcgaagcgcgacagaattggacaagcattgaaaacataaaggagttaaaggccggttacatctctcaggtcgttcataagatctgcgagcttgtcgagaaatatgatgctgtgatcgcactcgaagacctcaatagtggctttaagaactctagggtgaaagtagagaaacaagtttaccaaaagttcgagaagatgctgatcgataagctgaattacatggtcgacaaaaagtccaatccctgtgcaacaggaggagctctcaagggttaccaaatcacaaacaaatttgagagttttaagtcaatgtccacacagaacggatttattttttacattcctgcctggctgaccagtaagatcgatcccagcactggcttcgttaatcttctcaagaccaaatatacgagcatagcggactcaaaaaagtttatctcttcgtttgaccgcattatgtatgtgcccgaagaagacctctttgagtttgcactggactacaagaatttctcccgcactgatgccgactacatcaaaaagtggaagctctacagctatgggaaccggattcgcattttcaggaaccccaaaaagaacaacgtgttcgattgggaagaggtgtgcctgacgagcgcatacaaggagctgttcaacaaatacggaatcaactaccaacagggggacatccgggctctgttatgtgagcaaagcgataaggcattctacagctcgtttatggcactgatgtctctgatgctgcagatggccaactcgatcaccggcaggactgacgtggatttcctgattagtcccgttaaaaactcagacggtatcttctacgactctaggaactacgaagctcaggaaaacgccattttaccaaagaacgccgacgcaaatggcgcctacaatatcgcccggaaggtgctctgggcaatcggccagtttaaaaaggcagaggacgaaaagctcgacaaagttaagattgcaatttctaacaaagagtggctggagtatgcccagacaagcgtaaaacattccggctcaggcggttctagcggcggatcaatggcagagaacggagacaacgaaaaaatggctgccctcgaggccaaaatttgccaccagatcgaatattacttcggcgacttcaatctgccccgcgacaagttcctaaaagagcagattaagctggacgagggttgggttccactggaaatcatgattaagtttaatcggcttaacagactgaccacagactttaatgttatcgtcgaggctttgtcgaagagtaaggctgagcttatggaaattagtgaagataaaaccaaaattagaaggtccccatccaagccactgcccgaggtaaccgacgagtataaaaacgacgtgaagaacaggagcgtgtatattaagggatttccaacagacgcaacactcgatgacattaaagaatggttggaagacaagggccaggtgctcaacatccagatgaggcgcactcttcacaaggcattcaagggttctattttcgtggtttttgactctattgaatctgctaagaagtttgtggagacgccggggcagaagtataaggagaccgatttgttgattctgttcaaagatgactatttcgcaaagaagaacgaatcaggcggaagcaaaaggacagccgacggatccgaattcgaacccaaaaagaagcgcaaggtc

SEQ ID NO: 315 - DNA, 6567 bp, synthetic construct
  source 1..6567: mol_type = other DNA
atgaaacggaccgctgatggctccgagttcgagcctaagaaaaagcggaaggtgtccggcggttcatcaggcgggtcaaccctgaacattgaggatgaatataggctgcacgagacttccaaagagcctgatgtatcactaggttcaacgtggctctctgatttcccccaggcctgggccgagaccgggggaatgggccttgctgtaagacaggctcccctcatcatccctttgaaggctacaagcacacccgtcagtatcaaacagtaccctatgtcccaagaagctcgcctgggtatcaagccacacatccagagactcctggaccagggcatattagtaccctgccagtcaccatggaacacaccactcttacctgtcaagaagccaggaacaaacgattatagaccagtccaagatcttcgggaggtgaataaacgggtcgaagacatccaccccacagtaccaaatccatataacctgttgtcagggttacctccaagtcatcagtggtacacagtgctggacctgaaggatgcctttttctgcctgagactccaccctacatcccagcctctgtttgcattcgagtggagggacccagagatgggcatttcaggtcagcttacctggacccgcctaccacaggggtttaaaaattcaccaactctgttcaatgaggctttgcatcgtgatctggcagacttcagaattcagcaccccgatttgatccttctgcagtacgtggacgatcttctgctggctgctacatcagagttggactgccagcaaggtactcgcgctcttctgcaaacgctcggcaacctcggctacagagcatccgccaagaaagcccagatctgccagaagcaagttaagtacttggggtatctgctgaaagaagggcagagatggctcacagaggccagaaaggaaactgtgatgggccagccaacccccaagacccctaggcagctcagggagtttctgggtaaagcaggattttgccggcttttcatacctggattcgccgagatggctgcaccgctctaccccctgaccaaacctgggactctgtttaactggggcccagatcagcagaaagcctatcaggaaattaagcaggccctgttaactgcccctgccctcggcctgcctgacctaacaaaacctttcgagctgttcgtagacgagaagcaggggtacgctaagggcgtgcttacccagaagctcggtccatggcgtaggcctgtggcctatctctccaagaagctcgatcctgttgccgctggatggccaccatgtctgcgaatggtggccgcaatagcagtgctcactaaggatgcaggaaagctgaccatggggcagcctctggtcattctggctccccacgccgtcgaagctctcgttaagcagccacctgacaggtggctcagcaacgcccgcatgacgcattatcaggcacttctactagacactgaccgagttcagttcggacctgtggttgcccttaaccccgcgactctgttgcctcttccagaggaaggattgcagcataattgcctggatatcttggcagaagctcatggaacaaggcctgatctgacggatcagcccctgcctgatgccgaccatacttggtataccgacgggtctagtcttctgcaggaaggccagcggaaggcgggcgctgccgtgactactgaaacagaagtgatctgggcgaaagcactgcccgctgggacttctgcacaaagggccgagctgattgctctcacacaggccctgaaaatggctgagggtaaaaagttgaacgtgtacaccgatagtaggtacgctttcgcaacggcacatatccatggcgagatctacagacgcagggggtggctgacctccgagggcaaggagatcaaaaataaggacgaaatcctggctctgctgaaagccttgttcctgcccaaaagattgtcaatcattcattgcccaggccaccagaagggacacagtgcagaggctcggggtaacaggatggcggaccaggccgcacggaaagcagcgataacagaaacgccagatacttcaaccctgctcatcgaaaacagtagcccttctggaggctcttccggaggctcttccggatctgaaactcccggcactagtgagtctgcaacaccagagtccagcggcggatccagcggaggttctggatcctccaaactggaaaaatttaccaattgctactctctgagtaagacgctaagatttaaggcaatcccagttggcaagacacaggaaaacattgataataaaagattattagtggaagatgaaaagagagctgaagactacaagggtgtgaaaaagctcttggacaggtattacctgtcgtttatcaacgatgtcctacactccatcaaattgaagaacctgaataattacatcagcctattcagaaaaaagacaaggacagaaaaggagaacaaagaattggaaaatttggaaataaatctccgaaaggaaatagcgaaagcttttaagggaaacgaggggtataaatccctcttcaagaaagacataattgaaaccattctgcccgagttcctagatgacaaagatgaaatagcgctggtcaactctttcaacggctttaccactgccttcactgggttctttagaaatagagaaaatatgttcagcgaagaagccaagtctacgtccattgcttttcgttgcatcaatgaaaatctaactcgctatatttctaatatggacatctttgagaaggtggatgcgatctttgataaacatgaggtccaggaaatcaaagagaagatcttgaactctgattacgatgtggaagatttctttgaaggagaattctttaactttgtgctgacccaagaaggtatcgatgtgtataatgcaattatcggcgggttcgtgacagagagcggggaaaagatcaagggtctgaatgagtacatcaacttgtacaatcagaaaacaaagcagaaactacctaagttcaaaccactgtacaaacaggtgctgagtgaccgggaaagtcttagtttttatggtgagggctatacttcagatgaagaagtccttgaggtgttcagaaacacattgaacaagaattccgagatattctctagcattaagaaactcgagaaactgttcaagaacttcgacgaatattcaagcgccggcatttttgtcaagaacggcccagccatctccactatttccaaggatatctttggagaatggaacgtaattcgggataagtggaacgctgaatacgacgatatccatctaaaaaagaaggcggtggtaacagaaaagtacgaggatgatcggaggaaatcatttaaaaaaatcggcagtttctctcttgagcagctgcaggagtatgcggacgccgacctcagtgtagtggagaagctgaaagaaataattatccagaaggtggatgagatctacaaggtttatggatcaagcgaaaagttgtttgacgccgatttcgtgcttgagaagtcactgaaaaagaacgacgccgtggtggctatcatgaaggaccttcttgattctgtgaagtcatttgagaattatattaaggctttctttggagaaggaaaagagaccaatcgggatgaatcattttacggtgactttgttttggcctatgacatccttctgaaggtcgatcatatctacgacgcaatcaggaattatgtaacgcagaaaccatactcaaaagataagttcaagctatacttccagaacccccagttcatgggtgggtgggataaagacaaagagaccgactaccgagccaccatcctgaggtacgggtcaaagtattatcttgcaattatggacaagaagtacgctaagtgcttacagaagatcgataaggacgatgtcaacggcaattatgagaagataaactataaattgcttcctggcccaaacaagatgcttcccaaagtgttcttttccaagaaatggatggcatactataacccgagcgaagatatccaaaagatctataagaacggaacctttaagaaaggcgatatgttcaacctgaacgactgtcataaactgatagatttcttcaaggatagcatttcccgttaccctaagtggagcaacgcatatgactttaacttctccgaaaccgaaaagtacaaagacatcgccggcttttatcgtgaagtggaggaacagggatataaagtctcttttgagtcggctagtaaaaaagaagtggacaaactggttgaggagggcaaactctacatgttccagatctacaataaagacttctctgataagtcacatggcactcctaacttgcatactatgtacttcaagctgttatttgacgagaataatcatggccaaattagattatctggcggcgccgagctttttatgagaagggcatctttaaaaaaggaagagctcgtggtacatcccgctaatagccctattgctaacaaaaacccagacaaccccaagaaaacaaccacactgagctacgatgtgtacaaggataagcgcttttctgaagatcagtacgaactgcacattcctatcgctattaataagtgtcccaagaatatctttaaaatcaacaccgaggtgcgcgtgctgctgaagcacgatgacaacccgtatgtaatcgggatcgacagaggtgagcggaacctgctgtacatcgttgtcgttgacgggaaaggcaacattgtggagcagtatagcctgaacgagattataaacaattttaatggcattcgcataaagacagactatcactcattgctggataagaaggaaaaggagagattcgaggcgcggcaaaattggacttctattgaaaacatcaaggagttgaaggccggctacatttctcaagtagtgcataagatctgcgaactcgtggagaaatacgatgctgtgatcgctctggaagaccttaacagtgggtttaagaacagccgggtgaaggttgaaaagcaagtgtatcagaaattcgaaaaaatgctaatcgacaagctgaactacatggtcgacaaaaagtcaaacccatgtgcaacaggtggtgctctgaaaggttatcagattaccaataagttcgagtcattcaaaagtatgtctacccaaaacggatttatcttctatatcccagcttggctgaccagtaaaattgacccatccaccgggttcgtaaacctgctgaaaacaaaatatacaagtatagccgactctaagaagttcatcagctccttcgatcgcatcatgtacgtacctgaagaagacctttttgaattcgctctcgactacaagaacttctctaggaccgacgctgactacatcaagaaatggaaattatattcgtacggcaataggattaggattttccggaatccaaagaaaaacaacgtgtttgattgggaggaggtgtgcctcacttctgcatataaggaattgttcaacaagtacggcatcaattaccaacaaggagatattcgagcactgctgtgtgaacagtctgataaagccttctattcgtcattcatggctctcatgtcactaatgctgcagatggctaacagcataacaggaagaacagacgtagattttctgatctcacctgtcaagaattctgacggcatcttttacgactctaggaactacgaggctcaggagaacgccattctgcccaaaaacgcggacgccaacggagcttataatattgctcgtaaggtactatgggctataggccagttcaagaaggccgaagatgaaaagctggataaggtgaagatcgctatctctaataaagaatggctagagtacgcacagacatccgtcaagcactcgggaagtggtgggagctccggcggctccatggcagagaacggagataacgaaaagatggcggcactggaagcgaaaatctgccatcagattgaatattatttcggagactttaacctccccagggataaattcctgaaggaacagattaaactggacgagggctgggttcctctcgagattatgatcaaattcaacagactgaataggctgactactgacttcaacgtcatcgtcgaagccctctccaaaagcaaggctgagctgatggagattagtgaagacaagaccaagattcggaggtctcccagtaaaccactacccgaggttaccgacgaatataaaaacgacgtgaagaaccgctctgtgtacatcaaaggctttccaaccgatgccacactggacgacattaaagagtggctagaggacaaggggcaggttttgaacatccagatgagaaggaccctccacaaggcgttcaaaggcagcatcttcgtcgtcttcgacagtatcgaaagtgcaaaaaagttcgtggagaccccgggccaaaaatacaaagagactgatctgctgatcctgttcaaggatgattatttcgcaaaaaaaaatgagtctggggggtctaagagaactgctgatgggtctgaattcgagccgaagaaaaagaggaaggta

SEQ ID NO: 316 - DNA, 6567 bp, synthetic construct
  source 1..6567: mol_type = other DNA
atgaaaagaactgccgacggcagtgaatttgaacctaaaaagaagagaaaggtctcaggcggatcatccggcgggagcaccctgaacatcgaggatgagtacaggctccacgagacatctaaggagcccgacgtgagtttgggctccacatggctgagtgacttcccgcaagcctgggccgagaccgggggcatgggtctcgccgtgcgacaggcacctctcatcatccccctcaaagccacctctacaccagttagcattaagcagtaccctatgagtcaggaagccaggctgggcattaagccacacatccagaggctgttggaccaagggatcctggttccatgtcagtccccctggaacaccccactgctgccggtgaagaagcccggcacaaatgattaccgcccagttcaggatctccgagaagtgaacaagagggtcgaagatatccatccaacagtgcctaacccgtacaacctgctgtcaggactccccccaagccatcaatggtacaccgtactcgatctgaaggatgcctttttctgtcttcggctccaccctacatcccagccgctgttcgcctttgaatggcgagatccggaaatggggatctcgggccagctgacttggacaagactcccccagggtttcaagaattcacccacccttttcaacgaagctttacatcgcgacctggccgacttccgcatccaacaccccgatctaattctgctgcagtatgtggatgaccttctgcttgctgctaccagtgagcttgactgccagcaaggaacacgggcactcctgcaaacactggggaacctgggttaccgcgcgagtgccaaaaaagcccagatatgccagaagcaagtgaaatacctcgggtacctcctgaaggaaggccagcgttggcttacagaggccaggaaggaaacagttatgggtcagcccactcctaagacacctaggcagctgagagaattcctgggcaaggccggtttttgcagacttttcatccctggctttgccgagatggccgccccgttatatcccctcactaagcctggcaccctgttcaactggggaccagaccagcagaaagcgtatcaggagatcaagcaggcactcctgactgcgccagctctgggcctgcccgacctgacaaagccttttgagctttttgtagacgagaagcagggatacgctaaaggggtcttgactcagaagttgggcccgtggaggcgcccggtggcatacctgagtaaaaagctggaccctgtcgctgccggctggccaccctgcctgcgcatggtggccgcaatcgccgtcctcacgaaagacgctggcaagcttacgatggggcaaccattggttatcctggctccacatgcagttgaagccctcgtcaagcagccccccgatcgttggctgtccaatgcccgtatgacccactaccaggccctactcctggataccgaccgcgtgcagttcggccctgtggtggcactgaacccagcgaccctcctcccactgccggaggagggcttgcagcataactgtctggatatcctagccgaagctcacggaacccgtccagacttaactgaccaacccctgcctgatgctgaccacacgtggtacacggacggctcatccctgctgcaggaaggccagcgcaaagctggggcagccgttaccaccgagactgaagtgatatgggccaaagcactgcctgccggcacaagcgcacagcgggcagaacttatcgcactgacacaggcactgaaaatggccgaaggcaagaaattgaacgtgtacaccgatagccgctatgcattcgctaccgcccacatccacggtgaaatataccggaggaggggctggctgacttccgaagggaaggaaattaaaaataaagacgagatcttggccttgctgaaagcactcttcctgcctaaaagactgtccataatccattgtcccggtcaccagaagggtcacagcgcagaggctcgtggaaacagaatggccgaccaggcggctagaaaagccgccatcaccgagacccctgacacttcaacattgctcatcgagaactctagtccctcaggcggttccagcggcgggagtagcgggtctgaaacacccgggacatcagagtcggccacccctgaaagctcaggggggagctccggagggagcggctcctcaaaactggagaagttcacgaactgctacagtctgagcaaaaccttgaggtttaaagctatcccagtgggaaaaactcaagaaaacatcgacaacaagcggcttctggtcgaggacgagaagagagctgaggattacaaaggcgttaagaagctactggaccgctactacctgtctttcattaatgacgtcctgcacagcatcaagctgaagaacctgaataactacatctcactgtttcgcaagaaaacaagaacggagaaagagaacaaagagcttgaaaatttagaaatcaacctccgtaaggagatcgccaaagctttcaagggcaacgagggatacaaaagcctgtttaagaaagacatcattgaaactatactgcctgaatttctggacgacaaggacgagattgctctggtcaattctttcaacggcttcaccaccgctttcaccggtttttttcgcaacagagagaacatgttctccgaagaagccaaatcgacgtctattgctttcaggtgtatcaatgagaacttaacacgttacatcagcaatatggatatcttcgagaaggtggacgctattttcgataagcacgaggtgcaggagatcaaggagaagattctaaatagcgattacgacgttgaggacttcttcgaaggggagttctttaatttcgtgctgacccaggaaggcatcgacgtgtataacgcaatcattggggggttcgtcacagaaagcggggagaagattaagggattgaacgaatacatcaatctctataatcagaagacaaagcagaagcttcccaagttcaaacccttgtacaagcaggttttgtcagaccgtgaaagcttgagcttttatggcgagggctatacaagtgacgaggaagtcctcgaggtttttcggaataccttgaacaagaattccgaaattttttcatcgatcaagaagcttgaaaaactctttaagaactttgacgagtactcctctgccggcatttttgtgaaaaacgggccagcaatcagcactatcagtaaagacatcttcggagaatggaatgtgattcgcgacaaatggaacgccgagtatgatgatattcacctgaagaagaaggcagtggtgacagaaaagtacgaggacgacaggcggaaatcctttaaaaaaataggctctttctcgctggagcagttgcaagagtatgccgacgctgacctaagcgtggtggaaaagttgaaggagattataattcagaaggtcgacgaaatttataaggtgtacggcagctcggagaaactgttcgacgccgactttgtgttggaaaagtcgctgaaaaagaacgacgctgtggtcgctatcatgaaagacctgctcgattccgtgaagtcgttcgaaaactacatcaaggcctttttcggggaaggtaaagaaactaacagggatgagtccttctacggtgatttcgtgctggcatatgacatcctgctaaaggtggaccacatctatgatgccattcggaactatgttacccagaagccctattctaaggataagttcaagctgtacttccagaatccccagtttatgggtggctgggataaggataaagagaccgactatagagctaccatactacgctacggaagtaagtactatctagctatcatggataagaagtacgccaagtgcctgcagaaaattgataaagatgatgtgaacgggaattatgaaaaaatcaactataaactcttgcccgggcctaacaagatgctccctaaagtcttcttcagcaagaagtggatggcatactacaacccttctgaggatatccagaaaatttataagaatggcacattcaagaagggtgacatgtttaatcttaatgactgtcataagcttattgactttttcaaggattccatctctagataccccaaatggagcaacgcgtatgatttcaacttttcggagaccgagaaatataaagatattgcgggtttttaccgggaagtggaggaacagggctataaggtcagcttcgagtctgctagtaaaaaagaagtggataaactggtcgaggagggcaaactatatatgtttcagatctacaataaggacttcagtgacaaatcccatggcacacctaatctgcatactatgtacttcaaattgctgtttgatgagaacaatcacggccaaattcgcctaagcggcggcgccgagctgtttatgaggagagcctcattgaaaaaagaggagcttgttgtccacccggctaacagccccatcgcaaacaaaaaccctgataacccgaagaaaaccaccaccctgtcctatgacgtgtacaaagacaaaagattcagtgaagaccaatacgagctccacattccaatagcgatcaacaagtgccccaagaacatcttcaaaatcaataccgaagtcagagtgctgctcaagcacgacgataacccctatgtcatcggaatcgaccgaggagagagaaaccttctgtacattgtggtggtggatggaaagggcaatattgtggagcaatacagcctgaacgagatcatcaataacttcaacggaatcagaattaagaccgattaccattctctgctcgataagaaagagaaagagagatttgaagctaggcagaactggacaagcatcgaaaatattaaggaactgaaagccgggtacatttctcaggtagtccacaagatctgcgagctagtggagaaatacgatgccgtgatcgcactcgaggatctgaactccggatttaagaacagtagagtgaaagttgagaagcaggtgtaccagaaatttgagaaaatgctgatcgacaaattaaactacatggtggacaagaagtctaacccatgtgcgacgggtggggctctgaaggggtaccagatcaccaacaagttcgagtcttttaagagcatgtcaacacaaaacggcttcattttttatattccggcttggctgacctccaaaattgaccctagcaccggtttcgtcaacctgttgaaaaccaagtacacatccatcgctgactccaagaagtttatctcaagcttcgacaggattatgtacgtgcctgaggaggacctgttcgagttcgcgttagactacaaaaacttcagccgtactgacgctgattatatcaagaagtggaagctttacagctacgggaacagaatccgcattttcaggaacccaaagaagaacaacgtgttcgactgggaagaggtgtgcctcaccagtgcgtacaaggagctgttcaataagtatgggatcaactatcagcaaggcgacatcagggccctcctgtgtgagcagagtgacaaagctttttattcatcatttatggccctcatgagtctgatgttgcaaatggctaactccattaccgggaggaccgacgtagactttcttatcagcccggtgaagaacagcgacggcatcttctacgactccaggaattacgaggcccaggagaacgccatactgccaaaaaacgctgacgccaacggcgcctacaacatagcccgcaaagtgctgtgggcaatcgggcagttcaagaaagctgaagacgaaaagctggacaaagtgaaaatcgcaataagcaataaagagtggctcgaatatgctcaaacgagcgtcaagcactccgggtctggtggcagtagcggcgggtccatggccgagaatggtgacaacgagaagatggcagccctggaagcaaaaatatgtcaccagattgagtattattttggagactttaacctgccaagagacaaattcctgaaggagcagatcaaattagatgaagggtgggtccccctggaaatcatgatcaagtttaacaggctgaaccggcttactaccgactttaatgtcattgtcgaagcactgtctaagtctaaggcggagctgatggaaattagtgaagacaaaaccaaaatcagaaggagcccgtccaagcctctaccagaagttacagacgagtacaaaaatgatgtgaaaaaccgaagcgtctacatcaagggctttcctaccgatgccactctcgacgacatcaaggaatggttggaggataaaggacaggtcctgaatatccagatgagacgtactctgcataaggccttcaagggttccatcttcgttgtcttcgatagcatcgagtctgcaaaaaagtttgtcgaaacacctggacagaagtataaggaaaccgatctcctcatactctttaaggacgattattttgctaagaaaaacgaaagtggggggagtaaacgtaccgcagatgggtcggagtttgaacccaaaaaaaagaggaaagtg

SEQ ID NO: 317 - DNA, 6567 bp, synthetic construct
  source 1..6567: mol_type = other DNA
atgaagaggactgcggatggaagcgagtttgagcctaagaagaagagaaaagtgtccggcggctcctcgggcgggtctactcttaatatcgaggacgagtaccggttacacgagacgtccaaggagccagatgtcagcctgggctcaacctggctgagcgattttccccaggcttgggcagaaacagggggaatgggtttggcagttagacaggctcctctgattataccactgaaggcgaccagcacgcctgtcagcatcaaacagtaccctatgtcccaagaggccaggcttggaataaaaccacacatccagcgcctcctcgatcaagggatcctggtaccatgccagagcccttggaacacccctttgttgcctgtcaagaaaccgggcaccaacgattataggcccgttcaggaccttagagaagtcaataagcgcgtagaggacatccacccaacggtccccaacccgtacaacctgttatcaggcttgccccctagtcaccaatggtatacagttctcgatttgaaagacgcattcttttgcctccgtctgcatcctacgtctcaaccccttttcgctttcgaatggcgcgacccagagatgggtatctcgggccaacttacatggactcgtcttccccaaggcttcaaaaatagcccgactctcttcaacgaagctctgcatagggacttagctgatttccggatccaacacccggatcttatccttctgcagtatgtagatgatctactcctcgcggctacatcagagctggattgccagcagggtacacgagctctgctccagaccctcggtaacctcggttaccgcgcaagcgcaaagaaggcccagatttgccagaagcaggttaagtacctgggctacctcctgaaggaagggcagcgctggctgaccgaagcgcgtaaagaaactgtgatgggtcaacctacacctaagactccaagacagcttcgtgagtttctgggcaaagcaggattttgtagactgtttattcccggctttgcagagatggctgcacccctttatccgcttacgaaacccggcacactttttaattgggggcccgatcagcaaaaagcttatcaggagattaaacaggctttactgaccgctccagcgttgggactgccagacttaaccaaacctttcgagctcttcgtcgatgagaagcagggttacgccaaaggggttctgacacagaaattgggcccttggcggagaccggttgcttatctgagcaaaaagctggacccagtggcagccggatggccaccttgtctgagaatggttgccgcaattgccgtacttacgaaagacgctggcaagctgactatggggcaaccgctggttattcttgccccgcacgcagtggaagctctcgttaagcagcccccggataggtggttaagcaatgcacgtatgacgcactatcaagccctgctcctcgacaccgacagagtacagtttgggcccgttgtggctctgaacccagcgactctgctccccttgcccgaagaaggcctgcagcacaactgcttggatattctcgccgaagctcacggtacacgtcctgacctcactgaccagccactccccgatgccgaccacacttggtataccgatgggagctcgctgctgcaggaaggacagcgcaaggctggcgcggcggtgaccacggaaaccgaggtcatctgggctaaggccctgccggccggcactagcgcgcaacgggccgagttaattgctctcacacaggcactgaagatggccgaaggcaagaagctgaacgtctacaccgacagccgttatgcctttgcaactgcccacatccacggtgagatctataggcgcagagggtggctcacctctgagggtaaggaaattaaaaataaggatgaaatcttggctctgctgaaggccctgttcttgcccaagcggctctcaatcatccattgtccagggcaccagaagggccattccgcagaggcacggggcaatcgcatggctgaccaagccgccaggaaggccgcaatcaccgaaacaccggatacctcgactctcctgatcgagaatagcagtcctagtgggggctcctcaggaggcagcagcgggtctgaaacacctgggacgagcgaaagtgctacaccggagtcgagtggcgggagctcagggggcagtggctcgtctaagctcgagaaattcaccaactgctactctctgagcaaaacgctgcgcttcaaagcgatcccagtcgggaaaactcaagaaaatatcgacaacaagaggctgctcgtggaggatgagaaaagagcagaggactacaagggcgtgaaaaagctgttagaccgttactacttatctttcataaacgatgttctgcatagtatcaagctgaagaatctgaacaactatatctcactgtttcgtaaaaaaaccagaaccgagaaggagaataaagagctcgaaaatttagagatcaacctgcggaaggagatagctaaagcgtttaaaggaaatgaaggttataaatccctttttaagaaggatataattgagaccattcttcctgagttcttagacgacaaagatgagatagcattggtgaatagctttaatggcttcacgacagctttcaccggctttttcagaaacagggaaaacatgtttagtgaagaggctaaatcgacgtccatcgcatttcgctgcattaatgagaatctcacccggtacatttctaacatggatatcttcgaaaaggtcgatgcaattttcgacaaacatgaggtgcaggagatcaaggagaaaatcctgaattcagattacgatgtagaagacttcttcgagggggaattctttaactttgttctgactcaggagggaatcgacgtgtataatgctatcatcggcgggttcgttaccgaatccggcgagaagatcaaaggtttaaacgaatatataaatctctacaaccaaaagacgaagcagaagctgccaaagtttaagccattgtacaaacaggtcttatctgatcgcgagtctctgagtttttatggagaggggtacacgtcagacgaagaggttcttgaagtgtttcggaacaccctgaataaaaattctgagattttctctagcatcaaaaaacttgagaaactgttcaaaaactttgatgagtattcaagcgctggcatattcgtaaaaaatggcccagctatcagcactatctctaaggacatctttggtgagtggaatgttatccgtgataaatggaatgcagagtatgatgatattcacctgaagaaaaaggccgttgttacggagaaatacgaagatgatagacgcaagagctttaaaaagattggcagtttctctctggagcagttgcaggaatatgcggatgcggatctctctgtggtggaaaaactcaaagagatcatcattcaaaaggtggacgaaatctataaggtgtacgggtcttctgagaaattatttgacgctgatttcgtgcttgagaagagcttaaagaagaacgatgctgtcgtcgccatcatgaaggacctgctcgactcggtcaaatcctttgagaactatataaaggccttctttggagaaggcaaggagaccaatcgcgacgagagcttctacggggacttcgtgttagcttatgatatcttactaaaagttgaccacatttatgacgcaattcgaaactacgtaactcagaagccatatagcaaggataaatttaagctgtattttcaaaacccgcagtttatgggagggtgggataaggacaaggaaacagattatcgagccaccatcttgagatacggctcaaagtattatctggctatcatggataaaaagtacgccaagtgtctgcagaagatagacaaggacgacgtgaacgggaattatgaaaagattaattataaactcttgcccggcccaaacaaaatgctgccgaaggtattcttcagcaagaaatggatggcttattataacccatctgaggacatacaaaagatctacaaaaacggcaccttcaagaagggagatatgtttaatttgaatgattgccacaagctgattgactttttcaaggatagcatttcccggtacccgaaatggtcaaacgcatatgacttcaatttttctgagacagaaaagtataaggatatcgcgggcttctacagagaggtggaggaacaaggatacaaggtgagcttcgagtctgccagcaagaaggaagttgacaagctggtagaggagggcaaactatacatgttccaaatctataacaaggacttttcagataaatcccatgggacaccaaatctgcacacaatgtacttcaaactgctgttcgatgaaaataaccacggtcagatccgtttgtcagggggagccgaactcttcatgcgtagggcatcacttaagaaggaagagctggtcgtgcaccctgctaatagtccgatcgccaataagaatccagacaaccctaaaaaaactaccacattgtcttatgatgtgtacaaagacaaaagattttccgaagaccagtatgaacttcacattccgatcgccataaacaagtgtcccaaaaacatcttcaaaatcaacaccgaggtacgcgtgctgctcaaacacgatgacaatccctacgttatcgggatagacaggggcgaacgcaatcttctgtacatcgtggtcgtagacgggaagggcaatatagttgagcagtattctttgaacgaaataattaacaacttcaatggcatccggattaaaacagactatcactccctgcttgacaagaaggagaaggaacgttttgaggcgcggcaaaactggacatctatcgagaacattaaagaactgaaagccggttatataagccaggtcgtacataagatttgtgaactggtggaaaagtacgacgctgtaatcgccctcgaggatctgaactccggtttcaagaattcaagagtgaaagtggagaaacaggtttatcaaaagttcgagaaaatgctgattgataaactgaactacatggtggataagaaatcaaacccctgcgccactgggggtgcattaaagggataccagattacaaacaagttcgagtctttcaaatcaatgtccactcagaatggatttattttttatattcccgcctggcttacaagcaagatcgatccttcgacgggcttcgtgaacttgctaaaaacaaagtacacaagcattgctgattcaaaaaagtttatctctagctttgacagaatcatgtacgtgccagaagaggatctctttgaatttgcactggactacaaaaattttagcagaactgatgcggattacattaagaaatggaaactctattcctacggcaacaggattaggatattccggaatcccaagaagaacaacgtgttcgattgggaagaagtttgtctgactagcgcctataaggaactgttcaataagtatggcattaattaccagcaaggcgatatcagggctctgctgtgcgagcagtcagacaaagctttctactcaagtttcatggctttgatgtctctgatgctgcagatggcgaatagtatcacaggtcggactgatgtcgactttttgatttcccccgttaaaaactctgacggaatcttctatgattcccgcaactatgaagcccaggaaaacgctatcctgcctaaaaacgccgacgcaaacggcgcatataacatcgctaggaaggtactgtgggcaataggccaattcaagaaggctgaggatgaaaaactagataaggtgaaaatagccatatccaataaagagtggctcgagtacgcccagacgtccgtaaagcactcaggttcaggcggaagctccggaggcagcatggctgaaaacggagataatgaaaaaatggccgcgctagaggcaaagatctgtcaccagatcgagtactactttggagactttaacttgccaagggacaaattcctgaaagaacaaatcaaattggatgaaggctgggttcccctcgagattatgattaagtttaacagactcaatagactaacaacggatttcaatgttattgtggaagcactttcaaagagtaaggccgagctgatggagatctcggaagataaaacaaagatccgccggagcccatcgaagcctctgccagaggtaaccgacgagtacaaaaatgatgtgaagaaccgaagcgtctatattaagggcttcccaactgacgcgacacttgatgatattaaggagtggttggaagacaaaggccaggtgctgaacatccagatgcggcgaacactgcataaggcctttaagggctcaattttcgtggtgtttgactctattgaaagcgcgaagaagtttgtcgagacacctgggcaaaaatataaagagaccgacctgctgatcctatttaaggatgactattttgccaaaaagaatgaatctgggggatccaaaagaacggccgacggttccgagttcgagcctaagaagaaaagaaaggtg

SEQ ID NO: 318 - DNA, 22 bp, synthetic construct
  source 1..22: mol_type = other DNA
aagcgacctgccgccacaaaga

SEQ ID NO: 319 - DNA, 19 bp, synthetic construct
  source 1..19: mol_type = other DNA
ggtggcagcgctctagaac

SEQ ID NO: 320 - DNA, 41 bp, synthetic construct
  source 1..41: mol_type = other DNA
gttctagagcgctgccaccatgaaacggacagccgacggaa

SEQ ID NO: 321 - DNA, 43 bp, synthetic construct
  source 1..43: mol_type = other DNA
tctttgtggcggcaggtcgcttatgcttcactgaggtctgagc

SEQ ID NO: 322 - DNA, 19 bp, synthetic construct
  source 1..19: mol_type = other DNA
aacacacaagcagggagca

SEQ ID NO: 323 - DNA, 20 bp, synthetic construct
  source 1..20: mol_type = other DNA
cgcatagttaagccagtatc

SEQ ID NO: 324 - DNA, 9412 bp, synthetic construct
  source 1..9412: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatctcgagctcggtacctaatacgacacactataaggaaataagagagaaaagaagagtaagaagaaatataagagccaccatgaaacggacagccgacggaagcgagttcgagtcaccaaagaagaagcggaaagtctctggaggatctagcggaggatccaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggaggatctagcggaggatcctctggcagcgagacaccaggaacaagcgagtcagcaacaccagagagcagtggcggcagcagcggcggcagcagctcaaagctggagaaattcaccaactgttatagcctgtctaagaccctgcgcttcaaggcaatcccagtgggcaagacacaagagaacattgacaacaaacggctcctggtggaggatgagaagagggctgaagattacaagggcgttaagaagctgctggataggtactatctgtcattcatcaacgatgtcctccacagtatcaagctgaagaatctgaacaattacatttctctgttccggaagaagacacggaccgagaaggagaacaaagagctggagaatctggagatcaacctgaggaaagaaatagctaaggctttcaaagggaacgagggttacaagtccctgttcaagaaagacattatcgagactattctgcctgagttcctggacgataaagatgagatcgccctcgtcaattccttcaatgggtttaccacagcctttaccggcttcttcgacaatagagagaatatgttctctgaagaggccaaatccactagcatcgcctttcgctgcataaacgagaacctgactaggtacatcagcaatatggacatctttgagaaagtcgatgccatattcgacaaacatgaggtgcaggagattaaggagaagatcctgaactcagattacgatgtcgaagatttcttcgagggagagttcttcaacttcgtgctcacacaagagggcattgatgtgtacaatgcaatcattggagggttcgtgacagagagtggcgagaagataaagggcctgaacgagtatatcaacctctacaaccagaaaaccaagcagaaactgcctaagttcaagccactgtacaaacaagtgctctcagatagggaaagcctgagcttctacggtgaagggtatacatcagatgaagaagtgctcgaagtgttccgcaacaccctcaataagaacagtgaaatcttctcttcaatcaagaagctggagaaactgttcaagaatttcgatgagtactcctctgccggaatctttgtgaagaatggccctgcaatatccactattagcaaagacatctttggcgagtggaacgttatcagggataagtggaatgccgagtacgatgatattcatctcaagaagaaagccgtggttacagagaaatacgaggatgatagacgcaagagctttaagaagattggtagcttctctctcgaacagctgcaggagtacgccgacgctgacctgtcagtcgtggagaaactcaaggagatcataatccagaaggtggatgaaatctacaaagtgtatggaagctctgagaaactcttcgatgcagactttgttctggagaagagtctgaagaagaacgacgcagtggttgctatcatgaaggacctgctggattctgttaagtctttcgagaattacattaaggcattctttggtgaagggaaggagacaaatagggacgagagcttctatggcgactttgttctggcctacgacatcctcctcaaggttgaccacatctatgacgctatacggaattacgttacccagaagccctatagcaaagacaagttcaagctgtatttccagaatccacagtttatgggtgggtgggataaagacaaagaaacagattacagggccactatcctgcggtacggcagcaaatactatctggctatcatggataagaagtacgccaaatgcctccagaagatcgacaaggacgacgtgaacggtaactacgagaagatcaattacaagctcctgccaggacctaacaagatgctgcccaaggtgttcttctccaagaaatggatggcctactataacccaagcgaggacattcagaagatatacaagaatgggacattcaagaagggcgatatgttcaacctcaacgactgccacaagctgattgatttcttcaaggatagcatttctcgctatcccaagtggtctaatgcatacgatttcaacttcagcgagactgagaagtacaaagacatcgctggcttctaccgggaggtggaagagcaaggctataaggtgtcattcgaatccgcttctaagaaggaagtggataagctcgtggaagagggtaagctgtacatgttccagatatacaacaaagacttcagcgataagagccacggcactccaaacctccatactatgtatttcaagctgctgtttgacgagaacaaccacggacagattaggctgtcaggaggcgcagaactcttcatgcgcagagcttcactgaagaaggaggaactcgttgtccacccagccaatagccctatagccaataagaatccagacaatcctaagaaaaccactactctgtcttacgatgtgtataaggataagagattctctgaagatcagtacgaactgcacatacccattgccattaacaagtgccctaagaacatcttcaagattaacacagaggttagagtgctcctgaaacacgacgataacccttatgttataggcattgaccgcggagagagaaacctgctgtacatcgtcgtggtggacggcaaaggcaacatcgtggaacagtacagtctcaatgaaatcattaacaatttcaacggaatccgcattaagaccgactaccattctctcctcgacaagaaggagaaagaaaggttcgaagcaagacagaattggacaagtatagagaatatcaaagaactgaaggctgggtacatctctcaggttgtgcacaagatatgtgagctggtggagaagtacgacgctgttatcgccctcgaggacctgaatagctactgcagctccgtgtatcagaagttcgagaagatgctgatcgacaagctcaactatatggtggacaagaaatccaatccttgcgctactggtggagccctgaagggctatcaaatcaccaataagttcgaatctttcaagtctatgagcacccagaatggcttcatcttctacatacccgcatggctgacatccaagattgatccctctaccggatttgttaatctgctcaagactaagtacacctctattgctgactcaaagaagttcatatcatcatttgaccgcatcatgtacgtgccagaagaggacctgttcgagtttgccctggattacaagaatttctctcggactgacgccgactacatcaagaagtggaagctctactcttatggtaatcggattcgcatattccgcaatcccaagaagaataacgtgttcgattgggaggaagtttgcctcaccagcgcttacaaggagctgttcaataagtatgggattaactaccagcagggcgacataagagccctgctgtgcgaacaatctgataaggcattctattcctctttcatggcactgatgtcactgatgctgcaaatgcgcaattccatcaccggaagaacagacgtggactttctgatctctcctgtcaagaactcagatggcatcttctacgattcccgcaactatgaagcacaggagaatgctatcctgcctaagaatgccgatgcaaatggagcctataacatcgccagaaaggtcctctgggccataggacaattcaagaaagctgaagatgagaagctggacaaggtgaagatcgccatttcaaacaaagagtggctcgaatatgctcagacctcagtgaagcattctggcggctcaaaaagaaccgccgacggcagcgaattcgagcccaagaagaagaggaaagtctaattaattaagctgccttctgcggggcttgccttctggccatgcccttcttctctcccttgcacctgtacctcttggtctttgaataaagcctgagtaggaagcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtctactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 325 - DNA, 16637 bp, synthetic construct
  source 1..16637: mol_type = other DNA
aaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtccccccttttcttttaaaaagtggctaagatctacagctgccttgtaagtcattggtcttaaagtcgacgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacttaacgcgttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcggtccaggattctcttcgacatctccggcttgtttcagcagagagaagtttgttgcgccggatcccttatcgtcatcgtctttgtaatctttcttcttcttagcctgtccagccttctttgtggcggcaggtcgcttatgcttcactgaggtctgagcatattcgagccactctttgtttgaaatggcgatcttcaccttgtccagcttctcatcttcagctttcttgaattgtcctatggcccagaggacctttctggcgatgttataggctccatttgcatcggcattcttaggcaggatagcattctcctgtgcttcatagttgcgggaatcgtagaagatgccatctgagttcttgacaggagagatcagaaagtccacgtctgttcttccggtgatggaattgcgcatttgcagcatcagtgacatcagtgccatgaaagaggaatagaatgccttatcagattgttcgcacagcagggctcttatgtcgccctgctggtagttaatcccatacttattgaacagctccttgtaagcgctggtgaggcaaacttcctcccaatcgaacacgttattcttcttgggattgcggaatatgcgaatccgattaccataagagtagagcttccacttcttgatgtagtcggcgtcagtccgagagaaattcttgtaatccagggcaaactcgaacaggtcctcttctggcacgtacatgatgcggtcaaatgatgatatgaacttctttgagtcagcaatagaggtgtacttagtcttgagcagattaacaaatccggtagagggatcaatcttggatgtcagccatgcgggtatgtagaagatgaagccattctgggtgctcatagacttgaaagattcgaacttattggtgatttgatagcccttcagggctccaccagtagcgcaaggattggatttcttgtccaccatatagttgagcttgtcgatcagcatcttctcgaacttctgatacacggagctgcagtagctattcaggtcctcgagggcgataacagcgtcgtacttctccaccagctcacatatcttgtgcacaacctgagagatgtacccagccttcagttctttgatattctctatacttgtccaattctgtcttgcttcgaacctttctttctccttcttgtcgaggagagaatggtagtcggtcttaatgcggattccgttgaaattgttaatgatttcattgagactgtactgttccacgatgttgcctttgccgtccaccacgacgatgtacagcaggtttctctctccgcggtcaatgcctataacataagggttatcgtcgtgtttcaggagcactctaacctctgtgttaatcttgaagatgttcttagggcacttgttaatggcaatgggtatgtgcagttcgtactgatcttcagagaatctcttatccttatacacatcgtaagacagagtagtggttttcttaggattgtctggattcttattggctatagggctattggctgggtggacaacgagttcctccttcttcagtgaagctctgcgcatgaagagttctgcgcctcctgacagcctaatctgtccgtggttgttctcgtcaaacagcagcttgaaatacatagtatggaggtttggagtgccgtggctcttatcgctgaagtctttgttgtatatctggaacatgtacagcttaccctcttccacgagcttatccacttccttcttagaagcggattcgaatgacaccttatagccttgctcttccacctcccggtagaagccagcgatgtctttgtacttctcagtctcgctgaagttgaaatcgtatgcattagaccacttgggatagcgagaaatgctatccttgaagaaatcaatcagcttgtggcagtcgttgaggttgaacatatcgcccttcttgaatgtcccattcttgtatatcttctgaatgtcctcgcttgggttatagtaggccatccatttcttggagaagaacaccttgggcagcatcttgttaggtcctggcaggagcttgtaattgatcttctcgtagttaccgttcacgtcgtccttgtcgatcttctggaggcatttggcgtacttcttatccatgatagccagatagtatttgctgccgtaccgcaggatagtggccctgtaatctgtttctttgtctttatcccacccacccataaactgtggattctggaaatacagcttgaacttgtctttgctatagggcttctgggtaacgtaattccgtatagcgtcatagatgtggtcaaccttgaggaggatgtcgtaggccagaacaaagtcgccatagaagctctcgtccctatttgtctccttcccttcaccaaagaatgccttaatgtaattctcgaaagacttaacagaatccagcaggtccttcatgatagcaaccactgcgtcgttcttcttcagactcttctccagaacaaagtctgcatcgaagagtttctcagagcttccatacactttgtagatttcatccaccttctggattatgatctccttgagtttctccacgactgacaggtcagcgtcggcgtactcctgcagctgttcgagagagaagctaccaatcttcttaaagctcttgcgtctatcatcctcgtatttctctgtaaccacggctttcttcttgagatgaatatcatcgtactcggcattccacttatccctgataacgttccactcgccaaagatgtctttgctaatagtggatattgcagggccattcttcacaaagattccggcagaggagtactcatcgaaattcttgaacagtttctccagcttcttgattgaagagaagatttcactgttcttattgagggtgttgcggaacacttcgagcacttcttcatctgatgtatacccttcaccgtagaagctcaggctttccctatctgagagcacttgtttgtacagtggcttgaacttaggcagtttctgcttggttttctggttgtagaggttgatatactcgttcaggccctttatcttctcgccactctctgtcacgaaccctccaatgattgcattgtacacatcaatgccctcttgtgtgagcacgaagttgaagaactctccctcgaagaaatcttcgacatcgtaatctgagttcaggatcttctccttaatctcctgcacctcatgtttgtcgaatatggcatcgactttctcaaagatgtccatattgctgatgtacctagtcaggttctcgtttatgcagcgaaaggcgatgctagtggatttggcctcttcagagaacatattctctctattgtcgaagaagccggtaaaggctgtggtaaacccattgaaggaattgacgagggcgatctcatctttatcgtccaggaactcaggcagaatagtctcgataatgtctttcttgaacagggacttgtaaccctcgttccctttgaaagccttagctatttctttcctcaggttgatctccagattctccagctctttgttctccttctcggtccgtgtcttcttccggaacagagaaatgtaattgttcagattcttcagcttgatactgtggaggacatcgttgatgaatgacagatagtacctatccagcagcttcttaacgcccttgtaatcttcagccctcttctcatcctccaccaggagccgtttgttgtcaatgttctcttgtgtcttgcccactgggattgccttgaagcgcagggtcttagacaggctataacagttggtgaatttctccagctttgagctgctgccgccgctgctgccgccactgctctctggtgttgctgactcgcttgttcctggtgtctcgctgccagaggatcctccgctagatcctccagagggtgatgaattttctatgaggagggtagaggtgtctggagtctctgtgatggctgcctttcgggccgcttggtcagccatccggttgcctctagcctcggcgctgtgtcccttttgatgtcctggacaatggattatgctaagtcttttgggcagaaagagggcttttagtagggccaagatctcgtctttatttttgatctctttgccttctgatgtgagccacccacgccttctgtatatttctccatggatatgggcagtagcaaaagcataacggctatcagtataaacatttagcttcttaccttctgccatctttagggcctgggtgagtgctatcagttcagcccgctgagcggatgtcccggctggcagggctttagcccagattacctcggtctcggtggtcaccgcagctcccgccttacgctgtccctcttgtaagagactgcttccatccgtgtaccaggtgtggtcggcgtctgggagcggctggtccgttaggtcgggtcgggttccgtgggcttcggccaggatatcaaggcagttgtgttgcagcccttcctcaggcagtgggagcagcgtagccgggttcagggctaccaccggtccgaactggacccggtccgtgtccaaaagcaaggcctgatagtgagtcatccgggcgttggaaagccagcggtcggggggttgtttgactagtgcctctactgcatggggggccagaatgactagtggctgtcccatggttagcttgcctgcatcctttgtcagtacggcaatggctgctaccatccgtaggcaagggggccacccagctgctactgggtctagctttttggacaggtaggccaccggccgacgccaaggtcccagtttttgcgttaggacacctttggcgtagccctgcttctcgtcgacaaagagttcaaagggcttagtcaaatctggcaaccccagggctggggcagttagaagagcttgcttgatttcttgataggccttttgttggtctgggccccaattaaacagagtccccggtttggtgagagggtacaggggggctgccatttctgcaaacccagggatgaagaggcgacagaagcctgccttccctaggaactcccttagttgtcgaggggtcttcggagtaggctgccccatcacagtctcttttctggcctcagtcagccatctctgaccctcttttagaagataccccagatacttgacctgtttctggcaaatttgggctttcttggccgaggcccgatacccgaggttccctagggtttgtaacagggcccgagtaccttgttggcagtctagctcagaagtggcggccagcagtaagtcatccacgtactgtagcaggatcaagtctgggtgctggatccggaagtctgctaggtctctgtgcagtgcctcattaaacagggtgggactgtttttgaaaccctgtgggagtctggtccaggtcaattgtcctgagattcccatctctggatctctccactcaaaggcgaagagaggctgactggtggggtggagtctcaggcagaaaaaggcatcctttaaatcaagcacagtgtaccactggtgggacggtgggagcccgctcaagaggttgtaagggttgggcacggtggggtggatgtcttccacccgcttgttgacttctctcagatcctggacaggcctataatcattagtccctggtttcttaacgggtagcaggggcgtgttccagggggactggcagggtaccagtattccctggtccaacagtctctgtatgtggggcttgatccccagtctggcttcttgtgacatggggtattgttttatggacacgggggtagaggttgctttcagaggtatgatcagaggagcttggcgaactgccagtcccatgcccccggtttccgcccaggcctgaggaaaatcagacagccatgtggaccctagagaaacatctggctcttttgaggtctcatgtagccgatactcatcttctatatttagggtggatcctccgctagatcctccagagactttccgcttcttctttggtgactcgaactcgcttccgtcggctgtccgtttcatggtggcagcgctctagaaccggtcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagccaattcccactcctttcaagacctagctagcgaattcaaaaaagcaccgactcggtgccactttttcaagttgataacggactagccttattttaacttgctatttctagctctaaaacagagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcggtaccttaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaat

SEQ ID NO: 326 - DNA, 270 bp, synthetic construct
  source 1..270: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctaatttctactaagtgtagat

SEQ ID NO: 327 - DNA, 27 bp, synthetic construct
  source 1..27: mol_type = other DNA
tcgagctcggtacctaatacgactcac

SEQ ID NO: 328 - DNA, 149 bp, synthetic construct; identical to SEQ ID NO: 198
  source 1..149: mol_type = other DNA

SEQ ID NO: 329 - DNA, 20 bp, synthetic construct
  source 1..20: mol_type = other DNA
cctcactcctgctcggtgaa

SEQ ID NO: 330 - DNA, 23 bp, synthetic construct
  source 1..23: mol_type = other DNA
ctaggaatattgaagggggcagg

SEQ ID NO: 331 - DNA, 23 bp, synthetic construct
  source 1..23: mol_type = other DNA
gcggatgttccaatcagtacgca

SEQ ID NO: 332 - RNA, 116 nt, synthetic construct
  source 1..116: mol_type = other RNA
  modified_base 1: mod_base = um
  modified_base 2: mod_base = OTHER; note = 2'-O-methyladenosine
  modified_base 3: mod_base = OTHER; note = 2'-O-methyladenosine
  modified_base 114: mod_base = OTHER; note = 2'-O-methyladenosine
  modified_base 115: mod_base = um
  modified_base 116: mod_base = gm
  misc_feature 1^2: note = phosphorothioate bond
  misc_feature 2^3: note = phosphorothioate bond
  misc_feature 3^4: note = phosphorothioate bond
  misc_feature 113^114: note = phosphorothioate bond
  misc_feature 114^115: note = phosphorothioate bond
  misc_feature 115^116: note = phosphorothioate bond
taatttctactaagtgtagatctgatggtccatgtctgttaagggaaataaaaggaaaagtcactctggggaacacgcccggtgtcacgccacttgacaggccagtaacagacatg

SEQ ID NO: 333 - RNA, 115 nt, synthetic construct
  source 1..115: mol_type = other RNA
  misc_feature 1^2: note = phosphorothioate bond
  misc_feature 2^3: note = phosphorothioate bond
  misc_feature 3^4: note = phosphorothioate bond
  misc_feature 114^115: note = phosphorothioate bond
  misc_feature 113^114: note = phosphorothioate bond
  misc_feature 112^113: note = phosphorothioate bond
  modified_base 1: mod_base = um
  modified_base 2: mod_base = OTHER; note = 2'-O-methyladenosine
  modified_base 3: mod_base = OTHER; note = 2'-O-methyladenosine
  modified_base 113: mod_base = um
  modified_base 114: mod_base = um
  modified_base 115: mod_base = um
taatttctactaagtgtagattgacctcccaaacagctacaacactgtggcccctgtgcccagccctgggctctctgtacatgaagcaactccagtcccaattatgtagctgttt

SEQ ID NO: 334 - RNA, 116 nt, synthetic construct
  source 1..116: mol_type = other RNA
  modified_base 1: mod_base = um
  modified_base 2: mod_base = OTHER; note = 2'-O-methyladenosine
  modified_base 3: mod_base = OTHER; note = 2'-O-methyladenosine
  modified_base 114: mod_base = um
  modified_base 115: mod_base = gm
  modified_base 116: mod_base = gm
  misc_feature 1^2: note = phosphorothioate bond
  misc_feature 2^3: note = phosphorothioate bond
  misc_feature 3^4: note = phosphorothioate bond
  misc_feature 113^114: note = phosphorothioate bond
  misc_feature 114^115: note = phosphorothioate bond
  misc_feature 115^116: note = phosphorothioate bond
taatttctactaagtgtagatgcggatgttccaatcagtacgggattccatgaggtgcgcgaaggccctacttccgctttcaccttggagacggcgactctcagcgtactgattgg

SEQ ID NO: 335 - RNA, 124 nt, synthetic construct
  source 1..124: mol_type = other RNA
  misc_feature 1^2: note = phosphorothioate bond
  misc_feature 2^3: note = phosphorothioate bond
  misc_feature 3^4: note = phosphorothioate bond
  misc_feature 121^122: note = phosphorothioate bond
  misc_feature 122^123: note = phosphorothioate bond
  misc_feature 123^124: note = phosphorothioate bond
  modified_base 1: mod_base = um
  modified_base 2: mod_base = OTHER; note = 2'-O-methyladenosine
  modified_base 3: mod_base = OTHER; note = 2'-O-methyladenosine
  modified_base 122: mod_base = cm
  modified_base 123: mod_base = OTHER; note = 2'-O-methyladenosine
  modified_base 124: mod_base = gm
taatttctactaagtgtagatcctcactcctgctcggtgaaaaacggtccccagagggttctagacccagaggctcaagtgagcagctgaggcaggtgcctgctgaggatttcgcaccgagcag

SEQ ID NO: 336 - RNA, 132 nt, synthetic construct
  source 1..132: mol_type = other RNA
  modified_base 1: mod_base = um
  modified_base 2: mod_base = OTHER; note = 2'-O-methyladenosine
  modified_base 3: mod_base = OTHER; note = 2'-O-methyladenosine
  modified_base 130: mod_base = um
  modified_base 131: mod_base = cm
  modified_base 132: mod_base = cm
  misc_feature 1^2: note = phosphorothioate bond
  misc_feature 2^3: note = phosphorothioate bond
  misc_feature 3^4: note = phosphorothioate bond
  misc_feature 129^130: note = phosphorothioate bond
  misc_feature 130^131: note = phosphorothioate bond
  misc_feature 131^132: note = phosphorothioate bond
taatttctactaagtgtagatctaggaatattgaagggggcaggcactccaggattccaatagatctgtgtgtccctctccccacccgtccctgtccggctctccgccttgatggatccccttcaatattcc

SEQ ID NO: 337 - RNA, 125 nt, synthetic construct
  source 1..125: mol_type = other RNA
  misc_feature 1^2: note = phosphorothioate bond
  misc_feature 2^3: note = phosphorothioate bond
  misc_feature 3^4: note = phosphorothioate bond
  misc_feature 122^123: note = phosphorothioate bond
  misc_feature 123^124: note = phosphorothioate bond
  misc_feature 124^125: note = phosphorothioate bond
  modified_base 1: mod_base = um
  modified_base 2: mod_base = OTHER; note = 2'-O-methyladenosine
  modified_base 3: mod_base = OTHER; note = 2'-O-methyladenosine
  modified_base 123: mod_base = OTHER; note = 2'-O-methyladenosine
  modified_base 124: mod_base = um
  modified_base 125: mod_base = cm
taatttctactaagtgtagatgcggatgttccaatcagtacgcagggattccatgaggtgcgcgaaggccctacttccgctttcaccttggagacggcgactcatactcaactgattggaacatc

SEQ ID NO: 338 - DNA, 22 bp, synthetic construct
  source 1..22: mol_type = other DNA
ccttagcactctgccacttatt

SEQ ID NO: 339 - DNA, 19 bp, synthetic construct
  source 1..19: mol_type = other DNA
agcgaacctcacacaacag

SEQ ID NO: 340 - DNA, 20 bp, synthetic construct
  source 1..20: mol_type = other DNA
ccagtcactccagcctgttg

SEQ ID NO: 341 - DNA, 20 bp, synthetic construct
  source 1..20: mol_type = other DNA
acacgcacacactcactcac

SEQ ID NO: 342 - DNA, 23 bp, synthetic construct
  source 1..23: mol_type = other DNA
agagtcaaggaacacggataaag

SEQ ID NO: 343 - DNA, 20 bp, synthetic construct
  source 1..20: mol_type = other DNA
aagttcgctaatcccggaac

SEQ ID NO: 344 - DNA, 21 bp, synthetic construct
  source 1..21: mol_type = other DNA
gctgtgaggattgagtgagtt

SEQ ID NO: 345 - DNA, 21 bp, synthetic construct
  source 1..21: mol_type = other DNA
gcagcttcctcctcctttatt

SEQ ID NO: 346 - DNA, 17 bp, synthetic construct
  source 1..17: mol_type = other DNA
aatggagcgagcagcgt

SEQ ID NO: 347 - DNA, 22 bp, synthetic construct
  source 1..22: mol_type = other DNA
ggaaactctgtccagagacacg

SEQ ID NO: 348 - DNA, 23 bp, synthetic construct
  source 1..23: mol_type = other DNA
agagtcaaggaacacggataaag

SEQ ID NO: 349 - DNA, 20 bp, synthetic construct
  source 1..20: mol_type = other DNA
aagttcgctaatcccggaac

SEQ ID NO: 350 - DNA, 24 bp, synthetic construct
  source 1..24: mol_type = other DNA
cctgatggtccatgtctgttactg

SEQ ID NO: 351 - DNA, 21 bp, synthetic construct
  source 1..21: mol_type = other DNA
tgtcacgccacttgacaggcc

SEQ ID NO: 352 - DNA, 23 bp, synthetic construct
  source 1..23: mol_type = other DNA
catgaagcaactccagtcccaat

SEQ ID NO: 353 - DNA, 24 bp, synthetic construct
  source 1..24: mol_type = other DNA
ctgacctcccaaacagctacataa

SEQ ID NO: 354 - DNA, 24 bp, synthetic construct
  source 1..24: mol_type = other DNA
cgcggatgttccaatcagtacgct

SEQ ID NO: 355 - DNA, 22 bp, synthetic construct
  source 1..22: mol_type = other DNA
accttggagacggcgactctca

SEQ ID NO: 356 - DNA, 25 bp, synthetic construct
  source 1..25: mol_type = other DNA
cctcactcctgctcggtgcgaaatc

SEQ ID NO: 357 - DNA, 22 bp, synthetic construct
  source 1..22: mol_type = other DNA
caggtgcctgctgaggatttcg

SEQ ID NO: 358 - DNA, 17 bp, synthetic construct
  source 1..17: mol_type = other DNA
tctccgccttgatggat

SEQ ID NO: 359 - DNA, 20 bp, synthetic construct
  source 1..20: mol_type = other DNA
aatattgaaggggatccatc

SEQ ID NO: 360 - DNA, 23 bp, synthetic construct
  source 1..23: mol_type = other DNA
ttggagacggcgactcatactca

SEQ ID NO: 361 - DNA, 22 bp, synthetic construct
  source 1..22: mol_type = other DNA
gatgttccaatcagttgagtat

SEQ ID NO: 362 - DNA, 52 bp, synthetic construct
  source 1..52: mol_type = other DNA
tctggggaacacgcccggtgtcacgccacttgacaggccagtaacagacatg

SEQ ID NO: 363 - DNA, 55 bp, synthetic construct
  source 1..55: mol_type = other DNA
tctggggaacacgcccggtgtcacgccacttgacaggccagtaacagacatggac

SEQ ID NO: 364 - DNA, 63 bp, synthetic construct
  source 1..63: mol_type = other DNA
tctggggaacacgcccggtgtcacgccacttgacaggccagtaacagacatggaccatcagga

SEQ ID NO: 365 - DNA, 75 bp, synthetic construct
  source 1..75: mol_type = other DNA
agggaaataaaaggaaaagtcactctggggaacacgcccggtgtcacgccacttgacaggccagtaacagacatg

SEQ ID NO: 366 - DNA, 78 bp, synthetic construct
  source 1..78: mol_type = other DNA
agggaaataaaaggaaaagtcactctggggaacacgcccggtgtcacgccacttgacaggccagtaacagacatggac

SEQ ID NO: 367 - DNA, 86 bp, synthetic construct
  source 1..86: mol_type = other DNA
agggaaataaaaggaaaagtcactctggggaacacgcccggtgtcacgccacttgacaggccagtaacagacatggaccatcagga

SEQ ID NO: 368 - DNA, 95 bp, synthetic construct
  source 1..95: mol_type = other DNA
tcctcctttattttagctgaagggaaataaaaggaaaagtcactctggggaacacgcccggtgtcacgccacttgacaggccagtaacagacatg

SEQ ID NO: 369 - DNA, 98 bp, synthetic construct
  source 1..98: mol_type = other DNA
tcctcctttattttagctgaagggaaataaaaggaaaagtcactctggggaacacgcccggtgtcacgccacttgacaggccagtaacagacatggac

SEQ ID NO: 370 - DNA, 106 bp, synthetic construct
  source 1..106: mol_type = other DNA
tcctcctttattttagctgaagggaaataaaaggaaaagtcactctggggaacacgcccggtgtcacgccacttgacaggccagtaacagacatggaccatcagga

SEQ ID NO: 371 - DNA, 100 bp, synthetic construct
  source 1..100: mol_type = other DNA
agggaaataaaaggaaaagtcactctggggaacacgcccggtgtcacgccacttgacaggccagtaacagacatgtaaggagtttatatggaaaccctta

SEQ ID NO: 372 - DNA, 75 bp, synthetic construct
  source 1..75: mol_type = other DNA
agggaaataaaaggaaaagtcactctggggaacacgcccggtgtcacgccacttgacaggccagtaaagaatccg

SEQ ID NO: 373 - DNA, 53 bp, synthetic construct
  source 1..53: mol_type = other DNA
ggccctacttccgctttcaccttggagacggcgactctcagcgtactgattgg

SEQ ID NO: 374 - DNA, 56 bp, synthetic construct
  source 1..56: mol_type = other DNA
ggccctacttccgctttcaccttggagacggcgactctcagcgtactgattggaac

SEQ ID NO: 375 - DNA, 64 bp, synthetic construct
  source 1..64: mol_type = other DNA
ggccctacttccgctttcaccttggagacggcgactctcagcgtactgattggaacatccgcga

SEQ ID NO: 376 - DNA, 75 bp, synthetic construct
  source 1..75: mol_type = other DNA
gggattccatgaggtgcgcgaaggccctacttccgctttcaccttggagacggcgactctcagcgtactgattgg

SEQ ID NO: 377 - DNA, 78 bp, synthetic construct
  source 1..78: mol_type = other DNA
gggattccatgaggtgcgcgaaggccctacttccgctttcaccttggagacggcgactctcagcgtactgattggaac

SEQ ID NO: 378 - DNA, 86 bp, synthetic construct
  source 1..86: mol_type = other DNA
gggattccatgaggtgcgcgaaggccctacttccgctttcaccttggagacggcgactctcagcgtactgattggaacatccgcga

SEQ ID NO: 379 - DNA, 95 bp, synthetic construct
  source 1..95: mol_type = other DNA
gcgatccaggtgctgcagaagggattccatgaggtgcgcgaaggccctacttccgctttcaccttggagacggcgactctcagcgtactgattgg

SEQ ID NO: 380 - DNA, 98 bp, synthetic construct
  source 1..98: mol_type = other DNA
gcgatccaggtgctgcagaagggattccatgaggtgcgcgaaggccctacttccgctttcaccttggagacggcgactctcagcgtactgattggaac

SEQ ID NO: 381 - DNA, 106 bp, synthetic construct
  source 1..106: mol_type = other DNA
gcgatccaggtgctgcagaagggattccatgaggtgcgcgaaggccctacttccgctttcaccttggagacggcgactctcagcgtactgattggaacatccgcga

SEQ ID NO: 382 - DNA, 100 bp, synthetic construct
  source 1..100: mol_type = other DNA
gggattccatgaggtgcgcgaaggccctacttccgctttcaccttggagacggcgactctcagcgtactgattggtaaggagtttatatggaaaccctta

SEQ ID NO: 383 - DNA, 75 bp, synthetic construct
  source 1..75: mol_type = other DNA
gggattccatgaggtgcgcgaaggccctacttccgctttcaccttggagacggcgactctcagcgtcagtggtat

SEQ ID NO: 384 - DNA, 53 bp, synthetic construct
  source 1..53: mol_type = other DNA
gccctgggctctctgtacatgaagcaactccagtcccaattatgtagctgttt

SEQ ID NO: 385 - DNA, 56 bp, synthetic construct
  source 1..56: mol_type = other DNA
gccctgggctctctgtacatgaagcaactccagtcccaattatgtagctgtttggg

SEQ ID NO: 386 - DNA, 64 bp, synthetic construct
  source 1..64: mol_type = other DNA
gccctgggctctctgtacatgaagcaactccagtcccaattatgtagctgtttgggaggtcaga

SEQ ID NO: 387 - DNA, 74 bp, synthetic construct
  source 1..74: mol_type = other DNA
acactgtggcccctgtgcccagccctgggctctctgtacatgaagcaactccagtcccaattatgtagctgttt

SEQ ID NO: 388 - DNA, 77 bp, synthetic construct
  source 1..77: mol_type = other DNA
acactgtggcccctgtgcccagccctgggctctctgtacatgaagcaactccagtcccaattatgtagctgtttggg

SEQ ID NO: 389 - DNA, 85 bp, synthetic construct
  source 1..85: mol_type = other DNA
acactgtggcccctgtgcccagccctgggctctctgtacatgaagcaactccagtcccaattatgtagctgtttgggaggtcaga

SEQ ID NO: 390 - DNA, 95 bp, synthetic construct
  source 1..95: mol_type = other DNA
atggcacattgtcagagggacacactgtggcccctgtgcccagccctgggctctctgtacatgaagcaactccagtcccaattatgtagctgttt

SEQ ID NO: 391 - DNA, 98 bp, synthetic construct
  source 1..98: mol_type = other DNA
atggcacattgtcagagggacacactgtggcccctgtgcccagccctgggctctctgtacatgaagcaactccagtcccaattatgtagctgtttggg

SEQ ID NO: 392 - DNA, 106 bp, synthetic construct
  source 1..106: mol_type = other DNA
atggcacattgtcagagggacacactgtggcccctgtgcccagccctgggctctctgtacatgaagcaactccagtcccaattatgtagctgtttgggaggtcaga

SEQ ID NO: 393 - DNA, 99 bp, synthetic construct
  source 1..99: mol_type = other DNA
acactgtggcccctgtgcccagccctgggctctctgtacatgaagcaactccagtcccaattatgtagctgttttaaggagtttatatggaaaccctta

SEQ ID NO: 394 - DNA, 74 bp, synthetic construct
  source 1..74: mol_type = other DNA
acactgtggcccctgtgcccagccctgggctctctgtacatgaagcaactccagtcccaattatgtttgtacgt

SEQ ID NO: 395 - DNA, 266 bp, synthetic construct
  source 1..266: mol_type = other DNA
gccatcagtcgccggtcccaagcccggataaaatgggagggggcgggaaaccgcctaaccatgccgactgatggcagaactaagcacatgaggatcacccatgtgcaggcaggtgcctgctgaggatttcgcaccgagcagcaaggcacatgaggatcacccatgtgcaaattaacactgccatcagtcggcgtggactgtagaacactgccaatgccggtcccaagcccggataaaagtggagggtacagtccacgctttttttt

SEQ ID NO: 396 - DNA, 271 bp, synthetic construct
  source 1..271: mol_type = other DNA
gccatcagtcgccggtcccaagcccggataaaatgggagggggcgggaaaccgcctaaccatgccgactgatggcagaactaagcacatgaggatcacccatgtgctgtccggctctccgccttgatggatccccttcaatattcccaaggcacatgaggatcacccatgtgcaaattaacactgccatcagtcggcgtggactgtagaacactgccaatgccggtcccaagcccggataaaagtggagggtacagtccacgctttttttt

SEQ ID NO: 397 - DNA, 272 bp, synthetic construct
  source 1..272: mol_type = other DNA
gccatcagtcgccggtcccaagcccggataaaatgggagggggcgggaaaccgcctaaccatgccgactgatggcagaactaagcacatgaggatcacccatgtgcaccttggagacggcgactcatactcaactgattggaacatccaaggcacatgaggatcacccatgtgcaaattaacactgccatcagtcggcgtggactgtagaacactgccaatgccggtcccaagcccggataaaagtggagggtacagtccacgctttttttt

SEQ ID NO: 398 - DNA, 93 bp, synthetic construct
  source 1..93: mol_type = other DNA
gcacatgaggatcacccatgtgcaggcaggtgcctgctgaggatttcgcaccgagcagcaaggcacatgaggatcacccatgtgctttttttt

SEQ ID NO: 399 - DNA, 98 bp, synthetic construct
  source 1..98: mol_type = other DNA
gcacatgaggatcacccatgtgctgtccggctctccgccttgatggatccccttcaatattcccaaggcacatgaggatcacccatgtgctttttttt

SEQ ID NO: 400 - DNA, 99 bp, synthetic construct
  source 1..99: mol_type = other DNA
gcacatgaggatcacccatgtgcaccttggagacggcgactcatactcaactgattggaacatccaaggcacatgaggatcacccatgtgctttttttt

SEQ ID NO: 401 - RNA, 153 nt, synthetic construct
  source 1..153: mol_type = other RNA
taatttctactaagtgtagatcctcactcctgctcggtgaaaaacggtccccagagggttctagacccagaggctcaagtgagcagctgaggcaggtgcctgctgaggatttcgcaccgagcagtaatttctactaagtgtagattttttttt

SEQ ID NO: 402 - DNA, 190 bp, synthetic construct
  source 1..190: mol_type = other DNA
taatttctactaagtgtagatcctcactcctgctcggtgaaaaacggtccccagagggttctagacccagaggctcaagtgagcagctgaggcaggtgcctgctgaggatttcgcaccgagcagcgcggttctatctagttacgcgttaaaccaactagaataatttctactaagtgtagattttttttt

SEQ ID NO: 403 - DNA, 161 bp, synthetic construct
  source 1..161: mol_type = other DNA
taatttctactaagtgtagatctaggaatattgaagggggcaggcactccaggattccaatagatctgtgtgtccctctccccacccgtccctgtccggctctccgccttgatggatccccttcaatattcctaatttctactaagtgtagattttttttt

SEQ ID NO: 404 - DNA, 198 bp, synthetic construct
  source 1..198: mol_type = other DNA
taatttctactaagtgtagatctaggaatattgaagggggcaggcactccaggattccaatagatctgtgtgtccctctccccacccgtccctgtccggctctccgccttgatggatccccttcaatattcccgcggttctatctagttacgcgttaaaccaactagaataatttctactaagtgtagattttttttt

SEQ ID NO: 405 - DNA, 154 bp, synthetic construct
  source 1..154: mol_type = other DNA
taatttctactaagtgtagatgcggatgttccaatcagtacgcagggattccatgaggtgcgcgaaggccctacttccgctttcaccttggagacggcgactcatactcaactgattggaacatctaatttctactaagtgtagattttttttt

SEQ ID NO: 406 - DNA, 191 bp, synthetic construct
  source 1..191: mol_type = other DNA
taatttctactaagtgtagatgcggatgttccaatcagtacgcagggattccatgaggtgcgcgaaggccctacttccgctttcaccttggagacggcgactcatactcaactgattggaacatccgcggttctatctagttacgcgttaaaccaactagaataatttctactaagtgtagattttttttt

SEQ ID NO: 407 - DNA, 279 bp, synthetic construct
  source 1..279: mol_type = other DNA
tcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 408 - RNA, 91 nt, synthetic construct
  source 1..91: mol_type = other RNA
taatttctactaagtgtagatcgtcgccgtccagctcgaccagcccatcctggtcgagctggtaatttctactaagtgtagattttttttt

SEQ ID NO: 409 - RNA, 97 nt, synthetic construct
  source 1..97: mol_type = other RNA
taatttctactaagtgtagatcgtcgccgtccagctcgaccagctggtgcccatcctggtcgagctggtaatttctactaagtgtagattttttttt

SEQ ID NO: 410 - RNA, 104 nt, synthetic construct
  source 1..104: mol_type = other RNA
taatttctactaagtgtagatcgtcgccgtccagctcgaccagcaccggggtggtgcccatcctggtcgagctggtaatttctactaagtgtagattttttttt

SEQ ID NO: 411 - RNA, 149 nt, synthetic construct
  source 1..149: mol_type = other RNA
taatttctactaagtgtagatgaggtggtggagaagaagcagttagtgaagctgccaccgagccgatgagctaaacttcatgacgcgactttgctcggattcattcgctgcttcttctcctaatttctactaagtgtagattttttttt

SEQ ID NO: 412 - RNA, 150 nt, synthetic construct
  source 1..150: mol_type = other RNA
taatttctactaagtgtagatgaggtggtggagaagaagcagttagtgaagctgccaccgagccgatgagctaaacttcatgacgcgactttgctcggattcattcgctgcttcttctccataatttctactaagtgtagattttttttt

SEQ ID NO: 413 - RNA, 151 nt, synthetic construct
  source 1..151: mol_type = other RNA
taatttctactaagtgtagatgaggtggtggagaagaagcagttagtgaagctgccaccgagccgatgagctaaacttcatgacgcgactttgctcggattcattcgctgcttcttctccactaatttctactaagtgtagattttttttt

SEQ ID NO: 414 - RNA, 127 nt, synthetic construct
  source 1..127: mol_type = other RNA
taatttctactaagtgtagatgaggtggtggagaagaagcagtttgagctaaacttcatgacgcgactttgctcggattcattcgctgcttcttctcctaatttctactaagtgtagattttttttt

SEQ ID NO: 415 - RNA, 128 nt, synthetic construct
  source 1..128: mol_type = other RNA
taatttctactaagtgtagatgaggtggtggagaagaagcagtttgagctaaacttcatgacgcgactttgctcggattcattcgctgcttcttctccataatttctactaagtgtagattttttttt